(19) United States
(12) Patent Application Publication
(10) Pub. No.: US 2022/0380463 A1
(43) Pub. Date: Dec. 1, 2022

(54) ENGINEERED PH-DEPENDENT ANTI-CD3 ANTIBODIES, AND METHODS FOR THEIR GENERATION AND USE

(71) Applicant: ADIMAB, LLC, Lebanon, NH (US)

(72) Inventors: James GEOGHEGAN, Lebanon, NH (US); Bianka PRINZ, Lebanon, NH (US); Robert PEJCHAL, Lebanon, NH (US)

(21) Appl. No.: 17/617,019

(22) PCT Filed: Jun. 8, 2020
(86) PCT No.: PCT/US2020/036657

§ 371 Date: Dec. 7, 2021

Related U.S. Application Data
(60) Provisional application No. 62/858,968, filed on Jun. 7, 2019.

(51) Int. Cl. C07K 16/28

(52) U.S. Cl. CPC C07K 16/2809; C07K 2317/14; C07K 2317/24; C07K 2317/33; C07K 2317/55; C07K 2317/565; C07K 2317/92

(57) ABSTRACT

Engineered pH-dependent anti-CD3 binding domains and antibodies and/or antigen-binding domains comprising same, including multispecific antibodies, with, inter alia, desirable T-cell activation and (re)directed target cell killing potency and developability, profiles are provided, as well as methods for their identification, isolation, and generation, and methods for their preparation and use.

### RELATED APPLICATIONS

This application is a U.S. Nat'l Phase Appl. submitted under 35 U.S.C. 371 based on Int'l Appl. No. PCT/US2020/036657, filed Jun. 8, 2020, which claims ppage riority to U.S. Provisional Appl. No. 62/858,968, filed Jun. 7, 2019, all of which are hereby incorporated by reference in their entirety.

### SEQUENCE LISTING

The instant application contains a Sequence Listing which has been submitted in ASCII format via EFS-Web and is hereby incorporated by reference in its entirety. Said ASCII copy, created on Oct. 15, 2021, is named “11604300002001.txt” and is 631,273 bytes in size.

### FIELD OF THE INVENTION

The invention relates, inter alia, to engineered pH-dependent anti-Cluster of Differentiation 3 (CD3) antibodies, including multispecific antibodies, and functional fragments thereof, and methods and reagents for their identification, isolation, preparation, and use.

### BACKGROUND OF THE INVENTION

Cell proliferative disorders, such as cancer, are characterized by the uncontrolled growth of cell subpopulations. They are the leading cause of death in the developed world and the second leading cause of death in developing countries, with a total number of new cancer cases per year expected to rise to 23.6 million by 2030. The National Cancer Institute estimates that almost 2 million new cases of cancer will be diagnosed in the U.S. and greater than 600,000 Americans will die of cancer in 2018. Cancer care thus represents a significant and ever-increasing societal burden.

The idea of using the cytotoxic capacity of T cells to kill tumor cells through use of CD3 targeting bispecific antibodies dates back to the mid-1980s. (Staerz et al. Nature 1985 314: 628-32). Many bispecific antibodies developed to date contain a first binding site specific to CD3 for T-cell recruitment and activation, and a second binding site for a targeted disease-associated antigen, such as an antigen produced by a tumor cell. CD3 bispecific antibodies trigger the CD3 surface receptor on T cells by binding to their second target protein expressed on tumors such that available T cells can bind to target-expressing cells via bridging by the CD3 bispecific antibody, irrespective of the peptide/MHC specificity of their T-cell receptor. (See, e.g., Bassan, 2012, Blood 120:5094-95). Bridging of T cells and tumor cells using CD3 bispecific antibodies can induce dramatic regression of advanced-stage malignancies and, in some cases, lead to complete remission. Currently, more than 25 different CD3 bispecific antibodies are in clinical development for treatment of hematologic malignancies or solid cancers by targeting CD19, CD20, CD33, and CD123, or EpCAM, HER2, PSMA, and CEA, respectively. (See, e.g., Liu et al. Front Immunol 2017 8:38).

While bispecific antibodies have shown considerable benefits over monospecific antibodies for the treatment and the detection of cancer, broad commercial application of bispecific antibodies has been hampered by the lack of efficient/low-cost production methods, the lack of stability of bispecific polypeptides and the lack of long half-lives in humans. A large variety of methods have been developed over the last few decades to produce bispecific monoclonal antibodies. However, many candidate bispecific antibodies with exquisite selectivity and high potency toward the target of interest often have problems in downstream development and clinical efficacy activities, including polyspecific binding (or “polyspecificity”); off-target binding; nonspecific binding; poor expression levels or profiles in eukaryotic host cells, such as mammalian host cells and yeast cells; poor chemical and physical properties, such as poor stability during storage (e.g., poor/low “shelf-life” stability), poor (low) solubility, poor (high) viscosity, propensity to aggregate, and the like; and poor clinical and biophysical profiles, such as poor pharmacokinetic profiles, poor pharmacodynamic profiles, fast or poor in vivo clearance rates, short circulation half-life, some of which result in termination of their development.

Certain techniques and assays exist to assess many of the aforementioned developability characteristics for discovered antibodies in the context of downstream development activities (“post-discovery antibodies”), such as CIC, SIC, BVP-ELISA, TMA, and other assays; however, such assays are typically not amenable to high-throughput formats in early antibody discovery platforms. Furthermore, assessment of these attributes typically requires milligram to gram quantities of protein, thus often imposing a de facto limitation on the number of leads that can be pragmatically considered for development, and consequently reducing the likelihood of program success. Consequently, significant resources are often expended attempting to fix poorly behaving lead candidates with few backups available in later stages of development.

A variety of anti-CD3 antibodies are known in the art, including monoclonal and bispecific antibody formats. See, e.g., U.S. Pat. Nos. 7,262,276; 7,635,472; 7,862,813; 9,587,021; and 10,174,124. However, many of these anti-CD3 antibodies possess developability issues, such as those outlined above, and/or elicit production of cytokines, often leading to toxic cytokine release syndrome (CRS). Because the anti-CD3 binding domain of the bispecific antibody engages all T cells, the high cytokine-producing CD4 T cell subset is recruited. Accordingly, there is an unmet need for the provision for anti-CD3 antibodies that display desirable developability and/or CRS risk profiles and are safe and efficacious in, for example, binding specifically to CD3 expressed on T-cells, activating T-cells and (re)-directing the activated T-cells to kill target cells, and doing this with diminished risk of eliciting CRS.

One approach to developing CD3 binding domains that display desirable CRS risk profiles, is to engineer CD3 binding domains with pH-dependent antigen binding. Incorporation of histidines and/or other ionizable residues into the binding interfaces of antibodies and other proteins has previously been used to engineer pH-dependent antigen-binding (see, e.g., Igawa et al., Nature Biotechnology 28:1203-1207 (2010)). Protonation of histidine side chains in binding interfaces may alter electrostatic interactions and/or induce conformational changes that lead to pH-dependent differences in binding affinity (Gera et al., PLOS ONE 7(11) e48928. doi:10.1371/2012). Recognizing that the pH range of human blood is about 7.6-7.8, whereas tumor cells have an extracellular pH of about 6.3-6.5 due at least in part to accumulation of metabolic acids that are inefficiently cleared because of poor tumor vascularization, Applicant's engineered pH-dependent CD3 binding domains with preferential CD3 binding at low(er) pH values promote binding and activity in and around the tumor microenvironment. Without being bound by theory, it is believed that CD3 binding domains engineered to preferably bind to CD3 at a lower pH, e.g., pH˜ 6, may result in selective and sustained cytotoxic activity at or around the tumor site, thereby potentially reducing or eliminating off-target effects as well as improving half-life and dosing.

### SUMMARY OF THE INVENTION

The present disclosure relates to engineered pH-dependent anti-CD3 antibodies and antigen-binding fragments thereof, which antibodies and antigen-binding fragments optionally bind to CD3 and/or CD3-expressing cells with greater binding affinities at pH 6.0 than at physiological pH (pH 7.4) and methods of using the same.

In certain embodiments, the disclosure provides an antibody comprising a CD3 binding domain selected from the group consisting of ADI-48576, ADI-48577, ADI-48587, ADI-48592, ADI-48595, ADI-48635, ADI-48643, ADI-48645, ADI-48650, ADI-48652, and ADI-48666.

In certain embodiments, the disclosure provides an antibody comprising a CD3 binding domain selected from the group consisting of ADI-48592, ADI-48595, ADI-48650, ADI-48652, ADI-48662, and ADI-48666.

In certain embodiments, the disclosure provides an antibody comprising a CD3 binding domain selected from the group consisting of ADI-48588, ADI-48587, ADI-48577, ADI-48590, ADI-48581, ADI-48575, ADI-48593, ADI-48591, ADI-48647, ADI-48636, ADI-48586, ADI-48646, ADI-48638, ADI-48597, ADI-48601, ADI-48576, ADI-48643, ADI-48624, ADI-48632, ADI-48635, and ADI-48645.

Analysis of 258 unique clones identified using methodology described herein revealed consensus motifs within a CDRH3 region. In some embodiments, the disclosure provides an antibody comprising a CD3 binding domain, CDRH3, wherein the CDRH3 binding domain comprises a consensus motif, the consensus motif comprising the sequence AX₁DX₂YX₃HX₄FYDV, wherein X₁ is R or H, wherein X₂ is A or H, wherein X₃ is G, H, or P, wherein X₄ is Y, H, D, V, E, S, N, L, M, I, G, A, Q, or T, and wherein, optionally, at least one of X₁, X₂, X₃, and X₄ is substituted with H (SEQ ID NO: 1).

In some embodiments, the disclosure provides an antibody comprising a CD3 binding domain, CDRH3, wherein the CDRH3 binding domain has a consensus motif represented by the sequence ARDX₁YGX₂X₃X₄YDX₅ wherein X₁ is A or H, wherein X₂ is R or H, wherein X₃ is H or Y, wherein X₄ is F or H, wherein X₅ is H or V, and wherein, optionally, at least one of X₁, X₂, X₃, X₄, and X₅ is substituted with H (SEQ ID NO: 2).

In some embodiments, the disclosure provides an antibody comprising a CD3 binding domain, CDRH3, wherein the CDRH3 binding domain comprises a consensus motif, the consensus motif comprising the sequence ARDAHX₁X₂YX₃X₄DX₅, wherein X₁ is G, E, or R, wherein X₂ is R or H, wherein X₃ is F or H, wherein X₄ is Y or H, wherein X₅ is V or H, and wherein, optionally, at least one of X₂, X₃, X₄, and X₅ is substituted with H (SEQ ID NO: 3).

In some embodiments, the disclosure provides an antibody comprising a CD3 binding domain, CDRH3, wherein the CDRH3 binding domain comprises a consensus motif, the consensus motif comprising the sequence ARDAX₁HRX₂FYDV, wherein X₁ is H, Y, S, G, A, T, V, or R, wherein X₂ is Y or H, and wherein, optionally, at least one of X₁ and X₂ is substituted with H (SEQ ID NO: 4).

In some embodiments, the disclosure provides an antibody comprising a CD3 binding domain, CDRH3, wherein the CDRH3 binding domain comprises a consensus motif, the consensus motif comprising the sequence ARDX₁YHRYFYDX₂, wherein X₁ is H or A, wherein X₂ is H, V, or M, and wherein, optionally, at least one of X₁ and X₂ is substituted with H (SEQ ID NO: 5).

In some embodiments, the disclosure provides an antibody comprising a CD3 binding domain, CDRH3, wherein the CDRH3 binding domain comprises a consensus motif, the consensus motif comprising the sequence AX₁DAYX₂X₃X₄HX₅DV, wherein X₁ is R or H, wherein X₂ is G or H, wherein X₃ is H or R, wherein X₄ is N, F, or Y, wherein X₅ is Y or H, and wherein, optionally, at least one of X₁, X₂, X₃, and X₅ is substituted with H (SEQ ID NO: 6).

In some embodiments, the disclosure provides an antibody comprising a CD3 binding domain, CDRH3, wherein the CDRH3 binding domain comprises a consensus motif, the consensus motif comprising the sequence ARDX₁X₂GRYFYDV, wherein X₁ is M, Q, or H, wherein X₂ is R or H, and wherein, optionally, at least one of X₁ and X₂ is substituted with H (SEQ ID NO: 7).

In some embodiments, the disclosure provides an antibody comprising a CD3 binding domain, CDRH3, wherein the CDRH3 binding domain comprises a consensus motif, the consensus motif comprising the sequence ARDX₁X₂X₃RYFYDX₄, wherein X₁ is H or A, wherein X₂ is T, Y, or H, wherein X₃ is G or H, wherein X₄ is V or H, and wherein, optionally, at least one of X₁, X₂, X₃, and X₄ is substituted with H (SEQ ID NO: 8).

In some embodiments, the disclosure provides an antibody comprising a CD3 binding domain, CDRH3, wherein the CDRH3 binding domain comprises a consensus motif, the consensus motif comprising the sequence AX₁DX₂X₃X₄X₅X₆X₇X₈DX₉, wherein X₁ is R or H, wherein X₂ is A, H, M, or Q, wherein X₃ is Y, H, S, G, A, T, V, or R; wherein X₄ is G, H, P, E, or R; wherein X₅ is H or R, wherein X₆ is Y, N, F, H, D, E, S, L, M, I, G, A, Q, or T; wherein X₇ is F or H; wherein X₈ is Y or H; wherein X₉ is V, H, or M; and, optionally, wherein at least one of X₁, X₂, X₃, X₄, X₅, X₆, X₇, X₈, and X₉ is H (SEQ ID NO: 58).

In some embodiments, the disclosure provides an antibody comprising a CD3 binding domain, CDRH3, wherein the CDRH3 binding domain comprises a consensus motif, the consensus motif comprising the sequence ARDAX₁X₂X₃X₄FYDX₅, wherein X₁ is T, H, or Y, wherein X₂ is G or H, wherein X₃ is H or R, wherein X₄ is V or Y, wherein X₅ is V or H, and wherein, optionally, at least one of X₁, X₂, X₃, and X₅ is substituted with H (SEQ ID NO: 593).

In some embodiments, the disclosure provides an antibody comprising a CD3 binding domain, CDRH3, wherein the CDRH3 binding domain comprises a consensus motif, the consensus motif comprising the sequence AX₁DX₂X₃X₄X₅X₆X₇YDX₈, wherein X₁ is R or H, wherein X₂ is H or A, wherein X₃ is H or Y, wherein X₄ is H, G, or P, wherein X₅ is R or H, wherein X₆ is Y, I, or V, wherein X₇ is F or H, wherein X₈ is V or H, and wherein, optionally, at least one of X₁, X₂, X₃, X₄, X₅, X₇, and X₈ is substituted with H (SEQ ID NO: 596).

In some embodiments, the disclosure provides an antibody comprising a CD3 binding domain, CDRH2, wherein the CDRH2 binding domain comprises a consensus motif, the consensus motif comprising the sequence WIDLENANTIYDAKFQG (SEQ ID NO: 9).

In some embodiments, the disclosure provides an antibody comprising a CD3 binding domain, CDRH2, wherein the CDRH2 binding domain comprises a consensus motif, the consensus motif comprising the sequence X₁INPX₂TGX₃TX₄YSQKFQG, wherein X₁ is W or Y, wherein X₂ is A, S, D, G, N, L, V, H, or Q, wherein X₃ is A, T, or S, and wherein X₄ is K, V, T, D, Y, F, or A (SEQ ID NO: 10).

In some embodiments, the disclosure provides an antibody comprising a CD3 binding domain, CDRH2, wherein the CDRH2 binding domain comprises a consensus motif, the consensus motif comprising the sequence X₁IX₂AGTGX₃TX₄YSQKFQG, wherein X₁ is W, Y, or F, wherein X₂ is T, N, or D, wherein X₃ is A, T, or L, and wherein X₄ is A, K, V, H, T, or N (SEQ ID NO: 11).

In some embodiments, the disclosure provides an antibody comprising a CD3 binding domain, CDRH2, wherein the CDRH2 binding domain comprises a consensus motif, the consensus motif comprising the sequence X₁IDAGTGX₂TX₃YSQKFQG, wherein X₁ is S or W, wherein X₂ is L, N, D, or F, and wherein X₃ is D, Y, or K (SEQ ID NO: 12).

In some embodiments, the disclosure provides an antibody comprising a CD3 binding domain, CDRH2, wherein the CDRH2 binding domain comprises a consensus motif, the consensus motif comprising the sequence X₁IX₂AGTGATX₃YSQKFQG, wherein X₁ is G, D, or S, wherein X₂ is I or D, and wherein X₃ is K or D (SEQ ID NO: 13).

In some embodiments, the disclosure provides an antibody comprising a CD3 binding domain, wherein the CDRH2 binding domain comprises a consensus motif, the consensus motif comprising the sequence WINPX₁TGNTX₂YSQKFQG, wherein X₁ is D, T, L, S, or A, and wherein X₂ is D, V, L, or N (SEQ ID NO: 14).

In some embodiments, the disclosure provides an antibody comprising a CD3 binding domain, CDRH2, wherein the CDRH2 binding domain comprises a consensus motif, the consensus motif comprising the sequence X₁INAGTGX₂TX₃YSQKFQG, wherein X₁ is Y or W, wherein X₂ is N, D, or A, and wherein X₃ is I or V (SEQ ID NO: 15).

In some embodiments, the disclosure provides an antibody comprising a CD3 binding domain, CDRH2, wherein the CDRH2 binding domain comprises a consensus motif, the consensus motif comprising the sequence X₁INPX₂TGX₃TKYSQKFQG, wherein X₁ is W or Y, wherein X₂ is D, I or Y, and wherein X₃ is D, Y, or E (SEQ ID NO: 16).

In some embodiments, the disclosure provides an antibody comprising a CD3 binding domain, CDRH2, wherein the CDRH2 binding domain comprises a consensus motif, the consensus motif comprising the sequence SIX₁AGTGX₂TKYSQKFQG, wherein X₁ is N or V, and wherein X₂ is A or I (SEQ ID NO: 17).

In some embodiments, the disclosure provides an antibody comprising a CD3 binding domain, CDRH2, wherein the CDRH2 binding domain comprises a consensus motif, the consensus motif comprising the sequence SINAGTGX₁TX₂YSQKFQG, wherein X₁ is F or N, and wherein X₂ is Y or D (SEQ ID NO: 18).

In some embodiments, the disclosure provides an antibody comprising a CD3 binding domain, CDRH2, wherein the CDRH2 binding domain comprises a consensus motif, the consensus motif comprising the sequence X₁IX₂X₃GTGX₄TDYSQKFQG, wherein X₁ is D or W, wherein X₂ is N or H, wherein X₃ is A or S, and wherein X₄ is A or N (SEQ ID NO: 19).

In some embodiments, the disclosure provides an antibody comprising a CD3 binding domain, CDRH2, wherein the CDRH2 binding domain comprises a consensus motif, the consensus motif comprising the sequence: WIDPX₁TGATX₂YSQKFQG, wherein X₁ is N, H, or Y, and wherein X₂ is V or K (SEQ ID NO: 20).

In some embodiments, the disclosure provides an antibody comprising a CD3 binding domain, CDRH2, wherein the CDRH2 binding domain comprises a consensus motif, the consensus motif comprising the sequence WIX₁PX₂TGNTKYSQKFQG, wherein X₁ is D or N, and wherein X₂ is L, I, or V (SEQ ID NO: 21).

In some embodiments, the disclosure provides an antibody comprising a CD3 binding domain, CDRH2, wherein the CDRH2 binding domain comprises a consensus motif, the consensus motif comprising the sequence SINAGDANTKYSQKFQG (SEQ ID NO: 22).

In some embodiments, the disclosure provides an antibody comprising a CD3 binding domain, CDRH2, wherein the CDRH2 binding domain comprises a consensus motif, the consensus motif comprising the sequence X₁IDPX₂TGATX₃YSQKFQG, wherein X₁ is D or W, wherein X₂ is D or V, and wherein X₃ is E or D (SEQ ID NO: 23).

In some embodiments, the disclosure provides an antibody comprising a CD3 binding domain, CDRH2, wherein the CDRH2 binding domain comprises a consensus motif, the consensus motif comprising the sequence WINAGDAATVYSQKFQG (SEQ ID NO: 24).

In some embodiments, the disclosure provides an antibody comprising a CD3 binding domain, CDRH2, wherein the CDRH2 binding domain comprises a consensus motif, the consensus motif comprising the sequence X₁IX₂X₃X₄X₅X₆X₇TX₈YSQKFQG, wherein X₁ is W, S, Y, F, G, or D, wherein X₂ is N, T, D, V, or H, wherein X₃ is A, P, or S, wherein X₄ is G, A, S, N, D, L, V, H, Q, T, I, or Y, wherein X₅ is D or T, wherein X₆ is A or G, wherein X₇ is A, N, T, S, L, D, F, Y, or E, wherein X₈ is V, K, T, D, Y, F, A, H, N, L, I, or E, and, optionally, wherein at least one of X₁, X₂, X₃, X₄, X₅, X₆, X₇, and X₈ is H (SEQ ID NO: 59).

In some embodiments, the disclosure provides an antibody comprising a CD3 binding domain, CDRH2, wherein the CDRH2 binding domain comprises a consensus motif, the consensus motif comprising the sequence WIDAGTGX₁TX₂YSQKFQG, wherein X₁ is L, F, N, or A and wherein X₂ is T or K (SEQ ID NO: 595).

In some embodiments, the disclosure provides an antibody comprising a CD3 binding domain, CDRH1, wherein the CDRH1 binding domain comprises FNIKDYHMH (SEQ ID NO: 25), SNIKDYYMH (SEQ ID NO: 26), or SNIKDYHMH (SEQ ID NO: 27).

In some embodiments, the disclosure provides an antibody comprising a CD3 binding domain, CDRH1, wherein the CDRH1 binding domain comprises a consensus motif, the consensus motif comprising the sequence YTFX₁X₂X₃X₄MH, wherein X₁ is A, K, D, Q, E, N, T, L, Y, S, P, G, H or V, wherein X₂ is T, S, or A, wherein X₃ is Y or I, and wherein X₄ is A, D, N, S, Y, T, I, V, L, E, P, R, or G (SEQ ID NO: 28).

In some embodiments, the disclosure provides an antibody comprising a CD3 binding domain, CDRH1, wherein the CDRH1 binding domain comprises a consensus motif, the consensus motif comprising the sequence YTFX₁X₂X₃X₄MH, wherein X₁ is T, D, A, N, or V, wherein X₂ is D, E, G, or Q, wherein X₃ is Y or D, and wherein X₄ is D, A, E, N, S, Y, or V (SEQ ID NO: 29).

In some embodiments, the disclosure provides an antibody comprising a CD3 binding domain, CDRH1, wherein the CDRH1 binding domain comprises a consensus motif, the consensus motif comprising the sequence YTFTSX₁X₂MH, wherein X₁ is A, D, or T, and wherein X₂ is D, F, A, M, V, or Y (SEQ ID NO: 30).

In some embodiments, the disclosure provides an antibody comprising a CD3 binding domain, CDRH1, wherein the CDRH1 binding domain comprises a consensus motif, the consensus motif comprising the sequence YTFX₁X₂YX₃MH, wherein X₁ is N or T, X₂ is Q or N, and X₃ is S, T, or A (SEQ ID NO: 31).

In some embodiments, the disclosure provides an antibody comprising a CD3 binding domain, CDRH1, wherein the CDRH1 binding domain comprises a consensus motif, the consensus motif comprising the sequence YTFX₁X₂YVMH, wherein X₁ is I or N, and wherein X₂ is K or R (SEQ ID NO: 32).

In some embodiments, the disclosure provides an antibody comprising a CD3 binding domain, CDRH1, wherein the CDRH1 binding domain comprises a consensus motif, the consensus motif comprising the sequence FNIKDYYMH (SEQ ID NO: 47).

In some embodiments, the disclosure provides an antibody comprising a CD3 binding domain, CDRH1, wherein the CDRH1 binding domain comprises a consensus motif, the consensus motif comprising the sequence YTFX₁X₂YX₃MH, wherein X₁ is E, S, or T, wherein X₂ is S or D, and wherein X₃ is A or D (SEQ ID NO: 31).

In some embodiments, the disclosure provides an antibody comprising a CD3 binding domain, CDRL3, wherein the CDRL3 binding domain comprises a consensus motif, the consensus motif comprising the sequence X₁X₂SX₃X₄X₅RX₆, wherein X₁ is H, K, or G, wherein X₂ is Q or H, wherein X₃ is Y or H, wherein X₄ is S, H, D, T, V, M, or L, wherein X₅ is R or H, wherein X₆ is T or H, and wherein, optionally, at least one of X₁, X₂, X₃, X₄, X₅, and X₆ is substituted with H (SEQ ID NO: 33).

In some embodiments, the disclosure provides an antibody comprising a CD3 binding domain, CDRL3, wherein the CDRL3 binding domain comprises a consensus motif, the consensus motif comprising the sequence KQSYX₁X₂RT, wherein X₁ is H, V, K, W, R, L, G, Y, or Q, wherein X₂ is H, L, E, W, G, M, P, T, Q, or V, and wherein, optionally, at least one of X₁ and X₂ is substituted with H (SEQ ID NO: 34).

In some embodiments, the disclosure provides an antibody comprising a CD3 binding domain, CDRL3, wherein the CDRL3 binding domain comprises a consensus motif, the consensus motif comprising the sequence X₁QSX₂HX₃RT, wherein X₁ is K or H, wherein X₂ is H, Y, M, S, L, E, G, or W, wherein X₃ is R or K, and wherein, optionally, at least one of X₁ and X₂ is substituted with H (SEQ ID NO: 35).

In some embodiments, the disclosure provides an antibody comprising a CD3 binding domain, CDRL3, wherein the CDRL3 binding domain comprises a consensus motif, the consensus motif comprising the sequence KQSX₁X₂X₃RT, wherein X₁ is Y or H, X₂ is T, S, V, or K, X₃ is R or H, and wherein, optionally, at least one of X₁ and X₃ is substituted with H (SEQ ID NO: 36).

In some embodiments, the disclosure provides an antibody comprising a CD3 binding domain, CDRL3, wherein the CDRL3 binding domain comprises a consensus motif, the consensus motif comprising the sequence KQSX₁X₂X₃RT, wherein X₁ is H or Y, wherein X₂ is T, S, or Q, wherein X₃ is R or H, and wherein, optionally, at least one of X₁ and X₃ is substituted with H (SEQ ID NO: 36).

In some embodiments, the disclosure provides an antibody comprising a CD3 binding domain, CDRL3, wherein the CDRL3 binding domain comprises a consensus motif, the consensus motif comprising the sequence X₁QSX₂X₃X₄RT, wherein X₁ is K or H, wherein X₂ is Y or H, wherein X₃ is S, H, L, V, or K, wherein X₄ is H, R, or E, and wherein, optionally, at least one of X₁, X₂, X₃, and X₄ is substituted with H (SEQ ID NO: 598).

In some embodiments, the disclosure provides an antibody comprising a CD3 binding domain, CDRL2, wherein the CDRL2 binding domain comprises a consensus motif, the consensus motif comprising the sequence: WASTRES (SEQ ID NO: 37).

In some embodiments, the disclosure provides an antibody comprising a CD3 binding domain, CDRL1, wherein the CDRL1 binding domain comprises a consensus motif, the consensus motif comprising the sequence KSSQSLLX₁X₂X₃X₄GX₅NX₆LA, wherein X₁ is N or H, wherein X₂ is A, R, or T, wherein X₃ is R or H, wherein X₄ is T, P, or E, wherein X₅ is H or K, wherein X₆ is H or Y, and wherein, optionally, at least one of X₁, X₃, X₅, and X₆ is substituted with H (SEQ ID NO: 38).

In some embodiments, the disclosure provides an antibody comprising a CD3 binding domain, CDRL1, wherein the CDRL1 binding domain comprises a consensus motif, the consensus motif comprising the sequence KSSQSLLX₁AX₂THX₃NX₄LA, wherein X₁ is N or H, wherein X₂ is R or H, wherein X₃ is K or H, wherein X₄ is Y or H, and wherein, optionally, at least one of X₁, X₂, X₃, and X₄ is substituted with H (SEQ ID NO: 39).

In some embodiments, the disclosure provides an antibody comprising a CD3 binding domain, CDRL1, wherein the CDRL1 binding domain comprises a consensus motif, the consensus motif comprising the sequence KSSQSLLNASTAKNYLA (SEQ ID NO: 40) or KSSQSLLNARTRTNYLA (SEQ ID NO: 41).

In some embodiments, the disclosure provides an antibody comprising a CD3 binding domain, CDRL1, wherein the CDRL1 binding domain comprises a consensus motif, the consensus motif comprising the sequence KSSQSLLNX₁X₂X₃GX₄NX₅LA, wherein X₁ is S or A, wherein X₂ is R or H, wherein X₃ is E or T, wherein X₄ is H or K, wherein X₅ is H or Y, and wherein, optionally, at least one of X₂, X₄, and X₅ is substituted with H (SEQ ID NO: 42).

In some embodiments, the disclosure provides an antibody comprising a CD3 binding domain, CDRL1, wherein the CDRL1 binding domain comprises a consensus motif, the consensus motif comprising the sequence KSSQSLLNX₁X₂TGX₃NYLA, wherein X₁ is A or S, wherein X₂ is R or H, wherein X₃ is H or K, and, optionally, wherein at least one of X₂ and X₃ is substituted with H (SEQ ID NO: 594).

In some embodiments, the disclosure provides an antibody comprising a CD3 binding domain, CDRL1, wherein the CDRL1 binding domain comprises a consensus motif, the consensus motif comprising the sequence KSSQSLLX₁AX₂X₃X₄X₅NX₆LA, wherein X₁ is N or H, wherein X₂ is R or H, wherein X₃ is T or E, wherein X₄ is G or H, wherein X₅ is H or K, wherein X₆ is H or Y, and wherein, optionally, at least one of X₁, X₂, X₄, X₅, and X₆ is substituted with H (SEQ ID NO: 597).

In some embodiments, the disclosure provides an antibody or antigen-binding fragment comprising a CDRH3 binding domain comprising a consensus motif, the consensus motif comprising the sequence ARDAX₁X₂X₃X₄FYDX₅, wherein X₁ is T, H, or Y, wherein X₂ is G or H, wherein X₃ is H or R, wherein X₄ is V or Y, wherein X₅ is V or H, and wherein, optionally, at least one of X₁, X₂, X₃, and X₅ is H (SEQ ID NO: 593); a CDRH2 binding domain comprising a consensus motif, the consensus motif comprising the sequence WIDLENANTIYDAKFQG (SEQ ID NO: 9); a CDRH1 binding domain comprising a consensus motif, the consensus motif comprising the sequence FNIKDYYMH (SEQ ID NO: 47); a CDRL3 binding domain comprising a consensus motif, the consensus motif comprising the sequence KQSX₁X₂X₃RT, wherein X₁ is H or Y, wherein X₂ is T, S, or Q, wherein X₃ is R or H, and, optionally, wherein at least one of X₁ and X₃ is H (SEQ ID NO: 36); a CDRL2 binding domain comprising a consensus motif, the consensus motif comprising the sequence WASTRES (SEQ ID NO: 37); and/or a CDRL1 binding domain comprising a consensus motif, the consensus motif comprising the sequence KSSQSLLNX₁X₂TGX₃NYLA, wherein X₁ is A or S, wherein X₂ is R or H, wherein X₃ is H or K, and, optionally, wherein at least one of X₂ and X₃ is H (SEQ ID NO: 594). In some embodiments, said antibody or antigen-binding fragment is designated as a Group 1 binder comprising a CD3 binding domain selected from ADI-48592, ADI-48595, ADI-48650, ADI-48652, ADI-48662, and ADI-48666.

In some embodiments, the disclosure provides an antibody or antigen-binding fragment comprising a CDRH3 binding domain comprising a consensus motif, the consensus motif comprising the sequence AX₁DX₂X₃X₄X₅X₆X₇YDX₈, wherein X₁ is R or H, wherein X₂ is H or A, wherein X₃ is H or Y, wherein X₄ is H, G, or P, wherein X₅ is R or H, wherein X₆ is Y, I, or V, wherein X₇ is F or H, wherein X₈ is V or H, and, optionally, wherein at least one of X₁, X₂, X₃, X₄, X₅, X₇, and X₈ is H (SEQ ID NO: 596); a CDRH2 binding domain comprising a consensus motif, the consensus motif comprising the sequence WIDLENANTIYDAKFQG (SEQ ID NO: 9) or the sequence WIDAGTGX₁TX₂YSQKFQG, wherein X₁ is L, F, N, or A and wherein X₂ is T or K (SEQ ID NO: 595); a CDRH1 binding domain comprising a consensus motif, the consensus motif comprising the sequence FNIKDYYMH (SEQ ID NO: 47) or the sequence YTFX₁X₂YX₃MH, wherein X₁ is E, S, or T, wherein X₂ is S or D, and wherein X₃ is A or D (SEQ ID NO: 31); a CDRL3 binding domain comprising a consensus motif, the consensus motif comprising the sequence X₁QSX₂X₃X₄RT, wherein X₁ is K or H, wherein X₂ is Y or H, wherein X₃ is S, H, L, V, or K, wherein X₄ is H, R, or E, and, optionally, wherein at least one of X₁, X₂, X₃, and X₄ is H (SEQ ID NO: 598); a CDRL2 binding domain comprising a consensus motif, the consensus motif comprising the sequence WASTRES (SEQ ID NO: 37); and/or a CDRL1 binding domain comprising a consensus motif, the consensus motif comprising the sequence KSSQSLLX₁AX₂X₃X₄X₅NX₆LA, wherein X₁ is N or H, wherein X₂ is R or H, wherein X₃ is T or E, wherein X₄ is G or H, wherein X₅ is H or K, wherein X₆ is H or Y, and wherein, optionally, at least one of X₁, X₂, X₄, X₅, and X₆ is H (SEQ ID NO: 597). In some embodiments, said antibody or antigen-binding fragment is designated as a Group 2 binder comprising a CD3 binding domain selected from ADI-48588, ADI-48587, ADI-48577, ADI-48590, ADI-48581, ADI-48575, ADI-48593, ADI-48591, ADI-48647, ADI-48636, ADI-48586, ADI-48646, ADI-48638, ADI-48597, ADI-48601, ADI-48576, ADI-48643, ADI-48624, ADI-48632, ADI-48635, and ADI-48645.

In some embodiments, the disclosure provides an anti-CD3 antibody or antigen-binding fragment described herein, wherein said antibody or antigen-binding fragment may elicit T cell activation or T cell killing while displaying a decreased propensity to elicit cytokine production to levels capable of inducing cytokine release syndrome.

In some embodiments, the disclosure provides an anti-CD3 antibody or antigen-binding fragment described herein, wherein said antibody or antigen-binding fragment may comprise a multispecific antibody.

In some embodiments, the disclosure provides an anti-CD3 antibody or antigen-binding fragment described herein, wherein said antibody or antigen-binding fragment may comprise a bispecific antibody.

In some embodiments, the disclosure provides an anti-CD3 antibody or antigen-binding fragment described herein, wherein said antibody or antigen-binding fragment may comprise an scFv.

In some embodiments, the disclosure provides an anti-CD3 antibody or antigen-binding fragment described herein, wherein said antibody or antigen-binding fragment may comprise at least a second antigen-binding domain that specifically binds to an oncology target; an immune-oncology target; a neurodegenerative disease targets; an autoimmune disorder target; an infectious disease target; a metabolic disease target; a cognitive disorder target; a blood-brain barrier target; or a blood disease target.

In some embodiments, the disclosure provides an anti-CD3 antibody or antigen-binding fragment described herein, wherein said antibody or antigen-binding fragment may comprise at least a second antigen-binding domain that specifically binds to an antigen selected from the group consisting of: 17-IA, 4-1BB, 4Dc, 6-keto-PGF1a, 8-iso-PGF2a, 8-oxo-dG, A1 Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RUB, ADAM, ADAM10, ADAM12, ADAM 15, ADAM 17/T ACE, ADAM8, ADAM9, ADAMTS, ADAMTS4, ADAMTS5, Addressins, aFGF, ALCAM, ALK, ALK-1, ALK-7, alpha-1-antitrypsin, alpha-V/beta-1 antagonist, ANG, Ang, APAF-1, APE, APJ, APP, APRIL, AR, ARC, ART, Artemin, anti-Id, ASPARTIC, Atrial natriuretic factor, av/b3 integrin, Axl, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bel, BCMA, BDNF, b-ECGF, bFGF, BID, Bik, BFM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, C1O, CA125, CAD-8, Calcitonin, cAMP, carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/Z/P, CBL, CCI, CCK2, CCL, CCL1, CCL11, CCL12, CCL13, CCL 14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD28, CD29, CD30, CD30L, CD32, CD33 (p67 proteins), CD34, CD38, CD40, CD40L, CD44, CD45, CD46, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(1-3)-IGF-1 (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, EN A, endothelin receptor, Enkephalinase, eNOS, Eot, eotaxin1, EpCAM, Ephrin B2/EphB4, EPO, ERCC, E-selectin, ET-1, Factor IIa, Factor VII, Factor VTITc, Factor IX, fibroblast activation protein (FAP), Fas, FcR1, FEN-1, Ferritin, FGF, FGF-19, FGF-2, FGF3, FGF-8, FGFR, FGFR-3, Fibrin, FL, FLIP, Flt-3, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GFAP, GFRa-1, GFR-alpha1, GFR-alpha2, GFR-alpha3, GITR, Glucagon, Glut 4, glycoprotein IIb/IIIa (GP IIb/IIIa), GM-CSF, gp130, gp72, GRO, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gp120, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MAA), HIV gp120, HIV IIIB gp 120 V3 loop, HLA, HLA-DR, HM1.24, HMFG PEM, HRG, Hrk, human cardiac myosin, human cytomegalovirus (HCMV), human growth hormone (HGH), HVEM, 1-309, IAP, ICAM, ICAM-1, ICAM-3, ICE, ICOS, IFNg, Ig, IgA receptor, IgE, IGF, IGF binding proteins, IGF-TR, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-18, IL-18R, IL-23, interferon (INF)-alpha, INF-beta, INF-gamma, Inhibin, iNOS, Insulin A-chain, Insulin B-chain, Insulin-like growth factor 1, integrin alpha2, integrin alpha3, integrin alpha4, integrin alpha4/beta1, integrin, alpha4/beta7, integrin alpha5 (alphaV), integrin alpha5/beta1, integrin alpha5/beta3, integrin alpha6, integrin beta1, integrin beta2, interferon gamma, IP-10, 1-TAC, JE, Kallikrein 2, Kallikrein 5, Kallikrein 6, Kallikrein 11, Kallikrein 12, Kallikrein 14, Kallikrein 15, Kallikrein L1, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bpl, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, a metalloprotease, MGDF receptor, MGMT, MHC (HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Muc1), MUC18, Muellerian-inhibiting substance, Mug, MuSK, NAIP, NAP, NCAD, N-Cadherin, NCA 90, NCAM, NCAM, Neprilysin, Neurotrophin-3, -4, or -6, Neurturin, Neuronal growth factor (NGF), NGFR, NGF-beta, nNOS, NO, NOS, Npn, NRG-3, NT, NTN, OB, OGG1, OPG, OPN, OSM, OX40L, OX40R, p150, p95, PADPr, Parathyroid hormone, PARC, PARP, PBR, PBSF, PCAD, P-Cadherin, PCNA, PDGF, PDGF, PDK-1, PECAM, PEM, PF4, PGE, PGF, PGI2, PGJ2, PIN, PLA2, placental alkaline phosphatase (PLAP), PIGF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, Relaxin A-chain, Relaxin B-chain, renin, respiratory syncytial virus (RSV) F, RSV Fgp, Ret, Rheumatoid factors, RLIP76, RPA2, RSK, Si00, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta RI (ALK-5), TGF-beta RII, TGF-beta RIIb, TGF-beta RIII, TGF-beta1, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha, TNF-alpha beta, TNF-beta2, TNFc, TNF-RI, TNF-RII, TNFRSF10A (TRAIL Rb Apo-2, DR4), TNFRSFIOB (TRAIL R2 DR5, KILLER, TRICK-2A, TRICK-B), TNFRSF10C (TRAIL R3 DcR1, LIT, TRID), TNFRSF10D (TRAIL R4 DcR2, TRUNDD), TNFRSF11A (RANK ODF R, TRANCE R), TNFRSFIIB (OPG OC1F, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF 18 (GITR AITR), TNFRSF19 (TROY TAJ, TRADE), TNFRSF19L (RELT), TNFRSFIA (TNF R1 CD120a, p55-60), TNFRSFIB (TNF RII CD120b, p75-80), TNFRSF26 (TNFRH3), TNFRSF3 (LTbR TNF RIII, TNFC R), TNFRSF4 (OX40 ACT35, TXGP1 R), TNFRSF 5 (CD40 p50), TNFRSF6 (Fas Apo-1, APT1, CD95), TNFRSF6B (DcR3 M68, TR6), TNFRSF7 (CD27), TNFRSF8 (CD30), TNFRSF9 (4-1BB CD137, ILA), TNFRSF21 (DR6), TNFRSF22 (DcTRAIL R2 TNFRH2), TNFRST23 (DcTRAIL R1 TNFRH1), TNFRSF25 (DR3 Apo-3, LARD, TR-3, TRAMP, WSL-1), TNFSF10 (TRAIL Apo-2 Ligand, TL2), TNFSF11 (TRANCE/RANK Ligand ODF, OPG Ligand), TNFSF12 (TWEAK Apo-3 Ligand, DR3 Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TLIA/VEGI), TNFSF18 (GITR Ligand AITR Ligand, TL6), TNFSFIA (TNF-a Conectin, DIF, TNFSF2), TNFSF1B (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD30 Ligand CD153), TNFSF9 (4-1BB Ligand CD137 Ligand), TP-1, t-PA, Tpo, TRAIL, TRAIL R, TRAIL-R1, TRAIL-R2, TRANCE, transferring receptor, TRF, Trk, TROP-2, TSG, TSLP, tumor-associated antigen CA 125, tumor-associated antigen expressing Lewis Y related carbohydrate, TWEAK, TXB2, Ung, uPAR, uPAR-1, Urokinase, VCAM, VCAM-1, VECAD, VE-Cadherin, VE-cadherin-2, VEFGR-1 (flt-1), VEGF, VEGFR, VEGFR-3 (flt-4), VEGI, VFM, Viral antigens, VLA, VLA-1, VLA-4, VNR integrin, von Willebrands factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, X1AP, XPD, CTLA4 (cytotoxic T lymphocyte antigen-4), PD1 (programmed cell death protein 1), PD-L1 (programmed cell death ligand 1), LAG-3 (lymphocyte activation gene-3), TIM-3 (T cell immunoglobulin and mucin protein-3), hormone receptors and growth factors.

In some embodiments, the disclosure provides an anti-CD3 antibody or antigen-binding fragment described herein, wherein said antibody or antigen-binding fragment may comprise at least a second antigen-binding domain that specifically binds to an antigen selected from the group consisting of: BCMA, CTLA4 (cytotoxic T lymphocyte antigen-4), PD1 (programmed cell death protein 1), PD-L1 (programmed cell death ligand 1), LAG-3 (lymphocyte activation gene-3), TIM-3, CD20, CD2, CD19, Her2, EGFR, EpCAM, FcyRIIIa (CD16), FcyRIIa (CD32a), FcyRIIb (CD32b), FcyRI (CD64), Toll-like receptors (TLRs), TLR4, TLR9, cytokines, IL-2, IL-5, IL-13, IL-6, IL-17, IL-12, IL-23, TNFa, TGFb, cytokine receptors, IL-2R, chemokines, chemokine receptors, growth factors, VEGF, and HGF.

In some embodiments, the disclosure provides an anti-CD3 antibody or antigen-binding fragment described herein, wherein said antibody or antigen-binding fragment may be comprised in a chimeric antigen receptor (CAR), which optionally may comprise at least one transmembrane domain, and at least one intracellular domain from a T-cell receptor, optionally a CD3ζ subunit, and at least one co-stimulatory domain.

In some embodiments, the disclosure provides an anti-CD3 antibody or antigen-binding fragment described herein, wherein said antibody or antigen-binding fragment may comprise an scFv2-Fc2 and/or scFv-IgG.

In some embodiments, the disclosure provides an anti-CD3 antibody or antigen-binding fragment described herein, wherein said antibody or antigen-binding fragment may comprise an IgG constant domain.

In some embodiments, the disclosure provides an anti-CD3 antibody or antigen-binding fragment described herein, wherein said antibody or antigen-binding fragment may comprise at least a second antigen-binding domain that specifically binds to an antigen, wherein said antibody comprises a multispecific format selected from the group consisting of Fab-Fc-scFv, “bottle-opener”, Mab-scFv, Mab-Fv, Dual scFv, central Fv, central scFv, one-arm central scFv, Fab-Fab, Fab-Fv, mAb-Fv, mAb-Fab, DART, BiTE, common light chain-IgG, TandAb, Cross-Mab, SEED, BEAT, TrioMab, and DuetMab.

In some embodiments, the disclosure provides an isolated or recombinant nucleic acid sequence encoding an anti-CD3 antibody or antigen-binding fragment described herein.

In some embodiments, the disclosure provides an expression vector comprising an isolated or recombinant nucleic acid sequence encoding an anti-CD3 antibody or antigen-binding fragment described herein.

In some embodiments, the disclosure provides a host cell transfected, transformed, or transduced with a nucleic acid sequence encoding an anti-CD3 antibody or antigen-binding fragment described herein, or an expression vector comprising an isolated or recombinant nucleic acid sequence encoding an anti-CD3 antibody or antigen-binding fragment described herein, wherein the host cell may optionally be a mammalian cell or a yeast cell.

In some embodiments, the disclosure provides a pharmaceutical composition comprising an antibody or antigen-binding fragment described herein or a host cell described herein, and a pharmaceutically acceptable carrier and/or excipient.

In some embodiments, the disclosure provides a method of treating a disorder in a mammal in need of such treatment, wherein the disorder may comprise a proliferative disorder, an oncological disorder, an immuno-oncological disorder, a neurological disorder, a neurodegenerative disorder, or an autoimmune disorder, and wherein the method may comprise administering an effective amount of at least one antibody or antigen-binding fragment described herein or a host cell which expresses at least one of said antibody or antigen-binding fragment described herein, optionally an immune cell, further optionally a T or NK cell. In some embodiments, the method may further comprise administering to the mammal an additional therapeutic agent, optionally wherein the mammal is a human.

In other embodiments, the disclosure provides an anti-CD3 antibody or antigen-binding fragment thereof comprising one or more of a CDRL1, a CDRL2, and a CDRL3. Such an antibody, in some embodiments, further comprises a CDRH1, a CDRH2, and a CDRH3.

### BRIEF DESCRIPTION OF THE DRAWING

FIG. 1A and FIG. 1B show pre-saturation methods 1 and 2. FIG. 1A: CD3 Pre-saturation Method #1: yeast cells were pre-saturated with native (un-biotinylated) CD3 antigen at pH 7.4 for 10 minutes, and yeast cells were washed at pH 7.4 and incubated in pH 6.0 media for 10 minutes to allow dissociation of antigen. Control cells were washed and incubated at pH 7.4. Lastly, yeast cells were incubated with biotinylated CD3 antigen (shown as a starred green circle) at pH 6 for 10 minutes. Control cells were incubated with biotinylated CD3 antigen at pH 7.4. Binders labeled at pH 6 were then sorted and characterized. FIG. 1B: CD3 Pre-saturation Method #2: yeast cells were pre-saturated with native CD3 antigen at pH 6.0 for 10 minutes and washed at pH 6.0 and incubated at either pH 7.4 or pH 6.0 for 10 minutes. Lastly, yeast cells were incubated with biotinylated CD3 antigen at the opposite pH (cells incubated at pH 6.0 in the previous step were incubated at pH 7.4, whereas cells incubated at pH 7.4 in the previous step were incubated at pH 6.0) for 10 minutes. Binders labeled with biotinylated CD3 antigen were then sorted and characterized.

FIG. 2 shows exemplary FACS plots from Round 1 and Round 2 selections from one library. Similar binding profiles were observed for all libraries. Briefly, during Round 1 cells were positively sorted using 100 nM human CD3εδ heterodimer (HuCD3-hd) at pH 6. During Round 2, cells were positively sorted using 100 nM HuCD3-hd at pH 6.0, negatively sorted using 100 nM HuCD3-hd at pH 7.4, or pre-saturated using Method #2 described above. Binding was also confirmed for cynomolgus CD3 (CyCD3-hd) at pH 6.0. Arrows indicate sorted cells that were carried forward to the next round of sorting.

FIG. 3 shows exemplary FACS plots from Round 3 and compares inputs of pH 6.0-positive sort and pH 7.4-negative sort from Round 2. Briefly, the sorts from Round 2 were incubated with 100 nM HuCD3-hd at pH 6.0 and 7.4. The Overlay column shows that the input cell population (from Round 2 sorts) exhibits higher binding at pH 6.0 compared to pH 7.4. The pre-saturation/toggle Method #2 was used to carry forward cells into the next rounds of selections.

FIG. 4 shows exemplary FACS plots from Round 4 and Round 5. Round 4 compared cells incubated in 100 nM HuCD3-hd at pH 6 and pH 7.4. Round 4 also compared cells subjected to the pre-saturation/toggling method at pH 6 and pH 7.4. Round 5 compared cells incubated in either 100 nM HuCD3-hd or 100 nM CyCD3-hd at pH 6 (red) and pH 7.4 (grey).

FIG. 5A and FIG. 5B show HuCD3 binding response. FIG. 5A shows HuCD3 binding response at pH 6 (x-axis) compared to HuCD3 binding response at pH 7.4 (y-axis) for 236 unique clones from Round 2/3 sort outputs. FIG. 5B shows K_{D} values for HuCD3 at pH 6 (x-axis) compared to HuCD3 at pH 7.4 (y-axis) for the 236 unique clones from Round 2/3 sort outputs. Blue circles represent the Round 2/3 clones obtained via the pH 6.0 pre-saturation/toggle sort, yellow circles represent the Round 2/3 clones obtained via the pH 7.4 negative sort, and the red circles represent the parent clone ADI-26906. Results show that the pH 7.4 negative sorts at Round 2/3 tends to yield more pH selected binders but with weaker pH 6.0 response or affinity, designated as Group 2 binders. Positive selections at pH 6.0 and the pre-saturation/toggle sort yielded clones with a mix of selectivity but with higher response/affinity. Such clones with higher affinity at pH 6.0 (e.g., K_{D}<˜25 nM) are designated as Group 1 binders.

FIG. 6 provides exemplary kinetics from the ForteBio experiments for four clones compared to parent clone ADI-26906. The K_{D} for each clone was calculated at pH 7.4 and pH 6.0. A ratio K_{D} was obtained by dividing the K_{D} at pH 7.4 by the K_{D} at pH 6.0. The examples demonstrate that some clones which are designated as Group 1 binders, such as SAD10318_P02_A05 (ADI-48595) and SAD10318_P02_C04 (ADI-48592), bound stronger (with a lower K_{D}) at pH 6.0 compared to pH 7.4. Some clones which are designated as Group 2 binders, such as SAD10318_P01_A03 (ADI-48587) and SAD10318_P01_E01 (ADI-48577), were non-binders at pH 7.4 but bound at pH 6.0. Amino acid substitutions in the CDRH3, CDRL1, and CDRL3 regions that may contribute to the differential binding are highlighted the Sequence column of FIG. 6 (SEQ ID NOS 576-590, respectively, in order of appearance). Clones such as ADI-48576, ADI-48577, ADI-48587, ADI-48592, ADI-48595, ADI-48635, ADI-48650, ADI-48652, ADI-48666, ADI-48643, and ADI-48645 exhibit pH-dependent binding (with stronger binding at pH 6.0 relative to binding at pH 7.4), low PSR scores, and provide a range of affinities for CD3.

### DETAILED DESCRIPTION OF THE INVENTION

Unless defined otherwise, all technical and scientific terms used herein have the same meaning as commonly understood by one of ordinary skill in the art to which this disclosure belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

It is understood that aspects and embodiments of the disclosure described herein include “comprising,” “consisting,” and “consisting essentially of” aspects and embodiments.

Provided herein are anti-CD3 antibodies and antigen-binding fragments thereof that exhibit pH-dependent binding and favorable developability profiles.

“Cluster of Differentiation 3” or “CD3”, generally refers to any native CD3 from any vertebrate source, including mammals such as primates (e.g., humans) and rodents (e.g., mice and rats), unless otherwise indicated, including, for example, CD3ε, CD3γ, CD3α, and CD3β chains. The term encompasses “full-length,” unprocessed CD3 (e.g., unprocessed or unmodified CD3ε or CD3γ), as well as any form of CD3 that results from processing in the cell. The term also encompasses naturally occurring variants of CD3, including, for example, splice variants or allelic variants. CD3 includes, for example, human CD3ε protein (NCBI RefSeq No. NP_000724), which is 207 amino acids in length, and human CD3γ protein (NCBI RefSeq No. NP_000064), which is 182 amino acids in length. The term also refers to either the human or cynomolgus CD3epsilon protein, SEQ ID NOs: 591 and 592, respectively, (Table 4). “CD3εN27” and “CD3εN13” refer to the N-terminal 27 amino acids and the N-terminal 13 amino acids, respectively, of CD3, and optionally containing chemical modifications or conjugations made thereto.

An “anti-CD3 antibody” refers to an antibody or an antigen-binding fragment thereof capable of binding to CD3, e.g., CD3ε and/or CD3γ, e.g., human CD3ε and/or CD3γ with sufficient affinity and/or specificity such that the antibody is useful as a diagnostic and/or therapeutic agent in targeting CD3. In some embodiments, an anti-CD3 antibody binds to CD3 with a dissociation constant (K_{D}) of about 100×10⁻⁹ M or less, about 50×10⁻⁹ M or less, about 25×10⁻⁹ M or less, about 20×10⁻⁹ M or less, or about 10×10⁻⁹ M or less. In some embodiments, an anti-CD3 antibody binds to CD3 with a dissociation constant (K_{D}) of about 5×10⁻⁹ M or less. In some embodiments, an anti-CD3 antibody binds to CD3 with a dissociation constant (K_{D}) of about 2.5×10⁻⁹ M or less. In some embodiments, an anti-CD3 antibody binds to CD3 with a dissociation constant (K_{D}) of about 1×10⁻¹⁰ M or less. In some embodiments, K_{D} is measured by surface plasmon resonance, e.g., BIACORE, biolayer interferometry measurements using, e.g., a FORTEBIO Octet HTX instrument (Pall Life Sciences), or solution-affinity ELISA. In some embodiments, the K_{D} is measured using an scFv fragment of the anti-CD3 antibody. In some embodiments, the monovalent K_{D} is measured. In some embodiments, the anti-CD3 antibody binds to an epitope of CD3 that is conserved among CD3 from different species, e.g., human and cyno cross-reactive.

The term “engineered pH-dependent” refers to an antibody having a modified amino acid sequence that allows for preferential or selective antigen binding at a certain pH. For example, a parent antibody can be engineered (e.g., by modifying the amino acid sequence) for pH-dependent binding. pH-dependent binding refers to an antibody's preference to bind an antigen at a given pH (or given pH range) as compared to a different pH (or pH range). In one embodiment, pH-dependent antibodies preferentially or selectively bind to an antigen at a pH at around 6 as compared to a pH at around 7. An antibody sequence may be modified by, for example, substitution with one or more ionizable amino acid residues such as histidine, lysine, arginine, aspartic acid, and glutamic acid. Ionizable residues may be substituted into CDRs and/or the FRs. In some embodiments, 1-10 substitutions may be present per variant VH or VK. In some embodiments, 1-6 substitutions may be present per variant VH or VK.

The term “cytokine release syndrome” (or “CRS”) refers to a pro-inflammatory, positive feedback loop between cytokines and immune cells leading to excessive or uncontrolled release of pro-inflammatory cytokines by cells within the immune system (see, e.g., Lee et al., Blood, Vol. 124, pages 188-195 (2014) and Tisoncik et al., Microbiol Mol Biol Rev, Vol. 76, pages 16-32 (2012). Upon stimulation and activation, T cells release a series of cytokines to a level and degree that generates untoward biological/physiological effects or varying degree and severity, including acute inflammation characterized by, e.g., rubor (redness), swelling or edema, calor (heat), dolor (pain), and “functio laesa” (loss of function). When localized in skin or other tissue, biological/physiological effects comprise increased blood flow, enabling vascular leukocytes and plasma proteins to reach extravascular sites of injury, increasing local temperatures and generation of pain, tissue edema and extravascular pressure and a reduction in tissue perfusion. Other biological/physiological effects comprise organ and system dysfunction, such as cardiac dysfunction, adult respiratory distress syndrome, neurologic toxicity, renal and/or hepatic failure, and disseminated intravascular coagulation. Elevated levels of IFNγ, IL-6, TNFα, TGFbeta, IL-2, granulocyte macrophage-colony-stimulating factor (GM-CSF), IL-10, IL-8, IL-5, and/or fractalkine are implicated as predictive and/or causative of CRS or the propensity to elicit CRS upon T-cell stimulation.

In certain embodiments, the anti-CD3 antibodies and/or antigen-binding fragments thereof described herein are detuned and/or modified to reduce the likelihood or severity of CRS induced by the antibody. Non-limiting exemplary modifications may include silent Fc regions (e.g., removing the Fc completely or modifying the Fc region to reduce or eliminate effector function), and/or masking (e.g., a polypeptide mask that is positioned such that it reduces or inhibits the ability of the antibody or antigen-binding fragment thereof to specifically bind CD3).

The term “antibody” is used herein in the broadest sense and encompasses various antibody structures, including but not limited to monoclonal antibodies, polyclonal antibodies, multispecific antibodies (e.g., bispecific antibodies), and/or antibody fragments (preferably those fragments that exhibit the desired antigen-binding activity).

A “monoclonal antibody” or “mAb” refers to an antibody obtained from a population of substantially homogeneous antibodies, i.e., the individual antibodies comprising the population are identical and/or bind the same epitope, except for possible variant antibodies (e.g., containing naturally occurring mutations or arising during production of a monoclonal antibody preparation), such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

With regard to multispecific antibodies, such antibodies comprise at least two different antigen binding domains which recognize and specifically bind to at least two different antigens. With regard to bispecific antibodies, such antibodies comprise two different antigen binding domains which recognize and specifically bind to at least two different antigens.

A “different antigen” may refer to different and/or distinct proteins, polypeptides, or molecules; as well as different and/or distinct epitopes, which epitopes may be contained within one protein, polypeptide, or other molecule.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

In some instances, an antibody comprises four polypeptide chains: two heavy (H) chains and two light (L) chains interconnected by disulfide bonds. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

In other instances, an antibody may instead comprise multimers thereof (e.g., IgM) or antigen-binding fragments thereof. Each heavy chain is comprised of a heavy chain variable region (“VH”) and a heavy chain constant region (“CH”), which is comprised of domains CH1, CH2 and CH3. Each light chain is comprised of a light chain variable region (“VL”) and a light chain constant region (“CL”). The VH and VL regions can be further subdivided into regions of hypervariability, termed complementarity determining regions (CDRs), interspersed with regions that are more conserved, termed framework regions (FRs). Each VH and VL is composed of three CDRs and four FRs, arranged from amino-terminus to carboxy-terminus in the following order: FR1, CDR1, FR2, CDR2, FR3, CDR3, FR4. In certain embodiments of the disclosure, the FRs of the antibody (or antigen-binding fragment thereof) may be identical to the human germline sequences or may be naturally or artificially modified. An amino acid consensus sequence may be defined based on a side-by-side analysis of two or more CDRs. Accordingly, the CDRs in a heavy chain are designated “CDRH1”, “CDRH2”, and “CDRH3”, respectively, and the CDRs in a light chain are designated “CDRL1”, “CDRL2”, and “CDRL3”.

Unless specifically indicated otherwise, the term “antibody” as used herein encompasses molecules comprising two immunoglobulin heavy chains and two immunoglobulin light chains (i.e., “full-length antibody” or “intact antibodies” or “whole antibody”) as well as antigen-binding fragments thereof.

An “antigen-binding fragment” refers to a portion of an intact antibody that binds the antigen to which the intact antibody binds (in this case, CD3). The terms “full-length antibody,” “intact antibody,” and “whole antibody” or the like are used herein interchangeably and refer to an antibody having a structure substantially similar to a native antibody.

An antigen-binding fragment of an antibody includes any naturally occurring, enzymatically obtainable, synthetic, or genetically engineered polypeptide or glycoprotein that specifically binds an antigen to form a complex, including an antibody fragment. Exemplary antigen-binding fragments include, but are not limited to, Fv, Fab, Fab′, Fab′-SH, F(ab′)₂; diabodies; linear antibodies; single-chain antibody molecules (e.g. scFv or VH or VL domains only); and multispecific antibodies formed from antibody fragments. In some embodiments, the antigen-binding fragments of the anti-CD3 antibodies described herein are scFvs.

As with full antibody molecules, antigen-binding fragments may be mono-specific or multispecific (e.g., bispecific). A multi-specific antigen-binding fragment of an antibody may comprise at least two different variable domains, wherein each variable domain is capable of specifically binding to a separate antigen or to a different epitope on the same antigen. A variety of multi-specific antibody formats may be used in the context of an antigen-binding fragment of anti-CD3 antibody described herein. Non-limiting examples of multispecific and bispecific formats include, e.g., Fab-Fc-scFv (“bottle-opener”) (XENCOR), Mab-scFv (XENCOR), Mab-Fv (XENCOR), Dual scFv (XENCOR), central Fv (XENCOR), central scFv(XENCOR), one-arm central scFv (XENCOR), Fab-Fab (XENCOR), Fab-Fv (XENCOR), mAb-Fv (XENCOR), mAb-Fab (XENCOR), DART (MACROGENICS), BiTE (AMGEN/MICROMET), KiTE, common light chain-IgG (Genentech), TandAb (SFFIMED) Cross-Mab (ROCHE), SEED (EMD SERONO), BEAT (GLENMARK), TrioMab (TRION PHARMA/FRESENIUS BIOTECH), DuetMab (MEDIMMUNE), and others, as disclosed, e.g., in (WO 95/09917; WO 2008/119566; WO 2008/119567; WO2011/121110; WO 2010/037835; WO 2007/042261; WO 2007/110205; WO 2011/121110; WO 2012/055961; WO 2012/16067; WO 2016/086189; WO 2016/182751; WO 2015/006749; WO 2014/049003; WO 2013/177101; WO 2015/128509; U.S. Pat. No. 7,951,917; US 2009/0252729; US 2014/0348839; U.S. Pat. No. 7,183,076; Mazor et al., Mabs, Vol. 7, pages 377-389 (2015); Muda et al., Protein Engineering, Design, & Selection, Vol. 24, pages 447-454 (2011); and Del Bano et al., Antibodies, Vol. 5, pages 1-23 (2016). In some embodiments, the anti-CD3 scFv fragments described herein comprise one or more variable domains of a multispecific (e.g., bispecific), antibody.

In certain embodiments, the anti-CD3 antibodies and/or antigen-binding fragments thereof as described herein are contained in a multispecific antibody, in particular, a bispecific antibody that has binding specificity for a second antigen. Such a second antigen may be a different target altogether than the first target, or a different epitope present on the same target. In some embodiments, the binding specifities are to two different epitopes of CD3 (e.g., CD3ε or CD3γ). In other embodiments, one of the binding specificities is for CD3 (e.g., CD3ε or CD3γ) and the other is for a different biological molecule (e.g., a cell surface antigen, e.g., a tumor antigen).

Non-limiting examples of a second antigen toward which a bispecific antibody comprising anti-CD3 antibodies and/or antigen-binding fragments thereof as described herein, comprises targets selected from the group consisting of: 17-IA, 4-1BB, 4Dc, 6-keto-PGF1a, 8-iso-PGF2a, 8-oxo-dG, A1 Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RUB, ADAM, ADAM10, ADAM12, ADAM15, ADAM17/TACE, ADAM8, ADAM9, ADAMTS, ADAMTS4, ADAMTS5, Addressins, aFGF, ALCAM, ALK, ALK-1, ALK-7, alpha-1-antitrypsin, alpha-V/beta-1 antagonist, ANG, Ang, APAF-1, APE, APJ, APP, APRIL, AR, ARC, ART, Artemin, anti-Id, ASPARTIC, Atrial natriuretic factor, av/b3 integrin, Axl, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bel, BCMA, BDNF, b-ECGF, bFGF, BID, Bik, BIM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, CIO, CA125, CAD-8, Calcitonin, cAMP, carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/Z/P, CBL, CCI, CCK2, CCL, CCL1, CCL11, CCL12, CCL13, CCL 14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD28, CD29, CD30, CD30L, CD32, CD33 (p67 proteins), CD34, CD38, CD40, CD40L, CD44, CD45, CD46, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(1-3)-IGF-I (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, EN A, endothelin receptor, Enkephalinase, eNOS, Eot, eotaxin1, EpCAM, Ephrin B2/EphB4, EPO, ERCC, E-selectin, ET-1, Factor IIa, Factor VII, Factor VIIIc, Factor IX, fibroblast activation protein (FAP), Fas, FcR1, FEN-1, Ferritin, FGF, FGF-19, FGF-2, FGF3, FGF-8, FGFR, FGFR-3, Fibrin, FL, FLIP, Flt-3, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GDNF, GFAP, GFRa-1, GFR-alpha1, GFR-alpha2, GFR-alpha3, GITR, Glucagon, Glut 4, glycoprotein IIb/IIa (GP IIb/IIIa), GM-CSF, gp130, gp72, GRO, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gp120, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MAA), HIV gp120, HIV 111B gp 120 V3 loop, HLA, HLA-DR, HM1.24, HMFG PEM, HRG, Hrk, human cardiac myosin, human cytomegalovirus (HCMV), human growth hormone (HGH), HVEM, 1-309, IAP, ICAM, ICAM-1, ICAM-3, ICE, ICOS, IFNg, Ig, IgA receptor, IgE, IGF, IGF binding proteins, IGF-1R, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-18, IL-18R, IL-23, interferon (INF)-alpha, INF-beta, INF-gamma, Inhibin, iNOS, Insulin A-chain, Insulin B-chain, Insulin-like growth factor 1, integrin alpha2, integrin alpha3, integrin alpha4, integrin alpha4/beta1, integrin, alpha4/beta7, integrin alpha5 (alphaV), integrin alpha5/beta1, integrin alpha5/beta3, integrin alpha6, integrin beta1, integrin beta2, interferon gamma, IP-10, 1-TAC, JE, Kallikrein 2, Kallikrein 5, Kallikrein 6, Kallikrein 11, Kallikrein 12, Kallikrein 14, Kallikrein 15, Kallikrein L1, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bpl, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, METALLOPROTEASES, MGDF receptor, MGMT, MHC (HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Muc1), MUC18, Muellerian-inhibitin substance, Mug, MuSK, NAIP, NAP, NCAD, N-Cadherin, NCA 90, NCAM, NCAM, Neprilysin, Neurotrophin-3, -4, or -6, Neurturin, Neuronal growth factor (NGF), NGFR, NGF-beta, nNOS, NO, NOS, Npn, NRG-3, NT, NTN, OB, OGG1, OPG, OPN, OSM, OX40L, OX40R, p150, p95, PADPr, Parathyroid hormone, PARC, PARP, PBR, PBSF, PCAD, P-Cadherin, PCNA, PDGF, PDGF, PDK-1, PECAM, PEM, PF4, PGE, PGF, PGJ2, PGJ2, PIN, PLA2, placental alkaline phosphatase (PLAP), P1GF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, RANTES, Relaxin A-chain, Relaxin B-chain, renin, respiratory syncytial virus (RSV) F, RSV Fgp, Ret, Rheumatoid factors, RLIP76, RPA2, RSK, S100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-TT, TACE, TACT, TAG-72 (tumor-associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta RI (ALK-5), TGF-beta RII, TGF-beta RIIb, TGF-beta RIII, TGF-beta1, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha, TNF-alpha beta, TNF-beta2, TNFc, TNF-RI, TNF-RII, TNFRSF10A (TRAIL R1 Apo-2, DR4), TNFRSFIOB (TRAIL R2 DR5, KILLER, TRICK-2A, TRICK-B), TNFRSF10C (TRAIL R3 DcR1, LIT, TRID), TNFRSF10D (TRAIL R4 DcR2, TRUNDD), TNFRSF11A (RANK ODF R, TRANCE R), TNFRSF11B (OPG OC1F, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITR AITR), TNFRSF19 (TROY TAJ, TRADE), TNFRSF19L (RELT), TNFRSFIA (TNF RI CD120a, p55-60), TNFRSFIB (TNF RII CD120b, p75-80), TNFRSF26 (TNFRH3), TNFRSF3 (LTbR TNF RIII, TNFC R), TNFRSF4 (OX40 ACT35, TXGP1 R), TNFRSF5 (CD40 p50), TNFRSF6 (Fas Apo-1, APT1, CD95), TNFRSF6B (DcR3 M68, TR6), TNFRSF7 (CD27), TNFRSF8 (CD30), TNFRSF9 (4-1BB CD137, ILA), TNFRSF21 (DR6), TNFRSF22 (DcTRAIL R2 TNFRH2), TNFRST23 (DcTRAIL R1 TNFRH1), TNFRSF25 (DR3 Apo-3, LARD, TR-3, TRAMP, WSL-1), TNFSF10 (TRAIL Apo-2 Ligand, TL2), TNFSF11 (TRANCE/RANK Ligand ODF, OPG Ligand), TNFSF12 (TWEAK Apo-3 Ligand, DR3 Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TLIA/VEGI), TNFSF18 (GITR Ligand AITR Ligand, TL6), TNFSFIA (TNF-α Conectin, DIF, TNFSF2), TNFSF1B (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD30 Ligand CD153), TNFSF9 (4-1BB Ligand CD137 Ligand), TP-1, t-PA, Tpo, TRAIL, TRAIL R, TRAIL-R1, TRAIL-R2, TRANCE, transferring receptor, TRF, Trk, TROP-2, TSG, TSLP, tumor-associated antigen CA 125, tumor-associated antigen expressing Lewis Y related carbohydrate, TWEAK, TXB2, Ung, uPAR, uPAR-1, Urokinase, VCAM, VCAM-1, VECAD, VE-Cadherin, VE-cadherin-2, VEFGR-1 (fit-1), VEGF, VEGFR, VEGFR-3 (fit-4), VEGI, VIM, Viral antigens, VLA, VLA-1, VLA-4, VNR integrin, von Willebrands factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, X1AP, XPD, CTLA4 (cytotoxic T lymphocyte antigen-4), PD1 (programmed cell death protein 1), PD-L1 (programmed cell death ligand 1), LAG-3 (lymphocyte activation gene-3), TIM-3 (T cell immunoglobulin and mucin protein-3), receptors for hormones, and growth factors.

Multispecifics comprising anti-CD3 antibodies and antigen-binding fragments disclosed herein may be prepared according to a variety of techniques including, but are not limited to, recombinant co-expression of two immunoglobulin heavy chain-light chain pairs having different specificities (see, Milstein and Cuello, Nature 305: 537 (1983)), WO 93/08829, and Traunecker et al., EMBO J 10: 3655 (1991)), “knob-in-hole” engineering (see, e.g., U.S. Pat. No. 5,731,168); immunoglobulin crossover (also known as Fab domain exchange or CrossMab format) technology (see, e.g., WO2009/080253; Schaefer et al., Proc. Natl. Acad. Sci. USA, 108:11187-11192 (2011)); engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); leucine zippers (see, e.g., Kostelny et al., J. Immunol, 148(5):1547-1553 (1992)); “diabody” technology (see, e.g., Hollinger et al., Proc. Natl. Acad. Sci. USA, 90:6444-6448 (1993)); single-chain Fv (sFv) dimers (see, e.g. Gruber et al., J. Immunol, 152:5368 (1994)); and trispecific antibodies as described, e.g., in Tutt et al. J. Immunol 147: 60 (1991).

The present disclosure also contemplates modification of anti-CD3 antibodies disclosed herein, such modifications comprising one or more amino acid substitutions, insertions and/or deletions in the FR and/or CDR regions of the heavy and light chain variable domains. Once obtained, such derivative antibodies and/or antigen-binding fragments can be tested for one or more desired properties such as improved binding specificity, increased binding affinity, improved developability, etc.

In some embodiments, anti-CD3 antibodies and/or antigen-binding fragments thereof comprise a heavy chain (HC) sequence, light chain (LC) sequence, CDRH3 sequence, CDRH2 sequence, CDRH1 sequence, CDRL3 sequence, CDRL2 sequence, CDRL1 sequence, and/or framework sequence. In some embodiments, anti-CD3 antibodies and/or antigen-binding fragments thereof have amino acid sequence identity to corresponding sequences of anti-CD3 antibodies disclosed in Table 1 (Ab1-Ab258) by at least about 100%, at least about 99%, at least about 98%, at least about 97%, at least about 96%, at least about 95%, at least about 94%, at least about 93%, at least about 92%, at least about 91%, at least about 90%, at least about 89%, at least about 88%, at least about 87%, at least about 86%, at about 85%, at least about 84%, at least about 83%, at least about 82%, at least about 80%; and/or all percentages of identity in between. In some embodiments, percent identity is measured by any well-known algorithm of sequence identity, such as FASTA, BLAST or GAP.

In some embodiments, residue positions that are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. (See, e.g., Pearson (1994) Methods Mol. Biol. 24: 307-331). Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. In some embodiments, conservative amino acids substitution groups are: valine-leucine-isoleucine, phenylalanine-tyrosine, lysine-arginine, alanine-valine, glutamate-aspartate, and asparagine-glutamine. Alternatively, in some embodiments, a conservative replacement comprises any change having a positive value in the PAM250 log-likelihood matrix disclosed in Gonnet et al. (1992) Science 256: 1443 45. In some embodiments, a “moderately conservative” replacement comprises any change having a nonnegative value in a PAM250 log-likelihood matrix.

Substitution of one or more CDR residues or omission of one or more CDRs is also possible. Antibodies have been described in which one or two CDRs can be dispensed to alter binding in the scientific literature. Padlan et al. (1995 FASEB J. 9:133-139) analyzed contact regions between antibodies and their antigens, based on published crystal structures, and concluded that only about one fifth to one third of CDR residues actually contact their associated antigen. Padlan also found many antibodies in which one or two CDRs had zero amino acids in contact with an antigen (see also, Vajdos et al. 2002 J Mol Biol 320:415-428). CDR residues not contacting an antigen can be identified based on previous studies (for example residues H60-H65 in CDRH2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically.

In certain embodiments, substitutions, insertions, or deletions may occur within one or more CDRs of engineered pH-dependent CD3 binding antibodies described herein, so long as such alterations preserve pH sensitivity and do not substantially reduce the ability of the antibody to bind its antigen. For example, conservative alterations (e.g., conservative substitutions as provided herein) that do not substantially reduce binding affinity may be made in CDRs. Such alterations may, for example, be outside of antigen contacting residues in the CDRs. In certain embodiments of the variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of an antibody molecule include fusion to the N- or C-terminus of the antibody to an enzyme (e.g. for ADEPT) or a polypeptide which increases serum half-life of the antibody.

As described throughout, anti-CD3 antibodies and/or antigen-binding fragments thereof as provided herein possess favorable developability and are, thus, relatively developable.

The term “developable” refers to the extent to which one or more polypeptides in a plurality of polypeptides possess desirable characteristics, such as, e.g., desirable expression, for example, in mammalian cells; solubility; viscosity; aggregation; chemical and/or physical stability; desirable shelf-life; melting temperature; pharmacokinetic profiles; circulation half-life; and clearance characteristics. Such characteristics may serve as indicia, independently, as combinations of sub-sets of such indicia, or in totality, for the likelihood that such one or more polypeptides may be successfully developed as a therapeutic candidate, and ultimately an approved drug. Accordingly, as understood in the art, generally, polypeptides with desirable developability characteristics possess, e.g., relatively high solubility, relatively low viscosity, relatively low propensity for aggregation, relatively high chemical stability, relatively high physical stability, relatively long shelf life, relatively high melting temperature, relatively long circulation half-life, relatively long clearance time, and the like. Polypeptides with undesirable developability characteristics possess, e.g., relatively low solubility, relatively high viscosity, relatively high propensity for aggregation, relatively poor chemical stability, relatively poor physical stability, relatively short shelf life, relatively low melting temperature, relatively short circulation half-life, relatively short clearance time, and the like.

Methods and assays that may be employed to ascertain the degree to which polypeptides, such as anti-CD3 antibodies and/or antigen-binding fragments thereof as described herein, possess desirable developability characteristics are available in the art, and include, for example; PSR assays (WO 2014/179363 and Xu et al., Protein Eng Des Sel, Vol. 26, pages 663-670 (2013)); SMP and SCP assays and the like; cross interaction chromatography (CIC); self-interaction chromatography (SIC); dynamic light scattering; size exclusion chromatography (SEC), dynamic light scattering (DLS) spectroscopy; photon correlation spectroscopy; quasi-elastic light scattering, circular dichroism (CD), viscosity measurements; whole cell binding; tissue micro array methodologies; BVP ELISA assays; AC-SINS assays (Liu et al; MAbs, Vol. 6, pages 483-492 (2014); differential scanning calorimetry; and the like (see, e.g., He et al., J. Pharm. Sci., Vol. 100(4), pp. 1330-1340 (2011); Wagner et al., Pharm. Develop. & Technol (posted online 2012; hyper-text transfer protocol: informahealthcare.com/doi/abs/10.3109/10837450.2011.649851); Hotzel et al., MAbs, Vol. 4(6), pages 753-7601 (2012); Weiqiang et al., J. Pharm. Sci., Vol. 101(5), pp. 1701-1720 (2012); Banks et al., J. Pharm. Sci., Vol. 101(8), pp. 2720-2732 (2012); Lie et al., J. Pharm. Sci., Vol. 94(9), pp. 1928-1948 (2005); and Payne et al., Biopolymers, Vol. 85(5), pp. 527-533 (2006)).

In some embodiments, antibodies that are identified as possessing decreased developability are so detected by virtue of their interaction with a polyspecificity reagent (“PSR”) and, as such, are referred to as “polyspecific” polypeptides. Such polyspecific antibodies may be referred to as relatively “undevelopable” or relatively “non-developable”.

A “developability profile” refers to an index that may be assigned to antibodies upon assessing their developability. A developability profile is a measure or metric by which developability of anti-CD3 antibodies may be assessed, compared, and/or ranked. Such developability profiles serve as a measure of the degree of interaction of CD3 binders and antibodies comprising them. The degree of interaction may be assessed by any number of means available in the art that provides an output value that correlates with a strength or affinity of a polypeptide for a moiety to which it is bound. Exemplary means include flow cytometry means, such as FACS; ELISA; quantitative immunoaffinity assays or immunoprecipitation assays; mammalian two-hybrid or yeast two-hybrid assays, and the like. In the context of FACS, as demonstrated in the Examples, a degree of interaction between polypeptides in the plurality and the PSR may be ascertained by generating a mean fluorescence intensity (MFI) for each polypeptide-PSR interaction that is detected, and then ordering the MFI in either ascending or descending order, thereby ranking the polypeptides in the plurality according to the relative degree of interaction between each detected polypeptide and the PSR. Such a ranking provides for a ranking of polypeptides of the plurality such that those polypeptides possessing enhanced developability are readily ascertained, as are those polypeptides possessing decreased developability.

A developability profile may also take the form of a normalized score, for example, by normalizing developability of anti-CD3 antibodies described herein to the developability of a standard (or control) antibody, e.g., anti-HEL antibody.

In certain embodiments, inventive engineered pH-dependent CD3 binding domains and antibodies comprising them may be further modified to contain additional nonproteinaceous moieties that are known in the art and are readily available. Moieties suitable for derivatization of an antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In certain embodiments, engineered pH-dependent CD3 binding domains and antibodies comprising them display an enhanced developability profile. The developability profile for anti-CD3 antibodies is obtained by performing one or more of a PSR assay; an SCP assay; AC-SINS; an ELISA; a DSF assay; a Tm assay; a HIC assay; a CIC assay; or combinations thereof.

In other embodiments, anti-CD3 antibodies and/or antigen-binding fragments thereof as described herein display a poly-specificity reagent (PSR) score of between about 0.0 and about 0.45. In some embodiments, the PSR is between about 0.0 and about 0.4. In some embodiments, the PSR is between about 0.0 and about 0.35. In some embodiments, the PSR is between about 0.0 and about 0.3. In some embodiments, the PSR is between about 0.0 and about 0.25. In some embodiments, the PSR is between about 0.0 and about 0.2. In some embodiments, the PSR is between about 0.0 and about 0.15. In some embodiments, the PSR is between about 0.0 and about 0.1. In some embodiments, a score of 0.0-0.1 is “clean PSR”. In some embodiments, a score of 0.1 to 0.33 is “low PSR”. In some embodiments, a score of 0.33 to 0.66 is “medium PSR”. In some embodiments, a score of 0.66-1.00 is “high PSR”. In some embodiments, a high PSR score is indicative of decreased (or poor) developability. Generally, the lower the PSR score the more favorable the developability of the antibody.

In still other embodiments, anti-CD3 antibodies or antigen-binding fragment thereof as described herein display an HIC score of less than about 10.5 minutes (a clean to low HIC score). In some embodiments, an HIC score is between about 10.5 minutes and 11.5 minutes (a medium HIC score). In some embodiments, an HIC score is greater than about 11.5 minutes (a high HIC score). Generally, the lower the HIC score the more favorable the developability of the antibody.

In yet other embodiments, anti-CD3 antibodies and/or antigen-binding fragments thereof as described herein display an SEC score of less than about 95%, which indicates that the antibody is a monomer, i.e., not aggregating.

In still other embodiments, anti-CD3 antibodies and/or antigen-binding fragments thereof as described herein display a Tm of less than about 65° C.

In some embodiments, anti-CD3 antibodies and/or antigen-binding fragments thereof as described herein may be further modified to minimize effector function, e.g., a silent Fc.

“Effector function” refers to biological activities attributable to the Fc region of an antibody, which varies by antibody isotype. Exemplary effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

“Fc region” is a C-terminal region of an immunoglobulin heavy chain that contains at least a portion of the constant region, including native sequence Fc regions and variant Fc regions. A human IgG heavy chain Fc region can extend from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

In certain embodiments, one or more amino acid modifications may be introduced into the Fc region of an anti-CD3 antibody of the disclosure, thereby generating an Fc region variant (see, e.g., US 2012/0251531). An Fc region variant may comprise a human Fc region sequence (e.g., a human IgG1, IgG2, IgG3 or IgG4 Fc region) comprising an amino acid modification (e.g., a substitution) at one or more amino acid positions.

In certain embodiments, the disclosure contemplates an anti-CD3 antibody variant that possesses some but not all effector functions, which make it a desirable candidate for applications in which the half-life of an antibody in vivo is important yet certain effector functions (such as complement and ADCC) are unnecessary or deleterious. In vitro and/or in vivo cytotoxicity assays can be conducted to confirm reduction/depletion of CDC and/or ADCC activities. For example, Fc receptor (FcR) binding assays can be conducted to ensure that an antibody lacks FcγR binding (hence likely lacking ADCC activity) but retains FcRn binding ability. The primary cells for mediating ADCC (e.g. NK cells), express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see, Bruggemann, M. et al., J. Exp. Med. 166:1351-1361 (1987)). Alternatively, non-radioactive assay methods may be employed (see, for example, ACTI™ non-radioactive cytotoxicity assay for flow cytometry (Cell Technology, Inc. Mountain View, Calif.); and CytoTox 96® non-radioactive cytotoxicity assay (Promega, Madison, Wis.)). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that an antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al. J. Immunol Methods 202:163 (1996); Cragg, M. S. et al. Blood. 101:1045-1052 (2003); and Cragg, M. S. and M. J. Glennie Blood. 103:2738-2743 (2004)). FcRn binding and in vivo clearance/half-life determinations can also be performed using methods known in the art (see, e.g., Petkova, S. B. et al. Int'l. Immunol 18(12):1759-1769 (2006)).

In some embodiments, antibodies with reduced effector function include those with substitution of one or more of Fc region residues 238, 265, 269, 270, 297, 327 and 329 (U.S. Pat. Nos. 6,737,056 and 8,219,149). In some embodiments, Fc mutants include Fc mutants with substitutions at two or more of amino acid positions 265, 269, 270, 297 and 327, including the so-called “DANA” Fc mutant with substitution of residues 265 and 297 to alanine (U.S. Pat. Nos. 7,332,581 and 8,219,149).

In other embodiments, anti-CD3 antibodies and/or antigen-binding fragments thereof as described herein are further modified to include a masking agent, e.g., a polypeptide mask, attached via a cleavable linker.

In some embodiments, anti-CD3 antibodies and/or antigen-binding fragments thereof as described herein are conjugated to a therapeutic moiety thereby forming an immunoconjugate. An “immunoconjugate” is an antibody conjugated to one or more heterologous molecule(s) such as, e.g., an antibiotic, a second anti-CD3 antibody, a vaccine, or a toxoid, or any other therapeutic moiety.

In certain embodiments, anti-CD3 antibodies and/or antigen-binding fragments thereof as described herein are altered to increase or decrease the extent to which the antibody is glycosylated. Addition or deletion of glycosylation sites to an anti-CD3 antibody of the disclosure may be conveniently accomplished by altering the amino acid sequence such that one or more glycosylation sites is created or removed.

### Production of the Anti-CD3 Antibodies and Antigen-Binding Fragments Thereof

Anti-CD3 antibodies and/or antigen-binding fragments thereof may be produced using recombinant methods. For example, isolated nucleic acids encoding an anti-CD3 antibody as described herein is provided. Such nucleic acids may encode an amino acid sequence comprising the VL, and/or an amino acid sequence comprising the VH of the antibody (e.g., the light and/or heavy chains of the antibody). In a further embodiment, one or more vectors (e.g., expression vectors) comprising such nucleic acids are provided. In a further embodiment, a host cell comprising such nucleic acids is provided. In one such embodiment, a host cell comprises (e.g., has been transformed with): (1) a vector comprising a nucleic acid sequence that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti-CD3 antibody is provided, wherein the method comprises culturing a host cell comprising a nucleic acid encoding the antibody, as provided above, under conditions suitable for expression of the antibody, and optionally recovering the antibody from the host cell (or host cell culture medium).

The term “host cell” refers to cells into which an exogenous nucleic acid sequence has been introduced, including the progeny of such cells. Host cells include transformants and transformed cells, which include the primary transformed cell and progeny derived therefrom without regard to the number of passages.

For recombinant production of an anti-CD3 antibody, nucleic acids encoding an antibody, e.g., as described above, is isolated and inserted into one or more vectors for further cloning and/or expression in a host cell. Such nucleic acids may be readily isolated and sequenced using conventional procedures (e.g., by using oligonucleotide probes that are capable of binding specifically to genes encoding the heavy and light chains of the antibody).

Suitable host cells for cloning and/or expression of antibody-encoding vectors include prokaryotic or eukaryotic cells. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also, Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified. In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See, e.g., Gerngross, Nat. Biotech. 22:1409-1414 (2004), and Li et al., Nat. Biotech. 24:210-215 (2006); WO 2009/036379; WO 2010/105256; and WO 2012/009568.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N. Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp²/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

Anti-CD3 antibodies and/or antigen-binding fragments thereof may be identified, screened for, selected for or characterized for their physical/chemical properties and/or biological activities by various assays known in the art, e.g., ELISA, Western blot, etc. or competition assays may be used to identify an antibody that competes with an anti-CD3 antibody of the disclosure for binding to CD3. In an exemplary competition assay, immobilized CD3 is incubated in a solution comprising a first labeled antibody that binds to CD3 and a second unlabeled antibody that is being tested for its ability to compete with the first antibody for binding to CD3. The second antibody may be present in a hybridoma supernatant. As a control, immobilized CD3 is incubated in a solution comprising the first labeled antibody but not the second unlabeled antibody. After incubation under conditions permissive for binding of the first antibody to CD3, excess unbound antibody is removed, and the amount of label associated with immobilized CD3 is measured. If the amount of label associated with immobilized CD3 is substantially reduced in the test sample relative to the control sample, then that indicates that the second antibody is competing with the first antibody for binding to CD3. See, e.g., Harlow and Lane (1988) Antibodies: A Laboratory Manual. Ch. 14 (Cold Spring Harbor Laboratory, Cold Spring Harbor, N.Y.).

Anti-CD3 antibodies and/or antigen-binding fragments thereof possessing biological activity may be identified using standard approaches. Biological activity may include, e.g., binding to CD3 on the surface of a T cell either in vivo, in vitro, or ex vivo. In the case of a multispecific anti-CD3 antibody (such as a bispecific antibody with one arm that binds to CD3 and another arm that binds to a different target, e.g., a cell surface antigen, e.g., a tumor antigen), biological activity may also include effector cell activation (such as CD8+ and/or CD4+ T cell) activation), effector cell population expansion (i.e., an increase in T cell count), target cell population reduction (i.e., a decrease in the population of cells expressing the second biological molecule on their cell surfaces), and/or target cell killing.

### Diagnostic and Therapeutic Uses for the Anti-CD3 Antibodies and Antigen-Binding Fragments Thereof

Anti-CD3 antibodies and/or antigen-binding fragments described herein may be used for diagnosis and/or detection. “Detection” as used herein encompasses quantitative or qualitative detection.

In certain embodiments, labeled anti-CD3 antibodies are provided. Anti-CD3 antibodies and/or antigen-binding fragments thereof as described herein may include a label or moiety that is detected directly (such as fluorescent, chromophoric, electron-dense, chemiluminescent, and radioactive labels or indirectly (such as enzymes or ligands). Non-limiting exemplary labels include, radioisotopes such as 32P, 14C, 125I, 3H, and 131I; fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase; heterocyclic oxidases such as uricase and xanthine oxidase, coupled with an enzyme that employs hydrogen peroxide to oxidize a dye precursor such as HRP, lactoperoxidase, or microperoxidase; biotin/avidin; spin labels; bacteriophage labels; stable free radicals; and the like.

CD3 antibodies and/or antigen-binding fragments thereof as described herein, as well as pharmaceutical compositions of such antibodies, may be used in therapeutic methods. In one embodiment, anti-CD3 antibodies and/or antigen-binding fragments thereof as described herein or pharmaceutical compositions comprising such antibodies may be used for treating or delaying progression of a cell proliferative disorder or an autoimmune disorder. In some embodiments, the anti-CD3 antibodies and antigen-binding fragments thereof may be used in treating cancers. Tumor cells typically have an extracellular pH of around about 6.3-6.5; the anti-CD3 antibodies and antigen-binding fragments described herein promote preferential CD3 binding at low(er) pH values, e.g., around pH 6, and thereby promote binding and activity in and around the tumor microenvironment. In some embodiments, use of the anti-CD antibodies and antigen-binding fragments thereof may result in selective and sustained cytotoxic activity at or around the tumor site, thereby reducing or eliminating off-target effects.

A “disorder” refers to any condition or disease that would benefit from treatment including, but not limited to, chronic and acute disorders or diseases including those pathological conditions which predispose a mammal to the disorder in question.

The terms “cell proliferative disorder” and “proliferative disorder” refer to disorders that are associated with some degree of abnormal cell proliferation. Cell proliferative disorders include cancer, e.g., a tumor.

“Tumor” as used herein refers to all neoplastic cell growth and proliferation, whether malignant or benign, and all pre-cancerous and cancerous cells and tissues.

“Cancer” refers to a physiological condition in mammals characterized by unregulated cell growth. Examples of cancer include, but are not limited to, carcinoma, lymphoma, blastoma, sarcoma, and leukemia or lymphoid malignancies; with more particular examples including squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer and gastrointestinal stromal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, superficial spreading melanoma, lentigo maligna melanoma, acral lentiginous melanomas, nodular melanomas, multiple myeloma and B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); hairy cell leukemia; chronic myeloblastic leukemia; and post-transplant lymphoproliferative disorder (PTLD), as well as abnormal vascular proliferation associated with phakomatoses, edema (such as that associated with brain tumors), Meigs' syndrome, brain, as well as head and neck cancer, and associated metastases. In certain embodiments, cancers that are amenable to treatment by antibodies of the disclosure include breast cancer, colorectal cancer, rectal cancer, non-small cell lung cancer, glioblastoma, non-Hodgkins lymphoma (NHL), renal cell cancer, prostate cancer, liver cancer, pancreatic cancer, soft-tissue sarcoma, kaposi's sarcoma, carcinoid carcinoma, head and neck cancer, ovarian cancer, mesothelioma, and multiple myeloma. In some embodiments, the cancer is selected from: small cell lung cancer, gliblastoma, neuroblastomas, melanoma, breast carcinoma, gastric cancer, colorectal cancer (CRC), and hepatocellular carcinoma. Yet, in some embodiments, the cancer is selected from: non-small cell lung cancer, colorectal cancer, glioblastoma and breast carcinoma, including metastatic forms of those cancers. In other embodiments, the cancer is selected from a class of mature B-Cell cancers excluding Hodgkin's Lymphoma but including germinal-center B-cell-like (GCB) DLBCL, activated B-cell-like (ABC) DLBCL, follicular lymphoma (FL), mantle cell lymphoma (MCL), acute myeloid leukemia (AML), chronic lymphoid leukemia (CLL), marginal zone lymphoma (MZL), small lymphocytic leukemia (SLL), lymphoplasmacytic lymphoma (LL), Waldenstrom macroglobulinemia (WM), central nervous system lymphoma (CNSL), Burkitt's lymphoma (BL), B-cell prolymphocytic leukemia, Splenic marginal zone lymphoma, Hairy cell leukemia, Splenic lymphoma/leukemia, unclassifiable, Splenic diffuse red pulp small B-cell lymphoma, Hairy cell leukemia variant, Waldenstrom macroglobulinemia, Heavy chain diseases, a Heavy chain disease, γ Heavy chain disease, μ Heavy chain disease, Plasma cell myeloma, Solitary plasmacytoma of bone, Extraosseous plasmacytoma, Extranodal marginal zone lymphoma of mucosa-associated lymphoid tissue (MALT lymphoma), Nodal marginal zone lymphoma, Pediatric nodal marginal zone lymphoma, Pediatric follicular lymphoma, Primary cutaneous follicle centre lymphoma, T-cell/histiocyte rich large B-cell lymphoma, Primary DLBCL of the CNS, Primary cutaneous DLBCL, leg type, EBV-positive DLBCL of the elderly, DLBCL associated with chronic inflammation, Lymphomatoid granulomatosis, Primary mediastinal (thymic) large B-cell lymphoma, Intravascular large B-cell lymphoma, ALK-positive large B-cell lymphoma, Plasmablastic lymphoma, Large B-cell lymphoma arising in HHV8-associated multicentric Castleman disease, Primary effusion lymphoma: B-cell lymphoma, unclassifiable, with features intermediate between diffuse large B-cell lymphoma and Burkitt lymphoma, and B-cell lymphoma, unclassifiable, with features intermediate between diffuse large B-cell lymphoma and classical Hodgkin lymphoma.

As used herein, “treatment” or “treat” or “treating” refer to clinical intervention in an attempt to alter the natural course of an individual being treated and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the terms “prevent,” “preventing,” and “prevention” refer to the prevention or inhibition of the development or onset of a disorder or disease.

As used herein, the terms “ameliorate” and “alleviate” refer to a reduction or diminishment in the severity a condition or any symptoms thereof.

In some embodiments, antibodies of the disclosure are used to delay development of a disorder or disease or to delay the progression of a disorder or disease. As used herein, “delaying progression” of a disorder or disease means to defer, hinder, slow, retard, stabilize, and/or postpone development of the disease or disorder (e.g., a cell proliferative disorder, e.g., cancer). The delay can be of varying lengths of time, depending on the history of the disease and/or individual being treated.

An effective amount of such antibody or composition may be administered to an individual suffering from cancer or arthritis, rheumatoid arthritis, colitis, inflammatory bowel disease, autoimmune type I diabetes, etc. An “effective amount” of an anti-CD3 antibody disclosed herein or a composition (e.g., pharmaceutical composition) comprising such antibody, is at least the minimum amount required to achieve the desired therapeutic or prophylactic result, e.g., a measurable improvement or prevention of a particular disorder, e.g., a cell proliferative disorder, e.g., cancer, preferably with minimal or no toxic or detrimental effects. An effective amount may vary according to inter alia disease state, age, sex, and weight of the patient, and the ability of the antibody (or antigen-binding fragment thereof) to elicit a desired response in the individual and, in some instances, by co-administering one or more additional therapeutic agents.

In some embodiments, anti-CD3 antibodies and/or antigen-binding fragments thereof as described herein may be used to enhance immune function in an individual having a cell proliferative disorder or an autoimmune disorder. Following administration, such antibody or composition may enhance immune function in an individual having a cell proliferative disorder or an autoimmune disorder by activating effector cells (e.g., T cells, e.g., CD8+ and/or CD4+ T cells including Tregs), expanding (increasing) the effector cell population, reducing the population of target cells (e.g., a cell expressing a second biological molecule recognized by an anti-CD3 antibody of the disclosure, such as a bispecific antibody), and/or killing a target cell (e.g., target tumor cell).

Anti-CD3 antibodies and/or antigen-binding fragments thereof as disclosed herein may be used to treat disorders including, but not limited to, a proliferative disorder, an oncological disorder, an immune-oncological disorder, a neurological disorder, a cognitive disorder, a neurodegenerative disorder, an autoimmune disorder. In one embodiment, an effective amount of such anti-CD3 antibody may be administered, alone or in combination with at least one additional agent, to an individual having such disorder. Such “individual” may be a mammal and, in particular, a human.

Non-limiting exemplary additional therapeutic agents include a chemotherapy agent, an antibody-drug conjugate (ADC), and/or a biological modifier. Chemotherapy agents may be selected from cyclophosphamide, doxorubicin, vincristine, and prednisolone (CHOP). ADC may be selected from an anti-CD79b antibody drug conjugate (such as anti-CD79b-MC-vc-PAB-MMAE or the anti-CD79b antibody drug conjugate described in any one of U.S. Pat. No. 8,088,378 and/or US 2014/0030280, or polatuzumab vedotin), an anti-CD19 antibody drug conjugate, an anti-CD22 antibody drug conjugate, an anti-CD45 antibody drug conjugate, and an anti-CD32 drug conjugate. A biological modifier may be selected from a BCL-2 inhibitor (such as GDC-0199/ABT-199), lenalidomide (Revlimid®), a PI3K-delta inhibitor (such as idelalisib (Zydelig®)), a PD-1 axis binding antagonist, an agonist, e.g., agonist antibody, directed against an activating co-stimulatory molecule, e.g., CD40, CD226, CD28, OX40 (e.g., AgonOX), GITR, CD137 (also known as TNFRSF9, 4-1 BB, or ILA), CD27 (e.g., CDX-1127), HVEM, or CD127, an antagonist, e.g., antagonist antibody, directed against an inhibitory co-stimulatory molecule, e.g., CTLA-4 (also known as CD152), PD-1, TIM-3, BTLA, VISTA, LAG-3, B7-H3, B7-H4, IDO (e.g., 1-methyl-D-tryptophan (also known as 1-D-MT)), TIGIT, MICA/B, GITR (e.g., TRX518) or arginase, ipilimumab (also known as MDX-010, MDX-101, or Yervoy®), tremelimumab (also known as ticilimumab or CP-675,206, urelumab (also known as BMS-663513), MGA271, an antagonist directed against a TGF beta, e.g., metelimumab (also known as CAT-192), fresolimumab (also known as GC1008), LY2157299k, and an adoptive transfer of a T cell (e.g., a cytotoxic T cell or CTL) expressing a chimeric antigen receptor (CAR), e.g., adoptive transfer of a T cell comprising a dominant-negative TGF beta receptor, e.g, a dominant-negative TGF beta type II receptor.

Anti-CD3 antibodies and/or antigen-binding fragments thereof as disclosed herein may be used to enhancing immune function in an individual, e.g., a human, having a disorder in an individual having such disorder. In one embodiment, a method of enhancing immune function comprises administering to an individual an effective amount of an anti-CD3 antibody to activate effector cells (e.g., T cells, e.g., CD8+ and/or CD4+ T cells), expand (increase) an effector cell population, reduce a target cell population, and/or kill a target cell (e.g., target tumor cell).

In a further aspect, pharmaceutical formulations comprising anti-CD3 antibodies and/or antigen-binding fragments as described herein are also provided, e.g., for use in any of the above therapeutic methods. A “pharmaceutical formulation” refers to a preparation in such form as to permit the biological activity of an active ingredient contained therein, such as the anti-CD3 antibodies described herein, to be effective and which preferably contains no additional components which are unacceptably toxic to a subject to which the formulation would be administered.

In one embodiment, a pharmaceutical formulation comprises any of the anti-CD3 antibodies disclosed herein and a pharmaceutically acceptable carrier. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In another embodiment, a pharmaceutical formulation comprises any of the anti-CD3 antibodies provided herein and at least one additional therapeutic agent.

Antibodies of the disclosure can be used either alone or in combination with other agents in a therapy, e.g., an anti-CD3 antibody and/or antigen-binding fragment thereof may be co-administered with at least one additional therapeutic agent. In certain embodiments, an additional therapeutic agent is a chemotherapeutic agent, growth inhibitory agent, cytotoxic agent, agent used in radiation therapy, anti-angiogenesis agent, apoptotic agent, anti-tubulin agent, or other agent, such as a epidermal growth factor receptor (EGFR) antagonist (e.g., a tyrosine kinase inhibitor), HER1/EGFR inhibitor (e.g., erlotinib (Tarceva™)), platelet derived growth factor inhibitor (e.g., Gleevec™ (Imatinib Mesylate)), a COX-2 inhibitor (e.g., celecoxib), interferon, cytokine, antibody other than the anti-CD3 antibody of the disclosure, such as an antibody that bind to one or more of the following targets ErbB2, ErbB3, ErbB4, PDGFR-beta, BIyS, APRIL, BCMA VEGF, or VEGF receptor(s), TRAIL/Apo2, PD-1, PD-L1, PD-L2, or another bioactive or organic chemical agent.

In some embodiments, the disclosure provides a method wherein the additional therapeutic agent is a glucocorticoid. In one embodiment, the glucocorticoid is dexamethasone.

Such combination therapies noted above encompass combined administration (where two or more therapeutic agents are included in the same or separate formulations), and separate administration, in which case, administration of the antibody of the disclosure can occur prior to, simultaneously, and/or following, administration of additional therapeutic agent or agents. In one embodiment, administration of the anti-CD3 antibody and administration of an additional therapeutic agent occur within about one month, or within about one, two or three weeks, or within about one, two, three, four, five, or six days, of each other. Anti-CD3 antibodies of the disclosure (e.g., bispecific anti-CD3 antibodies of the disclosure that bind to CD3 and a second biological molecule, e.g., a cell surface antigen, e.g., a tumor antigen, such as a TDB antibody of the disclosure or variant thereof) can also be used in combination with radiation therapy.

An antibody of the disclosure (and/or any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. In some embodiments, the antibody is administered by subcutaneous administration. In some embodiments, an anti-CD3 antibody administered by subcutaneous injection exhibits a less toxic response in a patient than the same anti-CD3 antibody administered by intravenous injection. Dosing can be by any suitable route, for example, by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies of the disclosure would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not, but may optionally be, formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the disclosure (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments.

As a general proposition, a therapeutically effective amount of the anti-CD3 antibody administered to human will be in the range of about 0.01 to about 100 mg/kg of patient body weight whether by one or more administrations. In some embodiments, an antibody used is administered in about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg daily, for example. In one embodiment, an anti-CD3 antibody described herein is administered to a human at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg, or 10 mg/kg (or any combination thereof) may be administered to the patient. Such doses may be administered intermittently, for example, every week or every three weeks (e.g., such that the patient receives from about two to about twenty, or, for example, about six doses of the anti-CD3 antibody). An initial higher loading dose, followed by one or more lower doses, may be administered. The progress of this therapy is easily monitored by conventional techniques and assays.

In some embodiments, methods of the disclosure may further comprise an additional therapy. The additional therapy may be radiation therapy, surgery, chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy. In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy may be a separate administration of one or more of the therapeutic agents described above.

In another aspect of the disclosure, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the disclosure. The label or package insert indicates that the composition is used for treating the condition of choice. Moreover, the article of manufacture may comprise (a) a first container with a composition contained therein, wherein the composition comprises an antibody of the disclosure; and (b) a second container with a composition contained therein, wherein the composition comprises a further cytotoxic or otherwise therapeutic agent. The article of manufacture in this embodiment of the disclosure may further comprise a package insert indicating that the compositions can be used to treat a particular condition. Alternatively, or additionally, the article of manufacture may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Accordingly, manufacture and/or preparation of a pharmaceutical composition comprising anti-CD3 antibodies and/or antigen-binding fragments as disclosed herein is also contemplated. The composition may be used alone or in combination with other active agents to treat a cell proliferative disorder (e.g., cancer) or an autoimmune disorder (e.g., arthritis, rheumatoid arthritis, colitis, inflammatory bowel disease, autoimmune type I diabetes, etc.).

In some embodiments, pharmaceutical compositions comprising anti-CD3 antibodies and/or antigen-binding fragments thereof as described herein are prepared, e.g., by mixing such antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions, optionally prepared for modified (e.g., sustained) release. Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968.

Such formulations may contain more than one active ingredient as necessary for the particular indication being treated, preferably those with complementary activities that do not adversely affect each other and present in amounts that are effective for the purpose intended. For example, it may be desirable to further provide an additional therapeutic agent (e.g., a chemotherapeutic agent, a cytotoxic agent, a growth inhibitory agent, and/or an anti-hormonal agent).

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

### EXAMPLES

### Example 1: Construction of Engineered pH-Dependent CD3 Libraries

Combinatorial histidine substitution libraries were derived from parental anti-CD3 antibody clone ADI-26906 (Antibody No. 1 of Table 1). ADI-26906 was initially disclosed in PCT/US2018/031705, which is hereby incorporated by reference herein in its entirety (ADI-26906 was not pH-engineered). Three library designs were utilized to incorporate histidines: 1) H3+L3 jumping double plus L1 single or double histidine (His) substitutions (with and without NNK variegation adjacent to His), resulting in a theoretical diversity of 3.4×10⁵; 2) pre-made HT/H2 diversity library plus H3 jumping doublet, resulting in a theoretical diversity of 6.8×10⁸, and 3) H3+L3 NNK/His or His/NNK walking singlet, resulting a theoretical diversity of 1.2×10⁵. The libraries were generated and propagated as described previously (see, e.g., WO2009036379; WO2010105256; WO2012009568; Xu et al., Protein Eng Des Sel. 2013 October; 26(10):663-70). L1 designs were synthesized as full VKs using SGI BioXp (SGI-DNA, La Jolla, Calif.). Substitutions were restricted to the CDRs. However, it is contemplated that substitutions may also be designed into the FRs. Sequence analysis of variants from each library showed a total of 0-6 His substitutions per variant VH or VK.

Five rounds of selections were performed using three libraries against biotinylated CD3 antigen. For the first round of selection for the H1/H2 plus H3 library, a magnetic bead sorting technique utilizing the Miltenyi MACS system was performed, essentially as described (Siegel et al., J Immunol Methods. 2004 March; 286(1-2):141-53). Briefly, ˜10⁹ yeast cells were incubated with 1 mL of 100 nM biotinylated CD3 antigen at pH 6.0 for 15 minutes at room temperature in FACS wash buffer PBS with 0.1% BSA at pH 6.0. After washing once with 50 mL ice-cold wash buffer, the cell pellet was resuspended in 40 mL wash buffer, and 500 μl Streptavidin MicroBeads (Miltenyi Biotec, Bergisch Gladbach, Germany. Cat #130-048-101) were added to the yeast and incubated for 15 minutes at 4° C. Next, the yeast were pelleted, resuspended in 5 ml wash buffer, and loaded onto a MACS LS column (Miltenyi Biotec, Bergisch Gladbach, Germany. Cat. #130-042-401). After the 5 mL was loaded, the column was washed three times with 3 ml FACS wash buffer. The column was then removed from the magnetic field, and the yeast were eluted with 5 mL of growth media and then grown overnight. For the two lower diversity libraries, the first round of selection was performed using flow cytometry (FACS). Briefly, yeast cells (˜109 yeast cells/library) were incubated with 0.25 mL of 100 nM biotinylated CD3 antigen at pH 6.0 for 15 minutes at room temperature in FACS wash buffer PBS with 0.1% BSA at pH 6.0. Yeast were washed with FACS buffer and labeled for sorting.

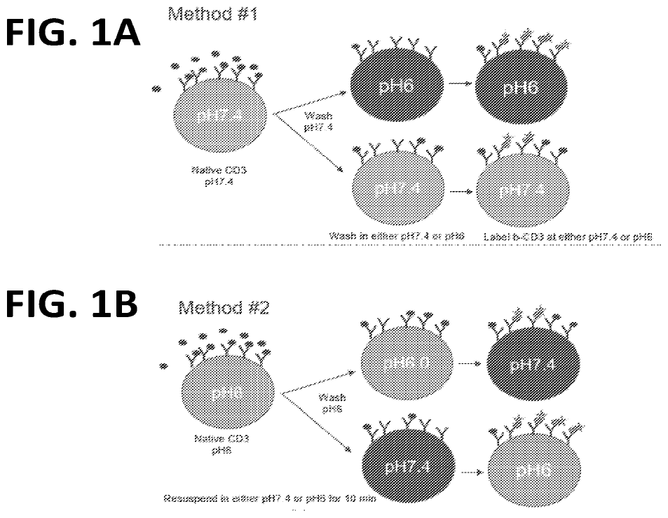

Subsequent to the first round of MACS or FACS, four rounds of sorting were performed using FACS and pH toggling selection methods (see, FIG. 1).

Purified CD3 protein antigen was biotinylated using the EZ-Link Sulfo-NHS-Biotinylation Kit (Thermo Scientific). CD3 antigens were concentrated to ˜1 mg/mL and buffer exchanged into PBS before addition of 1:7.5 molar ratio biotinylation reagent (EZ-Link Sulfo-NHS-Biotinylation Kit, Thermo Scientific, Cat #21425.). The mixture was held at 4° C. overnight prior to another buffer exchange to remove free biotin in the solution. Biotinylation was confirmed through Streptavidin sensor binding of the labeled proteins on a ForteBio. Successful biotinylation of the CD3 protein antigen was confirmed via detectable binding to a streptavidin-linked biosensor installed on ForteBio Octet™ Red384 Interferometer (Pall ForteBio, Menlo Park, Calif.) according to the manufacturer's guidelines (data not shown). In CD3 Pre-saturation Method #1 (shown in FIG. 1A), yeast cells were pre-saturated with native (un-biotinylated) CD3 antigen at pH 7.4 for 10 minutes. Next, yeast cells were washed at pH 7.4 and incubated in pH 6.0 media for 10 minutes to allow dissociation of antigen. Control cells were washed and incubated at pH 7.4. Lastly, yeast cells were incubated with biotinylated CD3 antigen (shown as a starred green circle in FIG. 1A) at pH 6 for 10 minutes. Control cells were incubated with biotinylated CD3 antigen at pH 7.4. Binders labeled at pH 6 were then sorted and characterized. In CD3 Pre-saturation Method #2 (shown in FIG. 1B), yeast cells were pre-saturated with native CD3 antigen at pH 6.0 for 10 minutes. Next, yeast cells were washed at pH 6.0 and incubated at either pH 7.4 or pH 6.0 for 10 minutes. Lastly, yeast cells were incubated with biotinylated CD3 antigen at the opposite pH (cells incubated at pH 6.0 in the previous step were incubated at pH 7.4, whereas cells incubated at pH 7.4 in the previous step were incubated at pH 6.0) for 10 minutes. Binders labeled with biotinylated CD3 antigen were then sorted and characterized.

The three libraries from the initial MACS/FACS selections were taken through four rounds of FACS selections. Approximately 1×10⁸ yeast per library were pelleted, washed three times with wash buffer, and incubated with 100 nM of biotinylated CD3 antigen separately for at least 10 minutes at room temperature at pH 6.0, pH 7.4, or processed through the pre-saturation and pH toggling of Method #2 discussed above. Yeast were then washed twice and stained with goat anti-human F(ab′)₂ kappa-FITC diluted 1:100 (Southern Biotech, Birmingham, Ala., Cat #2062-02) and either streptavidin-Alexa Fluor 633 (Life Technologies, Grand Island, N.Y., Cat #S21375) diluted 1:500, or Extravidin-phycoerthyrin (Sigma-Aldrich, St Louis, Cat #E4011) diluted 1:50, secondary reagents for 15 minutes at 4° C. After washing twice with ice-cold wash buffer, the cell pellets were resuspended in 0.4 mL wash buffer and transferred to strainer-capped sort tubes. Sorting was performed using a FACS ARIA sorter (BD Biosciences) and sort gates were determined to select either CD3 binders at pH 6 or non-binders at pH 7.4. The selected populations from the first round of FACS were brought forward into the next round.

The second, third, and fourth rounds of FACS for the above selected populations involved positive sorts for binders of CD3 at pH 6.0 and negative sorts to decrease pH 7.4 binders and polyspecific reagent binders (Xu et al., Protein Eng Des Sel. 2013 October; 26(10):663-70). In the second round of FACS (R3), cells were processed through the pre-saturation and pH toggling of Method #2 (discussed above) or negatively sorted to selected non-binders at pH 7.4. In the third round of FACS (R4), ouputs from R3 were pooled with the outputs from the CD3 pre-saturation Method #2 of R2 and subjected to CD3 pre-saturation Method #1. In the final round of FACS (R5), outputs from R4 were checked for PSR reactivity and for human and cynomolgus monkey (Cyno) CD3 binding at pH 6 and 7.4. The outputs of each round were plated and isolates were selected for sequencing and characterization.

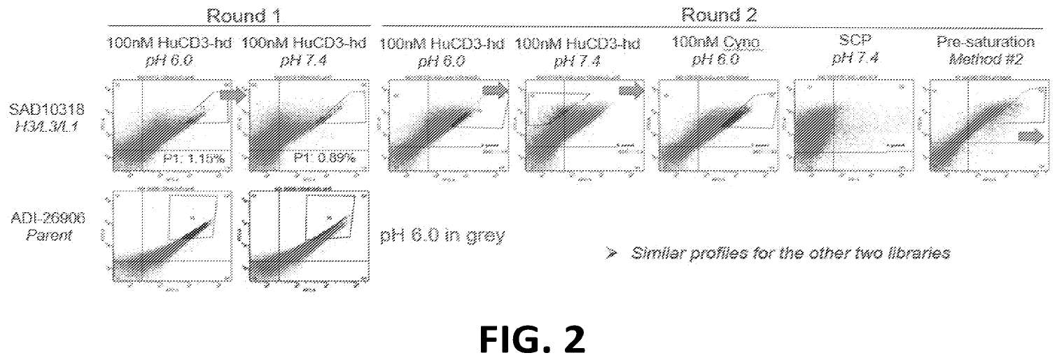

FIG. 2 shows exemplary FACS plots from Round 1 and Round 2 selections from one library. Similar binding profiles were observed for all libraries. Briefly, during Round 1 cells were positively sorted using 100 nM human CD3εδ heterodimer (HuCD3-hd) at pH 6. During Round 2, cells were positively sorted using 100 nM HuCD3-hd at pH 6.0, negatively sorted using 100 nM HuCD3-hd at pH 7.4, or pre-saturated using Method #2 described above. Binding was also confirmed for cynomolgus CD3 (CyCD3-hd) at pH 6.0. Arrows indicate sorted cells that were carried forward to the next round of sorting.

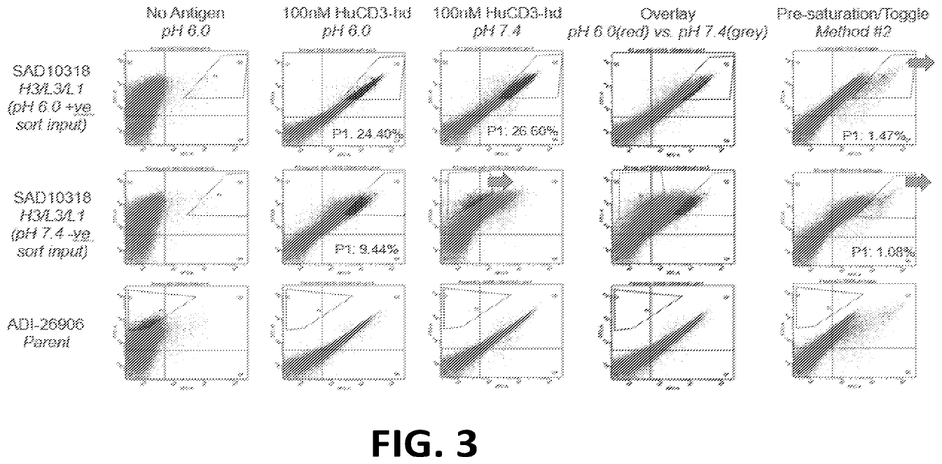

FIG. 3 shows exemplary FACS plots from Round 3 and compares inputs of pH 6.0-positive sort and pH 7.4-negative sort from Round 2. Briefly, the sorts from Round 2 were incubated with 100 nM HuCD3-hd at pH 6.0 and 7.4. The Overlay column shows that the input cell population (from Round 2 sorts) exhibits higher binding at pH 6.0 compared to pH 7.4. The pre-saturation/toggle Method #2 was used to carry forward cells into the next rounds of selections.

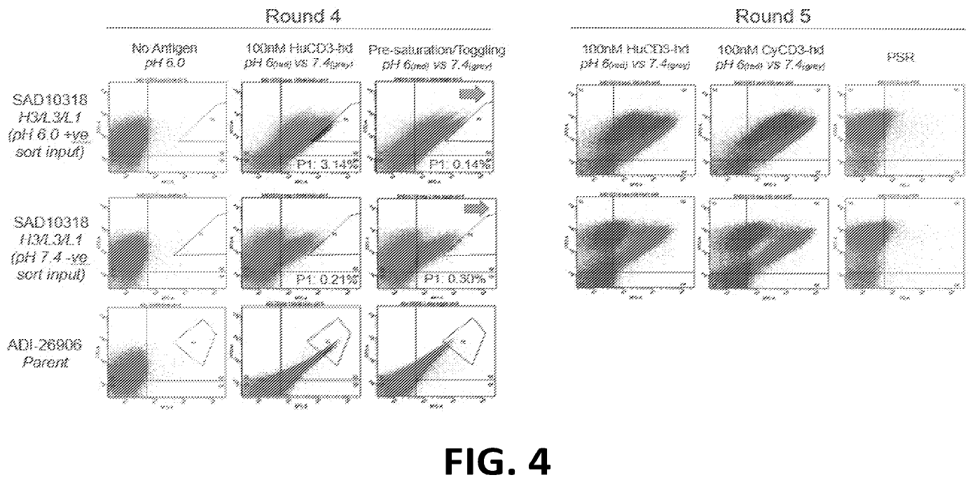

FIG. 4 shows exemplary FACS plots from Round 4 and Round 5. Round 4 compared cells incubated in 100 nM HuCD3-hd at pH 6 and pH 7.4. Round 4 also compared cells subjected to the pre-saturation/toggling method at pH 6 and pH 7.4. Round 5 compared cells incubated in either 100 nM HuCD3-hd or 100 nM CyCD3-hd at pH 6 (red) and pH 7.4 (grey).

### Example 2: Determination of Anti-CD3 Antibody Affinity to CD3

Affinity of the anti-CD3 antibodies for CD3 at pH 6.0 and 7.4 was determined by measuring their kinetic constants (kₐ, k_{d}, K_{D}) on ForteBio Octet. ForteBio affinity measurements were performed generally as previously described (Estep et al., MAbs. 2013 5(2):270-8). Briefly, ForteBio affinity measurements were performed by loading antibodies (IgGs) on-line onto AHC sensors. Sensors were equilibrated off-line in assay buffer for 30 minutes and then monitored on-line for 60 seconds for baseline establishment. For avid binding measurement, sensors with loaded IgGs were exposed to 100 nM antigen (human or cyno CD3) for 3 minutes, afterwards they were transferred to assay buffer for 3 minutes for off-rate measurement. Kinetics data were fit using a 1:1 binding model in the data analysis software provided by ForteBio. Table 2 provides kinetic constants for selected clones. Table 3 provides equilibrium dissociation constant (K_{D}) for human CD3 at pH 6.0 and 7.4 for selected clones.

Specificity of the anti-CD3 antibodies for human CD3+ Jurkat cells compared to CHO-S cells at pH 6.0 and 7.4 was determined using a FACS cell binding assay. Briefly, CD3+ human Jurkat cells and CHO-S cells were thawed and washed with cold PBSF buffer, pH 7.4 (PBS+0.1% BSA, pH 7.4). About 200,000 cells were aliquoted per well of a 96-well plate and pelleted by centrifugation (5 minutes at 500×g). The cells were washed with either PBSF pH 7.4 or PBSF pH 6.0 (PBS+0.1% BSA, pH 6.0), and then resuspended in 100 ul in either PBSF pH 7.4 or PBSF pH 6.0 with IgG antibody produced in yeast (100 nM). The mixture (cells+antibody) was incubated for 20 minutes on ice, then washed twice with either PBSF pH 7.4 or PBSF pH 6.0. Cells were resuspended in 50 ul of propidium iodide (1:500 dilution) and anti-human IgG-RPE (1:100 dilution) prepared in either PBSF pH 7.4 or PBSF pH 6.0, then incubated for 20 minutes on ice in the dark before cells were washed twice with either PBSF pH 7.4 or PBSF pH 6.0. Binding was analyzed on FACS Canto II. Mean fluorescence intensities (MFI) at pH 6.0 and 7.4 are shown in Table 3 for selected clones.

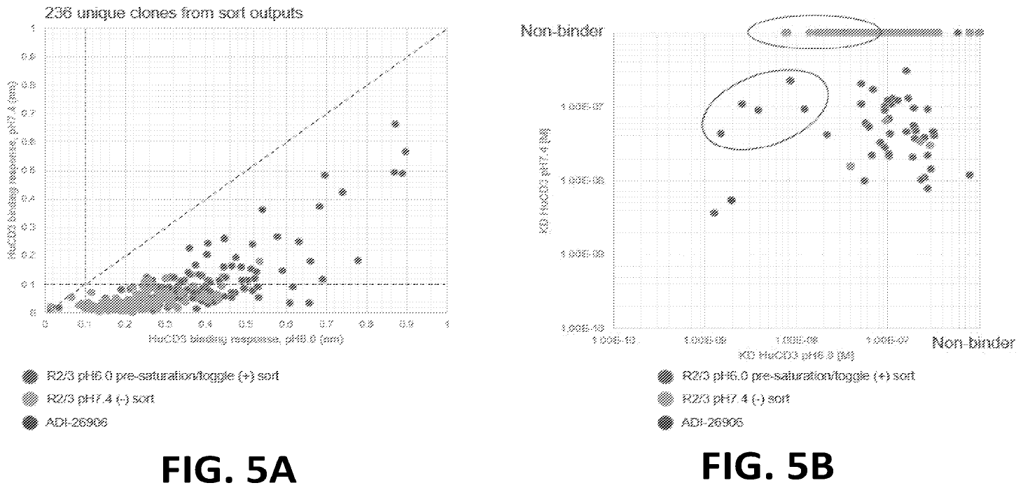

FIG. 5A shows HuCD3 binding response at pH 6 (x-axis) compared to HuCD3 binding response at pH 7.4 (y-axis) for 236 unique clones from Round 2/3 sort outputs. FIG. 5B shows K_{D} values for HuCD3 at pH 6 (x-axis) compared to HuCD3 at pH 7.4 (y-axis) for the 236 unique clones from Round 2/3 sort outputs. Blue circles represent the Round 2/3 clones obtained via the pH 6.0 pre-saturation/toggle sort, yellow circles represent the Round 2/3 clones obtained via the pH 7.4 negative sort, and the red circles represent the parent clone ADI-26906. Results show that the pH 7.4 negative sorts at Round 2/3 tends to yield more pH selective binders but with weaker pH 6.0 response or affinity, which are designated as Group 2 binders. Positive selections at pH 6.0 and the pre-saturation/toggle sort yielded clones with a mix of selectivity but with higher response/affinity, which are designated as Group 1 binders.

Group 1 binders may include, e.g., ADI-48592 (Ab125), ADI-48595 (Ab178), ADI-48650 (Ab77), ADI-48652 (Ab81), ADI-48662 (Ab116), and ADI-48666 (Ab177). Group 2 binders may include, e.g., ADI-48588 (Ab58), ADI-48587 (Ab36), ADI-48577 (Ab193), ADI-48590 (Ab91), ADI-48581 (Ab237), ADI-48575 (Ab113), ADI-48593 (Ab158), ADI-48591 (Ab102), ADI-48647 (Ab65), ADI-48636 (Ab230), ADI-48586 (Ab25), ADI-48646 (Ab53), ADI-48638 (Ab22), ADI-48597 (Ab180), ADI-48601 (Ab191), ADI-48576 (Ab182), ADI-48643 (Ab46), ADI-48624 (Ab241), ADI-48632 (Ab15), ADI-48635 (Ab17), and ADI-48645 (Ab49).

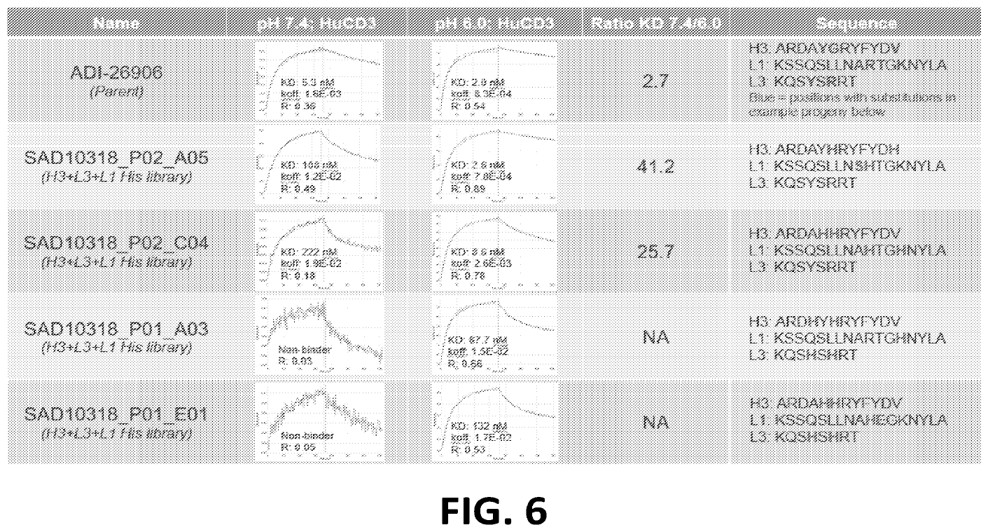

FIG. 6 provides exemplary kinetics from the ForteBio experiments for four clones compared to parent clone ADI-26906. The K_{D} for each clone was calculated at pH 7.4 and pH 6.0. A ratio K_{D} was obtained by dividing the K_{D} at pH 7.4 by the K_{D} at pH 6.0. The examples demonstrate that some clones designated as Group 1 binders, such as SAD10318_P02_A05 (ADI-48595) and SAD10318_P02_C04 (ADI-48592), bound stronger (with a lower K_{D}) at pH 6.0 compared to pH 7.4. Some clones designated as Group 2 binders, such as SAD10318_P01_A03 (ADI-48587) and SAD10318_P01_E01 (ADI-48577), were non-binders at pH 7.4 but bound at pH 6.0. Amino acid substitutions in the CDRH3, CDRL1, and CDRL3 regions that may contribute to the differential binding are highlighted the Sequence column of FIG. 6 (SEQ ID NOS 576-590, respectively, in order of appearance). Table 2 provides additional kinetics and PSR data for selected clones. Clones such as ADI-48576, ADI-48577, ADI-48587, ADI-48592, ADI-48595, ADI-48635, ADI-48650, ADI-48652, ADI-48666, ADI-48643, and ADI-48645 exhibit pH-dependent binding (with stronger binding at pH 6.0 relative to binding at pH 7.4), low PSR scores, and provide a range of affinities for CD3.

Analysis of 258 unique clones identified using methodology of the present disclosure revealed consensus motifs within CDR regions. In some embodiments, the disclosure provides an antibody comprising a CDRH3 binding domain comprising a consensus motif, the consensus motif comprising the sequence AX₁DX₂YX₃HX₄FYDV, wherein X₁ is R or H, wherein X₂ is A or H, wherein X₃ is G, H, or P, and wherein X₄ is Y, H, D, V, E, S, N, L, M, I, G, A, Q, or T (SEQ ID NO: 1). In some embodiments, at least one of X₁, X₂, X₃, and X₄ is H. The following 120 clones include this sequence motif: SAD10318_P01_A02; SAD10318_P01_G02; SAD10318_P01_D03; SAD10318_P01_G03; SAD10318_P01_H03; SAD10318_P02_D05; SAD10318_P02_H05; SAD10318_P02_G06; SAD10318_P03_C08; SAD10318_P03_H08; SAD10318_P03_G09; SAD10318_P04_H10; SAD10318_P04_D11; SAD10319_P01_A01; SAD10319_P01_C01; SAD10319_P01_E01; SAD10319_P01_A02; SAD10319_P01_C02; SAD10319_P01_F02; SAD10319_P01_H02; SAD10319_P01_B03; SAD10319_P01_C03; SAD10319_P01_D03; SAD10319_P01_F03; SAD10319_P02_A04; SAD10319_P02_C04; SAD10319_P02_E04; SAD10319_P02_F04; SAD10319_P02_A05; SAD10319_P02_B05; SAD10319_P02_C05; SAD10319_P02_G05; SAD10319_P02_A06; SAD10319_P02_B06; SAD10319_P02_C06; SAD10319_P02_D06; SAD10319_P02_F06; SAD10319_P02_G06; SAD10319_P03_C07; SAD10319_P03_H07; SAD10319_P03_D08; SAD10319_P03_E08; SAD10319_P03_E09; SAD10319_P03_F09; SAD10319_P04_A10; SAD10319_P04_G10; SAD10319_P04_E11; SAD10319_P04_F11; SAD10319_P04_G11; SAD10319_P04_C12; SAD10319_P04_D12; SAD10320_P01_B01; SAD10320_P01_D01; SAD10320_P01_E01; SAD10320_P01_G01; SAD10320_P01_H01; SAD10320_P01_A02; SAD10320_P01_F02; SAD10320_P01_G02; SAD10320_P01_H02; SAD10320_P01_C03; SAD10320_P01_D03; SAD10320_P01_E03; SAD10320_P01_F03; SAD10320_P01_G03; SAD10320_P02_A04; SAD10320_P02_B04; SAD10320_P02_E04; SAD10320_P02_H04; SAD10320_P02_A05; SAD10320_P02_B05; SAD10320_P02_C05; SAD10320_P02_B06; SAD10320_P02_D06; SAD10320_P02_E06; SAD10320_P03_B07; SAD10320_P03_H07; SAD10320_P03_C08; SAD10320_P03_D08; SAD10320_P03_F08; SAD10320_P03_H08; SAD10320_P03_A09; SAD10320_P03_C09; SAD10320_P03_D09; SAD10320_P03_F09; SAD10320_P04_A10; SAD10320_P04_C10; SAD10320_P04_D10; SAD10320_P04_E10; SAD10320_P04_G10; SAD10320_P04_D11; SAD10320_P04_E11; SAD10320_P04_F11; SAD10320_P04_H11; SAD10320_P04_A12; SAD10320_P04_D12; SAD10320_P04_E12; SAD10320_P04_F12; SAD10319_P05_A01; SAD10319_P05_A05; SAD10319_P05_B02; SAD10319_P05_C01; SAD10319_P05_C03; SAD10319_P05_C05; SAD10319_P05_D02; SAD10319_P05_D03; SAD10319_P05_D05; SAD10319_P05_E04; SAD10319_P05_F01; SAD10319_P06_B10; SAD10319_P06_B11; SAD10319_P06_C10; SAD10319_P06_C12; SAD10319_P06_E08; SAD10319_P06_F07; SAD10319_P06_F10; SAD10319_P06_G09; SAD10319_P06_H07; SAD10319_P06_H08; and SAD10319_P06_H10.

In some embodiments, the disclosure provides an antibody comprising a CDRH3 binding domain comprising a consensus motif, the consensus motif comprising the sequence ARDX₁YGX₂X₃X₄YDX₅ wherein X₁ is A or H, wherein X₂ is R or H, wherein X₃ is H or Y, wherein X₄ is F or H, and wherein X₅ is H or V (SEQ ID NO: 2). In some embodiments, at least one of X₁, X₂, X₃, X₄, and X₅ is H. The following 57 clones include this consensus motif: LAD5224_P03_A01; SAD10318_P01_B01; SAD10318_P01_F01; SAD10318_P02_B05; SAD10318_P02_F05; SAD10318_P02_G05; SAD10318_P03_B07; SAD10318_P03_G07; SAD10318_P03_A08; SAD10318_P03_A09; SAD10318_P04_E10; SAD10318_P04_E11; SAD10318_P04_H11; SAD10319_P01_D01; SAD10319_P01_F01; SAD10319_P01_G01; SAD10319_P01_D02; SAD10319_P01_E02; SAD10319_P02_B04; SAD10319_P02_E05; SAD10319_P02_E06; SAD10319_P02_H06; SAD10319_P03_G08; SAD10319_P03_B09; SAD10319_P03_G09; SAD10319_P04_B10; SAD10319_P04_C11; SAD10319_P04_D11; SAD10319_P04_F12; SAD10319_P04_H12; SAD10320_P01_E02; SAD10320_P02_C04; SAD10320_P02_C06; SAD10320_P02_G06; SAD10319_P05_A02; SAD10319_P05_B03; SAD10319_P05_B04; SAD10319_P05_D01; SAD10319_P05_G02; SAD10319_P05_G03; SAD10319_P05_H06; SAD10319_P06_A07; SAD10319_P06_A10; SAD10319_P06_A11; SAD10319_P06_E09; SAD10319_P06_E10; SAD10319_P06_G11; SAD10319_P06_H11; LAD9953_P01_H01; LAD9954_P01_B02; LAD9955_P01_G02; LAD9956_P01_C03; LAD9959_P01_E04; LAD9960_P01_D05; LAD9963_P01_E06; LAD9964_P01_C07; and LAD9966_P01_A08.

In some embodiments, the disclosure provides an antibody comprising a CDRH3 binding domain comprising a consensus motif, the consensus motif comprising the sequence ARDAHX₁X₂YX₃X₄DX₅, wherein X₁ is G, E, or R, wherein X₂ is R or H, wherein X₃ is F or H, wherein X₄ is Y or H, and wherein X₅ is V or H (SEQ ID NO: 3). In some embodiments, at least one of X₁, X₂, X₃, X₄, and X₅ is H. The following 23 clones include this consensus motif: SAD10318_P01_G01; SAD10318_P01_F02; SAD10318_P01_C03; SAD10318_P01_E03; SAD10318_P01_F03; SAD10318_P02_B04; SAD10318_P02_D04; SAD10318_P02_D06; SAD10318_P03_F07; SAD10318_P04_F11; SAD10318_P04_H12; SAD10319_P02_D04; SAD10319_P02_H04; SAD10319_P02_D05; SAD10319_P03_G07; SAD10319_P04_C10; SAD10319_P04_B11; SAD10319_P04_B12; SAD10320_P02_A06; SAD10319_P05_A03; SAD10319_P05_B05; SAD10319_P05_G04; and SAD10319_P06_D12.

In some embodiments, the disclosure provides an antibody comprising a CDRH3 binding domain comprising a consensus motif, the consensus motif comprising the sequence ARDAX₁HRX₂FYDV, wherein X₁ is H, Y, S, G, A, T, V, or R, and wherein X₂ is Y or H (SEQ ID NO: 4). In some embodiments, at least one of X₁ and X₂ is H. The following 19 clones include this consensus motif: SAD10318_P01_E01; SAD10318_P01_H01; SAD10318_P01_D02; SAD10318_P02_C04; SAD10318_P02_C05; SAD10318_P02_B06; SAD10318_P02_E06; SAD10318_P03_D09; SAD10318_P04_A12; SAD10319_P02_F05; SAD10319_P03_H08; SAD10320_P01_F01; SAD10320_P01_C02; SAD10320_P01_H03; SAD10320_P02_D05; SAD10320_P02_H05; SAD10320_P03_E07; SAD10320_P04_A11; and SAD10319_P05_G01.

In some embodiments, the disclosure provides an antibody comprising a CDRH3 binding domain comprising a consensus motif, the consensus motif comprising the sequence ARDX₁YHRYFYDX₂, wherein X₁ is H or A, and wherein X₂ is H, V, or M (SEQ ID NO: 5). In some embodiments, at least one of X₁ and X₂ is H. The following 15 clones include this consensus motif: SAD10318_P01_D01; SAD10318_P01_B02; SAD10318_P01_A03; SAD10318_P02_H04; SAD10318_P02_A05; SAD10318_P03_E07; SAD10318_P03_B08; SAD10318_P03_D08; SAD10318_P03_E08; SAD10318_P03_F08; SAD10318_P03_G08; SAD10318_P03_C09; SAD10318_P04_B11; SAD10319_P01_H01; and SAD10319_P04_E12.

In some embodiments, the disclosure provides an antibody comprising a CDRH3 binding domain comprising a consensus motif, the consensus motif comprising the sequence AX₁DAYX₂X₃X₄HX₅DV, wherein X₁ is R or H, wherein X₂ is G or H, wherein X₃ is H or R, wherein X₄ is N, F, or Y, and wherein X₅ is Y or H (SEQ ID NO: 6). In some embodiments, at least one of X₁, X₂, X₃, X₄, and X₅ is H. The following 14 clones include this consensus motif: SAD10318_P01_C01; SAD10318_P02_G04; SAD10318_P03_E09; SAD10318_P03_F09; SAD10318_P04_C10; SAD10318_P04_D10; SAD10318_P04_F10; SAD10318_P04_G11; SAD10318_P04_G12; SAD10320_P02_F05; SAD10320_P02_F06; SAD10320_P02_H06; SAD10320_P04_F10; and SAD10319_P05_D04.

In some embodiments, the disclosure provides an antibody comprising a CDRH3 binding domain comprising a consensus motif, the consensus motif comprising the sequence ARDX₁X₂GRYFYDV, wherein X₁ is M, Q, or H, and wherein X₂ is R or H (SEQ ID NO: 7). In some embodiments, at least one of X₁ and X₂ is H. The following 7 clones include this sequence motif: SAD10318_P02_E04; SAD10318_P04_C11; SAD10318_P04_F12; SAD10319_P02_H05; SAD10320_P01_A03; SAD10320_P01_B03; and SAD10320_P02_E05.

In some embodiments, the disclosure provides an antibody comprising a CDRH3 binding domain comprising a consensus motif, the consensus motif comprising the sequence ARDX₁X₂X₃RYFYDX₄, wherein X₁ is H or A, wherein X₂ is T, Y, or H, wherein X₃ is G or H, and wherein X₄ is V or H (SEQ ID NO: 8). In some embodiments, at least one of X₁, X₂, X₃, and X₄ is H. The following clones include this sequence motif: ADI-26906; ADI-48584; ADI-57317; ADI-57319; ADI-57323; ADI-57328; ADI-48639; ADI-57300; ADI-57333; ADI-57336; ADI-57337; ADI-48587; ADI-57343; ADI-48648; ADI-48650; ADI-48589; ADI-48652; ADI-48654; ADI-48592; ADI-57401; ADI-57406; ADI-57274; ADI-57413; ADI-57414; ADI-57415; ADI-57416; ADI-57417; ADI-57275; ADI-57427; ADI-57428; ADI-57437; ADI-57438; ADI-48594; ADI-57439; ADI-57440; ADI-57441; ADI-57442; ADI-57443; ADI-57444; ADI-57445; ADI-48666; ADI-48595; ADI-48597; ADI-48576; ADI-57277; ADI-57279; ADI-57280; ADI-57281; ADI-48601; ADI-48577; ADI-57284; ADI-48604; ADI-48606; ADI-57285; ADI-48608; ADI-48609; ADI-48610; ADI-48614; ADI-48615; ADI-48617; ADI-57295; ADI-48580; ADI-48622; ADI-57299; ADI-57300; ADI-48623; ADI-57303; ADI-48582; and ADI-57311.

In some embodiments, the disclosure provides an antibody comprising a CDRH3 binding domain comprising a consensus motif, the consensus motif comprising the sequence ARDX₁X₂X₃X₄YFYDX₅, wherein X₁ is H or A, wherein X₂ is T, Y, or H, wherein X₃ is G or H, X₄ is H, R, V, or I, and wherein X₅ is V or H (SEQ ID NO: 43). In some embodiments, at least one of X₁, X₂, X₃, X₄, and X₅ is H. The following 11 clones include this consensus motif: ADI-48576; ADI-48577; ADI-48587; ADI-48592; ADI-48595; ADI-48635; ADI-48650; ADI-48652; ADI-48666; ADI-48643; and ADI-48645.

In some embodiments, the disclosure provides an antibody comprising a CD3 binding domain, CDRH3, wherein the CDRH3 binding domain comprises a consensus motif, the consensus motif comprising the sequence AX₁DX₂X₃X₄X₅X₆X₇X₈DX₉, wherein X₁ is R or H, wherein X₂ is A, H, M, or Q, wherein X₃ is Y, H, S, G, A, T, V, or R; wherein X₄ is G, H, P, E, or R; wherein X₅ is H or R, wherein X₆ is Y, N, F, H, D, E, S, L, M, I, G, A, Q, or T; wherein X₇ is F or H; wherein X₈ is Y or H; and wherein X₉ is V, H, or M (SEQ ID NO: 58). In some embodiments, at least one of X₁, X₂, X₃, X₄, X₅, X₆, X₇, X₈, and X₉ is H. The following clones include this sequence motif: SAD10318_P01_A02; SAD10318_P01_G02; SAD10318_P01_D03; SAD10318_P01_G03; SAD10318_P01_H03; SAD10318_P02_D05; SAD10318_P02_H05; SAD10318_P02_G06; SAD10318_P03_C08; SAD10318_P03_H08; SAD10318_P03_G09; SAD10318_P04_H10; SAD10318_P04_D11; SAD10319_P01_A01; SAD10319_P01_C01; SAD10319_P01_E01; SAD10319_P01_A02; SAD10319_P01_C02; SAD10319_P01_F02; SAD10319_P01_H02; SAD10319_P01_B03; SAD10319_P01_C03; SAD10319_P01_D03; SAD10319_P01_F03; SAD10319_P02_A04; SAD10319_P02_C04; SAD10319_P02_E04; SAD10319_P02_F04; SAD10319_P02_A05; SAD10319_P02_B05; SAD10319_P02_C05; SAD10319_P02_G05; SAD10319_P02_A06; SAD10319_P02_B06; SAD10319_P02_C06; SAD10319_P02_D06; SAD10319_P02_F06; SAD10319_P02_G06; SAD10319_P03_C07; SAD10319_P03_H07; SAD10319_P03_D08; SAD10319_P03_E08; SAD10319_P03_E09; SAD10319_P03_F09; SAD10319_P04_A10; SAD10319_P04_G10; SAD10319_P04_E11; SAD10319_P04_F11; SAD10319_P04_G11; SAD10319_P04_C12; SAD10319_P04_D12; SAD10320_P01_B01; SAD10320_P01_D01; SAD10320_P01_E01; SAD10320_P01_G01; SAD10320_P01_H01; SAD10320_P01_A02; SAD10320_P01_F02; SAD10320_P01_G02; SAD10320_P01_H02; SAD10320_P01_C03; SAD10320_P01_D03; SAD10320_P01_E03; SAD10320_P01_F03; SAD10320_P01_G03; SAD10320_P02_A04; SAD10320_P02_B04; SAD10320_P02_E04; SAD10320_P02_H04; SAD10320_P02_A05; SAD10320_P02_B05; SAD10320_P02_C05; SAD10320_P02_B06; SAD10320_P02_D06; SAD10320_P02_E06; SAD10320_P03_B07; SAD10320_P03_H07; SAD10320_P03_C08; SAD10320_P03_D08; SAD10320_P03_F08; SAD10320_P03_H08; SAD10320_P03_A09; SAD10320_P03_C09; SAD10320_P03_D09; SAD10320_P03_F09; SAD10320_P04_A10; SAD10320_P04_C10; SAD10320_P04_D10; SAD10320_P04_E10; SAD10320_P04_G10; SAD10320_P04_D11; SAD10320_P04_E11; SAD10320_P04_F11; SAD10320_P04_H11; SAD10320_P04_A12; SAD10320_P04_D12; SAD10320_P04_E12; SAD10320_P04_F12; SAD10319_P05_A01; SAD10319_P05_A05; SAD10319_P05_B02; SAD10319_P05_C01; SAD10319_P05_C03; SAD10319_P05_C05; SAD10319_P05_D02; SAD10319_P05_D03; SAD10319_P05_D05; SAD10319_P05_E04; SAD10319_P05_F01; SAD10319_P06_B10; SAD10319_P06_B11; SAD10319_P06_C10; SAD10319_P06_C12; SAD10319_P06_E08; SAD10319_P06_F07; SAD10319_P06_F10; SAD10319_P06_G09; SAD10319_P06_H07; SAD10319_P06_H08; SAD10319_P06_H10; LAD5224_P03_A01; SAD10318_P01_B01; SAD10318_P01_F01; SAD10318P02_B05; SAD10318_P02_F05; SAD10318_P02 G05; SAD10318_P03_B07; SAD10318_P03_G07; SAD10318_P03_A08; SAD10318_P03_A09; SAD10318_P04_E10; SAD10318_P04_E11; SAD10318_P04_H11; SAD10319_P01_D01; SAD10319_P01_F01; SAD10319_P01_G01; SAD10319_P01_D02; SAD10319_P01_E02; SAD10319_P02_B04; SAD10319_P02_E05; SAD10319_P02_E06; SAD10319_P02_H06; SAD10319_P03_G08; SAD10319_P03_B09; SAD10319_P03_G09; SAD10319_P04_B10; SAD10319_P04_C11; SAD10319_P04_D11; SAD10319_P04_F12; SAD10319_P04_H12; SAD10320_P01_E02; SAD10320_P02_C04; SAD10320_P02_C06; SAD10320_P02_G06; SAD10319_P05_A02; SAD10319_P05_B03; SAD10319_P05_B04; SAD10319_P05_D01; SAD10319_P05_G02; SAD10319_P05_G03; SAD10319_P05_H06; SAD10319_P06_A07; SAD10319_P06_A10; SAD10319_P06_A11; SAD10319_P06_E09; SAD10319_P06_E10; SAD10319_P06_G11; SAD10319_P06_H11; LAD9953_P01_H01; LAD9954_P01_B02; LAD9955_P01_G02; LAD9956_P01_C03; LAD9959_P01_E04; LAD9960_P01_D05; LAD9963_P01_E06; LAD9964_P01_C07; LAD9966_P01_A08; SAD10318_P01_01; SAD10318_P01_F02; SAD10318_P01_C03; SAD10318_P01_E03; SAD10318_P01_F03; SAD10318_P02_B04; SAD10318_P02_D04; SAD10318_P02_D06; SAD10318_P03_F07; SAD10318_P04_F11; SAD10318_P04_H12; SAD10319_P02_D04; SAD10319_P02_H04; SAD10319_P02_D05; SAD10319_P03_G07; SAD10319_P04_C10; SAD10319_P04_B11; SAD10319_P04_B12; SAD10320_P02_A06; SAD10319_P05_A03; SAD10319_P05_B05; SAD10319_P05_G04; SAD10319_P06_D12; SAD10318_P01_E01; SAD10318_P01_H01; SAD10318_P01_D02; SAD10318_P02_C04; SAD10318_P02_C05; SAD10318_P02_B06; SAD10318_P02_E06; SAD10318_P03_D09; SAD10318_P04_A12; SAD10319_P02_F05; SAD10319_P03_H08; SAD10320_P01_F01; SAD10320_P01_C02; SAD10320_P01_H03; SAD10320_P02_D05; SAD10320_P02_H05; SAD10320_P03_E07; SAD10320_P04_A11; SAD10319_P05_G01; SAD10318_P01_D01; SAD10318_P01_B02; SAD10318_P01_A03; SAD10318_P02_H04; SAD10318_P02_A05; SAD10318_P03_E07; SAD10318_P03_B08; SAD10318_P03_D08; SAD10318_P03_E08; SAD10318_P03_F08; SAD10318_P03_G08; SAD10318_P03_C09; SAD10318_P04_B11; SAD10319_P01_H01; SAD10319_P04_E12; SAD10318_P01_D01; SAD10318_P01_B02; SAD10318_P01_A03; SAD10318_P02_H04; SAD10318_P02_A05; SAD10318_P03_E07; SAD10318_P03_B08; SAD10318_P03_D08; SAD10318_P03_E08; SAD10318_P03_F08; SAD10318_P03_G08; SAD10318_P03_C09; SAD10318_P04_B11; SAD10319_P01_H01; SAD10319_P04_E12; SAD10318_P02_E04; SAD10318_P04_C11; SAD10318_P04_F12; SAD10319_P02_H05; SAD10320_P01_A03; SAD10320_P01_B03; SAD10320_P02_E05; ADI-26906; ADI-48584; ADI-57317; ADI-57319; ADI-57323; ADI-57328; ADI-48639; ADI-57300; ADI-57333; ADI-57336; ADI-57337; ADI-48587; ADI-57343; ADI-48648; ADI-48650; ADI-48589; ADI-48652; ADI-48654; ADI-48592; ADI-57401; ADI-57406; ADI-57274; ADI-57413; ADI-57414; ADI-57415; ADI-57416; ADI-57417; ADI-57275; ADI-57427; ADI-57428; ADI-57437; ADI-57438; ADI-48594; ADI-57439; ADI-57440; ADI-57441; ADI-57442; ADI-57443; ADI-57444; ADI-57445; ADI-48666; ADI-48595; ADI-48597; ADI-48576; ADI-57277; ADI-57279; ADI-57280; ADI-57281; ADI-48601; ADI-48577; ADI-57284; ADI-48604; ADI-48606; ADI-57285; ADI-48608; ADI-48609; ADI-48610; ADI-48614; ADI-48615; ADI-48617; ADI-57295; ADI-48580; ADI-48622; ADI-57299; ADI-57300; ADI-48623; ADI-57303; ADI-48582; and ADI-57311.

In some embodiments, the disclosure provides an antibody comprising a CDRH3 binding domain comprising a consensus motif, the consensus motif comprising the sequence ARDAX₁X₂X₃X₄FYDX₅, wherein X₁ is T, H, or Y, wherein X₂ is G or H, wherein X₃ is H or R, wherein X₄ is V or Y, and wherein X₅ is V or H (SEQ ID NO: 593). In some embodiments, at least one of X₁, X₂, X₃, and X₅ is H. At least the following 6 clones include this consensus motif and are designated as Group 1 binders: ADI-48592, ADI-48595, ADI-48650, ADI-48652, ADI-48662, and ADI-48666.

In some embodiments, the disclosure provides an antibody comprising a CDRH3 binding domain comprising a consensus motif, the consensus motif comprising the sequence AX₁DX₂X₃X₄X₅X₆X₇YDX₈, wherein X₁ is R or H, wherein X₂ is H or A, wherein X₃ is H or Y, wherein X₄ is H, G, or P, wherein X₅ is R or H, wherein X₆ is Y, I, or V, wherein X₇ is F or H, and wherein X₈ is V or H (SEQ ID NO: 596). In some embodiments, at least one of X₁, X₂, X₃, X₄, X₅, X₇, and X₈ is H. At least the following 21 clones include this consensus motif and are designated as Group 2 binders: ADI-48588, ADI-48587, ADI-48577, ADI-48590, ADI-48581, ADI-48575, ADI-48593, ADI-48591, ADI-48647, ADI-48636, ADI-48586, ADI-48646, ADI-48638, ADI-48597, ADI-48601, ADI-48576, ADI-48643, ADI-48624, ADI-48632, ADI-48635, and ADI-48645.

In some embodiments, the disclosure provides an antibody comprising a CDRH2 binding domain comprising a consensus motif, the consensus motif comprising the sequence WIDLENANTIYDAKFQG (SEQ ID NO: 9). The following 148 clones include this consensus motif: LAD5224_P03_A01; SAD10318_P01_B01; SAD10318_P01_C01; SAD10318_P01_D01; SAD10318_P01_E01; SAD10318_P01_F01; SAD10318_P01_G01; SAD10318_P01_H01; SAD10318_P01_A02; SAD10318_P01_B02; SAD10318_P01_D02; SAD10318_P01_F02; SAD10318_P01_G02; SAD10318_P01_A03; SAD10318_P01_C03; SAD10318_P01_D03; SAD10318_P01_E03; SAD10318_P01_F03; SAD10318_P01_03; SAD10318_P01_H03; SAD10318_P02_B04; SAD10318_P02_C04; SAD10318_P02_D04; SAD10318_P02_E04; SAD10318_P02 G04; SAD10318_P02_H04; SAD10318_P02_A05; SAD10318_P02_B05; SAD10318_P02_C05; SAD10318_P02_D05; SAD10318_P02_F05; SAD10318_P02_G05; SAD10318_P02_H05; SAD10318_P02_B06; SAD10318_P02_D06; SAD10318_P02_E06; SAD10318_P02_G06; SAD10318_P03_B07; SAD10318_P03_E07; SAD10318_P03_F07; SAD10318_P03 G07; SAD10318_P03_A08; SAD10318_P03_B08; SAD10318_P03_C08; SAD10318_P03_D08; SAD10318_P03_E08; SAD10318_P03_F08; SAD10318_P03_G08; SAD10318_P03_H08; SAD10318_P03_A09; SAD10318_P03_C09; SAD10318_P03_D09; SAD10318_P03_E09; SAD10318_P03_F09; SAD10318_P03 G09; SAD10318_P04_C10; SAD10318_P04_D10; SAD10318_P04_E10; SAD10318_P04_F10; SAD10318_P04_H10; SAD10318_P04_A11; SAD10318_P04_B11; SAD10318_P04_C11; SAD10318_P04_D11; SAD10318_P04_E11; SAD10318_P04_F11; SAD10318_P04_G11; SAD10318_P04_H11; SAD10318_P04_A12; SAD10318_P04_F12; SAD10318_P04 G12; SAD10318_P04_H12; SAD10320_P01_B01; SAD10320_P01_D01; SAD1032_P01_E01; SAD10320_P01_F01; SAD10320_P01_G01; SAD10320_P01_H01; SAD10320_P01_A02; SAD10320_P01_C02; SAD10320_P01_E02; SAD10320_P01_F02; SAD10320_P01_G02; SAD10320_P01_H02; SAD10320_P01_A03; SAD10320_P01_B03; SAD10320_P01_C03; SAD10320_P01_D03; SAD10320_P01_E03; SAD10320_P01_F03; SAD10320_P01_G03; SAD10320_P01_H03; SAD10320_P02_A04; SAD10320_P02_B04; SAD10320_P02_C04; SAD10320_P02_E04; SAD10320_P02_H04; SAD10320_P02_A05; SAD10320_P02_B05; SAD10320_P02_C05; SAD10320_P02_D05; SAD10320_P02_E05; SAD10320_P02_F05; SAD10320_P02_H05; SAD10320_P02_A06; SAD10320_P02_B06; SAD10320_P02_C06; SAD10320_P02_D06; SAD10320_P02_E06; SAD10320_P02_F06; SAD10320_P02_G06; SAD10320_P02_H06; SAD10320_P03_B07; SAD10320_P03_E07; SAD10320_P03_H07; SAD10320_P03_C08; SAD10320_P03_D08; SAD10320_P03_F08; SAD10320_P03_H08; SAD10320_P03_A09; SAD10320_P03_C09; SAD10320_P03_D09; SAD10320_P03_F09; SAD10320_P04_A10; SAD10320_P04_C10; SAD10320_P04_D10; SAD10320_P04_E10; SAD10320_P04_F10; SAD10320_P04_G10; SAD10320_P04_A11; SAD10320_P04_D11; SAD10320_P04_E11; SAD10320_P04_F11; SAD10320_P04_G11; SAD10320_P04_H11; SAD10320_P04_A12; SAD10320_P04_D12; SAD10320_P04_E12; SAD10320_P04_F12; LAD9953_P01_H01; LAD9954_P01_B02; LAD9955_P01_G02; LAD9956_P01_C03; LAD9959_P01_E04; LAD9960_P01_D05; LAD9963_P01_E06; LAD9964_P01_C07; and LAD9966_P01_A08. Additionally, at least the following 16 clones include this consensus motif and are designated as Group 2 binders: ADI-48575, ADI-48576, ADI-48577, ADI-48581, ADI-48586, ADI-48587, ADI-48588, ADI-48590, ADI-48591, ADI-48593, ADI-48601, ADI-48646, ADI-48647, ADI-48597, ADI-48643, and ADI-48645.

In some embodiments, the disclosure provides an antibody comprising a CDRH2 binding domain comprising a consensus motif, the consensus motif comprising the sequence X₁INPX₂TGX₃TX₄YSQKFQG, wherein X₁ is W or Y, wherein X₂ is A, S, D, G, N, L, V, H, or Q, wherein X₃ is A, T, or S, and wherein X₄ is K, V, T, D, Y, F, or A (SEQ ID NO: 10). In some embodiments, at least one of X₁, X₂, X₃, and X₄ is H. The following 24 clones include this consensus motif: SAD10319_P01_E02; SAD10319_P01_H02; SAD10319_P01_B03; SAD10319_P02_A04 SAD10319_P02_B04; SAD10319_P02_C04; SAD10319_P02_F04; SAD10319_P02_H04; SAD10319_P02_A05; SAD10319_P02_C05; SAD10319_P02_C06; SAD10319_P02_E06; SAD10319_P02_F06; SAD10319_P02_G06; SAD10319_P03_D08; SAD10319_P03_F09; SAD10319_P04_G10; SAD10319_P04_C11; SAD10319_P05_A01; SAD10319_P05_A05; SAD10319_P05_G03; SAD10319_P06_A10; SAD10319_P06_C12; and SAD10319_P06_E09.

In some embodiments, the disclosure provides an antibody comprising a CDRH2 binding domain comprising a consensus motif, the consensus motif comprising the sequence X₁IX₂AGTGX₃TX₄YSQKFQG, wherein X₁ is W, Y, or F, wherein X₂ is T, N, or D, wherein X₃ is A, T, or L, and wherein X₄ is A, K, V, H, T, or N (SEQ ID NO: 11). In some embodiments, at least one of X₁, X₂, X₃, and X₄ is H. The following 23 clones include this consensus motif: SAD10319_P01_E01; SAD10319_P01_G01; SAD10319_P01_D02; SAD10319_P01_D03; SAD10319_P02_E05; SAD10319_P02_A06; SAD10319_P03_C07; SAD10319_P03_G07; SAD10319_P03_B09; SAD10319_P03_E09; SAD10319_P04_A10; SAD10319_P04_B10; SAD10319_P04_B11; SAD10319_P04_E12; SAD10319_P05_A02; SAD10319_P05_C05; SAD10319_P05_D01; SAD10319_P05_H06; SAD10319_P06_A07; SAD10319_P06_B11; SAD10319_P06_F07; SAD10319_P06_G09; and SAD10319_P06_H08.

In some embodiments, the disclosure provides an antibody comprising a CDRH2 binding domain comprising a consensus motif, the consensus motif comprising the sequence X₁IDAGTGX₂TX₃YSQKFQG, wherein X₁ is S or W, wherein X₂ is L, N, D, or F, and wherein X₃ is D, Y, or K (SEQ ID NO: 12). In some embodiments, at least one of X₁, X₂, and X₃ is H. The following 17 clones include this consensus motif: SAD10319_P01_C01; SAD10319_P01_D01; SAD10319_P01_H01; SAD10319_P01_F02; SAD10319_P02_D04; SAD10319_P02_D05; SAD10319_P02_F05; SAD10319_P02_H06; SAD10319_P03_G08; SAD10319_P04_D11; SAD10319_P05_A03; SAD10319_P05_B05; SAD10319_P05_C01; SAD10319_P05_D03; SAD10319_P05_F01; SAD10319_P05_G01; and SAD10319_P06_H10.

In some embodiments, the disclosure provides an antibody comprising a CDRH2 binding domain comprising a consensus motif, the consensus motif comprising the sequence X₁IX₂AGTGATX₃YSQKFQG, wherein X₁ is G, D, or S, wherein X₂ is I or D, and wherein X₃ is K or D (SEQ ID NO: 13). In some embodiments, at least one of X₁, X₂, and X₃ is H. The following 7 clones include this consensus motif: SAD10319_P02_G05; SAD10319_P05_B02; SAD10319_P05_C03; SAD10319_P05_D05; SAD10319_P06_B10; SAD10319_P06_C10; and SAD10319_P06_D12.

In some embodiments, the disclosure provides an antibody comprising a CDRH2 binding domain comprising a consensus motif, the consensus motif comprising the sequence WINPX₁TGNTX₂YSQKFQG, wherein X₁ is D, T, L, S, or A, and wherein X₂ is D, V, L, or N (SEQ ID NO: 14). In some embodiments, at least one of X₁ and X₂ is H. The following 6 clones include this consensus motif: SAD10319_P01_A01; SAD10319_P01_F01; SAD10319_P01_C02; SAD10319_P04_F12; SAD10319_P05_E04; and SAD10319_P06_A11.

In some embodiments, the disclosure provides an antibody comprising a CDRH2 binding domain comprising a consensus motif, the consensus motif comprising the sequence X₁INAGTGX₂TX₃YSQKFQG, wherein X₁ is Y or W, wherein X₂ is N, D, or A, and wherein X₃ is I or V (SEQ ID NO: 15). In some embodiments, at least one of X₁, X₂, and X₃ is H. The following 5 clones include this consensus motif: SAD10319_P01_F03; SAD10319_P02_H05; SAD10319_P02_D06; SAD10319_P03_E08; and SAD10319_P03_H08.

In some embodiments, the disclosure provides an antibody comprising a CDRH2 binding domain comprising a consensus motif, the consensus motif comprising the sequence X₁INPX₂TGX₃TKYSQKFQG, wherein X₁ is W or Y, wherein X₂ is D, I or Y, and wherein X₃ is D, Y, or E (SEQ ID NO: 16). In some embodiments, at least one of X₁, X₂, and X₃ is H. The following 5 clones include this consensus motif: SAD10319_P03_H07; SAD10319_P04_E11; SAD10319_P04_F11; SAD10319_P04_B12; and SAD10319_P04_D12.

In some embodiments, the disclosure provides an antibody comprising a CDRH2 binding domain comprising a consensus motif, the consensus motif comprising the sequence SIX₁AGTGX₂TKYSQKFQG, wherein X₁ is N or V, and wherein X₂ is A or I (SEQ ID NO: 17). In some embodiments, at least one of X₁ and X₂ is H. The following 3 clones include this consensus motif: SAD10319_P02_E04; SAD10319_P04_C10; and SAD10319_P04_H12.

In some embodiments, the disclosure provides an antibody comprising a CDRH2 binding domain comprising a consensus motif, the consensus motif comprising the sequence SINAGTGX₁TX₂YSQKFQG, wherein X₁ is F or N, and wherein X₂ is Y or D (SEQ ID NO: 18). In some embodiments, at least one of X₁ and X₂ is H. The following 3 clones include this consensus motif: SAD10319_P02_B05; SAD10319_P02_B06; and SAD10319_P05_D02.

In some embodiments, the disclosure provides an antibody comprising a CDRH2 binding domain comprising a consensus motif, the consensus motif comprising the sequence X₁IX₂X₃GTGX₄TDYSQKFQG, wherein X₁ is D or W, wherein X₂ is N or H, wherein X₃ is A or S, and wherein X₄ is A or N (SEQ ID NO: 19). In some embodiments, at least one of X₁, X₂, X₃, and X₄ is H. The following 3 clones include this consensus motif: SAD10319_P05_B03 SAD10319_P05_B04; and SAD10319_P05_D04.

In some embodiments, the disclosure provides an antibody comprising a CDRH2 binding domain comprising a consensus motif, the consensus motif comprising the sequence WIDPX₁TGATX₂YSQKFQG, wherein X₁ is N, H, or Y, and wherein X₂ is V or K (SEQ ID NO: 20). In some embodiments, at least one of X₁ and X₂ is H. The following 3 clones include this consensus motif: SAD10319_P01_C03; SAD10319_P03_G09; and SAD10319_P06_F10.

In some embodiments, the disclosure provides an antibody comprising a CDRH2 binding domain comprising a consensus motif, the consensus motif comprising the sequence WIX₁PX₂TGNTKYSQKFQG, wherein X₁ is D or N, and wherein X₂ is L, I, or V (SEQ ID NO: 21). In some embodiments, at least one of X₁ and X₂ is H. The following 3 clones include this consensus motif: SAD10319_P01_A02; SAD10319_P04_C12; and SAD10319_P05_G02.

In some embodiments, the disclosure provides an antibody comprising a CDRH2 binding domain comprising a consensus motif, the consensus motif comprising the sequence SINAGDANTKYSQKFQG (SEQ ID NO: 22). The following 2 clones include this consensus motif: SAD10319_P04_G11 and SAD10319_P06_H07.

In some embodiments, the disclosure provides an antibody comprising a CDRH2 binding domain comprising a consensus motif, the consensus motif comprising the sequence X₁IDPX₂TGATX₃YSQKFQG, wherein X₁ is D or W, wherein X₂ is D or V, and wherein X₃ is E or D (SEQ ID NO: 23). In some embodiments, at least one of X₁, X₂, and X₃ is H. The following 2 clones include this consensus motif: SAD10319_P05_G04 and SAD10319_P06_E08.

In some embodiments, the disclosure provides an antibody comprising a CDRH2 binding domain comprising a consensus motif, the consensus motif comprising the sequence WINAGDAATVYSQKFQG (SEQ ID NO: 24). The following 2 clones include this consensus motif: SAD10319_P06_G11 and SAD10319_P06_H11.

In some embodiments, the disclosure provides an antibody comprising a CDRH2 binding domain comprising a consensus motif, the consensus motif comprising the sequence X₁IX₂X₃X₄X₅X₆X₇TX₈YSQKFQG, wherein X₁ is W, S, Y, F, G, or D, wherein X₂ is N, T, D, V, or H, wherein X₃ is A, P, or S, wherein X₄ is G, A, S, N, D, L, V, H, Q, T, I, or Y, wherein X₅ is D or T, wherein X₆ is A or G, wherein X₇ is A, N, T, S, L, D, F, Y, or E, and wherein X₈ is V, K, T, D, Y, F, A, H, N, L, I, or E (SEQ ID NO: 59). In some embodiments, at least one of X₁, X₂, X₃, X₄, X₅, X₆, X₇, and X₈ is H. The following clones include this consensus motif: SAD10319_P01_E02; SAD10319_P01_H02; SAD10319_P01_B03; SAD10319_P02_A04 SAD10319_P02_B04; SAD10319_P02_C04; SAD10319_P02_F04; SAD10319_P02_H04; SAD10319_P02_A05; SAD10319_P02_C05; SAD10319_P02_C06; SAD10319_P02_E06; SAD10319_P02_F06; SAD10319_P02_G06; SAD10319_P03_D08; SAD10319_P03_F09; SAD10319_P04_G10; SAD10319_P04_C11; SAD10319_P05_A01; SAD10319_P05_A05; SAD10319_P05_G03; SAD10319_P06_A10; SAD10319_P06_C12; SAD10319_P06_E09; SAD10319_P01_E01; SAD10319_P01_G01; SAD10319_P01_D02; SAD10319_P01_D03; SAD10319_P02_E05; SAD10319_P02_A06; SAD10319_P03_C07; SAD10319_P03_G07; SAD10319_P03_B09; SAD10319_P03_E09; SAD10319_P04_A10; SAD10319_P04_B10; SAD10319_P04_B11; SAD10319_P04_E12; SAD10319_P05_A02; SAD10319_P05_C05; SAD10319_P05_D01; SAD10319_P05_H06; SAD10319_P06_A07; SAD10319_P06_B11; SAD10319_P06_F07; SAD10319_P06_G09; SAD10319_P06_H08; SAD10319_P01_C01; SAD10319_P01_D01; SAD10319_P01_H01; SAD10319_P01_F02; SAD10319_P02_D04; SAD10319_P02_D05; SAD10319_P02_F05; SAD10319_P02_H06; SAD10319_P03 G08; SAD10319_P04_D11; SAD10319_P05_A03; SAD10319_P05_B05; SAD10319_P05_C01; SAD10319_P05_D03; SAD10319_P05_F01; SAD10319_P05_G01; SAD10319_P06_H10; SAD10319_P02_G05; SAD10319_P05_B02; SAD10319_P05_C03; SAD10319_P05_D05; SAD10319_P06_B10; SAD10319_P06_C10; SAD10319_P06_D12; SAD10319_P01_A01; SAD10319_P01_F01; SAD10319_P01_C02; SAD10319_P04_F12; SAD10319_P05_E04; SAD10319_P06_A11; SAD10319_P01_F03; SAD10319_P02_H05; SAD10319_P02_D06; SAD10319_P03_E08; SAD10319_P03_H08; SAD10319_P03_H07; SAD10319_P04_E11; SAD10319_P04_F11; SAD10319_P04_B12; SAD10319_P04_D12; SAD10319_P02_E04; SAD10319_P04_C10; SAD10319_P04_H12; SAD10319_P02_B05; SAD10319_P02_B06; SAD10319_P05_D02; SAD10319_P05_B03; SAD10319_P05_B04; SAD10319_P05_D04; SAD10319_P01_C03; SAD10319_P03_G09; SAD10319_P06_F10; SAD10319_P01_A02; SAD10319_P04_C12; SAD10319_P05_G02; SAD10319_P04_G11; SAD10319_P06_H07; SAD10319_P05_G04; SAD10319_P06_E08; SAD10319_P06_G1; and SAD10319_P06_H11.

In some embodiments, the disclosure provides an antibody comprising a CDRH2 binding domain comprising a consensus motif, the consensus motif comprising the sequence WIDAGTGX₁TX₂YSQKFQG, wherein X₁ is L, F, N, or A and wherein X₂ is T or K (SEQ ID NO: 595). At least the following 4 clones include this consensus motif and are designated as Group 2 binders: ADI-48636, ADI-48638, ADI-48624, and ADI-48635.

In some embodiments, the disclosure provides an antibody comprising a CDRH1 binding domain comprising a consensus motif, the consensus motif comprising the sequence X₁NIKDYX₂MH, wherein X₁ is F or S, and wherein X₂ is Y or H (SEQ ID NO: 44). In some embodiments, at least one of X₁ and X₂ is H. In some embodiments, the sequence is FNIKDYHMH (SEQ ID NO: 25), SNIKDYYMH (SEQ ID NO: 26), or SNIKDYHMH (SEQ ID NO: 27). The following 148 clones include this consensus motif: LAD5224_P03_A01; SAD10318_P01_B01; SAD10318_P01_C01; SAD10318_P01_D01; SAD10318_P01_E01; SAD10318_P01_F01; SAD10318_P01_G01; SAD10318_P01_H01; SAD10318_P01_A02; SAD10318_P01_B02; SAD10318_P01_D02; SAD10318_P01_F02; SAD10318_P01_G02; SAD10318_P01_A03; SAD10318_P01_C03; SAD10318_P01_D03; SAD10318_P01_E03; SAD10318_P01_F03; SAD10318_P01_G03; SAD10318_P01_H03; SAD10318_P02_B04; SAD10318_P02_C04; SAD10318_P02_D04; SAD10318_P02_E04; SAD10318_P02_G04; SAD10318_P02_H04; SAD10318_P02_A05; SAD10318_P02_B05; SAD10318_P02_C05; SAD10318_P02_D05; SAD10318_P02_F05; SAD10318_P02_G05; SAD10318_P02_H05; SAD10318_P02_B06; SAD10318_P02_D06; SAD10318_P02_E06; SAD10318_P02 G06; SAD10318_P03_B07; SAD10318_P03_E07; SAD10318_P03_F07; SAD10318_P03_G07; SAD10318_P03_A08; SAD10318_P03_B08; SAD10318_P03_C08; SAD10318_P03_D08; SAD10318_P03_E08; SAD10318_P03_F08; SAD10318_P03_G08; SAD10318_P03_H08; SAD10318_P03_A09; SAD10318_P03_C09; SAD10318_P03_D09; SAD10318_P03_E09; SAD10318_P03_F09; SAD10318_P03_G09; SAD10318_P04_C10; SAD10318_P04_D10; SAD10318_P04_E10; SAD10318_P04_F10; SAD10318_P04_H10; SAD10318_P04_A11; SAD10318_P04_B11 SAD10318_P04_C11; SAD10318_P04_D11; SAD10318_P04_E11; SAD10318_P04_F11; SAD10318_P04_G11; SAD10318_P04_H11; SAD10318_P04_A12; SAD10318_P04_F12; SAD10318_P04_G12; SAD10318_P04_H12; SAD10320_P01_B01; SAD10320_P01_D01; SAD10320_P01_E01; SAD10320_P01_F01; SAD10320_P01_01; SAD10320_P01_H01; SAD10320_P01_A02; SAD10320_P01_C02; SAD10320_P01_E02; SAD10320_P01_F02; SAD10320_P01_G02; SAD10320_P01_H02; SAD10320_P01_A03; SAD10320_P01_B03; SAD10320_P01_C03; SAD10320_P01_D03; SAD10320_P01_E03; SAD10320_P01_F03; SAD10320_P01_G03; SAD10320_P01_H03; SAD10320_P02_A04; SAD10320_P02_B04; SAD10320_P02_C04; SAD10320_P02_E04; SAD10320_P02_H04; SAD10320_P02_A05; SAD10320_P02_B05; SAD10320_P02_C05; SAD10320_P02_D05; SAD10320_P02_E05; SAD10320_P02_F05; SAD10320_P02_H05; SAD10320_P02_A06; SAD10320_P02_B06; SAD10320_P02_C06; SAD10320_P02_D06; SAD10320_P02_E06; SAD10320_P02_F06; SAD10320_P02_G06; SAD10320_P02_H06; SAD10320_P03_B07; SAD10320_P03_E07; SAD10320_P03_H07; SAD10320_P03_C08; SAD10320_P03_D08; SAD10320_P03_F08; SAD10320_P03_H08; SAD10320_P03_A09; SAD10320_P03_C09; SAD10320_P03_D09; SAD10320_P03_F09; SAD10320_P04_A10; SAD10320_P04_C10; SAD10320_P04_D10; SAD10320_P04_E10; SAD10320_P04_F10; SAD10320_P04_G10; SAD10320_P04_A11; SAD10320_P04_D11; SAD10320_P04_E11; SAD10320_P04_F11; SAD10320_P04_G11; SAD10320_P04_H11; SAD10320_P04_A12; SAD10320_P04_D12; SAD10320_P04_E12; SAD10320_P04_F12; LAD9953_P01_H01; LAD9954_P01_B02; LAD9955_P01_G02; LAD9956_P01_C03; LAD9959_P01_E04; LAD9960_P01_D05; LAD9963_P01_E06; LAD9964_P01_C07; and LAD9966_P01_A08.

In some embodiments, the disclosure provides an antibody comprising a CDRH1 binding domain comprising a consensus motif, the consensus motif comprising the sequence YTFX₁X₂X₃X₄MH, wherein X₁ is A, K, D, Q, E, N, T, L, Y, S, P, G, H or V, wherein X₂ is T, S, or A, wherein X₃ is Y or I, and wherein X₄ is A, D, N, S, Y, T, I, V, L, E, P, R, or G (SEQ ID NO: 28). In some embodiments, at least one of X₁, X₂, X₃, and X₄ is H. The following 61 clones include this consensus motif: SAD10319_P01_A0; SAD10319_P01_D01 SAD10319_P01_E01; SAD10319_P01_F01; SAD10319_P01_F02; SAD10319_P01_B03; SAD10319_P01_D03; SAD10319_P01_F03; SAD10319_P02_A04; SAD10319_P02_C04; SAD10319_P02_D04; SAD10319_P02_E04; SAD10319_P02_F04; SAD10319_P02_H04; SAD10319_P02_A05; SAD10319_P02_E05; SAD10319_P02_A06; SAD10319_P02_B06; SAD10319_P02_C06; SAD10319_P02_F06; SAD10319_P02_G06; SAD10319_P02_H06; SAD10319_P03_C07; SAD10319_P03_G07; SAD10319_P03_H07; SAD10319_P03_D08; SAD10319_P03_G08; SAD10319_P03_H08; SAD10319_P03_E09; SAD10319_P03_F09; SAD10319_P03_G09; SAD10319_P04_B11; SAD10319_P04_C11; SAD10319_P04_D11; SAD10319_P04_G11; SAD10319_P04_B12; SAD10319_P04_C12; SAD10319_P04_D12; SAD10319_P04_E12; SAD10319_P05_A01; SAD10319_P05_A02; SAD10319_P05_A05; SAD10319_P05_B02; SAD10319_P05_B03; SAD10319_P05_C03; SAD10319_P05_C05; SAD10319_P05_D02; SAD10319_P05_G02; SAD10319_P05_G03; SAD10319_P05_G04; SAD10319_P06_A07; SAD10319_P06_A11; SAD10319_P06_B11; SAD10319_P06_C12; SAD10319_P06_E09; SAD10319_P06_F07; SAD10319_P06_F10; SAD10319_P06_G09; SAD10319_P06_H07; SAD10319_P06_H08; and SAD10319_P06_H10.

In some embodiments, the disclosure provides an antibody comprising a CDRH1 binding domain comprising a consensus motif, the consensus motif comprising the sequence YTFX₁X₂X₃X₄MH, wherein X₁ is T, D, A, N, or V, wherein X₂ is D, E, G, or Q, wherein X₃ is Y or D, and wherein X₄ is D, A, E, N, S, Y, or V (SEQ ID NO: 29). In some embodiments, at least one of X₁, X₂, X₃, and X₄ is H. The following 35 clones include this consensus motif: SAD10319_P01_C01; SAD10319_P01_H01; SAD10319_P01_A02; SAD10319_P01_C02; SAD10319_P01_D02; SAD10319_P01_H02; SAD10319_P01_C03; SAD10319_P02_B04; SAD10319_P02_B05; SAD10319_P02_C05; SAD10319_P02_D05; SAD10319_P02_D06; SAD10319_P02_E06; SAD10319_P03_E08; SAD10319_P03_B09; SAD10319_P04_C10; SAD10319_P04_G10; SAD10319_P04_E11; SAD10319_P04_F11; SAD10319_P04_F12; SAD10319_P05_A03; SAD10319_P05_B05; SAD10319_P05_C01; SAD10319_P05_D01; SAD10319_P05_D03; SAD10319_P05_D05; SAD10319_P05_E04; SAD10319_P05_F01; SAD10319_P05_H06; SAD10319_P06_A10; SAD10319_P06_B10; SAD10319_P06_C10; SAD10319_P06_E10; SAD10319_P06_G11; and SAD10319_P06_H11.

In some embodiments, the disclosure provides an antibody comprising a CDRH1 binding domain comprising a consensus motif, the consensus motif comprising the sequence YTFTSX₁X₂MH, wherein X₁ is A, D, or T, and wherein X₂ is D, F, A, M, V, or Y (SEQ ID NO: 30). In some embodiments, at least one of X₁ and X₂ is H. The following 7 clones include this consensus motif: SAD10319_P01_G01; SAD10319_P01_E02; SAD10319_P04_A10; SAD10319_P04_B10; SAD10319_P04_H12; SAD10319_P05_B04; and SAD10319_P05_D04.

In some embodiments, the disclosure provides an antibody comprising a CDRH1 binding domain comprising a consensus motif, the consensus motif comprising the sequence YTFX₁X₂YX₃MH, wherein X₁ is N or T, X₂ is Q or N, and X₃ is S, T, or A (SEQ ID NO: 31). In some embodiments, at least one of X₁, X₂, and X₃ is H. The following 4 clones include this consensus motif: SAD10319_P02_F05; SAD10319_P02_G05; SAD10319_P02_H05; and SAD10319_P05_G01.

In some embodiments, the disclosure provides an antibody comprising a CDRH1 binding domain comprising a consensus motif, the consensus motif comprising the sequence YTFX₁X₂YVMH, wherein X₁ is I or N, and wherein X₂ is K or R (SEQ ID NO: 32). In some embodiments, at least one of X₁ and X₂ is H. The following 2 clones include this consensus motif: SAD10319_P06_D12 and SAD10319_P06_E08.

In some embodiments, the disclosure provides an antibody comprising a CDRH1 binding domain comprising a consensus motif, the consensus motif comprising the sequence FNIKDYYMH (SEQ ID NO: 47). At least the following 6 clones include this consensus motif and are designated as Group 1 binders: ADI-48592, ADI-48595, ADI-48650, ADI-48652, ADI-48662, and ADI-48666. Additionally, at least the following 16 clones include this consensus motif and are designated as Group 2 binders: ADI-48575, ADI-48576, ADI-48577, ADI-48581, ADI-48586, ADI-48587, ADI-48588, ADI-48590, ADI-48591, ADI-48593, ADI-48601, ADI-48646, ADI-48647, ADI-48597, ADI-48643, and ADI-48645.

In some embodiments, the disclosure provides an antibody comprising a CDRH1 binding domain comprising a consensus motif, the consensus motif comprising the sequence YTFX₁X₂YX₃MH, wherein X₁ is E, S, or T, wherein X₂ is S or D, and wherein X₃ is A or D (SEQ ID NO: 31). At least the following 5 clones include this consensus motif and are designated as Group 2 binders: ADI-48636, ADI-48638, ADI-48624, ADI-48632, and ADI-48635.

In some embodiments, the disclosure provides an antibody comprising a CDRL3 binding domain comprising a consensus motif, the consensus motif comprising the sequence X₁X₂SX₃X₄X₅RX₆, wherein X₁ is H, K, or G, wherein X₂ is Q or H, wherein X₃ is Y or H, wherein X₄ is S, H, D, T, V, M, or L, wherein X₅ is R or H, and wherein X₆ is T or H (SEQ ID NO: 33). In some embodiments, at least one of X₁, X₂, X₃, X₄, and X₅ is H. The following 156 clones include this consensus motif: LAD5224_P03_A01; SAD10318_P01_B01; SAD10318_P01_C01; SAD10318_P01_D01; SAD10318_P01_E01; SAD10318_P01_G01; SAD10318_P01_B02; SAD10318_P01_D02; SAD10318_P01_F02; SAD10318_P01_G02; SAD10318_P01_A03; SAD10318_P01_C03; SAD10318_P01_D03; SAD10318_P01_E03; SAD10318_P01_F03; SAD10318_P02_B04; SAD10318_P02_C04; SAD10318_P02_D04; SAD10318_P02_E04; SAD10318_P02_H04; SAD10318_P02_A05; SAD10318_P02_B05; SAD10318_P02_C05; SAD10318_P02_F05; SAD10318_P02_G05; SAD10318_P02_H05; SAD10318_P02_D06; SAD10318_P02_E06; SAD10318_P02_G06; SAD10318_P03_E07; SAD10318_P03_F07; SAD10318_P03_A08; SAD10318_P03_B08; SAD10318_P03_C08; SAD10318_P03_D08; SAD10318_P03_E08; SAD10318_P03_F08; SAD10318_P03_G08; SAD10318_P03_H08; SAD10318_P03_D09; SAD10318_P03_G09; SAD10318_P04_D10; SAD10318_P04_F10; SAD10318_P04_H10; SAD10318_P04_B11; SAD10318_P04_C11; SAD10318_P04_D11; SAD10318_P04_E11; SAD10318_P04_F11; SAD10318_P04_G11; SAD10318_P04_H11; SAD10318_P04_F12; SAD10318_P04_G12; SAD10318_P04_H12; SAD10319_P01_A01; SAD10319_P01_C01; SAD10319_P01_D01; SAD10319_P01_E01; SAD10319_P01_F01; SAD10319_P01_G01; SAD10319_P01_H01; SAD10319_P01_A02; SAD10319_P01_C02; SAD10319_P01_D02; SAD10319_P01_E02; SAD10319_P01_F02; SAD10319_P01_H02; SAD10319_P01_B03; SAD10319_P01_C03; SAD10319_P01_D03; SAD10319_P01_F03; SAD10319_P02_A04; SAD10319_P02_B04; SAD10319_P02_C04; SAD10319_P02_D04; SAD10319_P02_E04; SAD10319_P02_F04; SAD10319_P02_H04; SAD10319_P02_A05; SAD10319_P02_B05 SAD10319_P02_C05; SAD10319_P02_D05; SAD10319_P02_E05; SAD10319_P02_F05; SAD10319_P02_G05; SAD10319_P02_H05; SAD10319_P02_A06; SAD10319_P02_B06; SAD10319_P02_C06; SAD10319_P02_D06; SAD10319_P02_E06; SAD10319_P02_F06; SAD10319_P02_G06; SAD10319_P02_H06; SAD10319_P03_C07; SAD10319_P03_G07; SAD10319_P03_H07; SAD10319_P03_D08; SAD10319_P03_E08; SAD10319_P03_G08; SAD10319_P03_H08; SAD10319_P03_B09; SAD10319_P03_E09; SAD10319_P03_F09; SAD10319_P03_G09; SAD10319_P04_A10; SAD10319_P04_B10; SAD10319_P04_C10; SAD10319_P04_G10; SAD10319_P04_B11; SAD10319_P04_C11; SAD10319_P04_D11; SAD10319_P04_E11; SAD10319_P04_F11; SAD10319_P04_G11; SAD10319_P04_B12; SAD10319_P04_C12; SAD10319_P04_D12; SAD10319_P04_E12; SAD10319_P04_F12; SAD10319_P04_H12; SAD10320_P01_D01; SAD10320_P01_F01; SAD10320_P01_C02; SAD10320_P01_E02; SAD10320_P01_B03; SAD1032_P01_H03; SAD10320_P02_C04; SAD10320_P02_E04; SAD10320_P02_H04; SAD10320_P02_B05; SAD10320_P02_D05; SAD10320_P02_H05; SAD10320_P02_A06; SAD10320_P02_E06; SAD10320_P02_F06; SAD10320_P02_G06; SAD10320_P03_B07; SAD10320_P03_H07; SAD10320_P03_F08; SAD10320_P04_A10; SAD10320_P04_E10; SAD10320_P04_G10; SAD10320_P04_A11; SAD10320_P04_F11; SAD10320_P04_D12; SAD10320_P04_F12; LAD9953_P01_H01; LAD9954_P01_B02; LAD9955_P01_G02; LAD9956_P01_C03; LAD9959_P01_E04; LAD9960_P01_D05; LAD9963_P01_E06; LAD9964_P01_C07; and LAD9966_P01_A08.

In some embodiments, the disclosure provides an antibody comprising a CDRL3 binding domain comprising a consensus motif, the consensus motif comprising the sequence KQSYX₁X₂RT, wherein X₁ is H, V, K, W, R, L, G, Y, or Q, and wherein X₂ is H, L, E, W, G, M, P, T, Q, or V (SEQ ID NO: 34). In some embodiments, at least one of X₁ and X₂ is H. The following 45 clones include this consensus motif: SAD10318_P01_F01; SAD10318_P01_H01; SAD10318_P01_A02; SAD10318_P01_G03; SAD10318_P01_H03; SAD10318_P02_G04; SAD10318_P02_D05; SAD10318_P02_B06; SAD10318_P03_G07; SAD10318_P03_A09; SAD10318_P03_C09; SAD10318_P03_E09; SAD10318_P04_A12; SAD10320_P01_B01; SAD10320_P01_E01; SAD10320_P01_G01; SAD10320_P01_A02; SAD10320_P01_F02; SAD10320_P01_G02; SAD10320_P01_C03; SAD10320_P01_D03; SAD10320_P01_E03; SAD10320_P01_F03; SAD10320_P01_G03; SAD10320_P02_B04; SAD10320_P02_A05; SAD10320_P02_C05; SAD10320_P02_F05; SAD10320_P02_B06; SAD10320_P02_D06; SAD10320_P02_H06; SAD10320_P03_E07; SAD10320_P03_C08; SAD10320_P03_D08; SAD10320_P03_H08; SAD10320_P03_C09; SAD10320_P04_C10; SAD10320_P04_D10; SAD10320_P04_F10; SAD10320_P04_D11; SAD10320_P04_E11; SAD10320_P04_G11; SAD10320_P04_H11; SAD10320_P04_A12; and SAD10320_P04_E12.

In some embodiments, the disclosure provides an antibody comprising a CDRL3 binding domain comprising a consensus motif, the consensus motif comprising the sequence X₁QSX₂HX₃RT, wherein X₁ is K or H, wherein X₂ is H, Y, M, S, L, E, G, or W, and wherein X₃ is R or K (SEQ ID NO: 35). In some embodiments, at least one of X₁, X₂, and X₃ is H. The following 14 clones include this consensus motif: SAD10318_P03_B07; SAD10318_P03_F09; SAD10318_P04_C10; SAD10318_P04_E10; SAD10318_P04_A11; SAD10320_P01_H01; SAD10320_P01_H02; SAD10320_P01_A03; SAD10320_P02_A04; SAD10320_P02_E05, SAD10320_P02_C06; SAD10320_P03_A09; SAD10320_P03_D09; and SAD10320_P03_F09.

In some embodiments, the disclosure provides an antibody comprising a CDRL3 binding domain comprising a consensus motif, the consensus motif comprising the sequence KQSX₁X₂X₃RT, wherein X₁ is Y or H, X₂ is T, S, V, or K, and X₃ is R or H (SEQ ID NO: 36). In some embodiments, at least one of X₁, X₂, and X₃ is H. The following 11 clones include this consensus motif: ADI-48576; ADI-48577; ADI-48587; ADI-48592, ADI-48595, ADI-48635, ADI-48650; ADI-48652; ADI-48666; ADI-48645; and ADI-48643.

In some embodiments, the disclosure provides an antibody comprising a CDRL3 binding domain comprising a consensus motif, the consensus motif comprising the sequence KQSX₁X₂X₃RT, wherein X₁ is H or Y, wherein X₂ is T, S, or Q, and wherein X₃ is R or H (SEQ ID NO: 36). In some embodiments, at least one of X₁ and X₃ is H. At least the following 6 clones include this consensus motif and are designated as Group 1 binders: ADI-48592, ADI-48595, ADI-48650, ADI-48652, ADI-48662, and ADI-48666.

In some embodiments, the disclosure provides an antibody comprising a CDRL3 binding domain comprising a consensus motif, the consensus motif comprising the sequence X₁QSX₂X₃X₄RT, wherein X₁ is K or H, wherein X₂ is Y or H, wherein X₃ is S, H, L, V, or K, and wherein X₄ is H, R, or E (SEQ ID NO: 598). In some embodiments, at least one of X₁, X₂, X₃, and X₄ is H. At least the following 21 clones include this consensus motif and are designated as Group 2 binders: ADI-48588, ADI-48587, ADI-48577, ADI-48590, ADI-48581, ADI-48575, ADI-48593, ADI-48591, ADI-48647, ADI-48636, ADI-48586, ADI-48646, ADI-48638, ADI-48597, ADI-48601, ADI-48576, ADI-48643, ADI-48624, ADI-48632, ADI-48635, and ADI-48645.

In some embodiments, the disclosure provides an antibody comprising a CDRL2 binding domain comprising a consensus motif, the consensus motif comprising the sequence WASTRES (SEQ ID NO: 37). The following 215 clones include this consensus motif: LAD5224_P03_A01; SAD10318_P01_B01; SAD10318_P01_C01; SAD10318_P01_D01; SAD10318_P01_E01; SAD10318_P01_F01; SAD10318_P01_G01; SAD10318_P01_H01; SAD10318_P01_A02; SAD10318_P01_B02; SAD10318_P01_D02; SAD10318_P01_F02; SAD10318_P01_G02; SAD10318_P01_A03; SAD10318_P01_C03; SAD10318_P01_D03; SAD10318_P01_E03; SAD10318_P01_F03; SAD10318_P01_G03; SAD10318_P01_H03; SAD10318_P02_B04; SAD10318_P02_C04; SAD10318_P02_D04; SAD10318_P02_E04; SAD10318_P02_G04; SAD10318_P02_H04; SAD10318_P02_A05; SAD10318_P02_B05; SAD10318_P02_C05; SAD10318_P02_D05; SAD10318_P02_F05; SAD10318_P02_G05; SAD10318_P02_H05; SAD10318_P02_B06; SAD10318_P02_D06; SAD10318_P02_E06; SAD10318_P02_G06; SAD10318_P03_B07; SAD10318_P03_E07; SAD10318_P03_F07; SAD10318_P03_G07; SAD10318_P03_A08; SAD10318_P03_B08; SAD10318_P03_C08; SAD10318_P03_D08; SAD10318_P03_E08; SAD10318_P03_F08; SAD10318_P03_G08; SAD10318_P03_H08; SAD10318_P03_A09; SAD10318_P03_C09; SAD10318_P03_D09; SAD10318_P03_E09; SAD10318_P03_F09; SAD10318_P03_G09; SAD10318_P04_C10; SAD10318_P04_D10; SAD10318_P04_E10; SAD10318_P04_F10; SAD10318_P04_H10; SAD10318_P04_A11; SAD10318_P04_111; SAD10318_P04_C11; SAD10318_P04_D11; SAD10318_P04_E11; SAD10318_P04_F11; SAD10318_P04_G11; SAD10318_P04_H11; SAD10318_P04_A12; SAD10318_P04_F12; SAD10318_P04_G12; SAD10318_P04_H12; SAD10319_P01_A01; SAD10319_P01_C01; SAD10319_P01_D01; SAD10319_P01_E01; SAD10319_P01_F01; SAD10319_P01_G01; SAD10319_P01_H01; SAD10319_P01_A02; SAD10319_P01_C02; SAD10319_P01_D02; SAD10319_P01_E02; SAD10319_P01_F02; SAD10319_P01_H02; SAD10319_P01_B03; SAD10319_P01_C03; SAD10319_P01_D03; SAD10319_P01_F03; SAD10319_P02_A04; SAD10319_P02_B04; SAD10319_P02_C04; SAD10319_P02_D04; SAD10319_P02_E04; SAD10319_P02_F04; SAD10319_P02_H04; SAD10319_P02_A05; SAD10319_P02_B05; SAD10319_P02_C05; SAD10319_P02_D05; SAD10319_P02_E05; SAD10319_P02_F05; SAD10319_P02_G05; SAD10319_P02_H05; SAD10319_P02_A06; SAD10319_P02_B06; SAD10319_P02_C06; SAD10319_P02_D06; SAD10319_P02_E06; SAD10319_P02_F06; SAD10319_P02_G06; SAD10319_P02_H06; SAD10319_P03_C07; SAD10319_P03_G07; SAD10319_P03_H07; SAD10319_P03_D08; SAD10319_P03_E08; SAD10319_P03_G08; SAD10319_P03_H08; SAD10319_P03_B09; SAD10319_P03_E09; SAD10319_P03_F09; SAD10319_P03_G09; SAD10319_P04_A10; SAD10319_P04_B10; SAD10319_P04_C10; SAD10319_P04_G10; SAD10319_P04_B11; SAD10319_P04_C11; SAD10319_P04_D11; SAD10319_P04_E11; SAD10319_P04_F11; SAD10319_P04_G11; SAD10319_P04_B12; SAD10319_P04_C12; SAD10319_P04_D12; SAD10319_P04_E12; SAD10319_P04_F12; SAD10319_P04_H12; SAD10320_P01_B01; SAD10320_P01_D01; SAD1032_P01_E01; SAD10320_P01_F01; SAD10320_P01_G01; SAD10320_P01_H01; SAD10320_P01_A02; SAD10320_P01_C02; SAD10320_P01_E02; SAD10320_P01_F02; SAD10320_P01_G02; SAD10320_P01_H02; SAD10320_P01_A03; SAD10320_P01_B03; SAD10320_P01_C03; SAD10320_P01_D03; SAD10320_P01_E03; SAD10320_P01_F03; SAD10320_P01_G03; SAD10320_P01_H03; SAD10320_P02_A04; SAD10320_P02_B04; SAD10320_P02_C04; SAD10320_P02_E04; SAD10320_P02_H04; SAD10320_P02_A05; SAD10320_P02_B05; SAD10320_P02_C05; SAD10320_P02_D05; SAD10320_P02_E05; SAD10320_P02_F05; SAD10320_P02_H05; SAD10320_P02_A06; SAD10320_P02_B06; SAD10320_P02_C06; SAD10320_P02_D06; SAD10320_P02_E06; SAD10320_P02_F06; SAD10320_P02_G06; SAD10320_P02_H06; SAD10320_P03_B07; SAD10320_P03_E07; SAD10320_P03_H07; SAD10320_P03_C08; SAD10320_P03_D08; SAD10320_P03_F08; SAD10320_P03_H08; SAD10320_P03_A09; SAD10320_P03_C09; SAD10320_P03_D09; SAD10320_P03_F09; SAD10320_P04_A10; SAD10320_P04_C10; SAD10320_P04_D10; SAD10320_P04_E10; SAD10320_P04_F10; SAD10320_P04_G10; SAD10320_P04_A11; SAD10320_P04_D11; SAD10320_P04_E11; SAD10320_P04_F11; SAD10320_P04_G11; SAD10320_P04_H11; SAD10320_P04_A12; SAD10320_P04_D12; SAD10320_P04_E12; SAD10320_P04_F12; LAD9953_P01_H01; LAD9954_P01_B02; LAD9955_P01_G02; LAD9956_P01_C03; LAD9959_P01_E04; LAD9960_P01_D05; LAD9963_P01_E06; LAD9964_P01_C07; and LAD9966_P01_A08. Additionally, at least the following 6 clones include this consensus motif and are designated as Group 1 binders: ADI-48592, ADI-48595, ADI-48650, ADI-48652, ADI-48662, and ADI-48666; and at least the following 21 clones include this consensus motif and are designated as Group 2 binders: ADI-48588, ADI-48587, ADI-48577, ADI-48590, ADI-48581, ADI-48575, ADI-48593, ADI-48591, ADI-48647, ADI-48636, ADI-48586, ADI-48646, ADI-48638, ADI-48597, ADI-48601, ADI-48576, ADI-48643, ADI-48624, ADI-48632, ADI-48635, and ADI-48645.

In some embodiments, the disclosure provides an antibody comprising a CDRL1 binding domain comprising a consensus motif, the consensus motif comprising the sequence KSSQSLLX1X₂X₃X₄GX₅NX₆LA, wherein X₁ is N or H, wherein X₂ is A, R, or T, wherein X₃ is R or H, wherein X₄ is T, P, or E, wherein X₅ is H or K, and wherein X₆ is H or Y (SEQ ID NO: 38). In some embodiments, at least one of X₁, X₂, X₃, X₄, X₅, and X₆ is H. The following 203 clones include this consensus motif: LAD5224_P03_A01; SAD10318_P01_B01; SAD10318_P01_D01; SAD10318_P01_E01; SAD10318_P01_F01; SAD10318_P01_G01; SAD10318_P01_H01; SAD10318_P01_B02; SAD10318_P01_D02; SAD10318_P01_F02; SAD10318_P01_G02; SAD10318_P01_A03; SAD10318_P01_C03; SAD10318_P01_D03; SAD10318_P01_E03; SAD10318_P01_F03; SAD10318_P01_G03; SAD10318_P01_H03; SAD10318_P02_B04; SAD10318_P02_C04; SAD10318_P02_D04; SAD10318_P02_E04; SAD10318_P02_G04; SAD10318_P02_H04; SAD10318_P02_A05; SAD10318_P02_B05; SAD10318_P02_C05; SAD10318_P02_D05; SAD10318_P02_F05; SAD10318_P02_H05; SAD10318_P02_B06; SAD10318_P02_D06; SAD10318_P02_G06; SAD10318_P03_B07; SAD10318_P03_E07; SAD10318_P03_F07; SAD10318_P03_G07; SAD10318_P03_A08; SAD10318_P03_B08; SAD10318_P03_C08; SAD10318_P03_D08; SAD10318_P03_E08; SAD10318_P03_F08; SAD10318_P03_G08; SAD10318_P03_H08; SAD10318_P03_A09; SAD10318_P03_C09; SAD10318_P03_D09; SAD10318_P03_E09; SAD10318_P03_F09; SAD10318_P03_G09; SAD10318_P04_C10; SAD10318_P04_D10; SAD10318_P04_E10; SAD10318_P04_H10; SAD10318_P04_A11; SAD10318_P04_B11; SAD10318_P04_C11; SAD10318_P04_D11; SAD10318_P04_E11; SAD10318_P04_F11; SAD10318_P04_G11; SAD10318_P04_H11; SAD10318_P04_A12; SAD10318_P04_F12; SAD10318_P04_G12; SAD10318_P04_H12; SAD10319_P01_A01; SAD10319_P01_C01; SAD10319_P01_D01; SAD10319_P01_E01; SAD10319_P01_F01; SAD10319_P01_G01; SAD10319_P01_H01; SAD10319_P01_A02; SAD10319_P01_C02; SAD10319_P01_D02; SAD10319_P01_E02; SAD10319_P01_F02; SAD10319_P01_H02; SAD10319_P01_B03; SAD10319_P01_C03; SAD10319_P01_D03; SAD10319_P01_F03; SAD10319_P02_A04; SAD10319_P02_B04; SAD10319_P02_C04; SAD10319_P02_D04; SAD10319_P02_E04; SAD10319_P02_F04; SAD10319_P02_H04; SAD10319_P02_A05; SAD10319_P02_B05; SAD10319_P02_C05; SAD10319_P02_D05; SAD10319_P02_E05; SAD10319_P02_F05; SAD10319_P02_G05; SAD10319_P02_H05; SAD10319_P02_A06; SAD10319_P02_B06; SAD10319_P02_C06; SAD10319_P02_D06; SAD10319_P02_E06; SAD10319_P02_F06; SAD10319_P02_G06; SAD10319_P02_H06; SAD10319_P03_C07; SAD10319_P03_G07; SAD10319_P03_H07; SAD10319_P03_D08; SAD10319_P03_E08; SAD10319_P03_G08; SAD10319_P03_H08; SAD10319_P03_B09; SAD10319_P03_E09; SAD10319_P03_F09; SAD10319_P03_G09; SAD10319_P04_A10; SAD10319_P04_B10; SAD10319_P04_C10; SAD10319_P04_G10; SAD10319_P04_111; SAD10319_P04_C11; SAD10319_P04_D11; SAD10319_P04_E11; SAD10319_P04_F11; SAD10319_P04_G11; SAD10319_P04_B12; SAD10319_P04_C12; SAD10319_P04_D12; SAD10319_P04_E12; SAD10319_P04_F12; SAD10319_P04_H12; SAD10320_P01_B01; SAD10320_P01_D01; SAD1032_P01_E01; SAD10320_P01_F01; SAD10320_P01_G01; SAD10320_P01_H01; SAD10320_P01_A02; SAD10320_P01_C02; SAD10320_P01_E02; SAD10320_P01_F02; SAD10320_P01_G02; SAD10320_P01_H02; SAD10320_P01_A03; SAD10320_P01_B03; SAD10320_P01_C03; SAD10320_P01_D03; SAD10320_P01_E03; SAD10320_P01_F03; SAD10320_P01_G03; SAD10320_P01_H03; SAD10320_P02_A04; SAD10320_P02_B04; SAD10320_P02_C04; SAD10320_P02_E04; SAD10320_P02_H04; SAD10320_P02_A05; SAD10320_P02_B05; SAD10320_P02_C05; SAD10320_P02_D05; SAD10320_P02_E05; SAD10320_P02_F05; SAD10320_P02_H05; SAD10320_P02_A06; SAD10320_P02_B06; SAD10320_P02_C06; SAD10320_P02_D06; SAD10320_P02_E06; SAD10320_P02_F06; SAD10320_P02_H06; SAD10320_P03_B07; SAD10320_P03_E07; SAD10320_P03_H07; SAD10320_P03_C08; SAD10320_P03_D08; SAD10320_P03_F08; SAD10320_P03_A09; SAD10320_P03_C09; SAD10320_P03_D09; SAD10320_P03_F09; SAD10320_P04_A10; SAD10320_P04_C10; SAD10320_P04_D10; SAD10320_P04_E10; SAD10320_P04_F10; SAD10320_P04_G10; SAD10320_P04_A11; SAD10320_P04_D11; SAD10320_P04_E11; SAD10320_P04_F11; SAD10320_P04_G1; SAD10320_P04_H11; SAD10320_P04_A12; SAD10320_P04_D12; SAD10320_P04_E12; SAD10320_P04_F12; LAD9954_P01_B02; LAD9955_P01 G02; LAD9963_P01_E06; and LAD9966_P01_A08.

In some embodiments, the disclosure provides an antibody comprising a CDRL1 binding domain comprising a consensus motif, the consensus motif comprising the sequence KSSQSLLX₁AX₂THX₃NX₄LA, wherein X₁ is N or H, wherein X₂ is R or H, wherein X₃ is K or H, and wherein X₄ is Y or H (SEQ ID NO: 39). In some embodiments, at least one of X₁, X₂, X₃, and X₄ is H. The following 10 clones include this consensus motif: SAD10318_P01_C01; SAD10318_P01_A02; SAD10318_P02_G05; SAD10318_P02_E06; SAD10318_P04_F10; LAD9953_P01_H01; LAD9956_P01_C03; LAD9959_P01_E04; LAD9960_P01_D05; and LAD9964_P01_C07.

In some embodiments, the disclosure provides an antibody comprising a CDRL1 binding domain comprising a consensus motif, the consensus motif comprising the sequence KSSQSLLNASTAKNYLA (SEQ ID NO: 40) or KSSQSLLNARTRTNYLA (SEQ ID NO: 41).

In some embodiments, the disclosure provides an antibody comprising a CDRL1 binding domain comprising a consensus motif, the consensus motif comprising the sequence KSSQSLLNX₁X₂X₃GX₄NX₅LA, wherein X₁ is S or A, X₂ is R or H, X₃ is E or T, X₄ is H or K, and X₅ is H or Y (SEQ ID NO: 42). In some embodiments, at least one of X₁, X₂, X₃, X₄, and X₅ is H. The following 11 clones include this consensus motif: ADI-48576; ADI-48577; ADI-48587; ADI-48592; ADI-48595; ADI-48635; ADI-48645; ADI-48650; ADI-48652; ADI-48643; and ADI-48666.

In some embodiments, the disclosure provides an antibody comprising a CDRL1 binding domain comprising a consensus motif, the consensus motif comprising the sequence KSSQSLLNX₁X₂TGX₃NYLA, wherein X₁ is A or S, wherein X₂ is R or H, and wherein X₃ is H or K (SEQ ID NO: 594). In some embodiments, at least one of X₂ and X₃ is H. At least the following 6 clones include this consensus motif and are designated as Group 1 binders: ADI-48592, ADI-48595, ADI-48650, ADI-48652, ADI-48662, and ADI-48666.

In some embodiments, the disclosure provides an antibody comprising a CDRL1 binding domain comprising a consensus motif, the consensus motif comprising the sequence KSSQSLLX₁AX₂X₃X₄X₅NX₆LA, wherein X₁ is N or H, wherein X₂ is R or H, wherein X₃ is T or E, wherein X₄ is G or H, wherein X₅ is H or K, and wherein X₆ is H or Y (SEQ ID NO: 597). In some embodiments, at least one of X₁, X₂, X₄, X₅, and X₆ is H. At least the following 21 clones include this consensus motif and are designated as Group 2 binders: ADI-48588, ADI-48587, ADI-48577, ADI-48590, ADI-48581, ADI-48575, ADI-48593, ADI-48591, ADI-48647, ADI-48636, ADI-48586, ADI-48646, ADI-48638, ADI-48597, ADI-48601, ADI-48576, ADI-48643, ADI-48624, ADI-48632, ADI-48635, and ADI-48645.

In some embodiments, the disclosure provides an antibody comprising a CDRH3 binding domain comprising a consensus motif, the consensus motif comprising the sequence AX₁DX₂YX₃HX₄FYDV, wherein X₁ is R or H, wherein X₂ is A or H, wherein X₃ is G, H, or P, and wherein X₄ is Y, H, D, V, E, S, N, L, M, I, G, A, Q, or T (SEQ ID NO: 1); a CDRH2 binding domain comprising a consensus motif, the consensus motif comprising the sequence WIDLENANTIYDAKFQG (SEQ ID NO: 9); WINPX₁TGATX₂YSQKFQG, wherein X₁ is S, D, A, N, L, or Q and wherein X₂ is V, T, D, Y, or K (SEQ ID NO: 45); or X₁IDAGTGATX₂YSQKFQG, wherein X₁ is W, S, or D and wherein X₂ is A, H, K, T, or D (SEQ ID NO: 46); a CDRH1 binding domain comprising a consensus motif, the consensus motif comprising the sequence FNIKDYYMH (SEQ ID NO: 47); a CDRL3 binding domain comprising a consensus motif, the consensus motif comprising the sequence KSQYX₁X₂RT, wherein X₁ is S, H, V, K, W, L, G, T, R, or Q and wherein X₂ is H, R, L, K, E, W, G, M, T, or V (SEQ ID NO: 48); a CDRL2 binding domain comprising a consensus motif, the consensus motif comprising the sequence WASTRES (SEQ ID NO: 37), and a CDRL1 binding domain comprising a consensus motif, the consensus motif comprising the sequence KSSQSLLNARTGKNYLA (SEQ ID NO: 49).

In some embodiments, the disclosure provides an antibody comprising a CDRH3 binding domain binding domain comprising a consensus motif, the consensus motif comprising the sequence ARDX₁YGX₂X₃X₄YDX₅ wherein X₁ is A or H, wherein X₂ is R or H, wherein X₃ is H or Y, wherein X₄ is F or H, and wherein X₅ is H or V (SEQ ID NO: 2); a CDRH2 binding domain comprising a consensus motif, the consensus motif comprising the sequence WIDLENANTIYDAKFQG (SEQ ID NO: 9) or WIX₁AGTGATX₂YSQKGQG, wherein X₁ is T, N, or D, and wherein X₂ is V or K (SEQ ID NO: 50); a CDRH1 binding domain comprising a consensus motif, the consensus motif comprising the sequence FNIKDYYMH (SEQ ID NO: 47) or YTFX₁X₂YX₃MH, wherein X₁ is T or A, X₂ is E, D, A, S, G or Q, and X₃ is D, A, V, or E (SEQ ID NO: 51); a CDRL3 binding domain comprising a consensus motif, the consensus motif comprising the sequence KQSX₁SRRT, wherein X₁ is H or Y (SEQ ID NO: 52); a CDRL2 binding domain comprising a consensus motif, the consensus motif comprising the sequence WASTRES (SEQ ID NO: 37); and a CDRL1 binding domain comprising a consensus motif, the consensus motif comprising the sequence KSSQSLLX₁AX₂TX₃X₄NX₅LA, wherein X₁ is N or H, X₂ is R or H, X₃ is G or H, X₄ is K or H, and X₅ is H or Y (SEQ ID NO: 53).

In some embodiments, the disclosure provides an antibody comprising a CDRH3 binding domain comprising a consensus motif, the consensus motif comprising the sequence ARDAHX₁X₂YX₃X₄DX₅, wherein X₁ is G, E, or R, wherein X₂ is R or H, wherein X₃ is F or H, wherein X₄ is Y or H, and wherein X₅ is V or H (SEQ ID NO: 3); a CDRH2 binding domain comprising a consensus motif, the consensus motif comprising the sequence WIDLENANTIYDAKFQG (SEQ ID NO: 9); a CDRH1 binding domain comprising a consensus motif, the consensus motif comprising the sequence FNIKDYYMH (SEQ ID NO: 47); a CDRL3 binding domain comprising a consensus motif, the consensus motif comprising the sequence KQSYSRRT (SEQ ID NO: 54); a CDRL2 binding domain comprising a consensus motif, the consensus motif comprising the sequence WASTRES (SEQ ID NO: 37); and a CDRL1 binding domain comprising a consensus motif, the consensus motif comprising the sequence KSSQSLLNAX₁TGX₂NX₃LA, wherein X₁ is H or R, wherein X₂ is H or K, and X₃ is H or Y (SEQ ID NO: 55).

In some embodiments, the disclosure provides an antibody or antigen-binding fragment comprising a CDRH3 binding domain comprising a consensus motif, the consensus motif comprising the sequence ARDAX₁X₂X₃X₄FYDX₅, wherein X₁ is T, H, or Y, wherein X₂ is G or H, wherein X₃ is H or R, wherein X₄ is V or Y, wherein X₅ is V or H, and wherein, optionally, at least one of X₁, X₂, X₃, and X₅ is H (SEQ ID NO: 593); a CDRH2 binding domain comprising a consensus motif, the consensus motif comprising the sequence WIDLENANTIYDAKFQG (SEQ ID NO: 9); a CDRH1 binding domain comprising a consensus motif, the consensus motif comprising the sequence FNIKDYYMH (SEQ ID NO: 47); a CDRL3 binding domain comprising a consensus motif, the consensus motif comprising the sequence KQSX₁X₂X₃RT, wherein X₁ is H or Y, wherein X₂ is T, S, or Q, wherein X₃ is R or H, and, optionally, wherein at least one of X₁ and X₃ is H (SEQ ID NO: 36); a CDRL2 binding domain comprising a consensus motif, the consensus motif comprising the sequence WASTRES (SEQ ID NO: 37); and/or a CDRL1 binding domain comprising a consensus motif, the consensus motif comprising the sequence KSSQSLLNX₁X₂TGX₃NYLA, wherein X₁ is A or S, wherein X₂ is R or H, wherein X₃ is H or K, and, optionally, wherein at least one of X₂ and X₃ is H (SEQ ID NO: 594). In some embodiments, said antibody or antigen-binding fragment is designated as a Group 1 binder comprising a CD3 binding domain selected from ADI-48592, ADI-48595, ADI-48650, ADI-48652, ADI-48662, and ADI-48666.

In some embodiments, the disclosure provides an antibody or antigen-binding fragment comprising a CDRH3 binding domain comprising a consensus motif, the consensus motif comprising the sequence AX₁DX₂X₃X₄X₅X₆X₇YDX₈, wherein X₁ is R or H, wherein X₂ is H or A, wherein X₃ is H or Y, wherein X₄ is H, G, or P, wherein X₅ is R or H, wherein X₆ is Y, I, or V, wherein X₇ is F or H, wherein X₈ is V or H, and, optionally, wherein at least one of X₁, X₂, X₃, X₄, X₅, X₇, and X₈ is H (SEQ ID NO: 596); a CDRH2 binding domain comprising a consensus motif, the consensus motif comprising the sequence WIDLENANTIYDAKFQG (SEQ ID NO: 9) or the sequence WIDAGTGX₁TX₂YSQKFQG, wherein X₁ is L, F, N, or A and wherein X₂ is T or K (SEQ ID NO: 595); a CDRH1 binding domain comprising a consensus motif, the consensus motif comprising the sequence FNIKDYYMH (SEQ ID NO: 47) or the sequence YTFX₁X₂YX₃MH, wherein X₁ is E, S, or T, wherein X₂ is S or D, and wherein X₃ is A or D (SEQ ID NO: 31); a CDRL3 binding domain comprising a consensus motif, the consensus motif comprising the sequence X₁QSX₂X₃X₄RT, wherein X₁ is K or H, wherein X₂ is Y or H, wherein X₃ is S, H, L, V, or K, wherein X₄ is H, R, or E, and, optionally, wherein at least one of X₁, X₂, X₃, and X₄ is H (SEQ ID NO: 598); a CDRL2 binding domain comprising a consensus motif, the consensus motif comprising the sequence WASTRES (SEQ ID NO: 37); and/or a CDRL1 binding domain comprising a consensus motif, the consensus motif comprising the sequence KSSQSLLX₁AX₂X₃X₄X₅NX₆LA, wherein X₁ is N or H, wherein X₂ is R or H, wherein X₃ is T or E, wherein X₄ is G or H, wherein X₅ is H or K, wherein X₆ is H or Y, and wherein, optionally, at least one of X₁, X₂, X₄, X₅, and X₆ is H (SEQ ID NO: 597). In some embodiments, said antibody or antigen-binding fragment is designated as a Group 2 binder comprising a CD3 binding domain selected from ADI-48588, ADI-48587, ADI-48577, ADI-48590, ADI-48581, ADI-48575, ADI-48593, ADI-48591, ADI-48647, ADI-48636, ADI-48586, ADI-48646, ADI-48638, ADI-48597, ADI-48601, ADI-48576, ADI-48643, ADI-48624, ADI-48632, ADI-48635, and ADI-48645.

### Materials and Methods

In addition to the description provided above, the following Materials and Methods were employed in the Examples.

Hu and Cy CD3εϵ Fc heterodimer antigen production. Recombinant heterodimeric CD3 Fc fusion antigens were produced in HEK 293 cells by co-transfection of plasmids encoding Hu CD3ε Fc (ectodomain, ECD, residues 22-126) and CD3δ Fc-HIS (ECD residues 22-100) or Cy CD3ε Fc (ECD residues 22-117) and CD3δ Fc-HIS (ECD residues 22-100) utilizing a heterologous signal peptide sequence. Chromatographic separations were performed on a computer controlled ÄKTA Avant 150 preparative chromatography system (GE Healthcare Life Sciences) equipped with an integrated conductivity sensor, enabling in-line salt concentration monitoring during the run. Clarified culture supernatants were purified by Ni Sepharose 6 Fast Flow (GE Healthcare Life Sciences), which removes the CD3εε Fc-HIS homodimer. CD3εδ Fc-HIS heterodimer was resolved from CD366 Fc-HIS homodimer by Mono Q 10/100 GL by a linear Tris-buffered KCl gradient at pH 8.5.

Peptides. C-terminally biotinylated CD3ε N-terminal peptides were obtained from New England Peptide. All peptides were delivered with a purity of ≥95%. Peptides were designed based on the primary sequence of Hu CD3ε and the crystal structure of Hu CD3εδ bound to OKT3 (Kjer-Nielsen L. et al. PNAS 2004). The CD3εN27 peptide has the sequence H2N-QDGNEEMGSITQTPYQVSISGTTVILT[K/SCBiot(dPEG4)]-amide (SEQ ID NO: 56) and the CD3εN13 peptide has the sequence H2N-QDGNEEMGGITQT[K/SCBiot(dPEG4)]-amide (SEQ ID NO: 57).

Antigen biotinylation. CD3 antigens were biotinylated using the EZ-Link Sulfo-NHS-Biotinylation Kit from Pierce. Goat anti-human F(ab′)2 kappa-FITC (LC-FITC), Extravidin-PE (EA-PE) and streptavidin-633 (SA-633) were obtained from Southern Biotech, Sigma and Molecular Probes, respectively. Streptavidin MicroBeads and MACS LC separation columns were purchased from Miltenyi Biotec.

Cell line propagation and cell labeling assays. Human Jurkat CD3+ cells (ATCC TIB-152) and Jurkat CD3− cells (ATCC TIB-153) were obtained from ATCC. Cyno HSC-F cells were obtained from the NIH Non-human Primate Reagent Resource. All cell lines were cultured in RPMI 1640 GlutaMax media supplemented with 10% fetal bovine serum (FBS).

Cell labeling was conducted by aliquoting 100,000-200,000 cells per well in a 96-well assay plate. Cells were centrifugated at 500×g for 5 min at 4° C., then resuspended in 100 μl of 100 nM IgG and incubated at room temperature for 20 min. Cells were then washed in buffer (phosphate-buffered saline (PBS)/0.1% bovine serum albumin (BSA) three times and resuspended in secondary reagent, typically goat anti-human R-PE (Southern Biotech). The plate was assayed on a FACSCanto (BD Biosciences) using an HTS sample injector. Flow cytometry data was analyzed for median fluorescence intensity in the R-PE channel.

FACS affinity pressured selection methods. Briefly, yeast cells (at least ˜2×10⁷ cells/labeling condition) were incubated with a volume of biotinylated antigen sufficient to represent a stoichiometric excess with respect to the average IgG presentation number. Antigen labeling conditions are 100 to 1 nM under equilibrium conditions, typically carried out for 20 min to several hours at room temperature in FACS wash buffer (phosphate-buffered saline (PBS)/0.1% bovine serum albumin (BSA)). After washing three times with wash buffer, yeast are then stained with secondary reagents anti-human light chain FITC conjugate (LC-FITC) diluted 1:100 and either streptavidin-633 (SA-633) diluted 1:500 or extravidin-phycoerythrin (EA-PE) diluted 1:50 for 15 min at 4° C. After washing twice with ice-cold wash buffer, the cell pellets are resuspended in wash buffer in a typical volume of at least 1 mL per 1×10⁷ yeast and transferred to strainer-capped sort tubes. Sorting is performed using a FACS ARIA sorter (BD Biosciences) and sort gates are determined to select for binders. After the final round of sorting, yeast were plated and individual colonies picked for characterization.

Antibody yeast production and purification. Yeast clones were grown to saturation and then induced for 48 h at 30° C. with shaking. After induction, yeast cells were pelleted and the supernatants were harvested for purification. IgGs were purified using a Protein A column and eluted with acetic acid, pH 2.0. Fab fragments were generated by papain digestion and purified over KappaSelect or CaptureSelect IgG-CH1 (GE Healthcare LifeSciences).

Antibody HEK production and purification. Mammalian expression of IgG was done by sub-cloning antibodies into a new expression vector followed by transient transfection and expression in HEK293ADI1, a monoclonal cell line derived from HEK293 (DSMZ) selected for clump-free growth, growth rate, and transfectability. Briefly, expression vectors containing the antibody of interest were transfected by complexing with a transfection reagent followed by exposure to HEK cells for one hour followed by dilution of culture media to a final density of 4 million cells per mL. The cells were then cultured for 7 days with fresh feed media every 48 hours. After 7 days, the supernatant was collected following centrifugation and purification was performed using protein A. If necessary, a CHT column purification was added to reach >95% monomer.

Cell binding assays. CD3+ human Jurkat cells (ATCC) and CHO-S cells (Invitrogen/ThermoFisher) were thawed and washed with cold PBSF buffer, pH 7.4 (PBS+0.1% BSA, pH 7.4). About 200,000 cells were aliquoted per well of a 96-well plate (FACS Assay Plate VWR BD 353263) and pelleted by centrifugation (5 minutes at 500×g). The cells were washed with either PBSF pH 7.4 or PBSF pH 6.0 (PBS+0.1% BSA, pH 6.0), and then resuspended in 100 ul in either PBSF pH 7.4 or PBSF pH 6.0 with IgG antibody (100 nM) produced in yeast as described above. The mixture (cells+antibody) was incubated for 20 minutes on ice, then washed twice with either PBSF pH 7.4 or PBSF pH 6.0. Cells were resuspended in 50 ul of propidium iodide (Roche; 1:500 dilution) and anti-human IgG-RPE (Southern Biotech; 1:100 dilution) prepared in either PBSF pH 7.4 or PBSF pH 6.0, then incubated for 20 minutes on ice in the dark before cells were washed twice with either PBSF pH 7.4 or PBSF pH 6.0. Binding was analyzed on FACS Canto II.

ForteBio K_{D} measurements (Biolayer interferometry; BLI). ForteBio affinity measurements were performed generally as previously described (Estep, P., et al., High throughput solution-based measurement of antibody-antigen affinity and epitope binning. MAbs, 2013. 5(2): p. 270-8.). Briefly, ForteBio affinity measurements were performed by loading IgGs online onto AHC sensors. Sensors were equilibrated off-line in assay buffer for 30 min and then monitored on-line for 60 seconds for baseline establishment. Sensors with loaded IgGs were exposed to 100 nM antigen (e.g., CD3) for 5 min, afterwards they were transferred to assay buffer for 5 min for off-rate measurement. Kinetics were analyzed using the 1:1 binding model.

PSR Preparation. Polyspecific reactivity reagent (PSR) was prepared as described in, e.g., WO 2014/179363 and Xu et. al., Protein Eng Des Sel, 26(10):663-670 (2013). In brief, 2.5 liters CHO-S cells were used as starting material. The cells were pelleted at 2,400×g for 5 min in 500 mL centrifuge bottles filled to 400 mL. Cell pellets were combined and then resuspended in 25 ml Buffer B and pelleted at 2,400×g for 3 min. The buffer was decanted and the wash repeated one time. Cell pellets were resuspended in 3× the pellet volume of Buffer B containing 1× protease inhibitors (Roche, Complete, EDTA-free) using a polytron homogenizer with the cells maintained on ice. The homogenate was then centrifuged at 2,400×g for 5 min and the supernatant retained and pelleted one additional time (2,400×g/5 min) to ensure the removal of unbroken cells, cell debris and nuclei; the resultant supernatant is the total protein preparation. The supernatant was then transferred into two Nalgene Oak Ridge 45 mL centrifuge tubes and pelleted at 40,000×g for 40 min at 4° C. The supernatants containing the Separated Cytosolic Proteins (SCPs) were then transferred into clean Oak Ridge tubes, and centrifuged at 40,000×g one more time. In parallel, the pellets containing the membrane fraction (EMF) were retained and centrifuged at 40,000 for 20 min to remove residual supernatant. The EMF pellets were then rinsed with Buffer B. 8 mL Buffer B was then added to the membrane pellets to dislodge the pellets and transfer into a Dounce Homogenizer. After the pellets were homogenized, they were transferred to a 50 mL conical tube and represented the final EMF preparation.

One billion mammalian cells (e.g. CHO, HEK293, Sf9) at ˜10⁶-10⁷ cells/mL were transferred from tissue culture environment into 4×250 mL conical tubes and pelleted at 550×g for 3 min. All subsequent steps were performed at 4° C. or on ice with ice-cold buffers. Cells were washed with 100 mL of PBSF (lx PBS+1 mg/mL BSA) and combined into one conical tube. After removing the supernatant, the cell pellet was then re-suspended in 30 mL Buffer B (50 mM HEPES, 0.15 M NaCl, 2 mM CaCl2, 5 mM KCl, 5 mM MgCl2, 10% Glycerol, pH 7.2) and pelleted at 550×g for 3 min. Buffer B supernatant was decanted and cells re-suspended in 3× pellet volume of Buffer B plus 2.5× protease inhibitor (Roche, cOmplete, EDTA-free). Protease inhibitors in Buffer B were included from here on forward. Cells were homogenized four times for 30 sec pulses (Polyton homogenizer, PT1200E) and the membrane fraction was pelleted at 40,000×g for 1 hour at 4° C. The pellet is rinsed with 1 mL Buffer B; the supernatant is retained and represents the s. The pellet is transferred into a Dounce homogenizer with 3 mL of Buffer B and re-suspended by moving the pestle slowly up and down for 30-35 strokes. The enriched membrane fraction (EMF) is moved into a new collection tube, rinsing the pestle to collect all potential protein. Determine the protein concentration of the purified EMF using the Dc-protein assay kit (BioRad). To solubilize the EMF, transfer into Solubilization Buffer (50 mM HEPES, 0.15 M NaCl, 2 mM CaCl2, 5 mM KCl, 5 mM MgCl2, 1% n-Dodecyl-b-D-Maltopyranoside (DDM), 1× protease inhibitor, pH 7.2) to a final concentration of 1 mg/mL. Rotate the mixture overnight at 4° C. rotating followed by centrifugation in a 50 mL Oak Ridge tube (Fisher Scientific, 050529-ID) at 40,000×g for 1 hour. Collect the supernatant which represents the soluble membrane proteins (SMPs) and quantify the protein yield as described above.

For biotinylation, prepare the NHS-LC-Biotin stock solution according to manufacturer's protocol (Pierce, Thermo Fisher). In brief, 20 ul of biotin reagent is added for every 1 mg of EMF sample and incubated at 4° C. for 3 hours with gentle agitation. Adjust the volume to 25 mL with Buffer B and transfer to an Oak Ridge centrifuge tube. Pellet the biotinylated EMF (b-EMF) at 40,000×g for 1 hour, and rinse two times with 3 mL of Buffer C (Buffer B minus the glycerol) without disturbing the pellet. Remove the residual solution. Re-suspended the pellet with a Dounce homogenizer in 3 mL of Buffer C as described previously. The re-suspended pellet now represents biotinylated EMF (b-EMF). Solubilized as described above to prepare b-SMPs.

PSR Binding Analyses. Assays were performed generally as described in, e.g., Xu et al. Protein Eng Des Sel, 26(10):663-670 (2013). To characterize the PSR profile of monoclonal antibodies presented on yeast, two million IgG-presenting yeast were transferrred into a 96-well assay plate and pellet at 3000×g for 3 min to remove supernatant. Re-suspend the pellet in 50 ul of freshly prepared 1:10 dilution of stock b-PSRs and incubate on ice for 20 minutes. Wash the cells twice with 200 ul of cold PBSF and pellet re-suspended in 50 ul of secondary labeling mix (Extravidin-R-PE, anti-human LC-FITC, and propidium iodide). Incubate the mix on ice for 20 minutes followed by two washes with 200 ul ice-cold PBSF. Re-suspend the cells in 100 ul of ice-cold PBSF and run the plate on a FACSCanto (BD Biosciences) using HTS sample injector. Flow cytometry data was analyzed for mean fluorescence intensity in the R-PE channel and normalized to proper controls in order to assess non-specific binding. Numerous methods for presentation or display of antibodies or antibody fragments on the surface of yeast have been described previously, all of which are consistent with this protocol (Blaise et al., Gene, 342(2):211-8 (2004), Boder and Wittrup, Nat Biotechnol., 15(6):553-7 (1997), Kuroda and Ueda, Biotechnol Lett., 33(1):1-9 (2011), Orcutt and Wittrup, Springer Protocols: Antibody Engineering, 1:207-233 (2010), Rakestraw et al., Protein Eng Des Sel., 24(6):525-30 (2011), Sazinsky et al., Proc Natl Acad Sci USA., 105(51):20167-72 (2008), Tasumi et al., Proc Natl Acad Sci USA., 106(31):12891-6 (2009)).

ForteBio Kinetics. FortBio Octet HTX instruments were used in 12 channel mode (8 sensors per channel, 96 sensors per experiment) with either AHC, SA, or AHQ sensors. Instrumentation was driven by manufacturer supplied software (versions 8.2 and 9.0). Sample names and concentrations were input into the plate data page, and sensor associated proteins were identified in the “information” column on the sensor data page. Kinetic experiments were collected with either a 90 or 180 s baseline, 180 s association phase, and 180 s dissociation phase. All files were saved into a shared network drive with a naming convention that identifies the format of the experiment.

HIC. IgG1 samples were buffer exchanged into 1 M ammonium sulfate and 0.1 M sodium phosphate at pH 6.5 using a Zeba 40 kDa 0.5 mL spin column (Thermo Pierce, cat #87766). A salt gradient was established on a Dionex ProPac HIC-10 column from 1.8 M ammonium sulfate, 0.1 M sodium phosphate at pH 6.5 to the same condition without ammonium sulfate. The gradient ran for 17 min at a flow rate of 0.75 ml/min. An acetonitrile wash step was added at the end of the run to remove any remaining protein and the column was re-equilibrated over 7 column volumes before the next injection cycle. Peak retention times were monitored at A280 absorbance and concentrations of ammonium sulfate at elution were calculated based on gradient and flow rate.

LCMS. mAb samples were reduced by DTT, followed by middle down LCMS analysis on a Bruker maXis4G mass spectrometer coupled with an Agilent 1100 HPLC (Agilent). A POROS R2 10 m (2.1×30 mm) reversed phase column was used to remove salt in the samples. A fast LC flow at 2 mL/min allows the separation between sample and salt and elution of samples and regeneration of column to finish within a 2.1 min cycle. A T-junction is used to deliver only 0.15 mL/min sample flow into the mass spectrometer for sample analysis. The Bruker maXis 4G mass spectrometer was run in positive ion mode with detection in the range of 750 to 2500 m/z. The remaining source parameters were set as follows; the capillary was set at 5500V, the Nebulizer at 4.0 Bar, dry gas at 4.0 l/min, and dry temp set at 200° C.

The MS spectra were analyzed using Bruker Data Analysis version 4.1 and the deconvolution was accomplished using maximum entropy deconvolution with a mass range of 20 to 30 kDa.

An informal sequence listing is provided in Table 1. The informal sequence listing provides the heavy chain variable region (“HC”) amino acid sequence, with each of the heavy chain variable region CDRs underlined, and the light chain variable region (“LC”), with each of the light chain variable region CDRs underlined.

TABLE 1
Informal Sequence Listing

| Antibody | SEQ ID | | | |
|---|---|---|---|---|
| No. | NO: | Sequence | Clone # (ADI) | Descriptors |
| Ab1 | 60 | QVQLVQSGAEVKKPGASVKVSCKASGFNIKDYYMH | LAD5224_P03_A01 | HC amino acid sequence |
| | | WVRQAPGQRLEWMGWIDLENANTIYDAKFQGRVTI | (ADI-26906) | |
| | | TRDTSASTAYMELSSLRSEDTAVYYCARDAYGRYFY | | |
| | | DVWGQGTLVTVSS | | |
| Ab1 | 61 | DIVMTQSPDSLAVSLGERATINCKSSQSLLNARTGKN | LAD5224_P03_A01 | LC amino acid sequence |
| | | YLAWYQQKPGQPPKLLIYWASTRESGVPDRFSGSGS | (ADI-26906) | |
| | | GTDFTLTISSLQAEDVAVYYCKQSYSRRTFGGGTKVE | | |
| | | IK | | |
| Ab2 | 62 | QVQLVQSGAEVKKPGASVKVSCKASGFNIKDYYMH | SAD10318_P01_B01 | HC amino acid sequence |
| | | WVRQAPGQRLEWMGWIDLENANTIYDAKFQGRVTI | (ADI-48574) | |
| | | TRDTSASTAYMELSSLRSEDTAVYYCARDAYGRYHY | | |
| | | DHWGQGTLVTVSS | | |
| Ab2 | 63 | DIVMTQSPDSLAVSLGERATINCKSSQSLLNAHTGHN | SAD10318_P01_B01 | LC amino acid sequence |
| | | YLAWYQQKPGQPPKLLIYWASTRESGVPDRFSGSGS | (ADI-48574) | |
| | | GTDFTLTISSLQAEDVAVYYCKQSYSRRTFGGGTKVE | | |
| | | IK | | |
| Ab3 | 64 | QVQLVQSGAEVKKPGASVKVSCKASGFNIKDYYMH | SAD10318_P01_D02 | HC amino acid sequence |
| | | WVRQAPGQRLEWMGWIDLENANTIYDAKFQGRVTI | (ADI-48584) | |
| | | TRDTSASTAYMELSSLRSEDTAVYYCARDAHHRYFY | | |
| | | DVWGQGTLVTVSS | | |
| Ab3 | 65 | DIVMTQSPDSLAVSLGERATINCKSSQSLLNARTGKN | SAD10318_P01_D02 | LC amino acid sequence |
| | | HLAWYQQKPGQPPKLLIYWASTRESGVPDRFSGSGS | (ADI-48584) | |
| | | GTDFTLTISSLQAEDVAVYYCKQSHSHRTFGGGTKVE | | |
| | | IK | | |
| Ab4 | 66 | QVQLVQSGAEVKKPGASVKVSCKASGYTFDSYIMH | SAD10319_P02_E05 | HC amino acid sequence |
| | | WVRQAPGQRLEWMGWIDAGTGATVYSQKFQGRVTI | (ADI-48630) | |
| | | TRDTSASTAYMELSSLRSEDTAVYYCARDAYGHYFY | | |
| | | DHWGQGTLVTVSS | | |
| Ab4 | 67 | DIVMTQSPDSLAVSLGERATINCKSSQSLLNARTGKN | SAD10319_P02_E05 | LC amino acid sequence |
| | | YLAWYQQKPGQPPKLLIYWASTRESGVPDRFSGSGS | (ADI-48630) | |
| | | GTDFTLTISSLQAEDVAVYYCKQSYSRRTFGGGTKVE | | |
| | | IK | | |
| Ab5 | 68 | QVQLVQSGAEVKKPGASVKVSCKASGYTFNNYAMH | SAD10319_P02_F05 | HC amino acid sequence |
| | | WVRQAPGQRLEWMGWIDAGTGNTDYSQKFQGRVTI | (ADI-57317) | |
| | | TRDTSASTAYMELSSLRSEDTAVYYCARDAHHRYFY | | |
| | | DVWGQGTLVTVSS | | |
| Ab5 | 69 | DIVMTQSPDSLAVSLGERATINCKSSQSLLNARTGKN | SAD10319_P02_F05 | LC amino acid sequence |
| | | YLAWYQQKPGQPPKLLIYWASTRESGVPDRFSGSGS | (ADI-57317) | |
| | | GTDFTLTISSLQAEDVAVYYCKQSYSRRTFGGGTKVE | | |
| | | IK | | |
| Ab6 | 70 | QVQLVQSGAEVKKPGASVKVSCKASGYTFTQYTMH | SAD10319_P02_G05 | HC amino acid sequence |
| | | WVRQAPGQRLEWMGSIDAGTGATKYSQKFQGRVTI | (ADI-57318) | |
| | | TRDTSASTAYMELSSLRSEDTAVYYCARDAYHHYFY | | |
| | | DVWGQGTLVTVSS | | |
| Ab6 | 71 | DIVMTQSPDSLAVSLGERATINCKSSQSLLNARTGKN | SAD10319_P02_G05 | LC amino acid sequence |
| | | YLAWYQQKPGQPPKLLIYWASTRESGVPDRFSGSGS | (ADI-57318) | |
| | | GTDFTLTISSLQAEDVAVYYCKQSYSRRTFGGGTKVE | | |
| | | IK | | |
| Ab7 | 72 | QVQLVQSGAEVKKPGASVKVSCKASGYTFTQYSMH | SAD10319_P02_H05 | HC amino acid sequence |
| | | WVRQAPGQRLEWMGWINAGTGDTVYSQKFQGRVTI | (ADI-57319) | |
| | | TRDTSASTAYMELSSLRSEDTAVYYCARDHHGRYFY | | |
| | | DVWGQGTLVTVSS | | |
| Ab7 | 73 | DIVMTQSPDSLAVSLGERATINCKSSQSLFNARTGKN | SAD10319_P02_H05 | LC amino acid sequence |
| | | YLAWYQQKPGQPPKLLIYWASTRESGVPDRFSGSGS | (ADI-57319) | |
| | | GTDFTLTISSLQAEDVAVYYCKQSYSRRTFGGGTKVE | | |
| | | IK | | |
| Ab8 | 74 | QVQLVQSGAEVKKPGASVKVSCKASGYTFDAYAMH | SAD10319_P02_A06 | HC amino acid sequence |
| | | WVRQAPGQRLEWMGWIDAGTGATKYSQKFQGRVTI | (ADI-57320) | |
| | | TRDTSASTAYMELSSLRSEDTAVYYCARDHYGHYFY | | |
| | | DVWGQGTLVTVSS | | |
| Ab8 | 75 | DIVMTQSPDSLAVSLGERATINCKSSQSLLNARTGKN | SAD10319_P02_A06 | LC amino acid sequence |
| | | YLAWYQQKPGQPPKLLIYWASTRESGVPDRFSGSGS | (ADI-57320) | |
| | | GTDFTLTISSLQAEDVAVYYCKQSYSRRTFGGGTKVE | | |
| | | IK | | |
| Ab9 | 76 | QVQLVQSGAEVKKPGASVKVSCKASGYTFTSYTMH | SAD10319_P02_B06 | HC amino acid sequence |
| | | WVRQAPGQRPEWMGSINAGTGFTDYSQKFQGRVTIT | (ADI-57321) | |
| | | RDTSASTAYMELSSLRSEDTAVYYCARDHYGHYFYD | | |
| | | VWGQGTLVTVSS | | |
| Ab9 | 77 | DIVMTQSPDSLAVSLGERATINCKSSQSLLNARTGKN | SAD10319_P02_B06 | LC amino acid sequence |
| | | YLAWYQQKPGQPPKLLIYWASTRESGVPDRFSGSGS | (ADI-57321) | |
| | | GTDFTLTISSLQAEDVAVYYCKQSYSRRTFGGGTKVE | | |
| | | IK | | |
| Ab10 | 78 | QVQLVQSGAEVKKPGASVKVSCKASGYTFTSYAMH | SAD10319_P02_C06 | HC amino acid sequence |
| | | WVRQAPGQRLEWMGWINPVTGATVYSQKFQGRVTI | (ADI-57322) | |
| | | TRDTSASTAYMELSSLRSEDTAVYYCARDAYGHYFY | | |
| | | DVWGQGTLVTVSS | | |
| Ab10 | 79 | DIVMTQSPDSLAVSLGERATINCKSSQSLLNARTGKN | SAD10319_P02_C06 | LC amino acid sequence |
| | | YLAWYQQKPGQPPKLLIYWASTRESGVPDRFSGSGS | (ADI-57322) | |
| | | GTDFTLTISSLQAEDVAVYYCKQSYSRRTFGGGTKVE | | |
| | | IK | | |
| Ab11 | 80 | QVQLVQSGAEVKKPGASVKVSCKASGYTFTDYAMH | SAD10319_P02_D06 | HC amino acid sequence |
| | | WVRQAPGQRLEWMGWINAGTGATIYSQKFQGRVTI | (ADI-48631) | |
| | | TRDTSASTAYMELSSLRSEDTAVYYCARDAYHHYFY | | |
| | | DVWGQGTLVTVSS | | |
| Ab11 | 81 | DIVMTQSPDSLAVSLGERATINCKSSQSLLNARTGKN | SAD10319_P02_D06 | LC amino acid sequence |
| | | YLAWYQQKPGQPPKLLIYWASTRESGVPDRFSGSGS | (ADI-48631) | |
| | | GTDFTLTISSLQAEDVAVYYCKQSYSRRTFGGGTKVE | | |
| | | IK | | |
| Ab12 | 82 | QVQLVQSGAEVKKPGASVKVSCKASGYTFADYAMH | SAD10319_P02_E06 | HC amino acid sequence |
| | | WVRQAPGQRLAWMGWINPHTGATFYSQKFQGRVTI | (ADI-57323) | |
| | | TRDTSASTAYMELSSLRSEDTAVYYCARDAYGRYFY | | |
| | | DHWGQGTLVTVSS | | |
| Ab12 | 83 | DIVMTQSPDSLAVSLGERATINCKSSQSLLNARTGKN | SAD10319_P02_E06 | LC amino acid sequence |
| | | YLAWYQQKPGQPPKLLIYWASTRESGVPDRFSGSGS | (ADI-57323) | |
| | | GTDFTLTISSLQAEDVAVYYCKQSYSRRTFGGGTKVE | | |
| | | IK | | |
| Ab13 | 84 | QVQLVQSGAEVKKPGASVKVSCKASGYTFLSYAMH | SAD10319_P02_F06 | HC amino acid sequence |
| | | WVRQAPGQRLERMGWINPSTGATDYSQKFQGRVTIT | (ADI-57324) | |
| | | RDTSASTAYMELSSLRSEDTAVYYCARDAYGHYFYD | | |
| | | VWGQGTLVTVSS | | |
| Ab13 | 85 | DIVMTQSPDSLAVSLGERATINCKSSQSLLNARTGKN | SAD10319_P02_F06 | LC amino acid sequence |
| | | YLAWYQQKPGQPPKLLIYWASTRESGVPDRFSGSGS | (ADI-57324) | |
| | | GTDFTLTISSLQAEDVAVYYCKQSYSRRTFGGGTKVE | | |
| | | IK | | |
| Ab14 | 86 | QVQLVQSGAEVKKPGASVKVSCKASGFNIKDYYMH | SAD10318_P01_F02 | HC amino acid sequence |
| | | WVRQAPGQRLEWMGWIDLENANTIYDAKFQGRVTI | (ADI-48585) | |
| | | TRDTSASTAYMELSSLRSEDTAVYYCARDAHGHYFY | | |
| | | DVWGQGTLVTVSS | | |
| Ab14 | 87 | DIVMTQSPDSLAVSLGERATINCKSSQSLLNAHTGKN | SAD10318_P01_F02 | LC amino acid sequence |
| | | HLAWYQQKPGQPPKLLIYWASTRESGVPDRFSGSGS | (ADI-48585) | |
| | | GTDFTLTISSLQAEDVAVYYCKQSHSHRTFGGGTKVE | | |
| | | IK | | |
| Ab15 | 88 | QVQLVQSGAEVKKPGASVKVSCKASGYTFTSYAMH | SAD10319_P02_G06 | HC amino acid sequence |
| | | WVRQAPGQRLEWMGWINPDTGATVYSQKFQGRVTI | (ADI-48632) | |
| | | TRDTSASTAYMELSSLRSEDTAVYYCARDAYHHYFY | | |
| | | DVWGQGTLVTVSS | | |
| Ab15 | 89 | DIVMTQSPDSLAVSLGERATINCKSSQSLLNARTGKN | SAD10319_P02_G06 | LC amino acid sequence |
| | | YLAWYQQKPGQPPKLLIYWASTRESGVPDRFSGSGS | (ADI-48632) | |
| | | GTDFTLTISSLQAEDVAVYYCKQSYSRRTFGGGTKVE | | |
| | | IK | | |
| Ab16 | 90 | QVQLVQSGAEVKKPGASVKVSCKASGYTFTAYVMH | SAD10319_P02_H06 | HC amino acid sequence |
| | | WVRQAPGQRLEWMGSIDAGTGNTKYSQKFQGRVTI | (ADI-48633) | |
| | | TRDTSASTAYMELSSLRSEDTAVYYCARDAYGHYFY | | |
| | | DHWGQGTLVTVSS | | |
| Ab16 | 91 | DIVMTQSPDSLAVSLGERATINCKSSQSLLNARTGKN | SAD10319_P02_H06 | LC amino acid sequence |
| | | YLAWYQQKPGQPPKLLIYWASTRESGVPDRFSGSGS | (ADI-48633) | |
| | | GTDFTLTISSLQAEDVAVYYCKQSYSRRTFGGGTKVE | | |
| | | IK | | |
| Ab17 | 92 | QVQLVQSGAEVKKPGASVKVSCKASGYTFESYDMH | SAD10319_P03_C07 | HC amino acid sequence |
| | | WVRQAPGQRLEWMGWIDAGTGATTYSQKFQGRVTI | (ADI-48634 and | |
| | | TRDTSASTAYMELSSLRSEDTAVYYCARDAYHHYFY | ADI-48635) | |
| | | DVWGQGTLVTVSS | | |
| Ab17 | 93 | DIVMTQSPDSLAVSLGERATINCKSSQSLLNARTGKN | SAD10319_P03_C07 | LC amino acid sequence |
| | | YLAWYQQKPGQPPKLLIYWASTRESGVPDRFSGSGS | (ADI-48634 and | |
| | | GTDFTLTISSLQAEDVAVYYCKQSYSRRTFGGGTKVE | ADI-48635) | |
| | | IK | | |
| Ab18 | 94 | QVQLVQSGAEVKKPGASVKVSCKASGYTFDSYLMH | SAD10319_P03_G07 | HC amino acid sequence |
| | | WVRQAPGQRLEWMGWIDAGTGATNYSQKFQGRVTI | (ADI-57325) | |
| | | TRDTSASTAYMELSSLRSEDTAVYYCARDAHGHYFY | | |
| | | DVWGQGTLVTVSS | | |
| Ab18 | 95 | DIVMTQSPDSLAVSLGERATINCKSSQSLLNARTGKN | SAD10319_P03_G07 | LC amino acid sequence |
| | | YLAWYQQKPGQPPKLLIYWASTRESGVPDRFSGSGS | (ADI-57325) | |
| | | GTDFTLTISSLQAEDVAVYYCKQSYSRRTFGGGTKVE | | |
| | | IK | | |
| Ab19 | 96 | QVQLVQSGAEVKKPGASVKVSCKASGYTFNSYDMH | SAD10319_P03_H07 | HC amino acid sequence |
| | | WVRQAPGQRLEWMGWINPYTGETKYSQKFQGRVTI | (ADI-57326) | |
| | | TRDTSASTAYMELSSLRSEDTAVYYCARDHYGHYFY | | |
| | | DVWGQGTLVTVSS | | |
| Ab19 | 97 | DIVMTQSPDSLAVSLGERATINCKSSQSLLNARTGKN | SAD10319_P03_H07 | LC amino acid sequence |
| | | YLAWYQQKPGQPPKLLIYWASTRESGVPDRFSGSGS | (ADI-57326) | |
| | | GTDFTLTISSLQAEDVAVYYCKQSYSRRTFGGGTKVE | | |
| | | IK | | |
| Ab20 | 98 | QVQLVQSGAEVKKPGASVKVSCKASGYTFYSYEMH | SAD10319_P03_D08 | HC amino acid sequence |
| | | WVRQAPGQRLDWMGWINPQTGATKYSQKFQGRVTI | (ADI-57327) | |
| | | TRDTSASTAYMELSSLRSEDTAVYYCARDAYHHYFY | | |
| | | DVWGQGTLVTVSS | | |
| Ab20 | 99 | DIVMTQSPDSLAVSLGERATINCKSSQSLLNARTGKN | SAD10319_P03_D08 | LC amino acid sequence |
| | | YLAWYQQKPGQPPKLLIYWASTRESGVPDRFSGSGS | (ADI-57327) | |
| | | GTDFTLTISSLQAEDVAVYYCKQSYSRRTFGGGTKVE | | |
| | | IK | | |
| Ab21 | 100 | QVQLVQSGAEVKKPGASVKVSCKASGYTFNDYAMH | SAD10319_P03_E08 | HC amino acid sequence |
| | | WVRQAPGQRLEWMGYINGTGATVYSQKFQGRVTI | (ADI-48637) | |
| | | TRDTSASTAYMELSSLRSEDTAVYYCARDAYHHYFY | | |
| | | DVWSQGTLVTVSS | | |
| Ab21 | 101 | DIVMTQSPDSLAVSLGERATINCKSSQSLLNARTGKN | SAD10319_P03_E08 | LC amino acid sequence |
| | | YLAWYQQKPGQPPKLLIYWASTRESGVPDRFSGSGS | (ADI-48637) | |
| | | GTDFTLTISSLQAEDVAVYYCKQSYSRRTFGGGTKVE | | |
| | | IK | | |
| Ab22 | 102 | QVQLVQSGAEVKKPGASVKVSCKASGYTFSSYDMH | SAD10319_P03_G08 | HC amino acid sequence |
| | | WVRQAPGQRLEWMGWIDAGTGFTKYSQKFQGRVTI | (ADI-48638) | |
| | | TRDTSASTAYMELSSLRSEDTAVYYCARDAYGHYFY | | |
| | | DHWGQGTLVTVSS | | |
| Ab22 | 103 | DIVMTQSPDSLAVSLGERATINCKSSQSLLNARTGKN | SAD10319_P03_G08 | LC amino acid sequence |
| | | YLAWYQQKPGQPPKLLIYWASTRESGVPDRFSGSGS | (ADI-48638) | |
| | | GTDFTLTISSLQAEDVAVYYCKQSYSRRTFGGGTKVE | | |
| | | IK | | |
| Ab23 | 104 | QVQLVQSGAEVKKPGASVKVSCKASGYTFKSYDMH | SAD10319_P03_H08 | HC amino acid sequence |
| | | WVRQAPGQRPEWMGWINAGTGNTVYSQKFQGRVTI | (ADI-57328) | |
| | | TRDTSASTAYMELSSLRSEDTAVYYCARDAYHRYFY | | |
| | | DVWGQGTLVTVSS | | |
| Ab23 | 105 | DIVMTQSPDSLAVSLGERATINCKSSQSLLNARTGKN | SAD10319_P03_H08 | LC amino acid sequence |
| | | YLAWYQQKPGQPPKLLIYWASTRESGVPDRFSGSGS | (ADI-57328) | |
| | | GTDFTLTISSLQAEDVAVYYCKQSYSRRTFGGGTKVE | | |
| | | IK | | |
| Ab24 | 106 | QVQLVQSGAEVKKPGASVKVSCKASGYTFTEYDMH | SAD10319_P03_B09 | HC amino acid sequence |
| | | WVRQAPGQRLEWMGWIDAGTGATVYSQKFQGRVTI | (ADI-48639) | |
| | | TRDTSASTAYMELSSLRSEDTAVYYCARDAYGRYFY | | |
| | | DHWGQGTLVTVSS | | |
| Ab24 | 107 | DIVMTQSPDSLAVSLGERATINCKSSQSLLNARTGKN | SAD10319_P03_B09 | LC amino acid sequence |
| | | YLAWYQQKPGQPPKLLIYWASTRESGVPDRFSGSGS | (ADI-48639) | |
| | | GTDFTLTISSLQAEDVAVYYCKQSYSRRTFGGGTKVE | | |
| | | IK | | |
| Ab25 | 108 | QVQLVQSGAEVKKPGASVKVSCKASGFNIKDYYMH | SAD10318_P01_G02 | HC amino acid sequence |
| | | WVRQAPGQRLEWMGWIDLENANTIYDAKFQGRVTI | (ADI-48586) | |
| | | TRDTSASTAYMELSSLRSEDTAVYYCARDAYHHYFY | | |
| | | DVWGQGTLVTVSS | | |
| Ab25 | 109 | DIVMTQSPDSLAVSLGERATINCKSSQSLLNARTGKN | SAD10318_P01_G02 | LC amino acid sequence |
| | | HLAWYQQKPGQPPKLLIYWASTRESGVPDRFSGSGS | (ADI-48586) | |
| | | GTDFTLTISSLQAEDVAVYYCKQSYSHRTFGGGTKVE | | |
| | | IK | | |
| Ab26 | 110 | QVQLVQSGAEVKKPGASVKVSCKASGYTFLSYDMH | SAD10319_P03_E09 | HC amino acid sequence |
| | | WVRQAPGQRLEWMGWIDAGTGATTYSQKFQGRVTI | (ADI-48640) | |
| | | TRDTSASTAYMELSSLRSEDTAVYYCARDAYHHYFY | | |
| | | DVWGQGTLVTVSS | | |
| Ab26 | 111 | DIVMTQSPDSLAVSLGERATINCKSSQSLLNARTGKN | SAD10319_P03_E09 | LC amino acid sequence |
| | | YLAWYQQKPGQPPKLLIYWASTRESGVPDRFSGSGS | (ADI-48640) | |
| | | GTDFTLTISSLQAEDVAVYYCKQSYSRRTFGGGTKVE | | |
| | | IK | | |
| Ab27 | 112 | QVQLVQSGAEVKKPGASVKVSCKASGYTFPSYPMH | SAD10319_P03_F09 | HC amino acid sequence |
| | | WVRQAPGQRLDWMGWINPDTGATVYSQKFQGRVTI | (ADI-57329) | |
| | | TRDTSASTAYMELSSLRSEDTAVYYCARDAYHHYFY | | |
| | | DVWGQGTLVTVSS | | |
| Ab27 | 113 | DIVMTQSPDSLAVSLGERATINCKSSQSPLNARTGKN | SAD10319_P03_F09 | LC amino acid sequence |
| | | YLAWYQQKPGQPPKLLIYWASTRESGVPDRFSGSGS | (ADI-57329) | |
| | | GTDFTLTISSLQAEDVAVYYCKQSYSRRTFGGGTKVE | | |
| | | IK | | |
| Ab28 | 114 | QVQLVQSGAEVKKPGASVKVSCKASGYTFTSYDMH | SAD10319_P03_G09 | HC amino acid sequence |
| | | WVRQAPGQRLEWMGWIDPHTGATKYSQKFQGRVTI | (ADI-57300) | |
| | | TRDTSASTAYMELSSLRSEDTAVYYCARDAYGRYFY | | |
| | | DHWGQGTLVTVSS | | |
| Ab28 | 115 | DIVMTQSPDSLAVSLGERATINCKSSQSLLNARTGKN | SAD10319_P03_G09 | LC amino acid sequence |
| | | YLAWYQQKPGQPPKLLIYWASTRESGVPDRFSGSGS | (ADI-57300) | |
| | | GTDFTLTISSLQAEDVAVYYCKQSYSRRTFGGGTKVE | | |
| | | IK | | |
| Ab29 | 116 | QVQLVQSGAEVKKPGASVKVSCKASGYTFTSDAMH | SAD10319_P04_A10 | HC amino acid sequence |
| | | WVRQAPGQRLAWMGYINAGTGTTKYSQKFQGRVTI | (ADI-57331) | |
| | | TRDTSASTAYMELSSLRSEDTAVYYCAHDAYGHYFY | | |
| | | DVWGQGTLVTVSS | | |
| Ab29 | 117 | DIVMTQSPDSLAVSLGERATINCKSSQSLLNARTGKN | SAD10319_P04_A10 | LC amino acid sequence |
| | | YLAWYQQKPGQPPKLLIYWASTRESGVPDRFSGSGS | (ADI-57331) | |
| | | GTDFTLTISSLQAEDVAVYYCKQSYSRRTFGGGTKVE | | |
| | | IK | | |
| Ab30 | 118 | QVQLVQSGAEVKKPGASVKVSCKASGYTFTSTFMH | SAD10319_P04_B10 | HC amino acid sequence |
| | | WVRQAPGQRLEWMGFIDAGTGATKYSQKFQGRVTI | (ADI-57332) | |
| | | TRDTSASTAYMELSSLRSEDTAVYYCARDAYGHYFY | | |
| | | DHWGQGTLVTVSS | | |
| Ab30 | 119 | DIVMTQSPDSLAVSLGERATINCKSSQSLLNARTGKN | SAD10319_P04_B10 | LC amino acid sequence |
| | | YLAWYQQKPGQPPKLLIYWASTRESGVPDRFSGSGS | (ADI-57332) | |
| | | GTDFTLTISSLQAEDVAVYYCKQSYSRRTFGGGTKVE | | |
| | | IK | | |
| Ab31 | 120 | QVQLVQSGAEVKKPGASVKVSCKASGYTFTDYSMH | SAD10319_P04_C10 | HC amino acid sequence |
| | | WVRQAPGQRLEWMGSINAGTGITKYSQKFQGRVTIT | (ADI-57333) | |
| | | RDTSASTAYMELSSLRSEDTAVYYCARDAHGRYFYD | | |
| | | VWGQGTLVTVSS | | |
| Ab31 | 121 | DIVMTQSPDSLAVSLGERATINCKSSQSLLNARTGKN | SAD10319_P04_C10 | LC amino acid sequence |
| | | YLAWYQQKPGQPPKLLIYWASTRESGVPDRFSGSGS | (ADI-57333) | |
| | | GTDFTLTISSLQAEDVAVYYCKQSYSRRTFGGGTKVE | | |
| | | IK | | |
| Ab32 | 122 | QVQLVQSGAEVKKPGASVKVSCKASGYTFTDYYMH | SAD10319_P04_G10 | HC amino acid sequence |
| | | WVRQAPGQRLEWMGWINPATGATAYSQKFQGRVTI | (ADI-57334) | |
| | | TRDTSASTAYMELSSLRSEDTAVYYCARDAYGHHFY | | |
| | | DVWGQGTLVTVSS | | |
| Ab32 | 123 | DIVMTQSPDSLAVSLGERATINCKSSQSLLNARTGKN | SAD10319_P04_G10 | LC amino acid sequence |
| | | YLAWYQQKPGQPPKLLIYWASTRESGVPDRFSGSGS | (ADI-57334) | |
| | | GTDFTLTISSLQAEDVAVYYCKQSYSRRTFGGGTKVE | | |
| | | IK | | |
| Ab33 | 124 | QVQLVQSGAEVKKPGASVKVSCKASGYTFGSYDMH | SAD10319_P04_B11 | HC amino acid sequence |
| | | WVRQAPGQRLEWMGWIDAGTGATKYSQKFQGRVTI | (ADI-57335) | |
| | | TRDTSASTAYMELSSLRSEDTAVYYCARDAHGHYFY | | |
| | | DVWGQGTLVTVSS | | |
| Ab33 | 125 | DIVMTQSPDSLAVSLGERATINCKSSQSLLNARTGKN | SAD10319_P04_B11 | LC amino acid sequence |
| | | YLAWYQQKPGQPPKLLIYWASTRESGVPDRFSGSGS | (ADI-57335) | |
| | | GTDFTLTISSLQAEDVAVYYCKQSYSRRTFGGGTKVE | | |
| | | IK | | |
| Ab34 | 126 | QVQLVQSGAEVKKPGASVKVSCKASGYTFHSYEMH | SAD10319_P04_C11 | HC amino acid sequence |
| | | WVRQAPGQRLEWMGWINPSTGSTKYSQKFQGRVTIT | (ADI-57336) | |
| | | RDTSASTAYMELSSLRSEDTAVYYCARDAYGRYFYD | | |
| | | HWGQGTLVTVSS | | |
| Ab34 | 127 | DIVMTQSPDSLAVSLGERATINCKSSQSLLNARTGKN | SAD10319_P04_C11 | LC amino acid sequence |
| | | YLAWYQQKPGQPPKLLIYWASTRESGVPDRFSGSGS | (ADI-57336) | |
| | | GTDFTLTISSLQAEDVAVYYCKQSYSRRTFGGGTKVE | | |
| | | IK | | |
| Ab35 | 128 | QVQLVQSGAEVKKPGASVKVSCKASGYTFNSYRMH | SAD10319_P04_D11 | HC amino acid sequence |
| | | WVRQAPGQRLEWMGSIDAGTGNTKYSQKFQGRVTI | (ADI-57337) | |
| | | TRDTSASTAYMELSSLRSEDTAVYYCARDAYGRYFY | | |
| | | DHWGQGTLVTVSS | | |
| Ab35 | 129 | DIVMTQSPDSLAVSLGERATINCKSSQSLLNARTGKN | SAD10319_P04_D11 | LC amino acid sequence |
| | | YLAWYQQKPGQPPKLLIYWASTRESGVPDRFSGSGS | (ADI-57337) | |
| | | GTDFTLTISSLQAEDVAVYYCKQSYSRRTFGGGTKVE | | |
| | | IK | | |
| Ab36 | 130 | QVQLVQSGAEVKKPGASVKVSCKASGFNIKDYYMH | SAD10318_P01_A03 | HC amino acid sequence |
| | | WVRQAPGQRLEWMGWIDLENANTIYDAKFQGRVTI | (ADI-48587) | |
| | | TRDTSASTAYMELSSLRSEDTAVYYCARDHYHRYFY | | |
| | | DVWGQGTLVTVSS | | |
| Ab36 | 131 | DIVMTQSPDSLAVSLGERATINCKSSQSLLNARTGHN | SAD10318_P01_A03 | LC amino acid sequence |
| | | YLAWYQQKPGQPPKLLIYWASTRESGVPDRFSGSGS | (ADI-48587) | |
| | | GTDFTLTISSLQAEDVAVYYCKQSHSHRTFGGGTKVE | | |
| | | IK | | |
| Ab37 | 132 | QVQLVQSGAEVKKPGASVKVSCKASGYTFTDYNMH | SAD10319_P04_E11 | HC amino acid sequence |
| | | WVRQAPGQRLEWMGWINPYTGYTKYSQKFQGRVTI | (ADI-57338) | |
| | | TRDTSASTAYMELSSLRSEDTAVYYCARDHYGHYFY | | |
| | | DVWGQGTLVTVSS | | |
| Ab37 | 133 | DIVMTQSPDSLAVSLGERATINCKSSQSLLNARTGKN | SAD10319_P04_E11 | LC amino acid sequence |
| | | YLAWYQQKPGQPPKLLIYWASTRESGVPDRFSGSGS | (ADI-57338) | |
| | | GTDFTLTISSLQAEDVAVYYCKQSYSRRTFGGGTKVE | | |
| | | IK | | |
| Ab38 | 134 | QVQLVQSGAEVKKPGASVKVSCKASGYTFTDYNMH | SAD10319_P04_F11 | HC amino acid sequence |
| | | WVRQAPGQRLQWMGWINPYTGYTKYSQKFQGRVTI | (ADI-57339) | |
| | | TRDTSASTAYMELSSLRSEDTAVYYCARDHYGHYFY | | |
| | | DVWGQGTLVTVSS | | |
| Ab38 | 135 | DIVMTQSPDSLAVSLGERATINCKSSQSLLNARTGKN | SAD10319_P04_F11 | LC amino acid sequence |
| | | YLAWYQQKPGQPPKLLIYWASTRESGVPDRFSGSGS | (ADI-57339) | |
| | | GTDFTLTISSLQAEDVAVYYCKQSYSRRTFGGGTKVE | | |
| | | IK | | |
| Ab39 | 136 | QVQLVQSGAEVKKPGASVKVYCKASGYTFTTYEMH | SAD10319_P04_G11 | HC amino acid sequence |
| | | WVRQAPGQRLEWMGSINAGDANTKYSQKFQGRVTI | (ADI-48641) | |
| | | TRDTSASTAYMELSSLRSEDTAVYYCARDAYHHYFY | | |
| | | DVWGQGTLVTVSS | | |
| Ab39 | 137 | DIVMTQSPDSLAVSLGERATINCKSSQSLLNARTGKN | SAD10319_P04_G11 | LC amino acid sequence |
| | | YLAWYQQKPGQPPKLLIYWASTRESGVPDRFSGSGS | (ADI-48641) | |
| | | GTDFTLTISSLQAEDVAVYYCKQSYSRRTFGGGTKVE | | |
| | | IK | | |
| Ab40 | 138 | QVQLVQSGAEVKKPGASVKVSCKASGYTFTSYAMH | SAD10319_P04_B12 | HC amino acid sequence |
| | | WVRQAPGQRLEWMGWINPITGYTKYSQKFQGRVTIT | (ADI-57340) | |
| | | RDTSASTAYMELSSLRSEDTAVYYCARDAHGRYHY | | |
| | | DVWGQGTLVTVSS | | |
| Ab40 | 139 | DIVMTQSPDSLAVSLGERATINCKSSQSLLNARTGKN | SAD10319_P04_B12 | LC amino acid sequence |
| | | YLAWYQQKPGQPPKLLIYWASTRESGVPDRFSGSGS | (ADI-57340) | |
| | | GTDFTLTISSLQAEDVAVYYCKQSYSRRTFGGGTKVE | | |
| | | IK | | |
| Ab41 | 140 | QVQLVQSGAEVKKPGASVKVSCKASGYTFTAYDMH | SAD10319_P04_C12 | HC amino acid sequence |
| | | WVRQAPGQRLERMGWIDPITGNTKYSQKFQGRVTIT | (ADI-57341) | |
| | | RDTSASTAYMELSSLRSEDTAVYYCAHDAYGHYFYD | | |
| | | VWGQGTLVTVSS | | |
| Ab41 | 141 | DIVMTQSPDSLAVSLGERATINCKSSQSLLNARTGKN | SAD10319_P04_C12 | LC amino acid sequence |
| | | YLAWYQQKPGQPPKLLIYWASTRESGVPDRFSGSGS | (ADI-57341) | |
| | | GTDFTLTISSLQAEDVAVYYCKQSYSRRTFGGGTKVE | | |
| | | IK | | |
| Ab42 | 142 | QVQLVQSGAEVKKPGASVKVSCKASGYTFTSYAMH | SAD10319_P04_D12 | HC amino acid sequence |
| | | WVRQAPGQRLDWMGYINPDTGDTKYSQKFQGRVTI | (ADI-57342) | |
| | | TRDTSASTAYMELSSLRSEDTAVYYCARDAYHHYFY | | |
| | | DVWGQGTLVTVSS | | |
| Ab42 | 143 | DIVMTQSPDSLAVSLGERATINCKSSQSLLNARTGKN | SAD10319_P04_D12 | LC amino acid sequence |
| | | YLAWYQQKPGQPPKLLIYWASTRESGVPDRFSGSGS | (ADI-57342) | |
| | | GTDFTLTISSLQAEDVAVYYCKQSYSRRTFGGGTKVE | | |
| | | IK | | |
| Ab43 | 144 | QVQLVQSGAEVKKPGASVKVSCKASGYTFTSYPMH | SAD10319_P04_E12 | HC amino acid sequence |
| | | WVRQAPGQRLEWMGWIDAGTGLTTYSQKFQGRVTI | (ADI-48642) | |
| | | TRDTSASTAYMELSSLRSEDTAVYYCARDHYHRYFY | | |
| | | DMWGQGTLVTVSS | | |
| Ab43 | 145 | DIVMTQSPDSLAVSLGERATINCKSSQSLLNARTGKN | SAD10319_P04_E12 | LC amino acid sequence |
| | | YLAWYQQKPGQPPKLLIYWASTRESGVPDRFSGSGS | (ADI-48642) | |
| | | GTDFTLTISSLQAEDVAVYYCKQSYSRRTFGGGTKVE | | |
| | | IK | | |
| Ab44 | 146 | QVQLVQSGAEVKKPGASVKVSCKASGYTFNDYAMH | SAD10319_P04_F12 | HC amino acid sequence |
| | | WVRQAPGQRLDWMGWINPDTGNTNYSQKFQGRVTI | (ADI-57343) | |
| | | TRDTSASTAYMELSSLRSEDTAVYYCARDHYGRYFY | | |
| | | DHWGQGTLVTVSS | | |
| Ab44 | 147 | DIVMTQSPDSLAVSLGERATINCKSSQSLLNARTGKN | SAD10319_P04_F12 | LC amino acid sequence |
| | | YLAWYQQKPGQPPKLLIYWASTRESGVPDRFSGSGS | (ADI-57343) | |
| | | GTDFTLTISSLQAEDVAVYYCKQSYSRRTFGGGTKVE | | |
| | | IK | | |
| Ab45 | 148 | QVQLVQSGAEVKKPGASVKVSCKASGYTFTSDHMH | SAD10319_P04_H12 | HC amino acid sequence |
| | | WVRQAPGQRLEWMGSINAGTGATKYSQKFQGRVTI | (ADI-57344) | |
| | | TRDTSASTAYMELSSLRSEDTAVYYCARDAYGHYFY | | |
| | | DHWGQGTLVTVSS | | |
| Ab45 | 149 | DIVMTQSPDSLAVSLGERATINCKSSQSLLNARTGKN | SAD10319_P04_H12 | LC amino acid sequence |
| | | YLAWYQQKPGQPPKLLIYWASTRESGVPDRFSGSGS | (ADI-57344) | |
| | | GTDFTLTISSLQAEDVAVYYCKQSYSRRTFGGGTKVE | | |
| | | IK | | |
| Ab46 | 150 | QVQLVQSGAEVKKPGASVKVSCKASGFNIKDYYMH | SAD10320_P01_B01 | HC amino acid sequence |
| | | WVRQAPGQRLEWMGWIDLENANTIYDAKFQGRVTI | (ADI-48643) | |
| | | TRDTSASTAYMELSSLRSEDTAVYYCARDAYGHIFY | | |
| | | DVWGQGTLVTVSS | | |
| Ab46 | 151 | DIVMTQSPDSLAVSLGERATINCKSSQSLLNARTGKN | SAD10320_P01_B01 | LC amino acid sequence |
| | | YLAWYQQKPGQPPKLLIYWASTRESGVPDRFSGSGS | (ADI-48643) | |
| | | GTDFTLTISSLQAEDVAVYYCKQSYVHRTFGGGTKV | | |
| | | EIK | | |
| Ab47 | 152 | QVQLVQSGAEVKKPGASVKVSCKASGFNIKDYYMH | SAD10318_P01_C03 | HC amino acid sequence |
| | | WVRQAPGQRLEWMGWIDLENANTIYDAKFQGRVTI | (ADI-57271) | |
| | | TRDTSASTAYMELSSLRSEDTAVYYCARDAHGRYHY | | |
| | | DVWGQGTLVTVSS | | |
| Ab47 | 153 | DIVMTQSPDSLAVSLGERATINCKSSQSLLNRHTGKN | SAD10318_P01_C03 | LC amino acid sequence |
| | | YLAWYQQKPGQPPKLLIYWASTRESGVPDRFSGSGS | (ADI-57271) | |
| | | GTDFTLTISSLQAEDVAVYYCKQSYSRRTFGGGTKVE | | |
| | | I | | |
| Ab48 | 154 | QVQLVQSGAEVKKPGASVKVSCKASGFNIKDYYMH | SAD10320_P01_D01 | HC amino acid sequence |
| | | WVRQAPGQRLEWMGWIDLENANTIYDAKFQGRVTI | (ADI-48644) | |
| | | TRDTSASTAYMELSSLRSEDTAVYYCARDAYGHGFY | | |
| | | DVWGQGTLVTVSS | | |
| Ab48 | 155 | DIVMTQSPDSLAVSLGERATINCKSSQSLLNARTGKN | SAD10320_P01_D01 | LC amino acid sequence |
| | | YLAWYQQKPGQPPKLLIYWASTRESGVPDRFSGSGS | (ADI-48644) | |
| | | GTDFTLTISSLQAEDVAVYYCKQSYSHRTFGGGTKVE | | |
| | | IK | | |
| Ab49 | 156 | QVQLVQSGAEVKKPGASVKVSCKASGFNIKDYYMH | SAD10320_P01_E01 | HC amino acid sequence |
| | | WVRQAPGQRLEWMGWIDLENANTIYDAKFQGRVTI | (ADI-48645) | |
| | | TRDTSASTAYMELSSLRSEDTAVYYCARDAYGHVFY | | |
| | | DVWGQGTLVTVSS | | |
| Ab49 | 157 | DIVMTQSPDSLAVSLGERATINCKSSQSLLNARTGKN | SAD10320_P01_E01 | LC amino acid sequence |
| | | YLAWYQQKPGQPPKLLIYWASTRESGVPDRFSGSGS | (ADI-48645) | |
| | | GTDFTLTISSLQAEDVAVYYCKQSYKHRTFGGGTKV | | |
| | | EIK | | |
| Ab50 | 158 | QVQLVQSGAEVKKPGASVKVSCKASGFNIKDYYMH | SAD10320_P01_F01 | HC amino acid sequence |
| | | WVRQAPGQRPAWMGWIDLENANTIYDAKFQGRVTI | (ADI-57345) | |
| | | TRDTSASTAYMELSSLRSEDTAVYYCARDASHRYFY | | |
| | | DVWGQGTLVTVSS | | |
| Ab50 | 159 | DIVMTQSPDSLAVSLGERATINCKSSQSLLNARTGKN | SAD10320_P01_F01 | LC amino acid sequence |
| | | YLAWYQQKPGQPPKLLIYWASTRESGVPDRFSGSGS | (ADI-57345) | |
| | | GTDFTLTISSLQAEDVAVYYCGHSYSRRTFGGGTKVE | | |
| | | IK | | |
| Ab51 | 160 | QVQLVQSGAEVKKPGASVKVSCKASGFNIKDYYMH | SAD10320_P01_G01 | HC amino acid sequence |
| | | WVRQAPGQRLEWMGWIDLENANTIYDAKFQGRVTI | (ADI-57346) | |
| | | TRDTSASTAYMELSSLRSEDTAVYYCARDAYGHSFY | | |
| | | DVWGQGTLVTVSS | | |
| Ab51 | 161 | DIVMTQSPDSLAVSLGERATINCKSSQSLLNARTGKN | SAD10320_P01_G01 | LC amino acid sequence |
| | | YLAWYQQKPGQPPKLLIYWASTRESGVPDRFSGSGS | (ADI-57346) | |
| | | GTDFTLTISSLQAEDVAVYYCKQSYHLRTFGGGTKV | | |
| | | EIK | | |
| Ab52 | 162 | QVQLVQSGAEVKKPGASVKVSCKASGFNIKDYYMH | SAD10320_P01_H01 | HC amino acid sequence |
| | | WVRQAPGQRLDWMGWIDLENANTIYDAKFQGRVTI | (ADI-57347) | |
| | | TRDTSASTAYMELSSLRSEDTAVYYCARDAYGHNFY | | |
| | | DVWGQGTLVTVSS | | |
| Ab52 | 163 | DIVMTQSPDSLAVSLGERATINCKSSQSLLNARTGKN | SAD10320_P01_H01 | LC amino acid sequence |
| | | YLAWYQQKPGQPPKLLIYWASTRESGVPDRFSGSGS | (ADI-57347) | |
| | | GTDFTLTISSLQAEDVAVYYCKQSYHKRTFGGGTKV | | |
| | | EIK | | |
| Ab53 | 164 | QVQLVQSGAEVKKPGASVKVSCKASGFNIKDYYMH | SAD10320_P01_A02 | HC amino acid sequence |
| | | WVRQAPGQRLEWMGWIDLENANTIYDAKFQGRVTI | (ADI-48646) | |
| | | TRDTSASTAYMELSSLRSEDTAVYYCARDAYPHYFY | | |
| | | DVWGQGTLVTVSS | | |
| Ab53 | 165 | DIVMTQSPDSLAVSLGERATINCKSSQSLLNARTGKN | SAD10320_P01_A02 | LC amino acid sequence |
| | | YLAWYQQKPGQPPKLLIYWASTRESGVPDRFSGSGS | (ADI-48646) | |
| | | GTDFTLTISSLQAEDVAVYYCKQSYHERTFGGGTKV | | |
| | | EIK | | |
| Ab54 | 166 | QVQLVQSGAEVKKPGASVKVSCKASGFNIKDYYMH | SAD10320_P01_C02 | HC amino acid sequence |
| | | WVRQAPGQRLDWMGWIDLENANTIYDAKFQGRVTI | (ADI-57348) | |
| | | TRDTSASTAYMELSSLRSEDTAVYYCARDAGHRYFY | | |
| | | DVWGQGTLVTVSS | | |
| Ab54 | 167 | DIVMTQSPDSLAVSLGERATINCKSSQSLLNARPGKN | SAD10320_P01_C02 | LC amino acid sequence |
| | | YLAWYQQKPGQPPKLLIYWASTRESGVPDRFSGSGS | (ADI-57348) | |
| | | GTDFTLTISSLQAEDVAVYYCGHSYSRRTFGGGTKVE | | |
| | | IK | | |
| Ab55 | 168 | QVQLVQSGAEVKKPGASVKVSCKASGFNIKDYYMH | SAD10320_P01_E02 | HC amino acid sequence |
| | | WVRQAPGQRLAWMGWIDLENANTIYDAKFQGRVTI | (ADI-57349) | |
| | | TRDTSASTAYMELSSLRSEDTAVYYCARDAYGRHFY | | |
| | | DVWGQGTLVTVSS | | |
| Ab55 | 169 | DIVMTQSPDSLAVSLGERATINCKSSQSLLNARTGKN | SAD10320_P01_E02 | LC amino acid sequence |
| | | YLAWYQQKPGQPPKLLIYWASTRESGVPDRFSGSGS | (ADI-57349) | |
| | | GTDFTLTISSLQAEDVAVYYCKQSYSRRTFGGGTKVE | | |
| | | IK | | |
| Ab56 | 170 | QVQLVQSGAEVKKPGASVKVSCKASGFNIKDYYMH | SAD10320_P01_F02 | HC amino acid sequence |
| | | WVRQAPGQRLERMGWIDLENANTIYDAKFQGRVTIT | (ADI-57350) | |
| | | RDTSASTAYMELSSLRSEDTAVYYCARDAYGHTFYD | | |
| | | VWGQGTLVTVSS | | |
| Ab56 | 171 | DIVMTQSPDSLAVSLGERATINCKSSQSLLNARTGKN | SAD10320_P01_F02 | LC amino acid sequence |
| | | YLAWYQQKPGQPPKLLIYWASTRESGVPDRFSGSGS | (ADI-57350) | |
| | | GTDFTLTISSLQAEDVAVYYCKQSYWHRTFGGGTKV | | |
| | | EIK | | |
| Ab57 | 172 | QVQLVQSGAEVKKPGASVKVSCKASGFNIKDYYMH | SAD10320_P01_G02 | HC amino acid sequence |
| | | WVRQAPGQRLQWMGWIDLENANTIYDAKFQGRVTI | (ADI-57351) | |
| | | TRDTSASTAYMELSSLRSEDTAVYYCARDAYGHSFY | | |
| | | DVWGQGTLVTVSS | | |
| Ab57 | 173 | DIVMTQSPDSLAVSLGERATINCKSSQSLLNARTGKN | SAD10320_P01_G02 | LC amino acid sequence |
| | | YLAWYQQKPGQPPKLLIYWASTRESGVPDRFSGSGS | (ADI-57351) | |
| | | GTDFTLTISSLQAEDVAVYYCKQSYHERTFGGGTKV | | |
| | | EIK | | |
| Ab58 | 174 | QVQLVQSGAEVKKPGASVKVSCKASGFNIKDYYMH | SAD10318_P01_D03 | HC amino acid sequence |
| | | WVRQAPGQRLEWMGWIDLENANTIYDAKFQGRVTI | (ADI-48588) | |
| | | TRDTSASTAYMELSSLRSEDTAVYYCARDHYGHYFY | | |
| | | DVWGQGTLVTVSS | | |
| Ab58 | 175 | DIVMTQSPDSLAVSLGERATINCKSSQSLLNAHTGHN | SAD10318_P01_D03 | LC amino acid sequence |
| | | YLAWYQQKPGQPPKLLIYWASTRESGVPDRFSGSGS | (ADI-48588) | |
| | | GTDFTLTISSLQAEDVAVYYCKQSHSHRTFGGGTKVE | | |
| | | IK | | |
| Ab59 | 176 | QVQLVQSGAEVKKPGASVKVSCKASGFNIKDYYMH | SAD10320_P01_H02 | HC amino acid sequence |
| | | WVRQAPGQRLEWMGWIDLENANTIYDAKFQGRVTI | (ADI-57352) | |
| | | TRDTSASTAYMELSSLRSEDTAVYYCARDAYGHSFY | | |
| | | DVWGQGTLVTVSS | | |
| Ab59 | 177 | DIVMTQSPDSLAVSLGERATINCKSSQSLLNARTGKN | SAD10320_P01_H02 | LC amino acid sequence |
| | | YLAWYQQKPGQPPKLLIYWASTRESGVPDRFSGSGS | (ADI-57352) | |
| | | GTDFTLTISSLQAEDVAVYYCKQSMHRRTFGGGTKV | | |
| | | EI | | |
| Ab60 | 178 | QVQLVQSGAEVKKPGASVKVSCKASGFNIKDYYMH | SAD10320_P01_A03 | HC amino acid sequence |
| | | WVRQAPGQRLEWMGWIDLENANTIYDAKFQGRVTI | (ADI-57353) | |
| | | TRDTSASTAYMELSSLRSEDTAVYYCARDHRGRYFY | | |
| | | DVWGQGTLVTVSS | | |
| Ab60 | 179 | DIVMTQSPDSLAVSLGERATINCKSSQSLLNARTGKN | SAD10320_P01_A03 | LC amino acid sequence |
| | | YLAWYQQKPGQPPKLLIYWASTRESGVPDRFSGSGS | (ADI-57353) | |
| | | GTDFTLTISSLQAEDVAVYYCKQSSHRRTFGGGTKVE | | |
| | | IK | | |
| Ab61 | 180 | QVQLVQSGAEVKKPGASVKVSCKASGFNIKDYYMH | SAD10320_P01_B03 | HC amino acid sequence |
| | | WVRQAPGQRLEWMGWIDLENANTIYDAKFQGRVTI | (ADI-57354) | |
| | | TRDTSASTAYMELSSLRSDDTAVYYCARDQHGRYFY | | |
| | | DVWGQGTLVTVSS | | |
| Ab61 | 181 | DIVMTQSPDSLAVSLGERATINCKSSQSLLNARTGKN | SAD10320_P01_B03 | LC amino acid sequence |
| | | YLAWYQQKPGQPPKLLIYWASTRESGVPDRFSGSGS | (ADI-57354) | |
| | | GTDFTLTISSLQAEDVAVYYCKQSYSRRTFGGGTKVE | | |
| | | IK | | |
| Ab62 | 182 | QVQLVQSGAEVKKPGASVKVSCKASGFNIKDYYMH | SAD10320_P01_C03 | HC amino acid sequence |
| | | WVRQAPGQRLEWMGWIDLENANTIYDAKFQGRVTI | (ADI-57355) | |
| | | TRDTSASTAYMELSSLRSEDTAVYYCARDAYGHSFY | | |
| | | DVWGQGTLVTVSS | | |
| Ab62 | 183 | DIVMTQSPDSLAVSLGERATINCKSSQSLLNARTGKN | SAD10320_P01_C03 | LC amino acid sequence |
| | | YLAWYQQKPGQPPKLLIYWASTRESGVPDRFSGSGS | (ADI-57355) | |
| | | GTDFTLTISSLQAEDVAVYYCKQSYHWRTFGGGTKV | | |
| | | EIK | | |
| Ab63 | 184 | QVQLVQSGAEVKKPGASVKVSCKASGFDIKDYYMH | SAD10320_P01_D03 | HC amino acid sequence |
| | | WVRQAPGQRLEWMGWIDLENANTIYDAKFQGRVTI | (ADI-57356) | |
| | | TRDTSASTAYMELSSLRSEDTAVYYCARDAYGHAFY | | |
| | | DVWGQGTLVTVSS | | |
| Ab63 | 185 | DIVMTQSPDSLAVSLGERATINCKSSQSLLNARTGKN | SAD10320_P01_D03 | LC amino acid sequence |
| | | YLAWYQQKPGQPPKLLIYWASTRESGVPDRFSGSGS | (ADI-57356) | |
| | | GTDFTLTISSLQAEDVAVYYCKQSYHGRTFGGGTKV | | |
| | | EIK | | |
| Ab64 | 186 | QVQLVQSGAEVKKPGASVKVSCKASGFNIKDYYMH | SAD10320_P01_E03 | HC amino acid sequence |
| | | WVRQAPGQRLERMGWIDLENANTIYDAKFQGRVTIT | (ADI-57357) | |
| | | RDTSASTAYMELSSLRSEDTAVYYCARDAYGHGFYD | | |
| | | VWGQGTLVTVSS | | |
| Ab64 | 187 | DIVMTQSPDSLAVSLGERATINCKSSQSLLNARTGKN | SAD10320_P01_E03 | LC amino acid sequence |
| | | YLAWYQQKPGQPPKLLIYWASTRESGVPDRFSGSGS | (ADI-57357) | |
| | | GTDFTLTISSLQAEDVAVYYCKQSYRHRTFGGGTKV | | |
| | | EIK | | |
| Ab65 | 188 | QVQLVQSGAEVKKPGASVKVSCKASGFNIKDYYMH | SAD10320_P01_F03 | HC amino acid sequence |
| | | WVRQAPGQRLEWMGWIDLENANTIYDAKFQGRVTI | (ADI-48647) | |
| | | TRDTSASTAYMELSSLRSEDTAVYYCARDAYPHYFY | | |
| | | DVWGQGTLVTVSS | | |
| Ab65 | 189 | DIVMTQSPDSLAVSLGERATINCKSSQSLLNARTGKN | SAD10320_P01_F03 | LC amino acid sequence |
| | | YLAWYQQKPGQPPKLLIYWASTRESGVPDRFSGSGS | (ADI-48647) | |
| | | GTDFTLTISSLQAEDVAVYYCKQSYLHRTFGGGTKV | | |
| | | EIK | | |
| Ab66 | 190 | QVQLVQSGAEVKKPGASVKVSCKASGFNIKDYYMH | SAD10320_P01_G03 | HC amino acid sequence |
| | | WVRQAPGQRPEWMGWIDLENANTIYDAKFQGRVTI | (ADI-57358) | |
| | | TRDTSASTAYMELSSLRSEDTAVYYCARDAYGHSFY | | |
| | | DVWGQGTLVTVSS | | |
| Ab66 | 191 | DIVMTQSPDSLAVSLGERATINCKSSQSLLNARTGKN | SAD10320_P01_G03 | LC amino acid sequence |
| | | YLAWYQQKPGQPPKLLIYWASTRESGVPDRFSGSGS | (ADI-57358) | |
| | | GTDFTLTISSLQAEDVAVYYCKQSYHWRTFGGGTKV | | |
| | | EIK | | |
| Ab67 | 192 | QVQLVQSGAEVKKPGASVKVSCKASGFNIKDYYMH | SAD10320_P01_H03 | HC amino acid sequence |
| | | WVRQAPGQRPEWMGWIDLENANTIYDAKFQGRVTI | (ADI-57359) | |
| | | TRDTSASTAYMELSSLRSEDTAVYYCARDAAHRYFY | | |
| | | DVWGQGTLVTVSS | | |
| Ab67 | 193 | DIVMTQSPDSLAVSLGERATINCKSSQSLLNARPRKN | SAD10320_P01_H03 | LC amino acid sequence |
| | | YLAWYQQKPGQPPKLLIYWASTRESGVPDRFSGSGS | (ADI-57359) | |
| | | GTDFTLTISSLQAEDVAVYYCKQSHDRRTFGGGTKV | | |
| | | EIK | | |
| Ab68 | 194 | QVQLVQSGAEVKKPGASVKVSCKASGFNIKDYYMH | SAD10320_P02_A04 | HC amino acid sequence |
| | | WVRQAPGQRLEWMGWIDLENANTIYDAKFQGRVTI | (ADI-57360) | |
| | | TRDTSASTAYMELSSLRSEDTAVYYCARDAYGHAFY | | |
| | | DVWGQGTLVTVSS | | |
| Ab68 | 195 | DIVMTQSPDSLAVSLGERATINCKSSQSLLNARTGKN | SAD10320_P02_A04 | LC amino acid sequence |
| | | YLAWYQQKPGQPPKLLIYWASTRESGVPDRFSGSGS | (ADI-57360) | |
| | | GTDFTLTISSLQAEDVAVYYCKQSLHRRTFGGGTKVE | | |
| | | IK | | |
| Ab69 | 196 | QVQLVQSGAEVKKPGASVKVSCKASGFNIKDYYMH | SAD10318_P01_E03 | HC amino acid sequence |
| | | WVRQAPGQRLEWMGWIDLENANTIYDAKFQGRVTI | (ADI-57272) | |
| | | TRDTSASTAYMELSSLRSEDTAVYYCARDAHGHYFY | | |
| | | DVWGQGTLVTVSS | | |
| Ab69 | 197 | DIVMTQSPDSLAVSLGERATINCKSSQSLLNAHTGKN | SAD10318_P01_E03 | LC amino acid sequence |
| | | HLAWYQQKPGQPPKLLIYWASTRESGVPDRFSGSGS | (ADI-57272) | |
| | | GTDFTLTISSLQAEDVAVYYCKQSYSRRTFGGGTKVE | | |
| | | IK | | |
| Ab70 | 198 | QVQLVQSGAEVKKPGASVKVSCKASGFNIKDYYMH | SAD10320_P02_B04 | HC amino acid sequence |
| | | WVRQAPGQRLERMGWIDLENANTIYDAKFQGRVTIT | (ADI-57273) | |
| | | RDTSASTAYMELSSLRSEDTAVYYCARDAYGHGFYD | | |
| | | VWGQGTLVTVSS | | |
| Ab70 | 199 | DIVMTQSPDSLAVSLGERATINCKSSQSLLNARTGKN | SAD10320_P02_B04 | LC amino acid sequence |
| | | YLAWYQQKPGQPPKLLIYWASTRESGVPDRFSGSGS | (ADI-57273) | |
| | | GTDFTLTISSLQAEDVAVYYCKQSYGHRTFGGGTKV | | |
| | | EIK | | |
| Ab71 | 200 | QVQLVQSGAEVKKPGASVKVSCKASGFNIKDYYMH | SAD10320_P02_C04 | HC amino acid sequence |
| | | WVRQAPGQRLEWMGWIDLENANTIYDAKFQGRVTI | (ADI-48648) | |
| | | TRDTSASTAYMELSSLRSEDTAVYYCARDAYGRYFY | | |
| | | DVWGQGTLVTVSS | | |
| Ab71 | 201 | DIVMTQSPDSLAVSLGERATINCKSSQSLLNARTGKN | SAD10320_P02_C04 | LC amino acid sequence |
| | | YLAWYQQKPGQPPKLLIYWASTRESGVPDRFSGSGS | (ADI-48648) | |
| | | GTDFTLTISSLQAEDVAVYYCKQSHTRRTFGGGTKVE | | |
| | | IK | | |
| Ab72 | 202 | QVQLVQSGAEVKKPGASVKVSCKASGFNIKDYYMH | SAD10320_P02_E04 | HC amino acid sequence |
| | | WVRQAPGQRLEWMGWIDLENANTIYDAKFQGRVTI | (ADI-48649) | |
| | | TRDTSASTAYMELSSLRSEDTAVYYCARDAYHEYFY | | |
| | | DVWGQGTLVTVSS | | |
| Ab72 | 203 | DIVMTQSPDSLAVSLGERATINCKSSQSLLNARTGKN | SAD10320_P02_E04 | LC amino acid sequence |
| | | YLAWYQQKPGQPPKLLIYWASTRESGVPDRFSGSGS | (ADI-48649) | |
| | | GTDFTLTISSLQAEDVAVYYCKQSHVRRTFGGGTKV | | |
| | | EIK | | |
| Ab73 | 204 | QVQLVQSGAEVKKPGASVKVSCKASGFNIKDYYMH | SAD10320_P02_H04 | HC amino acid sequence |
| | | WVRQAPGQRLDWMGWIDLENANTIYDAKFQGRVTI | (ADI-57362) | |
| | | TRDTSASTAYMELSSLRSEDTAVYYCARDAYGHLFY | | |
| | | DVWGQGTLVTVSS | | |
| Ab73 | 205 | DIVMTQSPDSLAVSLGERATINCKSSQSLLNARTGKN | SAD10320_P02_H04 | LC amino acid sequence |
| | | YLAWYQQKPGQPPKLLIYWASTRESGVPDRFSGSGS | (ADI-57362) | |
| | | GTDFTLTISSLQAEDVAVYYCKQSHMRRTFGGGTKV | | |
| | | EIK | | |
| Ab74 | 206 | QVQLVQSGAEVKKPGASVKVSCKASGFNIKDYYMH | SAD10320_P02_A05 | HC amino acid sequence |
| | | WVRQAPGQRLEWMGWIDLENANTIYDAKFQGRVTI | (ADI-57363) | |
| | | TRDTSASTAYMELSSLRSEDTAVYYCARDAYGHMFY | | |
| | | DVWGQGTLVTVS | | |
| Ab74 | 207 | DIVMTQSPDSLAVSLGERATINCKSSQSLLNARPGKN | SAD10320_P02_A05 | LC amino acid sequence |
| | | YLAWYQQKPGQPPKLLIYWASTRESGVPDRFSGSGS | (ADI-57363) | |
| | | GTDFTLTISSLQAEDVAVYYCKQSYHMRTFGGGTKV | | |
| | | EIK | | |
| Ab75 | 208 | QVQLVQSGAEVKKPGASVKVSCKASGFNIKDYYMH | SAD10320_P02_B05 | HC amino acid sequence |
| | | WVRQAPGQRLEWMGWIDLENANTIYDAKFQGRVTI | (ADI-57364) | |
| | | TRDTSASTAYMELSSLRSEDTAVYYCARDAYGHNFY | | |
| | | DVWGQGTLVTVSS | | |
| Ab75 | 209 | DIVMTQSPDSLAVSLGERATINCKSSQSLLNARTGKN | SAD10320_P02_B05 | LC amino acid sequence |
| | | YLAWYQQKPGQPPKLLIYWASTRESGVPDRFSGSGS | (ADI-57364) | |
| | | GTDFTLTISSLQAEDVAVYYCKQSYTHRTFGGGTKV | | |
| | | EIK | | |
| Ab76 | 210 | QVQLVQSGAEVKKPGASVKVSCKASGSNIKDYYMH | SAD10320_P02_C05 | HC amino acid sequence |
| | | WVRQAPGQRLEWMGWIDLENANTIYDAKFQGRVTI | (ADI-57365) | |
| | | TRDTSASTAYMELSSLRSEDTAVYYCARDAYGHSFY | | |
| | | DVWGQGTLVTVSS | | |
| Ab76 | 211 | DIVMTQSPDSLAVSLGERATINCKSSQSLLNARTGKN | SAD10320_P02_C05 | LC amino acid sequence |
| | | YLAWYQQKPGQPPKLLIYWASTRESGVPDRFSGSGS | (ADI-57365) | |
| | | GTDFTLTISSLQAEDVAVYYCKQSYHGRTFGGGTKV | | |
| | | EIK | | |
| Ab77 | 212 | QVQLVQSGAEVKKPGASVKVSCKASGFNIKDYYMH | SAD10320_P02_D05 | HC amino acid sequence |
| | | WVRQAPGQRLEWMGWIDLENANTIYDAKFQGRVTI | (ADI-48650) | |
| | | TRDTSASTAYMELSSLRSEDTAVYYCARDATHRYFY | | |
| | | DVWGQGTLVTVSS | | |
| Ab77 | 213 | DIVMTQSPDSLAVSLGERATINCKSSQSLLNARTGKN | SAD10320_P02_D05 | LC amino acid sequence |
| | | YLAWYQQKPGQPPKLLIYWASTRESGVPDRFSGSGS | (ADI-48650) | |
| | | GTDFTLTISSLQAEDVAVYYCKQSHTRRTFGGGTKVE | | |
| | | I | | |
| Ab78 | 214 | QVQLVQSGAEVKKPGASVKVSCKASGFNIKDYYMH | SAD10320_P02_E05 | HC amino acid sequence |
| | | WVRQAPGQRLEWMGWIDLENANTIYDAKFQGRVTI | (ADI-57366) | |
| | | TRDTSASTAYMELSSLRSEDTAVYYCARDMHGRYFY | | |
| | | DVWGQGTLVTVSS | | |
| Ab78 | 215 | DIVMTQSPDSLAVSLGERATINCKSSQSLLNARTGKN | SAD10320_P02_E05 | LC amino acid sequence |
| | | YLAWYQQKPGQPPKLLIYWASTRESGVPDRFSGSGS | (ADI-57366) | |
| | | GTDFTLTISSLQAEDVAVYYCKQSEHRRTFGGGTKVE | | |
| | | IK | | |
| Ab79 | 216 | QVQLVQSGAEVKKPGASVKVSCKASGFNIKDYYMH | SAD10320_P02_F05 | HC amino acid sequence |
| | | WVRQAPGQRLEWMGWIDLENANTIYDAKFQGRVTI | (ADI-48651) | |
| | | TRDTSASTAYMELSSLRSEDTAVYYCARDAYGRYHY | | |
| | | DVWGQGTLVTVSS | | |
| Ab79 | 217 | DIVMTQSPDSLAVSLGERATINCKSSQSLLNARTGKN | SAD10320_P02_F05 | LC amino acid sequence |
| | | YLAWYQQKPGQPPKLLIYWASTRESGVPDRFSGSGS | (ADI-48651) | |
| | | GTDFTLTISSLQAEDVAVYYCKQSYRHRTFGGGTKV | | |
| | | EIK | | |
| Ab80 | 218 | QVQLVQSGAEVKKPGASVKVSCKASGFNIKDYYMH | SAD10318_P01_F03 | HC amino acid sequence |
| | | WVRQAPGQRLEWMGWIDLENANTIYDAKFQGRVTI | (ADI-48589) | |
| | | TRDTSASTAYMELSSLRSEDTAVYYCARDAHGRYFY | | |
| | | DHWGQGTLVTVSS | | |
| Ab80 | 219 | DIVMTQSPDSLAVSLGERATINCKSSQSLLNAHTGHN | SAD10318_P01_F03 | LC amino acid sequence |
| | | YLAWYQQKPGQPPKLLIYWASTRESGVPDRFSGSGS | (ADI-48589) | |
| | | GTDFTLTISSLQAEDVAVYYCKQSYSHRTFGGGTKVE | | |
| | | IK | | |
| Ab81 | 220 | QVQLVQSGAEVKKPGASVKVSCKASGFNIKDYYMH | SAD10320_P02_H05 | HC amino acid sequence |
| | | WVRQAPGQRLEWMGWIDLENANTIYDAKFQGRVTI | (ADI-48652) | |
| | | TRDTSASTAYMELSSLRSEDTAVYYCARDAHHRYFY | | |
| | | DVWGQGTLVTVSS | | |
| Ab81 | 221 | DIVMTQSPDSLAVSLGERATINCKSSQSLLNARTGKN | SAD10320_P02_H05 | LC amino acid sequence |
| | | YLAWYQQKPGQPPKLLIYWASTRESGVPDRFSGSGS | (ADI-48652) | |
| | | GTDFTLTISSLQAEDVAVYYCKQSHSRRTFGGGTKVE | | |
| | | IK | | |
| Ab82 | 222 | QVQLVQSGAEVKKPGASVKVSCKASGFNIKDYYMH | SAD10320_P02_A06 | HC amino acid sequence |
| | | WVRQAPGQRLPWMGWIDLENANTIYDAKFQGRVTI | (ADI-57367) | |
| | | TRDTSASTAYMELSSLRSEDTAVYYCARDAHERYFY | | |
| | | DVWGQGTLVTVSS | | |
| Ab82 | 223 | DIVMTQSPDSLAVSLGERATINCKSSQSLLNARTGKN | SAD10320_P02_A06 | LC amino acid sequence |
| | | YLAWYQQKPGQPPKLLIYWASTRESGVPDRFSGSGS | (ADI-57367) | |
| | | GTDFTLTISSLQAEDVAVYYCKQSHLRRTFGGGTKVE | | |
| | | IK | | |
| Ab83 | 224 | QVQLVQSGAEVKKPGASVKVSCKASGFNIKDYYMH | SAD10320_P02_B06 | HC amino acid sequence |
| | | WVRQAPGQRLEWMGWIDLENANTIYDAKFQGRVTI | (ADI-48653) | |
| | | TRDTSASTAYMELSSLRSEDTAVYYCARDAYGHWF | | |
| | | YDVWGQGTLVTVSS | | |
| Ab83 | 225 | DIVMTQSPDSLAVSLGERATINCKSSQSLLNARTGKN | SAD10320_P02_B06 | LC amino acid sequence |
| | | YLAWYQQKPGQPPKLLIYWASTRESGVPDRFSGSGS | (ADI-48653) | |
| | | GTDFTLTISSLQAEDVAVYYCKQSYHHRTFGGGTKV | | |
| | | EIK | | |
| Ab84 | 226 | QVQLVQSGAEVKKPGASVKVSCKASGFNIKDYYMH | SAD10320_P02_C06 | HC amino acid sequence |
| | | WVRQAPGQRLEWMGWIDLENANTIYDAKFQGRVTI | (ADI-48654) | |
| | | TRDTSASTAYMELSSLRSEDTAVYYCARDAYGRYFY | | |
| | | DVWGQGTLVTVSS | | |
| Ab84 | 227 | DIVMTQSPDSLAVSLGERATINCKSSQSLLNARTGKN | SAD10320_P02_C06 | LC amino acid sequence |
| | | YLAWYQQKPGQPPKLLIYWASTRESGVPDRFSGSGS | (ADI-48654) | |
| | | GTDFTLTISSLQAEDVAVYYCKQSGHRRTFGGGTKV | | |
| | | EIK | | |
| Ab85 | 228 | QVQLVQSGAEVKKPGASVKVSCKASGFNIKDYYMH | SAD10320_P02_D06 | HC amino acid sequence |
| | | WVRQAPGQRPAWMGWIDLENANTIYDAKFQGRVTI | (ADI-57368) | |
| | | TRDTSASTAYMELSSLRSEDTAVYYCARDAYGHGFY | | |
| | | DVWGQGTLVTVSS | | |
| Ab85 | 229 | DIVMTQSPDSLAVSLGERATINCKSSQSLLNARTGKN | SAD10320_P02_D06 | LC amino acid sequence |
| | | YLAWYQQKPGQPPKLLIYWASTRESGVPDRFSGSGS | (ADI-57368) | |
| | | GTDFTLTISSLQAEDVAVYYCKQSYGHRTFGGGTKV | | |
| | | EIK | | |
| Ab86 | 230 | QVQLVQSEAEVKKPGASVKVSCKASGFNIKDYYMH | SAD10320_P02_E06 | HC amino acid sequence |
| | | WVRQAPGQRLQWMGWIDLENANTIYDAKFQGRVTI | (ADI-57369) | |
| | | TRDTSASTAYMELSSLRSEDTAVYYCARDAYGHGFY | | |
| | | DVWGQGTLVTVSS | | |
| Ab86 | 231 | DIVMTQSPDSLAVSLGERATINCKSSQSLLNARPGKN | SAD10320_P02_E06 | LC amino acid sequence |
| | | YLAWYQQKPGQPPKLLIYWASTRESGVPDRFSGSGS | (ADI-57369) | |
| | | GTDFTLTISSLQAEDVAVYYCKQSYSRRTFGGGTKVE | | |
| | | IK | | |
| Ab87 | 232 | QVQLVQSGAEVKKPGASVKVSCKASGFNIKDYYMH | SAD10320_P02_F06 | HC amino acid sequence |
| | | WVRQAPGQRLAWMGWIDLENANTIYDAKFQGRVTI | (ADI-57370) | |
| | | TRDTSASTAYMELSSLRSEDTAVYYCARDAYGRFHY | | |
| | | DVWGQGTLVTVSS | | |
| Ab87 | 233 | DIVMTQSPDSLAVSLGERATINCKSSQSLLNARPGKN | SAD10320_P02_F06 | LC amino acid sequence |
| | | YLAWYQQKPGQPPKLLIYWASTRESGVPDRFSGSGS | (ADI-57370) | |
| | | GTDFTLTISSLQAEDVAVYYCKQSYSRRTFGGGTKVE | | |
| | | IK | | |
| Ab88 | 234 | QVQLVQSGAEVKKPGASVKVSCKASGFNIKDYYMH | SAD10320_P02_G06 | HC amino acid sequence |
| | | WVRQAPGQRLDWMGWIDLENANTIYDAKFQGRVTI | (ADI-57371) | |
| | | TRDTSASTAYMELSSLRSEDTAVYYCARDAYGRHFY | | |
| | | DVWGQGTLVTVSS | | |
| Ab88 | 235 | DIVMTQSPDSLAVSLGERATINCKSSQSLLNARTRTN | SAD10320_P02_G06 | LC amino acid sequence |
| | | YLAWYQQKPGQPPKLLIYWASTRESGVPDRFSGSGS | (ADI-57371) | |
| | | GTDFTLTISSLQAEDVAVYYCKQSYSRRTFGGGTKVE | | |
| | | IK | | |
| Ab89 | 236 | QVQLVQSGAEVKKPGASVKVSCKASGFNIKDYYMH | SAD10320_P02_H06 | HC amino acid sequence |
| | | WVRQAPGQRLEWMGWIDLENANTIYDAKFQGRVTI | (ADI-57372) | |
| | | TRDTSASTAYMELSSLRSEDTAVYYCARDAYGRNHY | | |
| | | DVWGQGTLVTVSS | | |
| Ab89 | 237 | DIVMTQSPDSLAVSLGERATINCKSSQSLLNARTGKS | SAD10320_P02_H06 | LC amino acid sequence |
| | | YLAWYQQKPGQPPKLLIYWASTRESGVPDRFSGSGS | (ADI-57372) | |
| | | GTDFTLTISSLQAEDVAVYYCKQSYVHRTFGGGTKV | | |
| | | EIK | | |
| Ab90 | 238 | QVQLVQSGAEVKKPGASVKVSCKASGFNIKDYYMH | SAD10320_P03_B07 | HC amino acid sequence |
| | | WVRQAPGQRLAWMGWIDLENANTIYDAKFQGRVTI | (ADI-57373) | |
| | | TRDTSASTAYMELSSLRSEDTAVYYCARDAYGHDFY | | |
| | | DVWGQGTLVTVSS | | |
| Ab90 | 239 | DIVMTQSPDSLAVSLGERATINCKSSQSLLNARTGKN | SAD10320_P03_B07 | LC amino acid sequence |
| | | YLAWYQQKPGQPPKLLIYWASTRESGVPDRFSGSGS | (ADI-57373) | |
| | | GTDFTLTISSLQAEDVAVYYCKQSYSRRTFGGGTKVE | | |
| | | IK | | |
| Ab91 | 240 | QVQLVQSGAEVKKPGASVKVSCKASGFNIKDYYMH | SAD10318_P01_G03 | HC amino acid sequence |
| | | WVRQAPGQRLEWMGWIDLENANTIYDAKFQGRVTI | (ADI-48590) | |
| | | TRDTSASTAYMELSSLRSEDTAVYYCAHDAYGHYFY | | |
| | | DVWGQGTLVTVSS | | |
| Ab91 | 241 | DIVMTQSPDSLAVSLGERATINCKSSQSLLNAHTGHN | SAD10318_P01_G03 | LC amino acid sequence |
| | | YLAWYQQKPGQPPKLLIYWASTRESGVPDRFSGSGS | (ADI-48590) | |
| | | GTDFTLTISSLQAEDVAVYYCKQSYHHRTFGGGTKV | | |
| | | EIK | | |
| Ab92 | 242 | QVQLVQSGAEVKKPGASVKVSCKASGFNIKDYYMH | SAD10320_P03_E07 | HC amino acid sequence |
| | | WVRQAPGQRLEWMGWIDLENANTIYDAKFQGRVTI | (ADI-57374) | |
| | | TRDTSASTAYMELSSLRSEDTAVYYCARDAVHRYFY | | |
| | | DVWGQGTLVTVSS | | |
| Ab92 | 243 | DIVMTQSPDSLAVSLGERATINCKSSQSLLNARTGKN | SAD10320_P03_E07 | LC amino acid sequence |
| | | YLAWYQQKPGQPPKLLIYWASTRESGVPDRFSGSGS | (ADI-57374) | |
| | | GTDFTLTISSLQAEDVAVYYCKQSYHPRTFGGGTKVE | | |
| | | IK | | |
| Ab93 | 244 | QVQLVQSGAEVKKPGASVKVSCKASGFNIKDYYMH | SAD10320_P03_H07 | HC amino acid sequence |
| | | WVRQAPGQRLEWMGWIDLENANTIYDAKFQGRVTI | (ADI-57375) | |
| | | TRDTSASTAYMELSSLRSEDTAVYYCARDAYGHTFY | | |
| | | DVWGQGTLVTVSS | | |
| Ab93 | 245 | DIVMTQSPDSLAVSLGERATINCKSSQSLLNARTGKN | SAD10320_P03_H07 | LC amino acid sequence |
| | | YLAWYQQKPGQPPKLLIYWASTRESGVPDRFSGSGS | (ADI-57375) | |
| | | GTDFTLTISSLQAEDVAVYYCKQSYSHVTFGGGTKV | | |
| | | EIK | | |
| Ab94 | 246 | QVQLVQSGAEVKKPGASVKVSCEASGFNIKDYYMH | SAD10320_P03_C08 | HC amino acid sequence |
| | | WVRQAPGQTLAWMGWIDLENANTIYDAKFQGRVTI | (ADI-57376) | |
| | | TRDTSASTAYMELSSLRSEDTAVYYCARDAYGHVFY | | |
| | | DVWGQGTLVTVSS | | |
| Ab94 | 247 | DIVMTQSPDSLAVSLGERATINCKSSQSLLNARTGKN | SAD10320_P03_C08 | LC amino acid sequence |
| | | YLAWYQQKPGQPPKLLIYWASTRESGVPDRFSGSGS | (ADI-57376) | |
| | | GTDFTLTISSLQAEDVAVYYCKQSYLHRTFGGGTKV | | |
| | | EIK | | |
| Ab95 | 248 | QVQLVQSGAEVKKPGASVKVSCKASGFNIKDYYMH | SAD10320_P03_D08 | HC amino acid sequence |
| | | WVRQAPGQRLEWMGWIDLENANTIYDAKFQGRVTI | (ADI-57377) | |
| | | TRDTSASTAYMELSSLRSEDTAVYYCARDAYGHQFY | | |
| | | DVWGQGTLVTVSS | | |
| Ab95 | 249 | DIVMTQSPDSLAVSLGERATINCKSSQSLLNARTGKN | SAD10320_P03_D08 | LC amino acid sequence |
| | | YLAWYQQKPGQPPKLLIYWASTRESGVPDRFSGSGS | (ADI-57377) | |
| | | GTDFTLTISSLQAEDVAVYYCKQSYHTRTFGGGTKV | | |
| | | EIK | | |
| Ab96 | 250 | QVQLVQSGAEVKKPGASVKVSCKASGFNIKDYYMH | SAD10320_P03_F08 | HC amino acid sequence |
| | | WVRQAPGQRLEWMGWIDLENANTIYDAKFQGRVTI | (ADI-57378) | |
| | | TRDTSASTAYMELSSLRSEDTAVYYCARDAYGHDFY | | |
| | | DVWGQGTLVTVSS | | |
| Ab96 | 251 | DIVMTQSPDSLAVSLGERATINCKSSQSLLNARPGKN | SAD10320_P03_F08 | LC amino acid sequence |
| | | YLAWYQQKPGQPPKLLIYWASTRESGVPDRFSGSGS | (ADI-57378) | |
| | | GTDFTLTISSLQAEDVAVYYCKQSYSRRTFGGGTKVE | | |
| | | IK | | |
| Ab97 | 252 | QVQLVQSGAEVKKPGASVKVSCKASGFNIKDYYMH | SAD10320_P03_H08 | HC amino acid sequence |
| | | WVRQAPGQRLEWMGWIDLENANTIYDAKFQGRVTI | (ADI-57379) | |
| | | TRDTSASTAYMELSSLRSEDTAVYYCARDAYGHVFY | | |
| | | DVWGQGTLVTVSS | | |
| Ab97 | 253 | DIVMTQSPDSLAVSLGERATINCKSSQSLLNASTAKN | SAD10320_P03_H08 | LC amino acid sequence |
| | | YLAWYQQKPGQPPKLLIYWASTRESGVPDRFSGSGS | (ADI-57379) | |
| | | GTDFTLTISSLQAEDVAVYYCKQSYRHRTFGGGTKV | | |
| | | EIK | | |
| Ab98 | 254 | QVQLVQSGAEVKKPGASVKVSCKASGFNIKDYYMH | SAD10320_P03_A09 | HC amino acid sequence |
| | | WVRQAPGQRLEWMGWIDLENANTIYDAKFQGRVTI | (ADI-57380) | |
| | | TRDTSASTAYMELSSLRSEDTAVYYCARDAYGHVFY | | |
| | | DVWGQGTLVTVSS | | |
| Ab98 | 255 | DIVMTQSPDSLAVSLGERATINCKSSQSLLNARTGKN | SAD10320_P03_A09 | LC amino acid sequence |
| | | YLAWYQQKPGQPPKLLIYWASTRESGVPDRFSGSGS | (ADI-57380) | |
| | | GTDFTLTISSLQAEDVAVYYCKQSSHRRTFGGGTKVE | | |
| | | IK | | |
| Ab99 | 256 | QVQLVQSGAEVKKPGASVKVSCEASGFNIKDYYMH | SAD10320_P03_C09 | HC amino acid sequence |
| | | WVRQAPGQRLEWMGWIDLENANTIYDAKFQGRVTI | (ADI-57381) | |
| | | TRDTSASTAYMELSSLRSEDTAVYYCARDAYGHVFY | | |
| | | DVWGQGTLVTVSS | | |
| Ab99 | 257 | DIVMTQSPDSLAVSLGERATINCKSSQSLLNARTGKN | SAD10320_P03_C09 | LC amino acid sequence |
| | | YLAWYQQKPGQPPKLLIYWASTRESGVPDRFSGSGS | (ADI-57381) | |
| | | GTDFTLTISSLQAEDVAVYYCKQSYLHRTFGGGTKV | | |
| | | EIK | | |
| Ab100 | 258 | QVQLVQSGAEVKKPGASVKVSCKASGFNIKDYYMH | SAD10320_P03_D09 | HC amino acid sequence |
| | | WVRQAPGQRLEWMGWIDLENANTIYDAKFQGRVTI | (ADI-48655) | |
| | | TRDTSASTAYMELSSLRSEDTAVYYCARDAYGHDFY | | |
| | | DVWGQGTLVTVSS | | |
| Ab100 | 259 | DIVMTQSPDSLAVSLGERATINCKSSQSLLNARTGKN | SAD10320_P03_D09 | LC amino acid sequence |
| | | YLAWYQQKPGQPPKLLIYWASTRESGVPDRFSGSGS | (ADI-48655) | |
| | | GTDFTLTISSLQAEDVAVYYCKQSWHRRTFGGGTKV | | |
| | | EIK | | |
| Ab101 | 260 | QVQLVQSGAEVKKPGASVKVSCKASGFNIKDYYMH | SAD10320_P03_F09 | HC amino acid sequence |
| | | WVRQAPGQRLEWMGWIDLENANTIYDAKFQGRVTI | (ADI-48656) | |
| | | TRDTSASTAYMELSSLRSEDTAVYYCARDAYGHDFY | | |
| | | DVWGQGTLVTVSS | | |
| Ab101 | 261 | DIVMTQSPDSLAVSLGERATINCKSSQSLLNARTGKN | SAD10320_P03_F09 | LC amino acid sequence |
| | | YLAWYQQKPGQPPKLLIYWASTRESGVPDRFSGSGS | (ADI-48656) | |
| | | GTDFTLTISSLQAEDVAVYYCKQSYHRRTFGGGTKV | | |
| | | EIK | | |
| Ab102 | 262 | QVQLVQSGAEVKKPGASVKVSCKASGFNIKDYYMH | SAD10318_P01_H03 | HC amino acid sequence |
| | | WVRQAPGQRLEWMGWIDLENANTIYDAKFQGRVTI | (ADI-48591) | |
| | | TRDTSASTAYMELSSLRSEDTAVYYCAHDAYGHYFY | | |
| | | DVWGQGTLVTVSS | | |
| Ab102 | 263 | DIVMTQSPDSLAVSLGERATINCKSSQSLLNARTGHN | SAD10318_P01_H03 | LC amino acid sequence |
| | | YLAWYQQKPGQPPKLLIYWASTRESGVPDRFSGRGS | (ADI-48591) | |
| | | GTDFTLTISSLQAEDVAVYYCKQSYHHRTFGGGTKV | | |
| | | EIK | | |
| Ab103 | 264 | QVQLVQSGAEVKKPGASVKVSCKASGFNIKDYYMH | SAD10320_P04_A10 | HC amino acid sequence |
| | | WVRQAPGQRLDWMGWIDLENANTIYDAKFQGRVTI | (ADI-57382) | |
| | | TRDTSASTAYMELSSLRSEDTAVYYCARDAYGHDFY | | |
| | | DVWGQGTLVTVSS | | |
| Ab103 | 265 | DIVMTQSPDSLAVSLGERATINCKSSQSLLNARTGKN | SAD10320_P04_A10 | LC amino acid sequence |
| | | YLAWYQQKPGQPPKLLIYWASTRESGVPDRFSGSGS | (ADI-57382) | |
| | | GTDFTLTISSLQAEDVAVYYCKQSYSRRTFGGGTKVE | | |
| | | IK | | |
| Ab104 | 266 | QVQLVQSGAEVKKPGASVKVSCKASGFNIKDYYMH | SAD10320_P04_C10 | HC amino acid sequence |
| | | WVRQAPGQRLEWMGWIDLENANTIYDAKFQGRVTI | (ADI-57383) | |
| | | TRDTSASTAYMELSSLRSEDTAVYYCARDAYGHAFY | | |
| | | DVWGQGTLVTVSS | | |
| Ab104 | 267 | DIVMTQSPDSLAVSLGERATINCKSSQSLLNARTGKN | SAD10320_P04_C10 | LC amino acid sequence |
| | | YLAWYQQKPGQPPKLLIYWASTRESGVPDRFSGSGS | (ADI-57383) | |
| | | GTDFTLTISSLQAEDVAVYYCKQSYHQRTFGGGTKV | | |
| | | EIK | | |
| Ab105 | 268 | QVQLVQSGAEVKKPGASVKVSCEASGFNIKDYYMH | SAD10320_P04_D10 | HC amino acid sequence |
| | | WVRQAPGQRPEWMGWIDLENANTIYDAKFQGRVTI | (ADI-57384) | |
| | | TRDTSASTAYMELSSLRSEDTAVYYCARDAYGHVFY | | |
| | | DVWGQGTLVTVSS | | |
| Ab105 | 269 | DIVMTQSPDSLAVSLGERATINCKSSQSLLNARTGKN | SAD10320_P04_D10 | LC amino acid sequence |
| | | YLAWYQQKPGQPPKLLIYWASTRESGVPDRFSGSGS | (ADI-57384) | |
| | | GTDFTLTISSLQAEDVAVYYCKQSYLHRTFGGGTKV | | |
| | | EIK | | |
| Ab106 | 270 | QVQLVQSGAEVKKPGASVKVSCKASGFNIKDYYMH | SAD10320_P04_E10 | HC amino acid sequence |
| | | WVRQAPGQRPEWMGWIDLENANTIYDAKFQGRVTI | (ADI-48657) | |
| | | TRDTSASTAYMELSSLRSEDTAVYYCARDAYGHDFY | | |
| | | DVWGQGTLVTVSS | | |
| Ab106 | 271 | DIVMTQSPDSLAVSLGERATINCKSSQSLLNARTGKN | SAD10320_P04_E10 | LC amino acid sequence |
| | | YLAWYQQKPGQPPKLLIYWASTRESGVPDRFSGSGS | (ADI-48657) | |
| | | GTDFTLTISSLQAEDVAVYYCKQSYSRRTFGGGTKVE | | |
| | | IK | | |
| Ab107 | 272 | QVQLVQSGAEVKKPGASVKVSCKASGFNIKDYYMH | SAD10320_P04_F10 | HC amino acid sequence |
| | | WVRQAPGQTLDWMGWIDLENANTIYDAKFQGRVTI | (ADI-57385) | |
| | | TRDTSASTAYMELSSLRSEDTAVYYCARDAYGRNHY | | |
| | | DVWGQGTLVTVSS | | |
| Ab107 | 273 | DIVMTQSPDSLAVSLGERATINCKSSQSLLNARPGKN | SAD10320_P04_F10 | LC amino acid sequence |
| | | YLAWYQQKPGQPPKLLIYWASTRESGVPDRFSGSGS | (ADI-57385) | |
| | | GTDFTLTISSLQAEDVAVYYCKQSYLHRTFGGGTKV | | |
| | | EIK | | |
| Ab108 | 274 | QVQLVQSGAEVKKPGASVKVSCKASGFNIKDYYMH | SAD10320_P04_G10 | HC amino acid sequence |
| | | WVRQAPGQRLEWMGWIDLENANTIYDAKFQGRVTI | (ADI-48658) | |
| | | TRDTSASTAYMELSSLRSEDTAVYYCARDAYGHDFY | | |
| | | DVWGQGTLVTVSS | | |
| Ab108 | 275 | DIVMTQSPDSLAVSLGERATINCKSSQSLLNARTGKN | SAD10320_P04_G10 | LC amino acid sequence |
| | | YLAWYQQKPGQPPKLLIYWASTRESGVPDRFSGSGS | (ADI-48658) | |
| | | GTDFTLTISSLQAEDVAVYYCKQSYSRRTFGGGTKVE | | |
| | | IK | | |
| Ab109 | 276 | QVQLVQSGAEVKKPGASVKVSCKASGFNIKDYYMH | SAD10320_P04_A11 | HC amino acid sequence |
| | | WVRQAPGQRLQWMGWIDLENANTIYDAKFQGRVTI | (ADI-57386) | |
| | | TRDTSASTAYMELSSLRSEDTAVYYCARDARHRYFY | | |
| | | DVWGQGTLVTVSS | | |
| Ab109 | 277 | DIVMTQSPDSLAVSLGERATINCKSSQSLLNARTGKN | SAD10320_P04_A11 | LC amino acid sequence |
| | | YLAWYQQKPGQPPKLLIYWASTRESGVPDRFSGSGS | (ADI-57386) | |
| | | GTDFTLTISSLQAEDVAVYYCKQSYSHGTFGGGTKV | | |
| | | EIK | | |
| Ab110 | 278 | QVQLVQSGAEVKKPGASVKVSCKASGFNIKDYYMH | SAD10320_P04_D11 | HC amino acid sequence |
| | | WVRQAPGQRLQWMGWIDLENANTIYDAKFQGRVTI | (ADI-57387) | |
| | | TRDTSASTAYMELSSLRSEDTAVYYCARDAYGHVFY | | |
| | | DVWGQGTLVTVSS | | |
| Ab110 | 279 | DIVMTQSPDSLAVSLGERATINCKSSQSLLNARTGKN | SAD10320_P04_D11 | LC amino acid sequence |
| | | YLAWYQQKPGQPPKLLIYWASTRESGVPDRFSGSGS | (ADI-57387) | |
| | | GTDFTLTISSLQAEDVAVYYCKQSYHHRTFGGGTKV | | |
| | | EIK | | |
| Ab111 | 280 | QVQLVQSGAEVKKPGASVKVSCKASGFNIKDYYMH | SAD10320_P04_E11 | HC amino acid sequence |
| | | WVRQAPGQRLEWMGWIDLENANTIYDAKFQGRVTI | (ADI-57388) | |
| | | TRDTSASTAYMELSSLRSEDTAVYYCARDAYGHDFY | | |
| | | DVWGQGTLVTVSS | | |
| Ab111 | 281 | DIVMTQSPDSLAVSLGERATINCKSSQSLLNARTGKN | SAD10320_P04_E11 | LC amino acid sequence |
| | | YLAWYQQKPGQPPKLLIYWASTRESGVPDRFSGSGS | (ADI-57388) | |
| | | GTDFTLTISSLQAEDVAVYYCKQSYGHRTFGGGTKV | | |
| | | EIK | | |
| Ab112 | 282 | QVQLVQSGAEVKKPGASVKVSCKASGFNIKDYYMH | SAD10320_P04_F11 | HC amino acid sequence |
| | | WVRQAPGQRLEWMGWIDLENANTIYDAKFQGRVTI | (ADI-57389) | |
| | | TRDTSASTAYMELSSLRSEDTAVYYCARDAYGHEFY | | |
| | | DVWGQGTLVTVSS | | |
| Ab112 | 283 | DIVMTQSPDSLAVSLGERATINCKSSQSLLNARTGKN | SAD10320_P04_F11 | LC amino acid sequence |
| | | YLAWYQQKPGQPPKLLIYWASTRESGVPDRFSGSGS | (ADI-57389) | |
| | | GTDFTLTISSLQAEDVAVYYCKQGHSRRTFGGGTKV | | |
| | | EIK | | |
| Ab113 | 284 | QVQLVQSGAEVKKPGASVKVSCKASGFNIKDYYMH | SAD10318_P01_C01 | HC amino acid sequence |
| | | WVRQAPGQRLEWMGWIDLENANTIYDAKFQGRVTI | (ADI-48575) | |
| | | TRDTSASTAYMELSSLRSEDTAVYYCARDAYHRYHY | | |
| | | DVWGQGTLVTVSS | | |
| Ab113 | 285 | DIVMTQSPDSLAVSLGERATINCKSSQSLLNAHTHKN | SAD10318_P01_C01 | LC amino acid sequence |
| | | YLAWYQQKPGQPPKLLIYWASTRESGVPDRFSGSGS | (ADI-48575) | |
| | | GTDFTLTISSLQAEDVAVYYCKQSHSHRTFGGGTKVE | | |
| | | IK | | |
| Ab114 | 286 | QVQLVQSGAEVEKPGASVKVSCKASGFNIKDYYMH | SAD10318_P02_B04 | HC amino acid sequence |
| | | WVRQAPGQRLEWMGWIDLENANTIYDAKFQGRVTI | (ADI-57273) | |
| | | TRDTSASTAYMELSSLRSEDTAVYYCARDAHGRYHY | | |
| | | DVWGQGTLVTVSS | | |
| Ab114 | 287 | DIVMTQSPDSLAVSLGERATINCKSSQSLLNAHTGHN | SAD10318_P02_B04 | LC amino acid sequence |
| | | YLAWYQQKPGQPPKLLIYWASTRESGVPDRFSGSGS | (ADI-57273) | |
| | | GTDFTLTISSLQAEDVAVYYCKQSYSRRTFGGGTKVE | | |
| | | IK | | |
| Ab115 | 288 | QVQLVQSGAEVKKPGASVKVSCKASGFNIKDYYMH | SAD10320_P04_G11 | HC amino acid sequence |
| | | WVRQAPGQRLEWMGWIDLENANTIYDAKFQGRVTI | (ADI-57390) | |
| | | TRDTSASTAYMELSSLRSEDTAVYYCARDAYRHEFY | | |
| | | DVWGQGTLVTVSS | | |
| Ab115 | 289 | DIVMTQSPDSLAVSLGERATINCKSSQSLLNARTGKN | SAD10320_P04_G11 | LC amino acid sequence |
| | | YLAWYQQKPGQPPKLLIYWASTRESGVPDRFSGSGS | (ADI-57390) | |
| | | GTDFTLTISSLQAEDVAVYYCKQSYYHRTFGGGTKV | | |
| | | EIK | | |
| Ab116 | 290 | QVQLVQSGAEVKKPGASVKVSCKASGFNIKDYYMH | SAD10320_P04_H11 | HC amino acid sequence |
| | | WVRQAPGQRLEWMGWIDLENANTIYDAKFQGRVTI | (ADI-48662) | |
| | | TRDTSASTAYMELSSLRSEDTAVYYCARDAYGHVFY | | |
| | | DVWGQGTLVTVSS | | |
| Ab116 | 291 | DIVMTQSPDSLAVSLGERATINCKSSQSLLNARTGKN | SAD10320_P04_H11 | LC amino acid sequence |
| | | YLAWYQQKPGQPPKLLIYWASTRESGVPDRFSGSGS | (ADI-48662) | |
| | | GTDFTLTISSLQAEDVAVYYCKQSYQHRTFGGGTKV | | |
| | | EIK | | |
| Ab117 | 292 | QVQLVQSGAEVKKPGASVKVSCKASGFNIKDYYMH | SAD10320_P04_A12 | HC amino acid sequence |
| | | WVRQAPGQRLEWMGWIDLENANTIYDAKFQGRVTI | (ADI-57391) | |
| | | TRDTSASTAYMELSSLRSEDTAVYYCARDAYGHVFY | | |
| | | DVWGQGTLVTVSS | | |
| Ab117 | 293 | DIVMTQSPDSLAVSLGERATINCKSSQSLLNARTGKN | SAD10320_P04_A12 | LC amino acid sequence |
| | | YLAWYQQKPGQPPKLLIYWASTRESGVPDRFSGSGS | (ADI-57391) | |
| | | GTDFTLTISSLQAEDVAVYYCKQSYHHRTFGGGTKV | | |
| | | EIK | | |
| Ab118 | 294 | QVQLVQSGAEVKKPGASVKVSCKASGFNIKDYYMH | SAD10320_P04_D12 | HC amino acid sequence |
| | | WVRQAPGQRLEWMGWIDLENANTIYDAKFQGRVTI | (ADI-57392) | |
| | | TRDTSASTAYMELSSLRSEDTAVYYCARDAYGHDFY | | |
| | | DVWGQGTLVTVSS | | |
| Ab118 | 295 | DIVMTQSPDSLAVSLGERATINCKSSQSLLNARTGKN | SAD10320_P04_D12 | LC amino acid sequence |
| | | YLAWYQQKPGQPPKLLIYWASTRESGVPDRFSGSGS | (ADI-57392) | |
| | | GTDFTLTISSLQAEDVAVYYCKQSYSHRTFGGGTKVE | | |
| | | IK | | |
| Ab119 | 296 | QVQLVQSGAEVKKPGASVKVSCKASGFNIKDYYMH | SAD10320_P04_E12 | HC amino acid sequence |
| | | WVRQAPGQRLEWMGWIDLENANTIYDAKFQGRVTI | (ADI-57393) | |
| | | TRDTSASTAYMELSSLRSEDTAVYYCARDAYGHTFY | | |
| | | DVWGQGTLVTVSS | | |
| Ab119 | 297 | DIVMTQSPDSLAVSLGERATINCKSSQSLLNARTGKN | SAD10320_P04_E12 | LC amino acid sequence |
| | | YLAWYQQKPGQPPKLLIYWASTRESGVPDRFSGSGS | (ADI-57393) | |
| | | GTDFTLTISSLQAEDVAVYYCKQSYHVRTFGGGTKV | | |
| | | EIK | | |
| Ab120 | 298 | QVQLVQSGAEVKKPGASVKVSCKASGFNIKDYYMH | SAD10320_P04_F12 | HC amino acid sequence |
| | | WVRQAPGQRLERMGWIDLENANTIYDAKFQGRVTIT | (ADI-57394) | |
| | | RDTSASTAYMELSSLRSEDTAVYYCARDAYGHEFYD | | |
| | | VWGQGTLVTVSS | | |
| Ab120 | 299 | DIVMTQSPDSLAVSLGERATINCKSSQSLLNARTGKN | SAD10320_P04_F12 | LC amino acid sequence |
| | | YLAWYQQKPGQPPKLLIYWASTRESGVPDRFSGSGS | (ADI-57394) | |
| | | GTDFTLTISSLQAEDVAVYYCKQSYSRRTFGGGTKVE | | |
| | | IK | | |
| Ab121 | 300 | QVQLVQSGAEVKKPGASVKVSCKASGYTFTAYTMH | SAD10319_P05_A01 | HC amino acid sequence |
| | | WVRQAPGHRLEWMGWINPDTGATDYSQKFQGRVTI | (ADI-57395) | |
| | | TRDTSASTAYMELSSLRSEDTAVYYCARDAYHHYFY | | |
| | | DVWGQGTLVTVSS | | |
| Ab121 | 301 | DIVMTQSPDSLAVSLGERATINCKSSQSLLNARTGKN | SAD10319_P05_A01 | LC amino acid sequence |
| | | YLAWYQQKPGQPPKLLIYWASTRESGVPDRFSGSGS | (ADI-57395) | |
| | | GTDFTLTISSLQAEDVAVYYCKQSYSRRTFGGGTKVE | | |
| | | IK | | |
| Ab122 | 302 | QVQLVQSGAEVKKPGASVKVSCKASGYTFASYDMH | SAD10319_P05_A02 | HC amino acid sequence |
| | | WVRQAPGQRLEWMGWIDAGTGATVYSQKFQGRVTI | (ADI-57396) | |
| | | TRDTSASTAYMELSSLRSEDTAVYYCARDAYGHYFY | | |
| | | DHWGQGTLVTVSS | | |
| Ab122 | 303 | DIVMTQSPDSLAVSLGERATINCKSSQSLLNARTGKN | SAD10319_P05_A02 | LC amino acid sequence |
| | | YLAWYQQKPGQPPKLLIYWASTRESGVPDRFSGSGS | (ADI-57396) | |
| | | GTDFTLTISSLQAEDVAVYYCKQSYSRRTFGGGTKVE | | |
| | | IK | | |
| Ab123 | 304 | QVQLVQSGAEVKKPGASVKVSCKASGYTFNDYAMH | SAD10319_P05_A03 | HC amino acid sequence |
| | | WVRQAPGQRLEWMGWIDAGTGNTYYSQKFQGRVTI | (ADI-57397) | |
| | | TRDTSASTAYMELSSLRSEDTAVYYCARDAHGHYFY | | |
| | | DVWGQGTLVTVSS | | |
| Ab123 | 305 | DIVMTQSPDSLAVSLGERATINCKSSQSLLNARTGKN | SAD10319_P05_A03 | LC amino acid sequence |
| | | YLAWYQQKPGQPPKLLIYWASTRESGVPDRFSGSGS | (ADI-57397) | |
| | | GTDFTLTISSLQAEDVAVYYCKQSYSRRTFGGGTKVE | | |
| | | IK | | |
| Ab124 | 306 | QVQLVQSGAEVKKPGASVKVSCKASGYTFTAYTMH | SAD10319_P05_A05 | HC amino acid sequence |
| | | WVRQAPGQRLEWMGWINPDTGATDYSQKFQGRVTI | (ADI-57398) | |
| | | TRDTSASTAYMELSSLRSEDTAVYYCARDAYHHYFY | | |
| | | DVWGQGTLVTVSS | | |
| Ab124 | 307 | DIVMTQSPDSLAVSLGERATINCKSSQSLLNARTGKN | SAD10319_P05_A05 | LC amino acid sequence |
| | | YLAWYQQKPGQPPKLLIYWASTRESGVPDRFSGSGS | (ADI-57398) | |
| | | GTDFTLTISSLQAEDVAVYYCKQSYSRRTFGGGTKVE | | |
| | | IK | | |
| Ab125 | 308 | QVQLVQSGAEVKEPGASVKVSCKASGFNIKDYYMH | SAD10318_P02_C04 | HC amino acid sequence |
| | | WVRQAPGQRLEWMGWIDLENANTIYDAKFQGRVTI | (ADI-48592) | |
| | | TRDTSASTAYMELSSLRSEDTAVYYCARDAHHRYFY | | |
| | | DVWGQGTLVTVSS | | |
| Ab125 | 309 | DIVMTQSPDSLAVSLGERATINCKSSQSLLNAHTGHN | SAD10318_P02_C04 | LC amino acid sequence |
| | | YLAWYQQKPGQPPKLLIYWASTRESGVPDRFSGSGS | (ADI-48592) | |
| | | GTDFTLTISSLQAEDVAVYYCKQSYSRRTFGGGTKVE | | |
| | | IK | | |
| Ab126 | 310 | QVQLVQSGAEVKKPGASVKVSCKASGYTFTSYNMH | SAD10319_P05_B02 | HC amino acid sequence |
| | | WVRQAPGPSLEWMGSIDAGTGATDYSQKFQGRVTIT | (ADI-57399) | |
| | | RDTSASTAYMELSSLRSEDTAVYYCARDAYHHYFYD | | |
| | | VWGQGTLVTVSS | | |
| Ab126 | 311 | DIVMTQSPDSLAVSLGERATINCKSSQSLLNARTGKN | SAD10319_P05_B02 | LC amino acid sequence |
| | | YLAWYQQKPGQPPKLLIYWASTRESGVPDRFSGSGS | (ADI-57399) | |
| | | GTDFTLTISSLQAEDVAVYYCKQSYSRRTFGGGTKVE | | |
| | | IK | | |
| Ab127 | 312 | QVQLVQSGAEVKKPGASVKVSCKASGYTFESYVMH | SAD10319_P05_B03 | HC amino acid sequence |
| | | WVRQAPGQRLEWMGWIHSGTGNTDYSQKFQGRVTI | (ADI-57400) | |
| | | TRDTSASTAYMELSSLRSEDTAVYYCARDAYGHYFY | | |
| | | DHWGQGTLVTVSS | | |
| Ab127 | 313 | DIVMTQSPDSLAVSLGERATINCKSSQSLLNARTGKN | SAD10319_P05_B03 | LC amino acid sequence |
| | | YLAWYQQKPGQPPKLLIYWASTRESGVPDRFSGSGS | (ADI-57400) | |
| | | GTDFTLTISSLQAEDVAVYYCKQSYSRRTFGGGTKVE | | |
| | | IK | | |
| Ab128 | 314 | QVQLVQSGAEVKKPGASVKVSCKASGYTFTSAVMH | SAD10319_P05_B04 | HC amino acid sequence |
| | | WVRQAPGQRLEWMGDINAGTGATDYSQKFQGRVTI | (ADI-57401) | |
| | | TRDTSASTAYMELSSLRSEDTAVYYCARDAYGRYFY | | |
| | | DVWGQGTLVTVSS | | |
| Ab128 | 315 | DIVMTQSPDSLAVSLGERATINCKSSQSLLNARTGKN | SAD10319_P05_B04 | LC amino acid sequence |
| | | YLAWYQQKPGQPPKLLIYWASTRESGVPDRFSGSGS | (ADI-57401) | |
| | | GTDFTLTISSLQAEDVAVYYCKQSYSRRTFGGGTKVE | | |
| | | IK | | |
| Ab129 | 316 | QVQLVQSGAEVKKPGASVKVSCKASGYTFVDYAMH | SAD10319_P05_B05 | HC amino acid sequence |
| | | WVRQAPGQRLEWMGWIDAGTGNTDYSQKFQGRVTI | (ADI-57402) | |
| | | TRDTSASTAYMELSSLRSEDTAVYYCARDAHGHYFY | | |
| | | DVWGQGTLVTVSS | | |
| Ab129 | 317 | DIVMTQSPDSLAVSLGERATINCKSSQSLLNARTGKN | SAD10319_P05_B05 | LC amino acid sequence |
| | | YLAWYQQKPGQPPKLLIYWASTRESGVPDRFSGSGS | (ADI-57402) | |
| | | GTDFTLTISSLQAEDVAVYYCKQSYSRRTFGGGTKVE | | |
| | | IK | | |
| Ab130 | 318 | QVQLVQSGAEVKKPGASVKVSCKASGYTFTDDAMH | SAD10319_P05_C01 | HC amino acid sequence |
| | | WVRQAPGQRLEWMGWIDAGTGNTYYSQKFQGRVTI | (ADI-57403) | |
| | | TRDTSASTAYMELSSLRSEDTAVYYCAHDAYGHYFY | | |
| | | DVWGQGTLVTVSS | | |
| Ab130 | 319 | DIVMTQSPDSLAVSLGERATINCKSSQSLLNARTGKN | SAD10319_P05_C01 | LC amino acid sequence |
| | | YLAWYQQKPGQPPKLLIYWASTRESGVPDRFSGSGS | (ADI-57403) | |
| | | GTDFTLTISSLQAEDVAVYYCKQSYSRRTFGGGTKVE | | |
| | | IK | | |
| Ab131 | 320 | QVQLVQSGAEVKKPGASVKVSCKASGYTFTSYNMH | SAD10319_P05_C03 | HC amino acid sequence |
| | | WVRQAPGQRLEWMGSIDAGTGATDYSQKFQGRVTI | (ADI-57404) | |
| | | TRDTSASTAYMELSSLRSEDTAVYYCARDAYHHYFY | | |
| | | DVWGQGTLVTVSS | | |
| Ab131 | 321 | DIVMTQSPDSLAVSLGERATINCKSSQSLLNARTGKN | SAD10319_P05_C03 | LC amino acid sequence |
| | | YLAWYQQKPGQPPKLLIYWASTRESGVPDRFSGSGS | (ADI-57404) | |
| | | GTDFTLTISSLQAEDVAVYYCKQSYSRRTFGGGTKVE | | |
| | | IK | | |
| Ab132 | 322 | QVQLVQSGAEVKKPGASVKVSCKASGYTFTSYNMH | SAD10319_P05_C05 | HC amino acid sequence |
| | | WVRQAPGPRLEWMGWIDAGTGATHYSQKFQGRVTI | (ADI-57405) | |
| | | TRDTSASTAYMELSSLRSEDTAVYYCARDAYHHYFY | | |
| | | DVWGQGTLVTVSS | | |
| Ab132 | 323 | DIVMTQSPDSLAVSLGERATINCKSSQSLLNARTGKN | SAD10319_P05_C05 | LC amino acid sequence |
| | | YLAWYQQKPGQPPKLLIYWASTRESGVPDRFSGSGS | (ADI-57405) | |
| | | GTDFTLTISSLQAEDVAVYYCKQSYSRRTFGGGTKVE | | |
| | | IK | | |
| Ab133 | 324 | QVQLVQSGAEVKKPGASVKVSCKASGYTFTEYDMH | SAD10319_P05_D01 | HC amino acid sequence |
| | | WVRQAPGQTLEWMGWIDAGTGATVYSQKFQGRVTI | (ADI-57406) | |
| | | TRDTSASTAYMELSSLRSEDTAVYYCARDAYGRYFY | | |
| | | DHWGQGTLVTVSS | | |
| Ab133 | 325 | DIVMTQSPDSLAVSLGERATINCKSSQSLLNARTGKN | SAD10319_P05_D01 | LC amino acid sequence |
| | | YLAWYQQKPGQPPKLLIYWASTRESGVPDRFSGSGS | (ADI-57406) | |
| | | GTDFTLTISSLQAEDVAVYYCKQSYSRRTFGGGTKVE | | |
| | | IK | | |
| Ab134 | 326 | QVQLVQSGAEVKKPGASVKVSCKASGYTFDSYTMH | SAD10319_P05_D02 | HC amino acid sequence |
| | | WVRQAPGPRLEWMGSINAGTGNTDYSQKFQGRVTIT | (ADI-57407) | |
| | | RDTSASTAYMELSSLRSEDTAVYYCARDHYGHYFYD | | |
| | | VWGQGTLVTVSS | | |
| Ab134 | 327 | DIVMTQSPDSLAVSLGERATINCKSSQSLLNARTGKN | SAD10319_P05_D02 | LC amino acid sequence |
| | | YLAWYQQKPGQPPKLLIYWASTRESGVPDRFSGSGS | (ADI-57407) | |
| | | GTDFTLTISSLQAEDVAVYYCKQSYSRRTFGGGTKVE | | |
| | | IK | | |
| Ab135 | 328 | QVQLVQSGAEVKKPGASVKVSCKASGYTFTDDAMH | SAD10319_P05_D03 | HC amino acid sequence |
| | | WVRQAPGHRLEWMGWIDAGTGNTYYSQKFQGRVTI | (ADI-57408) | |
| | | TRDTSASTAYMELSSLRSEDTAVYYCAHDAYGHYFY | | |
| | | DVWGQGTLVTVSS | | |
| Ab135 | 329 | DIVMTQSPDSLAVSLGERATINCKSSQSLLNARTGKN | SAD10319_P05_D03 | LC amino acid sequence |
| | | YLAWYQQKPGQPPKLLIYWASTRESGVPDRFSGSGS | (ADI-57408) | |
| | | GTDFTLTISSLQAEDVAVYYCKQSYSRRTFGGGTKVE | | |
| | | IK | | |
| Ab136 | 330 | QVQLVQSGAEVKKPGASVKVSCKASGFNIKDYYMH | SAD10318_P02_D04 | HC amino acid sequence |
| | | WVRQAPGQRLEWMGWIDLENANTIYDAKFQGRVTI | (ADI-57274) | |
| | | TRDTSASTAYMELSSLRSEDTAVYYCARDAHGRYFY | | |
| | | DHWGQGTLVTVSS | | |
| Ab136 | 331 | DIVMTQSPDSLAVSLGERATINCKSSQSLLNAHTGKN | SAD10318_P02_D04 | LC amino acid sequence |
| | | HLAWYQQKPGQPPKLLIYWASTRESGVPDRFSGSGS | (ADI-57274) | |
| | | GTDFTLTISSLQAEDVAVYYCKQSYSRRTFGGGTKVE | | |
| | | IK | | |
| Ab137 | 332 | QVQLVQSGAEVKKPGASVKVSCKASGYTFTSDYMH | SAD10319_P05_D04 | HC amino acid sequence |
| | | WVRQAPGQRLEWMGWINAGTGATDYSQKFQGRVTI | (ADI-57409) | |
| | | TRDTSASTAYMELSSLRSEDTAVYYCAHDAYGRYHY | | |
| | | DVWGQGTLVTVSS | | |
| Ab137 | 333 | DIVMTQSPDSLAVSLGERATINCKSSQSLLNARTGKN | SAD10319_P05_D04 | LC amino acid sequence |
| | | YLAWYQQKPGQPPKLLIYWASTRESGVPDRFSGSGS | (ADI-57409) | |
| | | GTDFTLTISSLQAEDVAVYYCKQSYSRRTFGGGTKVE | | |
| | | IK | | |
| Ab138 | 334 | QVQLVQSGAEVKKPGASVKVSCKASGYTFADYAMH | SAD10319_P05_D05 | HC amino acid sequence |
| | | WVRQAPGHRLEWMGDIIAGTGATKYSQKFQGRVTIT | (ADI-57410) | |
| | | RDTSASTAYMELSSLRSEDTAVYYCARDHYGHYFYD | | |
| | | VWGQGTLVTVSS | | |
| Ab138 | 335 | DIVMTQSPDSLAVSLGERATINCKSSQSLLNARTGKN | SAD10319_P05_D05 | LC amino acid sequence |
| | | YLAWYQQKPGQPPKLLIYWASTRESGVPDRFSGSGS | (ADI-57410) | |
| | | GTDFTLTISSLQAEDVAVYYCKQSYSRRTFGGGTKVE | | |
| | | IK | | |
| Ab139 | 336 | QVQLVQSGAEVKKPGASVKVSCKASGYTFTGYEMH | SAD10319_P05_E04 | HC amino acid sequence |
| | | WVRQAPGQRLEWMGWINPSTGNTVYSQKFQGRVTI | (ADI-57411) | |
| | | TRDTSASTAYMELSSLRSEDTAVYYCARDHYGHYFY | | |
| | | DVWGQGTLVTVSS | | |
| Ab139 | 337 | DIVMTQSPDSLAVSLGERATINCKSSQSLLNARTGKN | SAD10319_P05_E04 | LC amino acid sequence |
| | | YLAWYQQKPGQPPKLLIYWASTRESGVPDRFSGSGS | (ADI-57411) | |
| | | GTDFTLTISSLQAEDVAVYYCKQSYSRRTFGGGTKVE | | |
| | | IK | | |
| Ab140 | 338 | QVQLVQSGAEVKKPGASVKVSCKASGYTFTDDAMH | SAD10319_P05_F01 | HC amino acid sequence |
| | | WVRQAPGQSLEWMGWIDAGTGNTYYSQKFQGRVTI | (ADI-57412) | |
| | | TRDTSASTAYMELSSLRSEDTAVYYCAHDAYGHYFY | | |
| | | DVWGQGTLVTVSS | | |
| Ab140 | 339 | DIVMTQSPDSLAVSLGERATINCKSSQSLLNARTGKN | SAD10319_P05_F01 | LC amino acid sequence |
| | | YLAWYQQKPGQPPKLLIYWASTRESGVPDRFSGSGS | 9ADI-57412) | |
| | | GTDFTLTISSLQAEDVAVYYCKQSYSRRTFGGGTKVE | | |
| | | IK | | |
| Ab141 | 340 | QVQLVQSGAEVKKPGASVKVSCKASGYTFNNYAMH | SAD10319_P05_G01 | HC amino acid sequence |
| | | WVRQAPGQSLEWMGWIDAGTGNTDYSQKFQGRVTI | (ADI-57413) | |
| | | TRDTSASTAYMELSSLRSEDTAVYYCARDAHHRYFY | | |
| | | DVWGQGTLVTVSS | | |
| Ab141 | 341 | DIVMTQSPDSLAVSLGERATINCKSSQSLLNARTGKN | SAD10319_P05_G01 | LC amino acid sequence |
| | | YLAWYQQKPGQPPKLLIYWASTRESGVPDRFSGSGS | (ADI-57413) | |
| | | GTDFTLTISSLQAEDVAVYYCKQSYSRRTFGGGTKVE | | |
| | | I | | |
| Ab142 | 342 | QVQLVQSGAEVKKPGASVKVSCKASGYTFTTYAMH | SAD10319_P05_G02 | HC amino acid sequence |
| | | WVRQAPGQRLEWMGWIDPVTGNTKYSQKFQGRVTI | (ADI-57414) | |
| | | TRDTSASTAYMELSSLRSEDTAVYYCARDHYGRYFY | | |
| | | DHWGQGTLVTVSS | | |
| Ab142 | 343 | DIVMTQSPDSLAVSLGERATINCKSSQSLLNARTGKN | SAD10319_P05_G02 | LC amino acid sequence |
| | | YLAWYQQKPGQPPKLLIYWASTRESGVPDRFSGSGS | (ADI-57414) | |
| | | GTDFTLTISSLQAEDVAVYYCKQSYSRRTFGGGTKVE | | |
| | | IK | | |
| Ab143 | 344 | QVQLVQSGAEVKKPGASVKVSCKASGYTFHSYNMH | SAD10319_P05_G03 | HC amino acid sequence |
| | | WVRQAPGHRLEWMGWINPDTGATDYSQKFQGRVTI | (ADI-57415) | |
| | | TRDTSASTAYMELSSLRSEDTAVYYCARDAYGRYFY | | |
| | | DVWGQGTLVTVSS | | |
| Ab143 | 345 | DIVMTQSPDSLAVSLGERATINCKSSQSLLNARTGKN | SAD10319_P05_G03 | LC amino acid sequence |
| | | YLAWYQQKPGQPPKLLIYWASTRESGVPDRFSGSGS | (ADI-57415) | |
| | | GTDFTLTISSLQAEDVAVYYCKQSYSRRTFGGGTKVE | | |
| | | IK | | |
| Ab144 | 346 | QVQLVQSGAEVKKPGASVKVSCKASGYTFTSYGMH | SAD10319_P05_G04 | HC amino acid sequence |
| | | WVRQAPGQRLEWMGWIDPVTGATDYSQKFQGRVTI | (ADI-57416) | |
| | | TRDTSASTAYMELSSLRSEDTAVYYCARDAHGRYFY | | |
| | | DHWGQGTLVTVSS | | |
| Ab144 | 347 | DIVMTQSPDSLAVSLGERATINCKSSQSLLNARTGKN | SAD10319_P05_G04 | LC amino acid sequence |
| | | YLAWYQQKPGQPPKLLIYWASTRESGVPDRFSGSGS | (ADI-57416) | |
| | | GTDFTLTISSLQAEDVAVYYCKQSYSRRTFGGGTKVE | | |
| | | IK | | |
| Ab145 | 348 | QVQLVQSGAEVKKPGASVKVSCKASGYTFTEYDMH | SAD10319_P05_H06 | HC amino acid sequence |
| | | WVRQAPGPTLEWMGWIDAGTGATVYSQKFQGRVTI | (ADI-57417) | |
| | | TRDTSASTAYMELSSLRSEDTAVYYCARDAYGRYFY | | |
| | | DHWGQGTLVTVSS | | |
| Ab145 | 349 | DIVMTQSPDSLAVSLGERATINCKSSQSLLNARTGKN | SAD10319_P05_H06 | LC amino acid sequence |
| | | YLAWYQQKPGQPPKLLIYWASTRESGVPDRFSGSGS | (ADI-57417) | |
| | | GTDFTLTISSLQAEDVAVYYCKQSYSRRTFGGGTKVE | | |
| | | IK | | |
| Ab146 | 350 | QVQLVQSGAEVKKPGASVKVSCKASGYTFHSYEMH | SAD10319_P06_A07 | HC amino acid sequence |
| | | WVRQAPGQRLEWMGWITAGTGSTKYSQKFQGRVTI | (ADI-57418) | |
| | | TRDTSASTAYMELSSLRSEDTAVYYCARDAYGHYFY | | |
| | | DHWGQGTLVTVSS | | |
| Ab146 | 351 | DIVMTQSPDSLAVSLGERATINCKSSQSLLNARTGKN | SAD10319_P06_A07 | LC amino acid sequence |
| | | YLAWYQQKPGQPPKLLIYWASTRESGVPDRFSGSGS | (ADI-57418) | |
| | | GTDFTLTISSLQAEDVAVYYCKQSYSRRTFGGGTKVE | | |
| | | IK | | |
| Ab147 | 352 | QVQLVQSGAEVKKPGASVKVSCKASGFNIKDYYMH | SAD10318_P02_E04 | HC amino acid sequence |
| | | WVRQAPGQRLEWMGWIDLENANTIYDAKFQGRVTI | (ADI-57275) | |
| | | TRDTSASTAYMELSSLRSEDTAVYYCARDHHGRYFY | | |
| | | DVWGQGTLVTVSS | | |
| Ab147 | 353 | DIVMTQSPDSLAVSLGERATINCKSSQSLLNAHTGKN | SAD10318_P02_E04 | LC amino acid sequence |
| | | YLAWYQQKPGQPPKLLIYWASTRESGVPDRFSGSGS | (ADI-57275) | |
| | | GTDFTLTISSLQAEDVAVYYCKQSYSRRTFGGGTKVE | | |
| | | IK | | |
| Ab148 | 354 | QVQLVQSGAEVKKPGASVKVSCKASGYTFTGYEMH | SAD10319_P06_A10 | HC amino acid sequence |
| | | WVRQAPGQRLEWMGWINPVTGATAYSQKFQGRVTI | (ADI-57419) | |
| | | TRDTSASTAYMELSSLRSEDTAVYYCARDAYGHYFY | | |
| | | DHWGQGTLVTVSS | | |
| Ab148 | 355 | DIVMTQSPDSLAVSLGERATINCKSSQSLLNARTGKN | SAD10319_P06_A10 | LC amino acid sequence |
| | | YLAWYQQKPGQPPKLLIYWASTRESGVPDRFSGSGS | (ADI-57419) | |
| | | GTDFTLTISSLQAEDVAVYYCKQSYSRRTFGGGTKVE | | |
| | | IK | | |
| Ab149 | 356 | QVQLVQSGAEVKKPGASVKVSCKASGYTFDSIAMH | SAD10319_P06_A11 | HC amino acid sequence |
| | | WVRQAPGQRLEWMGWINPATGNTDYSQKFQGRVTI | (ADI-57420) | |
| | | TRDTSASTAYMELSSLRSEDTAVYYCARDAYGHYFY | | |
| | | DHWGQGTLVTVSS | | |
| Ab149 | 357 | DIVMTQSPDSLAVSLGERATINCKSSQSLLNARTGKN | SAD10319_P06_A11 | LC amino acid sequence |
| | | YLAWYQQKPGQPPKLLIYWASTRESGVPDRFSGSGS | (ADI-57420) | |
| | | GTDFTLTISSLQAEDVAVYYCKQSYSRRTFGGGTKVE | | |
| | | IK | | |
| Ab150 | 358 | QVQLVQSGAEVKKPGASVKVSCKASGYTFTEYVMH | SAD10319_P06_B10 | HC amino acid sequence |
| | | WVRQAPGQRLEWMGDIDAGTGATKYSQKFQGRVTI | (ADI-57421) | |
| | | TRDTSASTAYMELSSLRSEDTAVYYCARDAYHHYFY | | |
| | | DVWGQGTLVTVSS | | |
| Ab150 | 359 | DIVMTQSPDSLAVSLGERATINCKSSQSLLNARTGKN | SAD10319_P06_B10 | LC amino acid sequence |
| | | YLAWYQQKPGQPPKLLIYWASTRESGVPDRFSGSGS | (ADI-57421) | |
| | | GTDFTLTISSLQAEDVAVYYCKQSYSRRTFGGGTKVE | | |
| | | IK | | |
| Ab151 | 360 | QVQLVQSGAEVKKPGASVKVSCKASGYTFHSYDMH | SAD10319_P06_B11 | HC amino acid sequence |
| | | WVRQAPGQRLEWMGWIDAGTGATTYSQKFQGRVTI | (ADI-57422) | |
| | | TRDTSASTAYMELSSLRSEDTAVYYCARDAYGHYFY | | |
| | | DVWGQGTLVTVSS | | |
| Ab151 | 361 | DIVMTQSPDSLAVSLGERATINCKSSQSLLNARTGKN | SAD10319_P06_B11 | LC amino acid sequence |
| | | YLAWYQQKPGQPPKLLIYWASTRESGVPDRFSGSGS | (ADI-57422) | |
| | | GTDFTLTISSLQAEDVAVYYCKQSYSRRTFGGGTKVE | | |
| | | IK | | |
| Ab152 | 362 | QVQLVQSGAEVKKPGASVKVSCKASGYTFTEYVMH | SAD10319_P06_C10 | HC amino acid sequence |
| | | WVRQAPGPRLEWMGDIDAGTGATKYSQKFQGRVTI | (ADI-57423) | |
| | | TRDTSASTAYMELSSLRSEDTAVYYCARDAYHHYFY | | |
| | | DVWGQGTLVTVSS | | |
| Ab152 | 363 | DIVMTQSPDSLAVSLGERATINCKSSQSLLNARTGKN | SAD10319_P06_C10 | LC amino acid sequence |
| | | YLAWYQQKPGQPPKLLIYWASTRESGVPDRFSGSGS | (ADI-57423) | |
| | | GTDFTLTISSLQAEDVAVYYCKQSYSRRTFGGGTKVE | | |
| | | IK | | |
| Ab153 | 364 | QVQLVQSGAEVKKPGASVKVSCKASGYTFDSYEMH | SAD10319_P06_C12 | HC amino acid sequence |
| | | WVRQAPGHRLEWMGWINPLTGATKYSQKFQGRVTI | (ADI-57424) | |
| | | TRDTSASTAYMELSSLRSEDTAVYYCARDHYGHYFY | | |
| | | DVWGQGTLVTVSS | | |
| Ab153 | 365 | DIVMTQSPDSLAVSLGERATINCKSSQSLLNARTGKN | SAD10319_P06_C12 | LC amino acid sequence |
| | | YLAWYQQKPGQPPKLLIYWASTRESGVPDRFSGSGS | (ADI-57424) | |
| | | GTDFTLTISSLQAEDVAVYYCKQSYSRRTFGGGTKVE | | |
| | | IK | | |
| Ab154 | 366 | QVQLVQSGAEVKKPGASVKVSCKASGYTFNRYVMH | SAD10319_P06_D12 | HC amino acid sequence |
| | | WVRQAPGQRLEWMGGIDAGTGATKYSQKFQGRVTI | (ADI-57425) | |
| | | TRDTSASTAYMELSSLRSEDTAVYYCARDAHRHYFY | | |
| | | DVWGQGTLVTVS | | |
| Ab154 | 367 | DIVMTQSPDSLAVSLGERATINCKSSQSLLNARTGKN | SAD10319_P06_D12 | LC amino acid sequence |
| | | YLAWYQQKPGQPPKLLIYWASTRESGVPDRFSGSGS | (ADI-57425) | |
| | | GTDFTLTISSLQAEDVAVYYCKQSYSRRTFGGGTKVE | | |
| | | IK | | |
| Ab155 | 368 | QVQLVQSGAEVKKPGASVKVSCKASGYTFIKYVMH | SAD10319_P06_E08 | HC amino acid sequence |
| | | WVRQAPGQRLEWMGDIDPDTGATEYSQKFQGRVTIT | (ADI-57426) | |
| | | RDTSASTAYMELSSLRSEDTAVYYCARDDYHHYFYD | | |
| | | VWGQGTLVTVSS | | |
| Ab155 | 369 | DIVMTQSPDSLAVSLGERATINCKSSQSLLNARTGKN | SAD10319_P06_E08 | LC amino acid sequence |
| | | YLAWYQQKPGQPPKLLIYWASTRESGVPDRFSGSGS | (ADI-57426) | |
| | | GTDFTLTISSLQAEDVAVYYCKQSYSRRTFGGGTKVE | | |
| | | IK | | |
| Ab156 | 370 | QVQLVQSGAEVKKPGASVKVSCKASGYTFTAYDMH | SAD10319_P06_E09 | HC amino acid sequence |
| | | WVRQAPGQRLEWMGWINPDTGATVYSQKFQGRVTI | (ADI-57427) | |
| | | TRDTSASTAYMELSSLRSEDTAVYYCARDAYGRYFY | | |
| | | DVWGQGTLVTVSS | | |
| Ab156 | 371 | DIVMTQSPDSLAVSLGERATINCKSSQSLLNARTGKN | SAD10319_P06_E09 | LC amino acid sequence |
| | | YLAWYQQKPGQPPKLLIYWASTRESGVPDRFSGSGS | (ADI-57427) | |
| | | GTDFTLTISSLQAEDVAVYYCKQSYSRRTFGGGTKVE | | |
| | | IK | | |
| Ab157 | 372 | QVQLVQSGAEVKKPGASVKVSCKASGYTFTQYDMH | SAD10319_P06_E10 | HC amino acid sequence |
| | | WVRQAPGQTLEWMGDINAGTGVTVYSQKFQGRVTI | (ADI-57428) | |
| | | TRDTSASTAYMELSSLRSEDTAVYYCARDAYGRYFY | | |
| | | DVWGQGTLVTVSS | | |
| Ab157 | 373 | DIVMTQSPDSLAVSLGERATINCKSSQSLLNARTGKN | SAD10319_P06_E10 | LC amino acid sequence |
| | | YLAWYQQKPGQPPKLLIYWASTRESGVPDRFSGSGS | (ADI-57428) | |
| | | GTDFTLTISSLQAEDVAVYYCKQSYSRRTFGGGTKVE | | |
| | | IK | | |
| Ab158 | 374 | QVQLVQSGAEVKKPGASVKVSCKASGFNIKDYYMH | SAD10318_P02_G04 | HC amino acid sequence |
| | | WVRQAPGQRLEWMGWIDLENANTIYDAKFQGRVTI | (ADI-48593) | |
| | | TRDTSASTAYMELSSLRSEDTAVYYCARDAYGHYHY | | |
| | | DVWGQGTLVTVSS | | |
| Ab158 | 375 | DIVMTQSPDSLAVSLGERATINCKSSQSLLHARTGKN | SAD10318_P02_G04 | LC amino acid sequence |
| | | YLAWYQQKPGQPPKLLIYWASTRESGVPDRFSGSGS | (ADI-48593) | |
| | | GTDFTLTISSLQAEDVAVYYCKQSYHHRTFGGGTKV | | |
| | | EIK | | |
| Ab159 | 376 | QVQLVQSGAEVKKPGASVKVSCKASGYTFESYDMH | SAD10319_P06_F07 | HC amino acid sequence |
| | | WVRQAPGQPLEWMGWIDAGTGATTYSQKFQGRVTI | (ADI-57429) | |
| | | TRDTSASTAYMELSSLRSEDTAVYYCARDAYHHYFY | | |
| | | DVWGQGTLVTVSS | | |
| Ab159 | 377 | DIVMTQSPDSLAVSLGERATINCKSSQSLLNARTGKN | SAD10319_P06_F07 | LC amino acid sequence |
| | | YLAWYQQKPGQPPKLLIYWASTRESGVPDRFSGSGS | (ADI-57429) | |
| | | GTDFTLTISSLQAEDVAVYYCKQSYSRRTFGGGTKVE | | |
| | | IK | | |
| Ab160 | 378 | QVQLVQSGAEVKKPGASVKVSCKASGYTFVSYDMH | SAD10319_P06_F10 | HC amino acid sequence |
| | | WVRQAPGPSLEWMGWIDPNTGATVYSQKFQGRVTI | (ADI-57430) | |
| | | TRDTSASTAYMELSSLRSEDTAVYYCARDHYGHYFY | | |
| | | DVWGQGTLVTVSS | | |
| Ab160 | 379 | DIVMTQSPDSLAVSLGERATINCKSSQSLLNARTGKN | SAD10319_P06_F10 | LC amino acid sequence |
| | | YLAWYQQKPGQPPKLLIYWASTRESGVPDRFSGSGS | (ADI-57430) | |
| | | GTDFTLTISSLQAEDVAVYYCKQSYSRRTFGGGTKVE | | |
| | | IK | | |
| Ab161 | 380 | QVQLVQSGAEVKKPGASVKVSCKASGYTFESYDMH | SAD10319_P06_G09 | HC amino acid sequence |
| | | WVRQAPGPRLEWMGWIDAGTGATTYSQKFQGRVTI | (ADI-57431) | |
| | | TRDTSASTAYMELSSLRSEDTAVYYCARDAYHHYFY | | |
| | | DVWGQGTLVTVSS | | |
| Ab161 | 381 | DIVMTQSPDSLAVSLGERATINCKSSQSLLNARTGKN | SAD10319_P06_G09 | LC amino acid sequence |
| | | YLAWYQQKPGQPPKLLIYWASTRESGVPDRFSGSGS | (ADI-57431) | |
| | | GTDFTLTISSLQAEDVAVYYCKQSYSRRTFGGGTKVE | | |
| | | IK | | |
| Ab162 | 382 | QVQLVQSGAEVKKPGASVKVSCKASGYTFTDYDMH | SAD10319_P06_G11 | HC amino acid sequence |
| | | WVRQAPGQRLEWMGWINAGDAATVYSQKFQGRVTI | (ADI-57432) | |
| | | TRDTSASTAYMELSSLRSEDTAVYYCARDAYGHYFY | | |
| | | DHWGQGTLVTVSS | | |
| Ab162 | 383 | DIVMTQSPDSLAVSLGERATINCKSSQSLLNARTGKN | SAD10319_P06_G11 | LC amino acid sequence |
| | | YLAWYQQKPGQPPKLLIYWASTRESGVPDRFSGSGS | (ADI-57432) | |
| | | GTDFTLTISSLQAEDVAVYYCKQSYSRRTFGGGTKVE | | |
| | | IK | | |
| Ab163 | 384 | QVQLVQSGAEVKKPGASVKVSCKASGYTFVSYNMH | SAD10319_P06_H07 | HC amino acid sequence |
| | | WVRQAPGQRLEWMGSINAGDANTKYSQKFQGRVTI | (ADI-57433) | |
| | | TRDTSASTAYMELSSLRSEDTAVYYCARDHYGHYFY | | |
| | | DVWGQGTLVTVSS | | |
| Ab163 | 385 | DIVMTQSPDSLAVSLGERATINCKSSQSLLNARTGKN | SAD10319_P06_H07 | LC amino acid sequence |
| | | YLAWYQQKPGQPPKLLIYWASTRESGVPDRFSGSGS | (ADI-57433) | |
| | | GTDFTLTISSLQAEDVAVYYCKQSYSRRTFGGGTKVE | | |
| | | IK | | |
| Ab164 | 386 | QVQLVQSGAEVKKPGASVKVSCKASGYTFESYDMH | SAD10319_P06_H08 | HC amino acid sequence |
| | | WVRQAPGQTLEWMGWIDAGTGATTYSQKFQGRVTI | (ADI-57434) | |
| | | TRDTSASTAYMELSSLRSEDTAVYYCARDAYHHYFY | | |
| | | DVWGQGTLVTVSS | | |
| Ab164 | 387 | DIVMTQSPDSLAVSLGERATINCKSSQSLLNARTGKN | SAD10319_P06_H08 | LC amino acid sequence |
| | | YLAWYQQKPGQPPKLLIYWASTRESGVPDRFSGSGS | (ADI-57434) | |
| | | GTDFTLTISSLQAEDVAVYYCKQSYSRRTFGGGTKVE | | |
| | | IK | | |
| Ab165 | 388 | QVQLVQSGAEVKKPGASVKVSCKASGYTFESYDMH | SAD10319_P06_H10 | HC amino acid sequence |
| | | WVRQAPGPRLEWMGWIDAGTGNTKYSQKFQGRVTI | (ADI-57435) | |
| | | TADESTSTAYMELSSLRSEDTAVYYCARDHYGHYFY | | |
| | | DVWGQGTLVTVSS | | |
| Ab165 | 389 | DIVMTQSPDSLAVSLGERATINCKSSQSLLNARTGKN | SAD10319_P06_H10 | LC amino acid sequence |
| | | YLAWYQQKPGQPPKLLIYWASTRESGVPDRFSGSGS | (ADI-57435) | |
| | | GTDFTLTISSLQAEDVAVYYCKQSYSRRTFGGGTKVE | | |
| | | IK | | |
| Ab166 | 390 | QVQLVQSGAEVKKPGASVKVSCKASGYTFTDYDMH | SAD10319_P06_H11 | HC amino acid sequence |
| | | WVRQAPGHRLEWMGWINAGDAATVYSQKFQGRVTI | (ADI-57436) | |
| | | TRDTSASTAYMELSSLRSEDTAVYYCARDAYGHYFY | | |
| | | DHWGQGTLVTVSS | | |
| Ab166 | 391 | DIVMTQSPDSLAVSLGERATINCKSSQSLLNARTGKN | SAD10319_P06_H11 | LC amino acid sequence |
| | | YLAWYQQKPGQPPKLLIYWASTRESGVPDRFSGSGS | (ADI-57436) | |
| | | GTDFTLTISSLQAEDVAVYYCKQSYSRRTFGGGTKVE | | |
| | | IK | | |
| Ab167 | 392 | QVQLVQSGAEVKKPGASVKVSCKASGFNIKDYYMH | LAD9953_P01_H01 | HC amino acid sequence |
| | | WVRQAPGQRLEWMGWIDLENANTIYDAKFQGRVTI | (ADI-57437) | |
| | | TRDTSASTAYMELSSLRSEDTAVYYCARDAYGRYFY | | |
| | | DVWGQGTLVTVSS | | |
| Ab167 | 393 | DIVMTQSPDSLAVSLGERATINCKSSQSLLHARTHKN | LAD9953_P01_H01 | LC amino acid sequence |
| | | YLAWYQQKPGQPPKLLIYWASTRESGVPDRFSGSGS | (ADI-57437) | |
| | | GTDFTLTISSLQAEDVAVYYCKQSYSRRTFGGGTKVE | | |
| | | IK | | |
| Ab168 | 394 | QVQLVQSGAEVKKPGASVKVSCKASGFNIKDYYMH | LAD9954_P01_B02 | HC amino acid sequence |
| | | WVRQAPGQRLEWMGWIDLENANTIYDAKFQGRVTI | (ADI-57438) | |
| | | TRDTSASTAYMELSSLRSEDTAVYYCARDAYGRYFY | | |
| | | DVWGQGTLVTVSS | | |
| Ab168 | 395 | DIVMTQSPDSLAVSLGERATINCKSSQSLLHARTGHN | LAD9954_P01_B02 | LC amino acid sequence |
| | | YLAWYQQKPGQPPKLLIYWASTRESGVPDRFSGSGS | (ADI-57438) | |
| | | GTDFTLTISSLQAEDVAVYYCKQSYSRRTFGGGTKVE | | |
| | | IK | | |
| Ab169 | 396 | QVQLVQSGAEVKKPGASVKVSCKASGFNIKDYYMH | SAD10318_P02_H04 | HC amino acid sequence |
| | | WVRQAPGQRLEWMGWIDLENANTIYDAKFQGRVTI | (ADI-48594) | |
| | | TRDTSASTAYMELSSLRSEDTAVYYCARDAYHRYFY | | |
| | | DHWGQGTLVTVSS | | |
| Ab169 | 397 | DIVMTQSPDSLAVSLGERATINCKSSQSLLHARTGHN | SAD10318_P02_H04 | LC amino acid sequence |
| | | YLAWYQQKPGQPPKLLIYWASTRESGVPDRFSGSGS | (ADI-48594) | |
| | | GTDFTLTISSLQAEDVAVYYCKQSYSRRTFGGGTKVE | | |
| | | IK | | |
| Ab170 | 398 | QVQLVQSGAEVKKPGASVKVSCKASGFNIKDYYMH | LAD9955_P01_G02 | HC amino acid sequence |
| | | WVRQAPGQRLEWMGWIDLENANTIYDAKFQGRVTI | (ADI-57439) | |
| | | TRDTSASTAYMELSSLRSEDTAVYYCARDAYGRYFY | | |
| | | DVWGQGTLVTVSS | | |
| Ab170 | 399 | DIVMTQSPDSLAVSLGERATINCKSSQSLLHARTGKN | LAD9955_P01_G02 | LC amino acid sequence |
| | | HLAWYQQKPGQPPKLLIYWASTRESGVPDRFSGSGS | (ADI-57439) | |
| | | GTDFTLTISSLQAEDVAVYYCKQSYSRRTFGGGTKVE | | |
| | | IK | | |
| Ab171 | 400 | QVQLVQSGAEVKKPGASVKVSCKASGFNIKDYYMH | LAD9956_P01_C03 | HC amino acid sequence |
| | | WVRQAPGQRLEWMGWIDLENANTIYDAKFQGRVTI | (ADI-57440) | |
| | | TRDTSASTAYMELSSLRSEDTAVYYCARDAYGRYFY | | |
| | | DVWGQGTLVTVSS | | |
| Ab171 | 401 | DIVMTQSPDSLAVSLGERATINCKSSQSLLNAHTHKN | LAD9956_P01_C03 | LC amino acid sequence |
| | | YLAWYQQKPGQPPKLLIYWASTRESGVPDRFSGSGS | (ADI-57440) | |
| | | GTDFTLTISSLQAEDVAVYYCKQSYSRRTFGGGTKVE | | |
| | | IK | | |
| Ab172 | 402 | QVQLVQSGAEVKKPGASVKVSCKASGFNIKDYYMH | LAD9959_P01_E04 | HC amino acid sequence |
| | | WVRQAPGQRLEWMGWIDLENANTIYDAKFQGRVTI | (ADI-57441) | |
| | | TRDTSASTAYMELSSLRSEDTAVYYCARDAYGRYFY | | |
| | | DVWGQGTLVTVSS | | |
| Ab172 | 403 | DIVMTQSPDSLAVSLGERATINCKSSQSLLNARTHHN | LAD9959_P01_E04 | LC amino acid sequence |
| | | YLAWYQQKPGQPPKLLIYWASTRESGVPDRFSGSGS | (ADI-57441) | |
| | | GTDFTLTISSLQAEDVAVYYCKQSYSRRTFGGGTKVE | | |
| | | IK | | |
| Ab173 | 404 | QVQLVQSGAEVKKPGASVKVSCKASGFNIKDYYMH | LAD9960_P01_D05 | HC amino acid sequence |
| | | WVRQAPGQRLEWMGWIDLENANTIYDAKFQGRVTI | (ADI-57442) | |
| | | TRDTSASTAYMELSSLRSEDTAVYYCARDAYGRYFY | | |
| | | DVWGQGTLVTVSS | | |
| Ab173 | 405 | DIVMTQSPDSLAVSLGERATINCKSSQSLLNARTHKN | LAD9960_P01_D05 | LC amino acid sequence |
| | | HLAWYQQKPGQPPKLLIYWASTRESGVPDRFSGSGS | (ADI-57442) | |
| | | GTDFTLTISSLQAEDVAVYYCKQSYSRRTFGGGTKVE | | |
| | | IK | | |
| Ab174 | 406 | QVQLVQSGAEVKKPGASVKVSCKASGFNIKDYYMH | LAD9963_P01_E06 | HC amino acid sequence |
| | | WVRQAPGQRLEWMGWIDLENANTIYDAKFQGRVTI | (ADI-57443) | |
| | | TRDTSASTAYMELSSLRSEDTAVYYCARDAYGRYFY | | |
| | | DVWGQGTLVTVSS | | |
| Ab174 | 407 | DIVMTQSPDSLAVSLGERATINCKSSQSLLNAHTGKN | LAD9963_P01_E06 | LC amino acid sequence |
| | | YLAWYQQKPGQPPKLLIYWASTRESGVPDRFSGSGS | (ADI-57443) | |
| | | GTDFTLTISSLQAEDVAVYYCKQSYSRRTFGGGTKVE | | |
| | | I | | |
| Ab175 | 408 | QVQLVQSGAEVKKPGASVKVSCKASGFNIKDYYMH | LAD9964_P01_C07 | HC amino acid sequence |
| | | WVRQAPGQRLEWMGWIDLENANTIYDAKFQGRVTI | (ADI-57444) | |
| | | TRDTSASTAYMELSSLRSEDTAVYYCARDAYGRYFY | | |
| | | DVWGQGTLVTVS | | |
| Ab175 | 409 | DIVMTQSPDSLAVSLGERATINCKSSQSLLNARTHKN | LAD9964_P01_C07 | LC amino acid sequence |
| | | YLAWYQQKPGQPPKLLIYWASTRESGVPDRFSGSGS | (ADI-57444) | |
| | | GTDFTLTISSLQAEDVAVYYCKQSYSRRTFGGGTKVE | | |
| | | IK | | |
| Ab176 | 410 | QVQLVQSGAEVKKPGASVKVSCKASGFNIKDYYMH | LAD9966_P01_A08 | HC amino acid sequence |
| | | WVRQAPGQRLEWMGWIDLENANTIYDAKFQGRVTI | (ADI-57445) | |
| | | TRDTSASTAYMELSSLRSEDTAVYYCARDAYGRYFY | | |
| | | DVWGQGTLVTVSS | | |
| Ab176 | 411 | DIVMTQSPDSLAVSLGERATINCKSSQSLLNARTGKN | LAD9966_P01_A08 | LC amino acid sequence |
| | | HLAWYQQKPGQPPKLLIYWASTRESGVPDRFSGSGS | (ADI-57445) | |
| | | GTDFTLTISSLQAEDVAVYYCKQSYSRRTFGGGTKVE | | |
| | | IK | | |
| Ab177 | 412 | QVQLVQSGAEVKKPGASVKVSCKASGFNIKDYYMH | LAD9965_P01_H07 | HC amino acid sequence |
| | | WVRQAPGQRLEWMGWIDLENANTIYDAKFQGRVTI | (ADI-48666) | |
| | | TRDTSASTAYMELSSLRSEDTAVYYCARDAYGRYFY | | |
| | | DVWGQGTLVTVSS | | |
| Ab177 | 413 | DIVMTQSPDSLAVSLGERATINCKSSQSLLNARTGHN | LAD9965_P01_H07 | LC amino acid sequence |
| | | YLAWYQQKPGQPPKLLIYWASTRESGVPDRFSGSGS | (ADI-48666) | |
| | | GTDFTLTISSLQAEDVAVYYCKQSYSRRTFGGGTKVE | | |
| | | IK | | |
| Ab178 | 414 | QVQLVQSGTEVKKPGASVKVSCKASGFNIKDYYMH | SAD10318_P02_A05 | HC amino acid sequence |
| | | WVRQAPGQRLEWMGWIDLENANTIYDAKFQGRVTI | (ADI-48595) | |
| | | TRDTSASTAYMELSSLRSEDTAVYYCARDAYHRYFY | | |
| | | DHWGQGTLVTVSS | | |
| Ab178 | 415 | DIVMTQSPDSLAVSLGERATINCKSSQSLLNSHTGKN | SAD10318_P02_A05 | LC amino acid sequence |
| | | YLAWYQQKPGQPPKLLIYWASTRESGVPDRFSGSGS | (ADI-48595) | |
| | | GTDFTLTISSLQAEDVAVYYCKQSYSRRTFGGGTKVE | | |
| | | IK | | |
| Ab179 | 416 | QVQLVQSGAEVKKPGASVKVSCKASGFNIKDYYMH | SAD10318_P02_B05 | HC amino acid sequence |
| | | WVRQAPGQRLEWMGWIDLENANTIYDAKFQGRVTI | (ADI-48596) | |
| | | TRDTSASTAYMELSSLRSEDTAVYYCARDAYGRHFY | | |
| | | DHWGQGTLVTVSS | | |
| Ab179 | 417 | DIVMTQSPDSLAVSLGERATINCKSSQSLLNAHTGKN | SAD10318_P02_B05 | LC amino acid sequence |
| | | YLAWYQQKPGQPPKLLIYWASTRESGVPDRFSGSGS | (ADI-48596) | |
| | | GTDFTLTISSLQAEDVAVYYCKQSYSRRTFGGGTKVE | | |
| | | IK | | |
| Ab180 | 418 | QVQLVQSGAEVKKPGASVKVSCKASGFNIKDYYMH | SAD10318_P02_C05 | HC amino acid sequence |
| | | WVRQAPGQRLEWMGWIDLENANTIYDAKFQGRVTI | (ADI-48597) | |
| | | TRDTSASTAYMELSSLRSEDTAVYYCARDAHHRYFY | | |
| | | DVWGQGTLVTVSS | | |
| Ab180 | 419 | DIVMTQSPDSLAVSLGERATINCKSSQSLLNARTGHN | SAD10318_P02_C05 | LC amino acid sequence |
| | | HLAWYQQKPGQPPKLLIYWASTRESGVPDRFSGSGS | (ADI-48597) | |
| | | GTDFTLTISSLQAEDVAVYYCKQSHSHRTFGGGTKVE | | |
| | | I | | |
| Ab181 | 420 | QVQLVQSGAEVKKPGASVKVSCKASGFNIKDYYMH | SAD10318_P02_D05 | HC amino acid sequence |
| | | WVRQAPGQRLEWMGWIDLENANTIYDAKFQGRVTI | (ADI-48598) | |
| | | TRDTSASTAYMELSSLRSEDTAVYYCARDHYGHYFY | | |
| | | DVWGQGTLVTVSS | | |
| Ab181 | 421 | DIVMTQSPDSLAVSLGERATINCKSSQSLLNARTGKN | SAD10318_P02_D05 | LC amino acid sequence |
| | | HLAWYQQKPGQPPKLLIYWASTRESGVPDRFSGSGS | (ADI-48598) | |
| | | GTDFTLTISSLQAEDVAVYYCKQSYHHRTFGGGTKV | | |
| | | EIK | | |
| Ab182 | 422 | QVQLVQSGAEVKKPGASVKVSCKASGFNIKDYYMH | SAD10318_P01_D01 | HC amino acid sequence |
| | | WVRQAPGQRLEWMGWIDLENANTIYDAKFQGRVTI | (ADI-48576) | |
| | | TRDTSASTAYMELSSLRSEDTAVYYCARDAYHRYFY | | |
| | | DHWGQGTLVTVSS | | |
| Ab182 | 423 | DIVMTQSPDSLAVSLGERATINCKSSQSLLNAHTGKN | SAD10318_P01_D01 | LC amino acid sequence |
| | | HLAWYQQKPGQPPKLLIYWASTRESGVPDRFSGSGS | (ADI-48576) | |
| | | GTDFTLTISSLQAEDVAVYYCKQSHSHRTFGGGTKVE | | |
| | | IK | | |
| Ab183 | 424 | QVQLVQSGAEVKKPGASVKVSCKASGFNIKDYYMH | SAD10318_P02_F05 | HC amino acid sequence |
| | | WVRQAPGQRLEWMGWIDLENANTIYDAKFQGRVTI | (ADI-57276) | |
| | | TRDTSASTAYMELSSLRSEDTAVYYCARDAYGHYFY | | |
| | | DHWGQGTLVTVSS | | |
| Ab183 | 425 | GIVMTQSPDSLAVSLGERATINCKSSQSLLNAHTGKN | SAD10318_P02_F05 | LC amino acid sequence |
| | | HLAWYQQKPGQPPKLLIYWASTRESGVPDRFSGSGS | (ADI-57276) | |
| | | GTDFTLTISSLQAEDVAVYYCKQSHSRRTFGGGTKVE | | |
| | | IK | | |
| Ab184 | 426 | QVQLVQSGAEVKKPGASVKVSCKASGFNIKDYYMH | SAD10318_P02_G05 | HC amino acid sequence |
| | | WVRQAPGQRLEWMGWIDLENANTIYDAKFQGRVTI | (ADI-57277) | |
| | | TRDTSASTAYMELSSLRSEDTAVYYCARDAYGRYFY | | |
| | | DVWGQGTLVTVSS | | |
| Ab184 | 427 | DIVMTQSPDSLAVSLGERATINCKSSQSLLNARTHKN | SAD10318_P02_G05 | LC amino acid sequence |
| | | HLAWYQQKPGQPPKLLIYWASTRESGVPDRFSGSGS | (ADI-57277) | |
| | | GTDFTLTISSLQAEDVAVYYCKQSHSRRHFGGGTKV | | |
| | | EIK | | |
| Ab185 | 428 | QVQLVQSGAEVKKPGASVKVSCKASGFNIKDYYMH | SAD10318_P02_H05 | HC amino acid sequence |
| | | WVRQAPGQRLEWMGWIDLENANTIYDAKFQGRVTI | (ADI-48599) | |
| | | TRDTSASTAYMELSSLRSEDTAVYYCARDAYGHHFY | | |
| | | DVWGQGTLVTVSS | | |
| Ab185 | 429 | DIVMTQSPDSLAVSLGERATINCKSSQSLLNRHTGKN | SAD10318_P02_H05 | LC amino acid sequence |
| | | YLAWYQQKPGQPPKLLIYWASTRESGVPDRFSGSGS | (ADI-48599) | |
| | | GTDFTLTISSLQAEDVAVYYCKQSYSRRTFGGGTKVE | | |
| | | IK | | |
| Ab186 | 430 | QVQLVQSGAEVKKPGASVKVSCKASGFNIKDYYMH | SAD10318_P02_B06 | HC amino acid sequence |
| | | WVRQAPGQRLEWMGWIDLENANTIYDAKFQGRVTI | (ADI-57278) | |
| | | TRDTSASTAYMELSSLRSEDTAVYYCARDAYHRHFY | | |
| | | DVWGQGTLVTVSS | | |
| Ab186 | 431 | DIVMTQSPDSLAVSLGERATINCKSSQSLLNAHTGKN | SAD10318_P02_B06 | LC amino acid sequence |
| | | YLAWYQQKPGQPPKLLIYWASTRESGVPDRFSGSGS | (ADI-57278) | |
| | | GTDFTLTISSLQAEDVAVYYCKQSYHHRTFGGGTKV | | |
| | | EIK | | |
| Ab187 | 432 | QVQLVQSGAEVKKPGASVKVSCKASGFNIKDYYMH | SAD10318_P02_D06 | HC amino acid sequence |
| | | WVRQAPGQRLEWMGWIDLENANTIYDAKFQGRVTI | (ADI-57279) | |
| | | TRDTSASTAYMELSSLRSEDTAVYYCARDAHGRYFY | | |
| | | DVWGQGTLVTVSS | | |
| Ab187 | 433 | DIVMTQSPDSLAVSLGERATINCKSSQSLLNARTGHN | SAD10318_P02_D06 | LC amino acid sequence |
| | | HLAWYQQKPGQPPKLLIYWASTRESGVPDRFSGSGS | (ADI-57279) | |
| | | GTDFTLTISSLQAEDVAVYYCKQSYSRRTFGGGTKVE | | |
| | | IK | | |
| Ab188 | 434 | QVQLVQSGAEVKKPGASVKVSCKASGFNIKDYYMH | SAD10318_P02_E06 | HC amino acid sequence |
| | | WVRQAPGQRLEWMGWIDLENANTIYDAKFQGRVTI | (ADI-57280) | |
| | | TRDTSASTAYMELSSLRSEDTAVYYCARDAHHRYFY | | |
| | | DVWGQGTLVTVSS | | |
| Ab188 | 435 | DIVMTQSPDSLAVSLGERATINCKSSQSLLNARTHKN | SAD10318_P02_E06 | LC amino acid sequence |
| | | HLAWYQQKPGQPPKLLIYWASTRESGVPDRFSGSGS | (ADI-57280) | |
| | | GTDFTLTISSLQAEDVAVYYCKQSHSHRTFGGGTKVE | | |
| | | IK | | |
| Ab189 | 436 | QVQLVQSGAEVKKPGASVKVSCKASGFNIKDYYMH | SAD10318_P02_G06 | HC amino acid sequence |
| | | WVRQAPGQRLEWMGWIDLENANTIYDAKFQGRVTI | (ADI-48600) | |
| | | TRDTSASTAYMELSSLRSEDTAVYYCAHDAYGHYFY | | |
| | | DVWGQGTLVTVSS | | |
| Ab189 | 437 | DIVMTQSPDSLAVSLGERATINCKSSQSLLHTRTGKN | SAD10318_P02_G06 | LC amino acid sequence |
| | | YLAWYQQKPGQPPKLLIYWASTRESGVPDRFSGSGS | (ADI-48600) | |
| | | GTDFTLTISSLQAEDVAVYYCKQSYSRRTFGGGTKVE | | |
| | | IK | | |
| Ab190 | 438 | QVQLVQSGAEVKKPGASVKVSCKASGFNIKDYYMH | SAD10318_P03_B07 | HC amino acid sequence |
| | | WVRQAPGQRLEWMGWIDLENANTIYDAKFQGRVTI | (ADI-57281) | |
| | | TRDTSASTAYMELSSLRSEDTAVYYCARDHYGRYFY | | |
| | | DHWGQGTLVTVSS | | |
| Ab190 | 439 | DIVMTQSPDSLAVSLGERATINCKSSQSLLNAHTGKN | SAD10318_P03_B07 | LC amino acid sequence |
| | | HLAWYQQKPGQPPKLLIYWASTRESGVPDRFSGSGS | (ADI-57281) | |
| | | GTDFTLTISSLQAEDVAVYYCKQSHHRRTFGGGTKV | | |
| | | EIK | | |
| Ab191 | 440 | QVQLVQSGAEVKKPGASVKVSCKASGFNIKDYYMH | SAD10318_P03_E07 | HC amino acid sequence |
| | | WVRQAPGQRLEWMGWIDLENANTIYDAKFQGRVTI | (ADI-48601) | |
| | | TRDTSASTAYMELSSLRSEDTAVYYCARDHYHRYFY | | |
| | | DVWGQGTLVTVSS | | |
| Ab191 | 441 | DIVMTQSPDSLAVSLGERATINCKSSQSLLNAHTGKN | SAD10318_P03_E07 | LC amino acid sequence |
| | | YLAWYQQKPGQPPKLLIYWASTRESGVPDRFSGSGS | (ADI-48601) | |
| | | GTDFTLTISSLQAEDVAVYYCHQSHSRRTFGGGTKVE | | |
| | | IK | | |
| Ab192 | 442 | QVQLVQSGAEVKKPGASVKVSCKASGFNIKDYYMH | SAD10318_P03_F07 | HC amino acid sequence |
| | | WVRQAPGQRLEWMGWIDLENANTIYDAKFQGRVTI | (ADI-48602) | |
| | | TRDTSASTAYMELSSLRSEDTAVYYCARDAHGRYHY | | |
| | | DVWGQGTLVTVSS | | |
| Ab192 | 443 | DIVMTQSPDCLAVSLGERATINCKSSQSLLNARTGHN | SAD10318_P03_F07 | LC amino acid sequence |
| | | YLAWYQQKPGQPPKLLIYWASTRESGVPDRFSGSGS | (ADI-48602) | |
| | | GTDFTLTISSLQAEDVAVYYCKQSHSHRTFGGGTKVE | | |
| | | IK | | |
| Ab193 | 444 | QVQLVQSGAEVKKPGASVKVSCKASGFNIKDYYMH | SAD10318_P01_E01 | HC amino acid sequence |
| | | WVRQAPGQRLEWMGWIDLENANTIYDAKFQGRVTI | (ADI-48577) | |
| | | TRDTSASTAYMELSSLRSEDTAVYYCARDAHHRYFY | | |
| | | DVWGQGTLVTVSS | | |
| Ab193 | 445 | DIVMTQSPDSLAVSLGERATINCKSSQSLLNAHEGKN | SAD10318_P01_E01 | LC amino acid sequence |
| | | YLAWYQQKPGQPPKLLIYWASTRESGVPDRFSGSGS | (ADI-48577) | |
| | | GTDFTLTISSLQAEDVAVYYCKQSHSHRTFGGGTKVE | | |
| | | IK | | |
| Ab194 | 446 | QVQLVQSGAEVKKPGASVKVSCKASGFNIKDYYMH | SAD10318_P03_G07 | HC amino acid sequence |
| | | WVRQAPGQRLEWMGWIDLENANTIYDAKFQGRVTI | (ADI-57282) | |
| | | TRDTSASTAYMELSSLRSEDTAVYYCARDAYGRYHY | | |
| | | DHWGQGTLVTVS | | |
| Ab194 | 447 | DIVMTQSPDSLAVSLGERATINCKSSQSLLHAHTGKN | SAD10318_P03_G07 | LC amino acid sequence |
| | | YLAWYQQKPGQPPKLLIYWASTRESGVPDRFSGSGS | (ADI-57282) | |
| | | GTDFTLTISSLQAEDVAVYYCKQSYHHRTFGGGTKV | | |
| | | EIK | | |
| Ab195 | 448 | QVQLVQSGAEVKKPGASVKVSCKASGFNIKDYYMH | SAD10318_P03_A08 | HC amino acid sequence |
| | | WVRQAPGQRLEWMGWIDLENANTIYDAKFQGRVTI | (ADI-57283) | |
| | | TRDTSASTAYMELSSLRSEDTAVYYCARDAYGRYHY | | |
| | | DHWGQGTLVTVSS | | |
| Ab195 | 449 | DIVMTQSPDSLAVSLGERATINCKSSQSLLNARTGHN | SAD10318_P03_A08 | LC amino acid sequence |
| | | HLAWYQQKPGQPPKLLIYWASTRESGVPDRFSGSGS | (ADI-57283) | |
| | | GTDFTLTISSLQAEDVAVYYCKQSYSRRTFGGGTKVE | | |
| | | IK | | |
| Ab196 | 450 | QVQLVQSGAEVKKPGASVKVSCKASGFNIKDYYMH | SAD10318_P03_B08 | HC amino acid sequence |
| | | WVRQAPGQRLEWMGWIDLENANTIYDAKFQGRVTI | 9ADI-57284) | |
| | | TRDTSASTAYMELSSLRSEDTAVYYCARDHYHRYFY | | |
| | | DVWGQGTLVTVSS | | |
| Ab196 | 451 | DIVMTQSPDSLAVSLGERATINCKSSQSLLNAHTGHN | SAD10318_P03_B08 | LC amino acid sequence |
| | | YLAWYQQKPGQPPKLLIYWASTRESGVPDRFSGSGS | (ADI-57284) | |
| | | GTDFTLTISSLQAEDVAVYYCHQSYSHRTFGGGTKVE | | |
| | | IK | | |
| Ab197 | 452 | QVQLVQSGAEVKKPGASVKVSCKASGFNIKDYYMH | SAD10318_P03_C08 | HC amino acid sequence |
| | | WVRQAPGQRLEWMGWIDLENANTIYDAKFQGRVTI | (ADI-48603) | |
| | | TRDTSASTAYMELSSLRSEDTAVYYCARDHYGHYFY | | |
| | | DVWGQGTLVTVSS | | |
| Ab197 | 453 | DIVMTQSPDSLAVSLGERATINCKSSQSLLHARTGKN | SAD10318_P03_C08 | LC amino acid sequence |
| | | HLAWYQQKPGQPPKLLIYWASTRESGVPDRFSGSGS | (ADI-48603) | |
| | | GTDFTLTISSLQAEDVAVYYCKQSHSHRTFGGGTKVE | | |
| | | IK | | |
| Ab198 | 454 | QVQLVQSGAEVKKPGASVKVSCKASGFNIKDYYMH | SAD10318_P03_D08 | HC amino acid sequence |
| | | WVRQAPGQRLEWMGWIDLENANTIYDAKFQGRVTI | (ADI-48604) | |
| | | TRDTAASTAYMELSSLRSEDTAVYYCARDHYHRYFY | | |
| | | DHWGQGTLVTVSS | | |
| Ab198 | 455 | DIVMTQSPDSLAVSLGERATINCKSSQSLLNAHTGKN | SAD10318_P03_D08 | LC amino acid sequence |
| | | HLAWYQQKPGQPPKLLIYWASTRESGVPDRFSGSGS | (ADI-48604) | |
| | | GTDFTLTISSLQAEDVAVYYCKQSYSRRTFGGGTKVE | | |
| | | IK | | |
| Ab199 | 456 | QVQLVQSGAEVKKPGASVKVSCKASGFNIKDYYMH | SAD10318_P03_E08 | HC amino acid sequence |
| | | WVRQAPGQRLEWMGWIDLENANTIYDAKFQGRVTI | (ADI-48605) | |
| | | TRDTSASTAYMELSSLRSEDTAVYYCARDHYHRYFY | | |
| | | DVWGQGTLVTVSS | | |
| Ab199 | 457 | DIVMTQSPDSLAVSLGERATINCKSSQSLLNAHTGKN | SAD10318_P03_E08 | LC amino acid sequence |
| | | HLAWYQQKPGQPPKLLIYWASTRESGVPDRFSGSGS | (ADI-48605) | |
| | | GTDFTLTISSLQAEDVAVYYCKQSYHRRHFGGGTKV | | |
| | | EIK | | |
| Ab200 | 458 | QVQLVQSGAEVKKPGASVKVSCKASGFNIKDYYMH | SAD10318_P03_F08 | HC amino acid sequence |
| | | WVRQAPGQRLEWMGWIDLENANTIYDAKFQGRVTI | (ADI-48606) | |
| | | TRDTSASTAYMELSSLRSEDTAVYYCARDHYHRYFY | | |
| | | DVWGQGTLVTVSS | | |
| Ab200 | 459 | DIVMTQSPDSLAVSLGERATINCKSSQSLLNAHTGKN | SAD10318_P03_F08 | LC amino acid sequence |
| | | HLAWYQQKPGQPPKLLIYWASTRESGVPDRFSGSGS | (ADI-48606) | |
| | | GTDFTLTISSLQAEDVAVYYCKQSYSRRHFGGGTKV | | |
| | | EIK | | |
| Ab201 | 460 | QVQLVQSGAEVKKPGASVKVSCKASGFNIKDYYMH | SAD10318_P03_G08 | HC amino acid sequence |
| | | WVRQAPGQRLEWMGWIDLENANTIYDAKFQGRVTI | (ADI-57285) | |
| | | TRDTSASTAYMELSSLRSEDTAVYYCARDHYHRYFY | | |
| | | DVWGQGTLVTVSS | | |
| Ab201 | 461 | DIVMTQSPDSLAVSLGERATINCKSSQSLLHARTGKN | SAD10318_P03_G08 | LC amino acid sequence |
| | | HLAWYQQKPGQPPKLLIYWASTRESGVPDRFSGSGS | (ADI-57285) | |
| | | GTDFTLTISSLQAEDVAVYYCKQSHSHRTFGGGTKVE | | |
| | | IK | | |
| Ab202 | 462 | QVQLVQSGAEVKKPGASVKVSCKASGFNIKDYYMH | SAD10318_P03_H08 | HC amino acid sequence |
| | | WVRQAPGQRLEWMGWIDLENANTIYDAKFQGRVTI | (ADI-48607) | |
| | | TRDTSASTAYMELSSLRSEDTAVYYCARDAYHHYFY | | |
| | | DVWGQGTLVTVSS | | |
| Ab202 | 463 | DIVMTQSPDSLAVSLGERATINCKSSQSLLNARTGHN | SAD10318_P03_H08 | LC amino acid sequence |
| | | HLAWYQQKPGQPPKLLIYWASTRESGVPDRFSGSGS | (ADI-48607) | |
| | | GTDFTLTISSLQAEDVAVYYCHQSHSRRTFGGGTKVE | | |
| | | IK | | |
| Ab203 | 464 | QVQLVQSGAEVKKPGASVKVSCKASGFNIKDYYMH | SAD10318_P03_A09 | HC amino acid sequence |
| | | WVRQAPGQRLEWMGWIDLENANTIYDAKFQGRVTI | (ADI-48608) | |
| | | TRDTSASTAYMELSSLRSEDTAVYYCARDHYGRYFY | | |
| | | DHWGQGTLVTVSS | | |
| Ab203 | 465 | DIVMTQSPDSLAVSLGERATINCKSSQSLLNARTGHN | SAD10318_P03_A09 | LC amino acid sequence |
| | | HLAWYQQKPGQPPKLLIYWASTRESGVPDRFSGSGS | (ADI-48608) | |
| | | GTDFTLTISSLQAEDVAVYYCKQSYHHRTFGGGTKV | | |
| | | EIK | | |
| Ab204 | 466 | QVQLVQSGAEVKKPGASVKVSCKASGFNIKDYYMH | SAD10318_P01_F01 | HC amino acid sequence |
| | | WVRQAPGQRLEWMGWIDLENANTIYDAKFQGRVTI | (ADI-48578) | |
| | | TRDTSASTAYMELSSLRSEDTAVYYCARDAYGHYFY | | |
| | | DHWGQGTLVTVSS | | |
| Ab204 | 467 | DIVMTQSPDSLAVSLGERATINCKSSQSLLNAHTGKN | SAD10318_P01_F01 | LC amino acid sequence |
| | | HLAWYQQKPGQPPKLLIYWASTRESGVPDRFSGSGS | (ADI-48578) | |
| | | GTDFTLTISSLQAEDVAVYYCKQSYHHRTFGGGTKV | | |
| | | EIK | | |
| Ab205 | 468 | QVQLVQSGAEVKKPGASVKVSCKASGFNIKDYYMH | SAD10318_P03_C09 | HC amino acid sequence |
| | | WVRQAPGQRLEWMGWIDLENANTIYDAKFQGRVTI | (ADI-48609) | |
| | | TRDTSASTAYMELSSLRSEDTAVYYCARDAYHRYFY | | |
| | | DHWGQGTLVTVSS | | |
| Ab205 | 469 | DIVMTQSPDSLAVSLGERATINCKSSQSLLNAHTGKN | SAD10318_P03_C09 | LC amino acid sequence |
| | | HLAWYQQKPGQPPKLLIYWASTRESGVPDRFSGSGS | (ADI-48609) | |
| | | GTDFTLTISSLQAEDVAVYYCKQSYHHRTFGGGTKV | | |
| | | EIK | | |
| Ab206 | 470 | QVQLVQSGAEVKKPGASVKVSCKASGFNIKDYHMH | SAD10318_P03_D09 | HC amino acid sequence |
| | | WVRQAPGQRLEWMGWIDLENANTIYDAKFQGRVTI | (ADI-48610) | |
| | | TRDTSASTAYMELSSLRSEDTAVYYCARDAHHRYFY | | |
| | | DVWGQGTLVTVSS | | |
| Ab206 | 471 | DIVMTQSPDSLAVSLGERATINCKSSQSLLNAHTGKN | SAD10318_P03_D09 | LC amino acid sequence |
| | | HLAWYQQKPGQPPKLLIYWASTRESGVPDRFSGSGS | (ADI-48610) | |
| | | GTDFTLTISSLQAEDVAVYYCKQSHSHRTFGGGTKVE | | |
| | | IK | | |
| Ab207 | 472 | QVQLVQSGAEVKKPGASVKVSCKASGFNIKDYYMH | SAD10318_P03_E09 | HC amino acid sequence |
| | | WVRQAPGQRLEWMGWIDLENANTIYDAKFQGRVTI | (ADI-48611) | |
| | | TRDTSASTAYMELSSLRSEDTAVYYCARDAYGHYHY | | |
| | | DVWGQGTLVTVS | | |
| Ab207 | 473 | DIVMTQSPDSLAVSLGERATINCKSSQSLLNARTGHN | SAD10318_P03_E09 | LC amino acid sequence |
| | | HLAWYQQKPGQPPKLLIYWASTRESGVPDRFSGSGS | (ADI-48611) | |
| | | GTDFTLTISSLQAEDVAVYYCKQSYHHRTFGGGTKV | | |
| | | EIK | | |
| Ab208 | 474 | QVQLVQSGAEVKKPGASVKVSCKASGFNIKDYYMH | SAD10318_P03_F09 | HC amino acid sequence |
| | | WVRQAPGQRLEWMGWIDLENANTIYDAKFQGRVTI | (ADI-57286) | |
| | | TRDTSASTAYMELSSLRSEDTAVYYCARDAYGHYHY | | |
| | | DVWGQGTLVTVSS | | |
| Ab208 | 475 | DIVMTQSPDSLAVSLGERATINCKSSQSLLNAHPGKN | SAD10318_P03_F09 | LC amino acid sequence |
| | | YLAWYQQKPGQPPKLLIYWASTRESGVPDRFSGSGS | (ADI-57286) | |
| | | GTDFTLTISSLQAEDVAVYYCHQSYHRRTFGGGTKV | | |
| | | EIK | | |
| Ab209 | 476 | QVQLVQSGAEVKKPGASVKVSCKASGFNIKDYYMH | SAD10318_P03_G09 | HC amino acid sequence |
| | | WVRQAPGQRLEWMGWIDLENANTIYDAKFQGRVTI | (ADI-57287) | |
| | | TRDTSASTAYMELSSLRSEDTAVYYCARDHYGHYFY | | |
| | | DVWGQGTLVTVSS | | |
| Ab209 | 477 | DIVMTQSPDSLAVSLGERATINCKSSQSLLNAHTGHN | SAD10318_P03_G09 | LC amino acid sequence |
| | | YLAWYQQKPGQPPKLLIYWASTRESGVPDRFSGSGS | (ADI-57287) | |
| | | GTDFTLTISSLQAEDVAVYYCHQSYSHRTFGGGTKVE | | |
| | | IK | | |
| Ab210 | 478 | QVQLVQSGAEVKKPGASVKVSCKASGFNIKDYYMH | SAD10318_P04_C10 | HC amino acid sequence |
| | | WVRQAPGQRLEWMGWIDLENANTIYDAKFQGRVTI | (ADI-48612) | |
| | | TRDTSASTAYMELSSLRSEDTAVYYCARDAYGRYHH | | |
| | | DVWGQGTLVTVSS | | |
| Ab210 | 479 | DIVMTQSPDSLAVSLGERATINCKSSQSLLNARTGHN | SAD10318_P04_C10 | LC amino acid sequence |
| | | HLAWYQQKPGQPPKLLIYWASTRESGVPDRFSGSGS | (ADI-48612) | |
| | | GTDFTLTISSLQAEDVAVYYCHQSYHRRTFGGGTKV | | |
| | | EIK | | |
| Ab211 | 480 | QVQLVQSGAEVKKPGASVKVSCKASGFNIKDYYMH | SAD10318_P04_D10 | HC amino acid sequence |
| | | WVRQAPGQRLEWMGWIDLENANTIYDAKFQGRVTI | (ADI-48613) | |
| | | TRDTSASTAYMELSSLRSEDTAVYYCARDAYGHYHY | | |
| | | DVWGQGTLVTVSS | | |
| Ab211 | 481 | DIVMTQSPDSLAVSLGERATINCKSSQSLLHARTGKN | SAD10318_P04_D10 | LC amino acid sequence |
| | | HLAWYQQKPGQPPKLLIYWASTRESGVPDRFSGSGS | (ADI-48613) | |
| | | GTDFTLTISSLQAEDVAVYYCKQSYSRRTFGGGTKVE | | |
| | | IK | | |
| Ab212 | 482 | QVQLVQSGAEVKKPGASVKVSCKASGFNIKDYYMH | SAD10318_P04_E10 | HC amino acid sequence |
| | | WVRQAPGQRLEWMGWIDLENANTIYDAKFQGRVTI | (ADI-57288) | |
| | | TRDTSASTAYMELSSLRSEDTAVYYCARDAYGRYHY | | |
| | | DHWGQGTLVTVSS | | |
| Ab212 | 483 | DIVMTQSPDSLAVSLGERATINCKSSQSLLNARTGHN | SAD10318_P04_E10 | LC amino acid sequence |
| | | YLAWYQQKPGQPPKLLISGSTRESGVPDRFSGSGS | (ADI-57288) | |
| | | GTDFTLTISSLQAEDVAVYYCHQSYHRRTFGGGTKVE | | |
| | | IK | | |
| Ab213 | 484 | QVQLVQSGAEVKKPGASVKVSCKASGFNIKDYYMH | SAD10318_P04_F10 | HC amino acid sequence |
| | | WVRQAPGQRLEWMGWIDLENANTIYDAKFQGRVTI | (ADI-57289) | |
| | | TRDTSASTAYMELSSLRSEDTAVYYCARDAYHRYHY | | |
| | | DVWGQGTLVTVSS | | |
| Ab213 | 485 | DIVMTQSPDSLAVSLGERATINCKSSQSLLNARTHKN | SAD10318_P04_F10 | LC amino acid sequence |
| | | HLAWYQQKPGQPPKLLIYWASTRESGVPDRFSGSGS | (ADI-57289) | |
| | | GTDFTLTISSLQAEDVAVYYCKQSYSRRTFGGGTKVE | | |
| | | IK | | |
| Ab214 | 486 | QVQLVQSGAEVKKPGASVKVSCKASGFNIKDYYMH | SAD10318_P04_H10 | HC amino acid sequence |
| | | WVRQAPGQRLEWMGWIDLENANTIYDAKFQGRVTI | (ADI-57290) | |
| | | TRDTSASTAYMELSSLRSEDTAVYYCARDHYGHYFY | | |
| | | DVWGQGTLVTVSS | | |
| Ab214 | 487 | DIVMTQSPDSLAVSLGERATINCKSSQSLLNAHTGKN | SAD10318_P04_H10 | LC amino acid sequence |
| | | HLAWYQQKPGQPPKLLIYWASTRESGVPDRFSGSGS | (ADI-57290) | |
| | | GTDFTLTISSLQAEDVAVYYCKQSYSRRTFGGGTKVE | | |
| | | IK | | |
| Ab215 | 488 | QVQLVQSGAEVKKPGASVKVSCKASGFNIKDYYMH | SAD10318_P01_G01 | HC amino acid sequence |
| | | WVRQAPGQRLEWMGWIDLENANTIYDAKFQGRVTI | (ADI-48579) | |
| | | TRDTSASTAYMELSSLRSEDTAVYYCARDAHGHYFY | | |
| | | DVWGQGTLVTVSS | | |
| Ab215 | 489 | DIVMTQSPDSLAVSLGERATINCKSSQSLLHAHTGKN | SAD10318_P01_G01 | LC amino acid sequence |
| | | YLAWYQQKPGQPPKLLIYWASTRESGVPDRFSGSGS | (ADI-48579) | |
| | | GTDFTLTISSLQAEDVAVYYCKQSHSHRTFGGGTKVE | | |
| | | IK | | |
| Ab216 | 490 | QVQLVQSGAEVKKPGASVKVSCKASGFNIKDYYMH | SAD10318_P04_A11 | HC amino acid sequence |
| | | WVRQAPGQRLEWMGWIDLENANTIYDAKFQGRVTI | (ADI-57291) | |
| | | TRDTSASTAYMELSSLRSEDTAVYYCARDHYGRHFY | | |
| | | DVWGQGTLVTVSS | | |
| Ab216 | 491 | DIVMTQSPDSLAVSLGERATINCKSSQSLLNARTGHN | SAD10318_P04_A11 | LC amino acid sequence |
| | | HLAWYQQKPGQPPKLLIYWASTRESGVPDRFSGSGS | (ADI-57291) | |
| | | GTDFTLTISSLQAEDVAVYYCKQSHHRRTFGGGTKV | | |
| | | EIK | | |
| Ab217 | 492 | QVQLVQSGAEVKKPGASVKVSCKASGFNIKDYYMH | SAD10318_P04_B11 | HC amino acid sequence |
| | | WVRQAPGQRLEWMGWIDLENANTIYDAKFQGRVTI | (ADI-48614) | |
| | | TRDTSASTAYMELSSLRSEDTAVYYCARDHYHRYFY | | |
| | | DVWGQGTLVTVSS | | |
| Ab217 | 493 | DIVMTQSPDSLAVSLGERATFNCKSSQSLLNAHTGKN | SAD10318_P04_B11 | LC amino acid sequence |
| | | HLAWYQQKPGQPPKLLIYWASTRESGVPDRFSGSGS | (ADI-48614) | |
| | | GTDFTLTISSLQAEDVAVYYCKQSYSRRTFGGGTKVE | | |
| | | IK | | |
| Ab218 | 494 | QVQLVQSGAEVKKPGASVKVSCKASGFNIKDYYMH | SAD10318_P04_C11 | HC amino acid sequence |
| | | WVRQAPGQRLEWMGWIDLENANTIYDAKFQGRVTI | (ADI-48615) | |
| | | TRDTSASTAYMELSSLRSEDTAVYYCARDHHGRYFY | | |
| | | DVWGQGTLVTVSS | | |
| Ab218 | 495 | DIVMTQSPDSLAVSLGERATINCKSSQSLLNARTGHN | SAD10318_P04_C11 | LC amino acid sequence |
| | | HLAWYQQKPGQPPKLLIYWASTRESGVPDRFSGSGS | (ADI-48615) | |
| | | GTDFTLTISSLQAEDVAVYYCKQSYSRRTFGGGTKVE | | |
| | | IK | | |
| Ab219 | 496 | QVQLVQSGAEVKKPGASVKVSCKASGFNIKDYYMH | SAD10318_P04_D11 | HC amino acid sequence |
| | | WVRQAPGQRLEWMGWIDLENANTIYDAKFQGRVTI | (ADI-48616) | |
| | | TRDTSASTAYMELSSLRSEDTAVYYCARDAYGHYFY | | |
| | | DVWGQGTLVTVSS | | |
| Ab219 | 497 | DIVMTQSPDSLAVSLGERATINCKSSQSLLNARTGHN | SAD10318_P04_D11 | LC amino acid sequence |
| | | HLAWYQQKPGQPPKLLIYWASTRESGVPDRFSGSGS | (ADI-48616) | |
| | | GTDFTLTISSLQAEDVAVYYCHQSYSHRTFGGGTKVE | | |
| | | IK | | |
| Ab220 | 498 | QVQLVQSGAEVKKPGASVKVSCKASGFNIKDYYMH | SAD10318_P04_E11 | HC amino acid sequence |
| | | WVRQAPGQRLEWMGWIDLENANTIYDAKFQGRVTI | (ADI-57292) | |
| | | TRDTSASTAYMELSSLRSEDTAVYYCARDAYGHYFY | | |
| | | DHWGQGTLVTVSS | | |
| Ab220 | 499 | DIVMTQSPDSLAVSLGERATINCKSSQSLLNARTGHN | SAD10318_P04_E11 | LC amino acid sequence |
| | | HLAWYQQKPGQPPKLLIYWASTRESGVPDRFSGSGS | (ADI-57292) | |
| | | GTDFTLTISSLQAEDVAVYYCHQSYSHRTFGGGTKVE | | |
| | | IK | | |
| Ab221 | 500 | QVQLVQSGAEVKKPGASVKVSCKASGFNTKDYYMH | SAD10318_P04_F11 | HC amino acid sequence |
| | | WVRQAPGQRLEWMGWIDLENANTIYDAKFQGRVTI | (ADI-57293) | |
| | | TRDTSASTAYMELSSLRSEDTAVYYCARDAHGRYHY | | |
| | | DVWGQGTLVTVSS | | |
| Ab221 | 501 | DIVMTQSPDSLAVSLGERATINCKSSQSLLNARTGHN | SAD10318_P04_F11 | LC amino acid sequence |
| | | HLAWYQQKPGQPPKLLIYWASTRESGVPDRFSGSGS | (ADI-57293) | |
| | | GTDFTLTISSLQAEDVAVYYCKQSYSRRTFGGGTKVE | | |
| | | IK | | |
| Ab222 | 502 | QVQLVQSGAEVKKPGASVKVSCKASGFNIKDYYMH | SAD10318_P04_G11 | HC amino acid sequence |
| | | WVRQAPGQRLEWMGWIDLENANTIYDAKFQGRVTI | (ADI-57294) | |
| | | TRDASASTAYMELSSLRSEDTAVYYCARDAYHRYH | | |
| | | YDVWGQGTLVTVSS | | |
| Ab222 | 503 | DIVMTQSPDSLAVSLGERATINCKSSQSLLNAHTGKN | SAD10318_P04_G11 | LC amino acid sequence |
| | | HLAWYQQKPGQPPKLLIYWASTRESGVPDRFSGSGS | (ADI-57294) | |
| | | GTDFTLTISSLQAEDVAVYYCKQSHSRRTFGGGTKVE | | |
| | | IK | | |
| Ab223 | 504 | QVQLVQSGAEVKKPGASVKVSCKASGFNIKDYYMH | SAD10318_P04_H11 | HC amino acid sequence |
| | | WVRQAPGQRLEWMGWIDLENANTIYDAKFQGRVTI | (ADI-48617) | |
| | | TRDTSASTAYMELSSLRSEDTAVYYCARDHYGRYFY | | |
| | | DHWGQGTLVTVSS | | |
| Ab223 | 505 | DIVMTQYPDSLAVSLGERATINCKSSQSLLNAHTGHN | SAD10318_P04_H11 | LC amino acid sequence |
| | | YLAWYQQKPGQPPKLLIYWASTRESGVPDRFSGSGS | (ADI-48617) | |
| | | GTDFTLTISSLQAEDVAVYYCHQSYSHRTFGGGTKVE | | |
| | | IK | | |
| Ab224 | 506 | QVQLVQSGAEVKKPGASVKVSCKASGFNIKDYYMH | SAD10318_P04_A12 | HC amino acid sequence |
| | | WVRQAPGQRLEWMGWIDLENANTIYDAKFQGRVTI | (ADI-48618) | |
| | | TRDTSASTAYMELSSLRSEDTAVYYCARDAYHRHFY | | |
| | | DVWGQGTLVTVSS | | |
| Ab224 | 507 | DIVMTQSPDSLAVSLGERATINCKSSQSLLHPRTGKN | SAD10318_P04_A12 | LC amino acid sequence |
| | | YLAWYQQKPGQPPKLLIYWASTRESGVPDRFSGSGS | (ADI-48618) | |
| | | GTDFTLTISSLQAEDVAVYYCKQSYHHRTFGGGTKV | | |
| | | EIK | | |
| Ab225 | 508 | QVQLVQSGAEVKKPGASVKVSCKASGFNIKDYYMH | SAD10318_P04_F12 | HC amino acid sequence |
| | | WVRQAPGQRLEWMGWIDLENANTIYDAKFQGRVTI | (ADI-57295) | |
| | | TRDTSASTAYMELSSLRSEDTAVYYCARDHHGRYFY | | |
| | | DVWGQGTLVTVSS | | |
| Ab225 | 509 | DIVMTQSPDSLAVSLGERATINCKSSQSLLHARTGHN | SAD10318_P04_F12 | LC amino acid sequence |
| | | YLAWYQQKPGQPPKLLIYWASTRESGVPDRFSGSGS | (ADI-57295) | |
| | | GTDFTLTISSLQAEDVAVYYCKQSHSHRTFGGGTKVE | | |
| | | IK | | |
| Ab226 | 510 | QVQLVQSGAEVKKPGASVKVSCKASGFNIKDYYMH | SAD10318_P01_H01 | HC amino acid sequence |
| | | WVRQAPGQRLEWMGWIDLENANTIYDAKFQGRVTI | (ADI-48580) | |
| | | TRDTSASTAYMELSSLRSEDTAVYYCARDAHHRYFY | | |
| | | DVWGQGTLVTVSS | | |
| Ab226 | 511 | DIVMTQSPDSLAVSLGERATINCKSSQSLLNAHTGKN | SAD10318_P01_H01 | LC amino acid sequence |
| | | HLAWYQQKPGQPPKLLIYWASTRESGVPDRFSGSGS | (ADI-48580) | |
| | | GTDFTLTISSLQAEDVAVYYCKQSYHHRTFGGGTKV | | |
| | | EIK | | |
| Ab227 | 512 | QVQLVQSGAEVKKPGASVKVSCKASGFNIKDYYMH | SAD10318_P04_G12 | HC amino acid sequence |
| | | WVRQAPGQRLEWMGWIDLENANTIYDAKFQGRVTI | (ADI-57296) | |
| | | TRDTSASTAYMELSSLRSEDTAVYYCARDAYGHYHY | | |
| | | DVWGQGTLVTVSS | | |
| Ab227 | 513 | DIVMTQSPDSLAVSLGERATINCKSSQSLLNAHTGKN | SAD10318_P04_G12 | LC amino acid sequence |
| | | HLAWYQQKPGQPPKLLIYWASTRESGVPDRFSGSGS | (ADI-57296) | |
| | | GTDFTLTISSLQAEDVAVYYCKQSYSRRTFGGGTKVE | | |
| | | IK | | |
| Ab228 | 514 | QVQLVQSGAEVKKPGASVKVSCKASGFNIKDYYMH | SAD10318_P04_H12 | HC amino acid sequence |
| | | WVRQAPGQRLEWMGWIDLENANTIYDAKFQGRVTI | (ADI-57297) | |
| | | TRDTSASTAYMELSSLRSEDTAVYYCARDAHGRYFH | | |
| | | DVWGQGTLVTVSS | | |
| Ab228 | 515 | DIVMTQSPDSLAVSLGERATINCKSSQSLLNAHTGKN | SAD10318_P04_H12 | LC amino acid sequence |
| | | HLAWYQQKPGQPPKLLIYWASTRESGVPDRFSGSGS | (ADI-57297) | |
| | | GTDFTLTISSLQAEDVAVYYCHQSYSHRTFGGGTKVE | | |
| | | IK | | |
| Ab229 | 516 | QVQLVQSGAEVKKPGASVKVSCKASGYTFASYAMH | SAD10319_P01_A01 | HC amino acid sequence |
| | | WVRQAPGQRLEWMGWINPDTGNTVYSQKFQGRVTI | (ADI-48619) | |
| | | TRDTSASTAYMELSSLRSEDTAVYYCARDAYHHYFY | | |
| | | DVWGQGTLVTVSS | | |
| Ab229 | 517 | DIVMTQSPDSLAVSLGERATINCKSSQSLLNARTGKN | SAD10319_P01_A01 | LC amino acid sequence |
| | | YLAWYQQKPGQPPKLLIYWASTRESGVPDRFSGSGS | (ADI-48619) | |
| | | GTDFTLTISSLQAEDVAVYYCKQSYSRRTFGGGTKVE | | |
| | | IK | | |
| Ab230 | 518 | QVQLVQSGAEVKKPGASVKVSCKASGYTFTDYDMH | SAD10319_P01_C01 | HC amino acid sequence |
| | | WVRQAPGQRLEWMGWIDAGTGLTKYSQKFQGRVTI | (ADI-48621 and | |
| | | TRDTSASTAYMELSSLRSEDTAVYYCARDHYGHYFY | ADI-48636) | |
| | | DVWGQGTLVTVSS | | |
| Ab230 | 519 | DIVMTQSPDSLAVSLGERATINCKSSQSLLNARTGKN | SAD10319_P01_C01 | LC amino acid sequence |
| | | YLAWYQQKPGQPPKLLIYWASTRESGVPDRFSGSGS | (ADI-48621 and | |
| | | GTDFTLTISSLQAEDVAVYYCKQSYSRRTFGGGTKVE | ADI-48636) | |
| | | IK | | |
| Ab231 | 520 | QVQLVQSGAEVKKPGASVKVSCKASGYTFKSYDMH | SAD10319_P01_D01 | HC amino acid sequence |
| | | WVRQAPGQRLEWMGWIDAGTGNTKYSQKFQGRVTI | (ADI-48622) | |
| | | TRDTSASTAYMELSSLRSEDTAVYYCARDAYGRYFY | | |
| | | DVWGQGTLVTVSS | | |
| Ab231 | 521 | DIVMTQSPDSLAVSLGERATINCKSSQSLLNARTGKN | SAD10319_P01_D01 | LC amino acid sequence |
| | | YLAWYQQKPGQPPKLLIYWASTRESGVPDRFSGSGS | (ADI-48622) | |
| | | GTDFTLTISSLQAEDVAVYYCKQSYSRRTFGGGTKVE | | |
| | | IK | | |
| Ab232 | 522 | QVQLVQSGAEVKKPGASVKVSCKASGYTFDAYAMH | SAD10319_P01_E01 | HC amino acid sequence |
| | | WVRQAPGQRLEWMGWIDAGTGATAYSQKFQGRVTI | (ADI-57298) | |
| | | TRDTSASTAYMELSSLRSEDTAVYYCARDHYGHYFY | | |
| | | DVWGQGTLVTVSS | | |
| Ab232 | 523 | DIVMTQSPDSLAVSLGERATINCKSSQSLLNARTGKN | SAD10319_P01_E01 | LC amino acid sequence |
| | | YLAWYQQKPGQPPKLLIYWASTRESGVPDRFSGSGS | (ADI-57298) | |
| | | GTDFTLTISSLQAEDVAVYYCKQSYSRRTFGGGTKVE | | |
| | | IK | | |
| Ab233 | 524 | QVQLVQSGAEVKKPGASVKVSCKASGYTFQSYDMH | SAD10319_P01_F01 | HC amino acid sequence |
| | | WVRQAPGQRLQWMGWINPTTGNTVYSQKFQGRVTI | (ADI-57299) | |
| | | TRDTSASTAYMELSSLRSEDTAVYYCARDAYGRYFY | | |
| | | DVWGQGTLVTVSS | | |
| Ab233 | 525 | DIVMTQSPDSLAVSLGERATINCKSSQSLLNARTGKN | SAD10319_P01_F01 | LC amino acid sequence |
| | | YLAWYQQKPGQPPKLLIYWASTRESGVPDRFSGSGS | (ADI-57299) | |
| | | GTDFTLTISSLQAEDVAVYYCKQSYSRRTFGGGTKVE | | |
| | | IK | | |
| Ab234 | 526 | QVQLVQSGAEVKKPGASVKVSCKASGYTFTSADMH | SAD10319_P01_G01 | HC amino acid sequence |
| | | WVRQAPGQRLQPMGWINAGTGATKYSQKFQGRVTI | (ADI-57300) | |
| | | TRDTSANTAYMELSSLRSEDTAVYYCARDAYGRYFY | | |
| | | DVWGQGTLVTVSS | | |
| Ab234 | 527 | DIVMTQSPDSLAVSLGERATINCKSSQSLLNARTGKN | SAD10319_P01_G01 | LC amino acid sequence |
| | | YLAWYQQKPGQPPKLLIYWASTRESGVPDRFSGSGS | (ADI-57300) | |
| | | GTDFTLTISSLQAEDVAVYYCKQSYSRRTFGGGTKVE | | |
| | | IK | | |
| Ab235 | 528 | QVQLVQSGAEVKKPGASVKVSCKASGYTFDDYAMH | SAD10319_P01_H01 | HC amino acid sequence |
| | | WVRQAPGQRLEWMGWIDAGTGDTKYSQKFQGRVTI | (ADI-48623) | |
| | | TRDTSASTAYMELSSLRSEDTAVYYCARDHYHRYFY | | |
| | | DVWGQGTLVTVSS | | |
| Ab235 | 529 | DIVMTQSPDSLAVSLGERATINCKSSQSLLNARTGKN | SAD10319_P01_H01 | LC amino acid sequence |
| | | YLAWYQQKPGQPPKLLIYWASTRESGVPDRFSGSGS | (ADI-48623) | |
| | | GTDFTLTISSLQAEDVAVYYCKQSYSRRTFGGGTKVE | | |
| | | IK | | |
| Ab236 | 530 | QVQLVQSGAEVKKPGASVKVSCKASGYTFTEYEMH | SAD10319_P01_A02 | HC amino acid sequence |
| | | WVRQAPGQRLEWMGWINPLTGNTKYSQKFQGRVTI | (ADI-57301) | |
| | | TRDTSASTAYMELSSLRSEDTAVYYCARDHYGHYFY | | |
| | | DVWGQGTLVTVSS | | |
| Ab236 | 531 | DIVMTQSPDSLAVSLGERATINCKSSQSLLNARTGKN | SAD10319_P01_A02 | LC amino acid sequence |
| | | YLAWYQQKPGQPPKLLIYWASTRESGVPDRFSGSGS | (ADI-57301) | |
| | | GTDFTLTISSLQAEDVAVYYCKQSYSRRTFGGGTKVE | | |
| | | IK | | |
| Ab237 | 532 | QVQLVQSGAEVKKPGASVKVSCKASGFNIKDYYMH | SAD10318_P01_A02 | HC amino acid sequence |
| | | WVRQAPGQRLEWMGWIDLENANTIYDAKFQGRVTI | (ADI-48581) | |
| | | TRDTSASTAYMELSSLRSEDTAVYYCARDAYHHYFY | | |
| | | DVWGQGTLVTVSS | | |
| Ab237 | 533 | DIVMTQSPDSLAVSLGERATINCKSSQSLLNARTHKN | SAD10318_P01_A02 | LC amino acid sequence |
| | | HLAWYQQKPGQPPKLLIYWASTRESGVPDRFSGSGS | (ADI-48581) | |
| | | GTDFTLTISSLQAEDVAVYYCKQSYHHRTFGGGTKV | | |
| | | EIK | | |
| Ab238 | 534 | QVQLVQSGAEVKKPGASVKVSCKASGYTFTEYNMH | SAD10319_P01_C02 | HC amino acid sequence |
| | | WVRQAPGQRLEWMGWINPLTGNTLYSQKFQGRVTI | (ADI-57302) | |
| | | TRDTSASTAYMELSSLRSEDTAVYYCARDAYHHYFY | | |
| | | DVWGQGTLVTVSS | | |
| Ab238 | 535 | DIVMTQSPDSLAVSLGERATINCKSSQSLLNARTGKN | SAD10319_P01_C02 | LC amino acid sequence |
| | | YLAWYQQKPGQPPKLLIYWASTRESGVPDRFSGSGS | (ADI-57302) | |
| | | GTDFTLTISSLQAEDVAVYYCKQSYSRRTFGGGTKVE | | |
| | | IK | | |
| Ab239 | 536 | QVQLVQSGAEVKKPGASVKVSCKASGYTFTEYDMH | SAD10319_P01_D02 | HC amino acid sequence |
| | | WVRQAPGQRPEWMGWIDAGTGATVYSQKFQGRVTI | (ADI-57303) | |
| | | TRDTSASTAYMELSSLRSEDTAVYYCARDAYGRYFY | | |
| | | DHWGQGTLVTVSS | | |
| Ab239 | 537 | DIVMTQSPDSLAVSLGERATINCKSSQSLLNARTGKN | SAD10319_P01_D02 | LC amino acid sequence |
| | | YLAWYQQKPGQPPKLLIYWASTRESGVPDRFSGSGS | (ADI-57303) | |
| | | GTDFTLTISSLQAEDVAVYYCKQSYSRRTFGGGTKVE | | |
| | | IK | | |
| Ab240 | 538 | QVQLVQSGAEVKKPGASVKVSCKASGYTFTSDFMH | SAD10319_P01_E02 | HC amino acid sequence |
| | | WVRQAPGQRLEWMGWINPATGATKYSQKFQGRVTI | (ADI-57304) | |
| | | TRDTSASTAYMELSSLRSEDTAVYYCARDAYGHYFY | | |
| | | DHWGQGTLVTVSS | | |
| Ab240 | 539 | DIVMTQSPDSLAVSLGERATINCKSSQSLLNARTGKN | SAD10319_P01_E02 | LC amino acid sequence |
| | | YLAWYQQKPGQPPKLLIYWASTRESGVPDRFSGSGS | (ADI-57304) | |
| | | GTDFTLTISSLQAEDVAVYYCKQSYSRRTFGGGTKVE | | |
| | | IK | | |
| Ab241 | 540 | QVQLVQSGAEVKKPGASVKVSCKASGYTFESYDMH | SAD10319_P01_F02 | HC amino acid sequence |
| | | WVRQAPGQRLEWMGWIDAGTGNTKYSQKFQGRVTI | (ADI-48624) | |
| | | TADESTSTAYMELSSLRSEDTAVYYCARDHYGHYFY | | |
| | | DVWGQGTLVTVSS | | |
| Ab241 | 541 | DIVMTQSPDSLAVSLGERATINCKSSQSLLNARTGKN | SAD10319_P01_F02 | LC amino acid sequence |
| | | YLAWYQQKPGQPPKLLIYWASTRESGVPDRFSGSGS | (ADI-48624) | |
| | | GTDFTLTISSLQAEDVAVYYCKQSYSRRTFGGGTKVE | | |
| | | IK | | |
| Ab242 | 542 | QVQLVQSGAEVKKPGASVKVSCKASGYTFTDYAMH | SAD10319_P01_H02 | HC amino acid sequence |
| | | WVRQAPGQRLEWMGWINPSTGATVYSQKFQGRVTI | (ADI-48625) | |
| | | TRDTSASTAYMELSSLRSEDTAVYYCARDAYHHYFY | | |
| | | DVWGQGTLVTVSS | | |
| Ab242 | 543 | DIVMTQSPDSLAVSLGERATINCKSSQSLLNARTGKN | SAD10319_P01_H02 | LC amino acid sequence |
| | | YLAWYQQKPGQPPKLLIYWASTRESGVPDRFSGSGS | (ADI-48625) | |
| | | GTDFTLTISSLQAEDVAVYYCKQSYSRRTFGGGTKVE | | |
| | | IK | | |
| Ab243 | 544 | QVQLVQSGAEVKKPGASVKVSCKASGYTFNAYAMH | SAD10319_P01_B03 | HC amino acid sequence |
| | | WVRQAPGQRLEWMGWINPDTGATTYSQKFQGRVTI | (ADI-57305) | |
| | | TRDTSASTAYMELSSLRSEDTAVYYCARDAYHHYFY | | |
| | | DVWGQGTLVTVSS | | |
| Ab243 | 545 | DIVMTQSPDSLAVSLGERATINCKSSQSLLNARTGKN | SAD10319_P01_B03 | LC amino acid sequence |
| | | YLAWYQQKPGQPPKLLIYWASTRESGVPDRFSGSGS | (ADI-57305) | |
| | | GTDFTLTISSLQAEDVAVYYCKQSYSRRTFGGGTKVE | | |
| | | IK | | |
| Ab244 | 546 | QVQLVQSGAEVKKPGASVKVSCKASGYTFTDYDMH | SAD10319_P01_C03 | HC amino acid sequence |
| | | WVRQAPGQRLEWMGWIDPYTGATKYSQKFQGRVTI | (ADI-48626) | |
| | | TRDTSASTAYMELSSLRSEDTAVYYCARDAYGHYFY | | |
| | | DVWGQGTLVTVSS | | |
| Ab244 | 547 | DIVMTQSPDSLAVSLGERATINCKSSQSLLNARTGKN | SAD10319_P01_C03 | LC amino acid sequence |
| | | YLAWYQQKPGQPPKLLIYWASTRESGVPDRFSGSGS | (ADI-48626) | |
| | | GTDFTLTISSLQAEDVAVYYCKQSYSRRTFGGGTKVE | | |
| | | IK | | |
| Ab245 | 548 | QVQLVQSGAEVKKPGASVKVSCKASGYTFTSYNMH | SAD10319_P01_D03 | HC amino acid sequence |
| | | WVRQAPGQRLEWMGWIDAGTGATHYSQKFQGRVTI | (ADI-57306) | |
| | | TRDTSASTAYMELSSLRSEDTAVYYCARDAYHHYFY | | |
| | | DVWGQGTLVTVSS | | |
| Ab245 | 549 | DIVMTQSPDSLAVSLGERATINCKSSQSLLNARTGKN | SAD10319_P01_D03 | LC amino acid sequence |
| | | YLAWYQQKPGQPPKLLIYWASTRESGVPDRFSGSGS | (ADI-57306) | |
| | | GTDFTLTISSLQAEDVAVYYCKQSYSRRTFGGGTKVE | | |
| | | IK | | |
| Ab246 | 550 | QVQLVQSGAEVKKPGASVKVSCKASGYTFESYDMH | SAD10319_P01_F03 | HC amino acid sequence |
| | | WVRQAPGQRLEWMGWINAGTGATVYSQKFQGRVTI | (ADI-48627) | |
| | | TRDTSASTAYMELSSLRSEDTAVYYCARDAYHHYFY | | |
| | | DVWGQGTLVTVSS | | |
| Ab246 | 551 | DIVMTQSPDSLAVSLGERATINCKSSQSLLNARTGKN | SAD10319_P01_F03 | LC amino acid sequence |
| | | YLAWYQQKPGQPPKLLIYWASTRESGVPDRFSGSGS | (ADI-48627) | |
| | | GTDFTLTISSLQAEDVAVYYCKQSYSRRTFGGGTKVE | | |
| | | IK | | |
| Ab247 | 552 | QVQLVQSGAEVKKPGASVKVSCKASGYTFDSYSMH | SAD10319_P02_A04 | HC amino acid sequence |
| | | WVRQAPGQRLEWMGWINPDTGATDYSQKFQGRVTI | (ADI-57307) | |
| | | TRDTSASTAYMELSSLRSEDTAVYYCARDHYGHYFY | | |
| | | DVWGQGTLVTVSS | | |
| Ab247 | 553 | DIVMTQSPDSLAVSLGERATINCKSSQSLLNARTGKN | SAD10319_P02_A04 | LC amino acid sequence |
| | | YLAWYQQKPGQPPKLLIYWASTRESGVPDRFSGSGS | (ADI-57307) | |
| | | GTDFTLTISSLQAEDVAVYYCKQSYSRRTFGGGTKVE | | |
| | | IK | | |
| Ab248 | 554 | QVQLVQSGAEVKKPGASVKVSCKASGFNIKDYYMH | SAD10318_P01_B02 | HC amino acid sequence |
| | | WVRQAPGQRLEWMGWIDLENANTIYDAKFQGRVTI | (ADI-48582) | |
| | | TRDTSASTAYMELSSLRSEDTAVYYCARDHYHRYFY | | |
| | | DVWGQGTLVTVSS | | |
| Ab248 | 555 | DIVMTQSPDSLAVSLGERATINCKSSQSLLNARTGHN | SAD10318_P01_B02 | LC amino acid sequence |
| | | HLAWYQQKPGQPPKLLIYWASTRESGVPDRFSGSGS | (ADI-48582) | |
| | | GTDFTLTISSLQAEDVAVYYCKQSHSHRTFGGGTKVE | | |
| | | IK | | |
| Ab249 | 556 | QVQLVQSGAEVKKPGASVKVSCKASGYTFADYAMH | SAD10319_P02_B04 | HC amino acid sequence |
| | | WVRQAPGQRLEWMGWINPGTGSTKYSQKFQGRVTI | (ADI-57308) | |
| | | TRDTSASTAYMELSSLRSEDTAVYYCARDAYGHYFY | | |
| | | DHWGQGTLVTVSS | | |
| Ab249 | 557 | DIVMTQSPDSLAVSLGERATINCKSSQSLLNARTGKN | SAD10319_P02_B04 | LC amino acid sequence |
| | | YLAWYQQKPGQPPKLLIYWASTRESGVPDRFSGSGS | (ADI-57308) | |
| | | GTDFTLTISSLQAEDVAVYYCKQSYSRRTFGGGTKVE | | |
| | | IK | | |
| Ab250 | 558 | QVQLVQSGAEVKKPGASVKVSCKASGYTFESYSMH | SAD10319_P02_C04 | HC amino acid sequence |
| | | WVRQAPGQRLEWMGWINPATGATDYSQKFQGRVTI | (ADI-57309) | |
| | | TRDTSASTAYMELSSLRSEDTAVYYCARDAYHHYFY | | |
| | | DVWGQGTLVTVSS | | |
| Ab250 | 559 | DIVMTQSPDSLAVSLGERATINCKSSQSLLNARTGKN | SAD10319_P02_C04 | LC amino acid sequence |
| | | YLAWYQQKPGQPPKLLIYWASTRESGVPDRFSGSGS | (ADI-57309) | |
| | | GTDFTLTISSLQAEDVAVYYCKQSYSRRTFGGGTKVE | | |
| | | IK | | |
| Ab251 | 560 | QVQLVQSGAEVKKPGASVKVSCKASGYTFTSYAMH | SAD10319_P02_D04 | HC amino acid sequence |
| | | WVRQAPGQRLQWMGWIDAGTGNTYYSQKFQGRVTI | (ADI-57310) | |
| | | TRDTSASTAYMELSSLRSEDTAVYYCARDAHGHYFY | | |
| | | DVWGQGTLVTVSS | | |
| Ab251 | 561 | DIVMTQSPDSLAVSLGERATINCKSSQSLLNARTGKN | SAD10319_P02_D04 | LC amino acid sequence |
| | | YLAWYQQKPGQPPKLLIYWASTRESGVPDRFSGSGS | (ADI-57310) | |
| | | GTDFTLTISSLQAEDVAVYYCKQSYSRRTFGGGTKVE | | |
| | | IK | | |
| Ab252 | 562 | QVQLVQSGAEVKKPGASVKVSCKASGYTFESYYMH | SAD10319_P02_E04 | HC amino acid sequence |
| | | WVRQAPGQRLERMGSIVAGTGATKYSQKFQGRVTIT | (ADI-57311) | |
| | | RDTSASTAYMELSSLRSEDTAVYYCARDHYGRYFYD | | |
| | | VWGQGTPVTVSS | | |
| Ab252 | 563 | DIVMTQSPDSLAVSLGERATINCKSSQSLLNARTGKN | SAD10319_P02_E04 | LC amino acid sequence |
| | | YLAWYQQKPGQPPKLLIYWASTRESGVPDRFSGSGS | (ADI-57311) | |
| | | GTDFTLTISSLQAEDVAVYYCKQSYSRRTFGGGTKVE | | |
| | | IK | | |
| Ab253 | 564 | QVQLVQSGAEVKKPGASVKVSCKASGYTFDSYTMH | SAD10319_P02_F04 | HC amino acid sequence |
| | | WVRQAPGQRLEWMGYINPDTGATYYSQKFQGRVTI | (ADI-57312) | |
| | | TRDTSASTAYMELSSLRSEDTAVYYCARDAYHHYFY | | |
| | | DVWGQGTLVTVSS | | |
| Ab253 | 565 | DIVMTQSPDSLAVSLGERATINCKSSQSLLNARTGKN | SAD10319_P02_F04 | LC amino acid sequence |
| | | YLAWYQQKPGQPPKLLIYWASTRESGVPDRFSGSGS | (ADI-57312) | |
| | | GTDFTLTISSLQAEDVAVYYCKQSYSRRTFGGGTKVE | | |
| | | IK | | |
| Ab254 | 566 | QVQLVQSGAEVKKPGASVKVSCKASGYTFTSYAMH | SAD10319_P02_H04 | HC amino acid sequence |
| | | WVRQAPGQRLEWMGWINPDTGATKYSQKFQGRVTI | (ADI-57313) | |
| | | TRDTSASTAYMELSSLRSEDTAVYYCARDAHGHYFY | | |
| | | DVWGQGTLVTVSS | | |
| Ab254 | 567 | DIVMTQSPDSLAVSLGERATINCKSSQSLLNARTGKN | SAD10319_P02_H04 | LC amino acid sequence |
| | | YLAWYQQKPGQPPKLLIYWASTRESGVPDRFSGSGS | (ADI-57313) | |
| | | GTDFTLTISSLQAEDVAVYYCKQSYSRRTFGGGTKVE | | |
| | | IK | | |
| Ab255 | 568 | QVQLVQSGAEVKKPGASVKVSCKASGYTFDSYAMH | SAD10319_P02_A05 | HC amino acid sequence |
| | | WVRQAPGQRLEWMGWINPNTGATTYSQKFQGRVTI | (ADI-48629) | |
| | | TRDTSASTAYMELSSLRSEDTAVYYCARDHYGHYFY | | |
| | | DVWGQGTLVTVSS | | |
| Ab255 | 569 | DIVMTQSPDSLAVSLGERATINCKSSQSLLNARTGKN | SAD10319_P02_A05 | LC amino acid sequence |
| | | YLAWYQQKPGQPPKLLIYWASTRESGVPDRFSGSGS | (ADI-48629) | |
| | | GTDFTLTISSLQAEDVAVYYCKQSYSRRTFGGGTKVE | | |
| | | IK | | |
| Ab256 | 570 | QVQLVQSGAEVKKPGASVKVSCKASGYTFADYAMH | SAD10319_P02_B05 | HC amino acid sequence |
| | | WVRQAPGQRLEWMGSINAGTGNTYYSQKFQGRVTI | (ADI-57314) | |
| | | TRDTSASTAYMELSSLRSEDTAVYYCARDAYGHYFY | | |
| | | DVWGQGTLVTVSS | | |
| Ab256 | 571 | DIVMTQSPDSLAVSLGERATINCKSSQSLLNARTGKN | SAD10319_P02_B05 | LC amino acid sequence |
| | | YLAWYQQKPGQPPKLLIYWASTRESGVPDRFSGSGS | (ADI-57314) | |
| | | GTDFTLTISSLQAEDVAVYYCKQSYSRRTFGGGTKVE | | |
| | | IK | | |
| Ab257 | 572 | QVQLVQSGAEVKKPGASVKVSCKASGYTFTDYNMH | SAD10319_P02_C05 | HC amino acid sequence |
| | | WVRQAPGQRLEWMGWINPLTGTTKYSQKFQGRVTI | (ADI-57315) | |
| | | TRDTSASTAYMELSSLRSEDTAVYYCARDAYHHYFY | | |
| | | DVWGQGTLVTVSS | | |
| Ab257 | 573 | DIVMTQSPDSLAVSLGERATINCKSSQSLLNARTGKN | SAD10319_P02_C05 | LC amino acid sequence |
| | | YLAWYQQKPGQPPKLLIYWASTRESGVPDRFSGSGS | (ADI-57315) | |
| | | GTDFTLTISSLQAEDVAVYYCKQSYSRRTFGGGTKVE | | |
| | | IK | | |
| Ab258 | 574 | QVQLVQSGAEVKKPGASVKVSCKASGYTFNDYAMH | SAD10319_P02_D05 | HC amino acid sequence |
| | | WVRQAPGQRLAWMGWIDAGTGNTYYSQKFQGRVTI | (ADI-57316) | |
| | | TRDTSASTAYMELSSLRSEDTAVYYCARDAHGHYFY | | |
| | | DVWGQGTLVTVSS | | |
| Ab258 | 575 | DIVMTQSPDSLAVSLGERATINCKSSQSLLNARTGKN | SAD10319_P02_D05 | LC amino acid sequence |
| | | YLAWYQQKPGQPPKLLIYWASTRESGVPDRFSGSGS | (ADI-57316) | |
| | | GTDFTLTISSLQAEDVAVYYCKQSYSRRTFGGGTKVE | | |
| | | IK | | |

TABLE 2
Binding and PSR Data

| | Human CD3 pH 6.0 | | | Human CD3 pH 7.4 | | | Cyno CD3 pH 6.0 | |
|---|---|---|---|---|---|---|---|---|
| Clone # | KD | kon | koff | KD | kon | koff | KD | kon |
| SAD10318_P01_D01 | 7.60E−08 | 2.82E+05 | 2.14E−02 | N.B. | | | 8.29E−08 | 2.22E+05 |
| SAD10318_P01_E01 | 1.16E−07 | 1.50E+05 | 1.72E−02 | N.B. | | | 9.44E−08 | 1.58E+05 |
| SAD10318_P01_A03 | 3.66E−08 | 3.43E+05 | 1.26E−02 | N.B. | | | 5.37E−08 | 2.87E+05 |
| SAD10318_P02_C04 | 2.35E−08 | 3.31E+05 | 7.78E−03 | 1.28E−07 | 1.64E+05 | 2.10E−02 | 3.00E−08 | 3.28E+05 |
| SAD10318_P02_A05 | 1.56E−09 | 4.55E+05 | 7.12E−04 | 3.77E−08 | 2.39E+05 | 9.00E−03 | 1.74E−09 | 4.90E+05 |
| SAD10319_P03_E07 | 4.09E−08 | 2.98E+05 | 1.22E−02 | N.B. | | | 5.53E−08 | 2.37E+05 |
| SAD10320_P01_B01 | 1.31E−07 | 1.22E+05 | 1.60E−02 | N.B. | | | 6.77E−08 | 1.64E+05 |
| SAD10320_P01_E01 | 5.01E−08 | 2.26E+05 | 1.13E−02 | N.B. | | | 1.27E−07 | 1.15E+05 |
| SAD10320_P02_D05 | 1.29E−09 | 4.69E+05 | 6.05E−04 | 3.17E−08 | 2.91E+05 | 9.23E−03 | 1.57E−09 | 4.87E+05 |
| SAD10320_P02_H05 | 2.13E−09 | 4.18E+05 | 8.88E−04 | 5.92E−08 | 2.41E+05 | 1.43E−02 | 2.86E−09 | 3.92E+05 |
| (ADI-48652) | | | | | | | | |
| LAD9965_P01_H07 | 1.64E−08 | 4.91E+05 | 8.07E−03 | 1.02E−07 | 2.55E+05 | 2.60E−02 | 1.66E−08 | 5.03E+05 |
| SAD10319_P03_C07 | 2.38E−08 | 3.31E+05 | 7.88E−03 | N.B. | | | 2.09E−07 | 8.54E+04 |
| LAD5224_P03_A01 | 1.99E−09 | 4.16E+05 | 8.29E−04 | 5.33E−09 | 3.00E+05 | 1.60E−03 | 1.27E−09 | 3.86E+05 |
| SAD10318_P01_F01 | 5.51E−08 | 3.18E+05 | 1.75E−02 | N.B. | | | 1.20E−07 | 1.40E+05 |
| SAD10318_P01_C03 | 1.61E−07 | 1.63E+05 | 2.62E−02 | 2.99E−07 | 6.56E+04 | 1.96E−02 | 3.47E−07 | 6.38E+04 |
| SAD10318_P01_E03 | 8.46E−08 | 2.43E+05 | 2.05E−02 | N.B. | | | 1.46E−08 | 2.67E+05 |
| SAD10318_P02_B04 | 1.71E−07 | 1.29E+05 | 2.20E−02 | N.B. | | | 1.06E−08 | 2.48E+05 |
| SAD10318_P02_D04 | 6.66E−08 | 2.86E+05 | 1.90E−02 | N.B. | | | 1.26E−07 | 1.29E+05 |
| SAD10318_P02_E04 | 1.93E−07 | 1.06E+05 | 2.05E−02 | 9.57E−08 | 1.42E+05 | 1.36E−02 | 3.21E−07 | 5.69E+04 |
| SAD10318_P02_F05 | 1.28E−07 | 2.20E+05 | 2.82E−02 | 1.20E−07 | 1.64E+05 | 1.98E−02 | 2.28E−07 | 1.06E+05 |
| SAD10318_P02_G05 | 9.46E−08 | 2.82E+05 | 2.67E−02 | 1.02E−07 | 2.32E+05 | 2.37E−02 | 2.61E−07 | 9.96E+04 |
| SAD10318_P02_B06 | 6.39E−08 | 3.65E+05 | 2.33E−02 | N.B. | | | 3.57E−08 | 2.54E+05 |
| SAD10318_P02_D06 | 2.07E−07 | 1.64E+05 | 3.38E−02 | N.B. | | | 1.26E−07 | 1.48E+05 |
| SAD10318_P02_E06 | 1.19E−07 | 1.93E+05 | 2.30E−02 | N.B. | | | 2.00E−07 | 9.14E+04 |
| SAD10318_P02_G06 | 1.06E−07 | 2.18E+05 | 2.31E−02 | 6.84E−08 | 2.46E+05 | 1.68E−02 | 2.22E−07 | 9.70E+04 |
| SAD10318_P03_B07 | 1.55E−07 | 1.67E+05 | 2.60E−02 | N.B. | | | 2.13E−08 | 2.26E+05 |
| SAD10318_P03_F07 | 4.08E−08 | 2.90E+05 | 1.18E−02 | N.B. | | | 1.06E−08 | 2.03E+05 |
| SAD10318_P03_G07 | 1.43E−08 | 2.51E+05 | 3.58E−03 | N.B. | | | 7.45E−09 | 2.46E+05 |
| SAD10318_P03_A08 | 8.11E−09 | 4.62E+05 | 3.75E−03 | N.B. | | | 2.43E−08 | 2.47E+05 |
| SAD10318_P03_B08 | 7.98E−08 | 2.15E+05 | 1.72E−02 | N.B. | | | 1.10E−08 | 2.76E+05 |
| SAD10318_P03_G08 | 5.18E−08 | 2.69E+05 | 1.39E−02 | N.B. | | | 2.83E−08 | 1.92E+05 |
| SAD10318_P03_F09 | 4.24E−08 | 2.23E+05 | 9.48E−03 | N.B. | | | 8.54E−09 | 1.63E+05 |
| SAD10318_P03_G09 | 6.02E−08 | 2.47E+05 | 1.49E−02 | N.B. | | | 2.78E−08 | 2.01E+05 |
| SAD10318_P04_D10 | 4.16E−08 | 3.40E+05 | 1.41E−02 | N.B. | | | 1.21E−08 | 2.07E+05 |
| SAD10318_P04_E10 | 8.39E−08 | 1.51E+05 | 1.27E−02 | N.B. | | | 1.28E−08 | 1.91E+05 |
| SAD10318_P04_F10 | 7.44E−08 | 1.99E+05 | 1.48E−02 | N.B. | | | 2.02E−08 | 2.15E+05 |
| SAD10318_P04_H10 | 5.01E−08 | 2.46E+05 | 1.23E−02 | N.B. | | | 1.03E−08 | 2.76E+05 |
| SAD10318_P04_A11 | N.B. | | | N.B. | | | N.B. | |
| SAD10318_P04_E11 | 7.88E−08 | 2.13E+05 | 1.68E−02 | N.B. | | | 1.06E−08 | 2.42E+05 |
| SAD10318_P04_F11 | 4.83E−08 | 2.56E+05 | 1.24E−02 | N.B. | | | 9.05E−09 | 2.02E+05 |
| SAD10318_P04_G11 | 2.72E−08 | 1.84E+05 | 5.00E−03 | N.B. | | | 1.06E−08 | 2.02E+05 |
| SAD10318_P04_F12 | N.B. | | | N.B. | | | N.B. | |
| SAD10318_P04_G12 | 4.14E−08 | 2.56E+05 | 1.06E−02 | N.B. | | | 1.12E−08 | 2.24E+05 |
| SAD10318_P04_H12 | 8.69E−08 | 1.79E+05 | 1.55E−02 | N.B. | | | 1.15E−08 | 2.21E+05 |
| SAD10319_P01_A01 | 9.14E−08 | 3.04E+05 | 2.78E−02 | N.B. | | | 4.92E−07 | 6.66E+04 |
| SAD10319_P01_C01 | 7.93E−08 | 2.44E+05 | 1.94E−02 | N.B. | | | 1.63E−08 | 2.47E+05 |
| SAD10319_P01_D01 | 7.87E−08 | 3.30E+05 | 2.60E−02 | N.B. | | | 3.22E−08 | 2.94E+05 |
| SAD10319_P01_E01 | 8.41E−08 | 3.40E+05 | 2.86E−02 | 3.23E−08 | 4.12E+05 | 1.33E−02 | 1.91E−07 | 1.47E+05 |
| SAD10319_P01_F01 | 6.63E−08 | 3.54E+05 | 2.35E−02 | N.B. | | | 8.16E−08 | 2.19E+05 |
| SAD10319_P01_G01 | 9.97E−08 | 2.24E+05 | 2.23E−02 | N.B. | | | 9.42E−09 | 2.64E+05 |
| SAD10319_P0l_A02 | 2.96E−08 | 3.49E+05 | 1.03E−02 | N.B. | | | 1.49E−07 | 1.11E+05 |
| SAD10319_P01_C02 | 2.40E−07 | 1.51E+05 | 3.62E−02 | N.B. | | | 2.20E−07 | 1.14E+05 |
| SAD10319_P01_D02 | 1.02E−07 | 2.01E+05 | 2.05E−02 | N.B. | | | 2.98E−08 | 2.30E+05 |
| SAD10319_P01_E02 | 1.14E−07 | 2.00E+05 | 2.28E−02 | 1.29E−07 | 2.20E+05 | 2.83E−02 | 1.62E−07 | 1.53E+05 |
| SAD10319_P01_F02 | 5.83E−08 | 3.32E+05 | 1.93E−02 | N.B. | | | 1.87E−08 | 2.79E+05 |
| SAD10319_P01_H02 | 8.15E−08 | 3.16E+05 | 2.57E−02 | N.B. | | | 2.94E−07 | 1.07E+05 |
| SAD10319_P01_B03 | 7.14E−08 | 3.43E+05 | 2.45E−02 | N.B. | | | 1.47E−07 | 1.66E+05 |
| SAD10319_P01_C03 | 8.13E−08 | 2.58E+05 | 2.10E−02 | N.B. | | | 1.47E−07 | 1.34E+05 |
| SAD10319_P01_D03 | 5.25E−08 | 3.42E+05 | 1.79E−02 | N.B. | | | 7.36E−09 | 3.00E+05 |
| SAD10319_P02_A04 | 4.36E−08 | 3.87E+05 | 1.69E−02 | N.B. | | | 1.86E−07 | 1.52E+05 |
| SAD10319_P02_B04 | 1.73E−07 | 1.93E+05 | 3.35E−02 | N.B. | | | 8.80E−08 | 2.31E+05 |
| SAD10319_P02_C04 | 3.56E−08 | 4.65E+05 | 1.66E−02 | N.B. | | | 1.11E−07 | 1.93E+05 |
| SAD10319_P02_D04 | 2.81E−08 | 4.70E+05 | 1.32E−02 | N.B. | | | 8.42E−09 | 3.83E+05 |
| SAD10319_P02_E04 | 5.79E−08 | 3.91E+05 | 2.26E−02 | 6.02E−08 | 4.32E+05 | 2.60E−02 | 1.02E−07 | 2.11E+05 |
| SAD10319_P02_F04 | 6.69E−08 | 3.66E+05 | 2.45E−02 | 2.21E−08 | 3.36E+05 | 7.43E−03 | 3.38E−07 | 9.38E+04 |
| SAD10319_P02_H04 | 1.15E−07 | 1.74E+05 | 2.00E−02 | N.B. | | | 1.20E−08 | 2.61E+05 |
| SAD10319_P02_A05 | 2.79E−07 | 1.52E+05 | 4.25E−02 | N.B. | | | 1.33E−07 | 2.02E+05 |
| SAD10319_P02_B05 | 5.58E−08 | 4.18E+05 | 2.33E−02 | 1.00E−08 | 5.20E+05 | 5.21E−03 | 3.43E−07 | 1.13E+05 |
| SAD10319_P02_C05 | 1.21E−07 | 1.94E+05 | 2.34E−02 | N.B. | | | 2.44E−08 | 2.86E+05 |
| SAD10319_P02_D05 | 2.78E−07 | 1.29E+05 | 3.59E−02 | 7.67E−09 | 3.58E+05 | 2.75E−03 | 1.00E−08 | 2.95E+05 |
| SAD10319_P02_F05 | 8.27E−08 | 2.85E+05 | 2.36E−02 | N.B. | | | 5.17E−07 | 5.08E+04 |
| SAD10319_P02_G05 | 2.75E−07 | 1.24E+05 | 3.42E−02 | N.B. | | | 1.67E−08 | 2.84E+05 |
| SAD10319_P02_H05 | 1.08E−07 | 2.16E+05 | 2.34E−02 | 4.31E−08 | 3.20E+05 | 1.38E−02 | 1.13E−07 | 1.68E+05 |
| SAD10319_P02_A06 | 2.78E−07 | 1.49E+05 | 4.13E−02 | 9.19E−08 | 2.15E+05 | 1.97E−02 | 2.56E−07 | 1.27E+05 |
| SAD10319_P02_B06 | 1.36E−07 | 2.20E+05 | 2.99E−02 | N.B. | | | 1.90E−08 | 2.80E+05 |
| SAD10319_P02_C06 | 6.21E−08 | 3.79E+05 | 2.36E−02 | 5.30E−08 | 5.14E+05 | 2.72E−02 | 9.63E−08 | 2.39E+05 |
| SAD10319_P02_D06 | 1.87E−07 | 1.73E+05 | 3.23E−02 | 2.04E−08 | 3.16E+05 | 6.45E−03 | 1.15E−07 | 1.85E+05 |
| SAD10319_P02_E06 | 2.19E−08 | 5.60E+05 | 1.22E−02 | 4.16E−08 | 5.57E+05 | 2.32E−02 | 4.84E−09 | 5.19E+05 |
| SAD10319_P02_F06 | 2.01E−07 | 1.94E+05 | 3.89E−02 | 3.64E−08 | 3.12E+05 | 1.14E−02 | 3.38E−08 | 3.60E+05 |
| SAD10319_P02_G06 | 6.22E−08 | 3.34E+05 | 2.08E−02 | N.B. | | | 1.49E−07 | 1.62E+05 |
| SAD10319_P02_H06 | 2.54E−07 | 1.37E+05 | 3.49E−02 | 1.07E−08 | 3.07E+05 | 3.29E−03 | 1.16E−07 | 1.62E+05 |
| SAD10319_P03_G07 | 1.27E−07 | 1.66E+05 | 2.11E−02 | N.B. | | | 2.48E−07 | 7.54E+04 |
| SAD10319_P03_H07 | 1.20E−07 | 1.95E+05 | 2.34E−02 | N.B. | | | 2.12E−07 | 9.48E+04 |
| SAD10319_P03_D08 | 2.20E−08 | 3.31E+05 | 7.27E−03 | N.B. | | | 1.80E−07 | 1.11E+05 |
| SAD10319_P03_E08 | 5.54E−08 | 3.14E+05 | 1.74E−02 | N.B. | | | 1.80E−07 | 1.10E+05 |
| SAD10319_P03_G08 | 3.09E−07 | 9.81E+04 | 3.03E−02 | N.B. | | | 1.33E−07 | 1.26E+05 |
| SAD10319_P03_H08 | 1.40E−07 | 1.98E+05 | 2.77E−02 | N.B. | | | 1.25E−08 | 2.77E+05 |
| SAD10319_P03_F09 | 4.10E−08 | 4.74E+05 | 1.95E−02 | N.B. | | | 1.05E−07 | 1.91E+05 |
| SAD10319_P03_G09 | 2.19E−07 | 1.36E+05 | 2.98E−02 | N.B. | | | 1.77E−07 | 1.24E+05 |
| SAD10319_P04_A10 | 1.21E−07 | 2.15E+05 | 2.61E−02 | N.B. | | | 3.05E−07 | 8.61E+04 |
| SAD10319_P04_B10 | 2.31E−07 | 1.50E+05 | 3.47E−02 | 3.34E−08 | 3.07E+05 | 1.03E−02 | 3.67E−08 | 3.46E+05 |
| SAD10319_P04_C10 | 1.84E−08 | 2.68E+05 | 4.94E−03 | N.B. | | | 1.06E−08 | 1.17E+05 |
| SAD10319_P04_G10 | 9.50E−08 | 2.42E+05 | 2.30E−02 | 6.31E−08 | 3.36E+05 | 2.12E−02 | 6.51E−08 | 2.30E+05 |
| SAD10319_P04_B11 | 9.84E−08 | 1.98E+05 | 1.95E−02 | N.B. | | | 8.69E−09 | 3.14E+05 |
| SAD10319_P04_C11 | 3.91E−08 | 3.18E+05 | 1.24E−02 | 1.58E−08 | 2.88E+05 | 4.56E−03 | 1.14E−08 | 2.78E+05 |
| SAD10319_P04_D11 | 9.79E−08 | 1.96E+05 | 1.92E−02 | N.B. | | | 1.43E−08 | 2.36E+05 |
| SAD10319_P04_E11 | 1.67E−07 | 1.39E+05 | 2.33E−02 | N.B. | | | 1.85E−07 | 1.29E+05 |
| SAD10319_P04_F11 | 4.43E−08 | 2.58E+05 | 1.14E−02 | N.B. | | | 6.07E−09 | 3.34E+05 |
| SAD10319_P04_G11 | 8.10E−09 | 3.32E+05 | 2.69E−03 | N.B. | | | 1.52E−08 | 2.08E+05 |
| SAD10319_P04_B12 | N.B. | | | N.B. | | | N.B. | |
| SAD10319_P04_C12 | 2.67E−07 | 9.92E+04 | 2.64E−02 | N.B. | | | 2.17E−07 | 9.01E+04 |
| SAD10319_P04_D12 | 1.40E−07 | 2.01E+05 | 2.82E−02 | N.B. | | | 6.37E−08 | 1.88E+05 |
| SAD10319_P04_E12 | 7.67E−09 | 3.83E+05 | 2.93E−03 | N.B. | | | 1.11E−08 | 2.76E+05 |
| SAD10319_P04_F12 | 4.32E−08 | 3.99E+05 | 1.72E−02 | N.B. | | | 1.04E−07 | 1.55E+05 |
| SAD10319_P04_H12 | 2.91E−07 | 1.10E+05 | 3.20E−02 | 2.93E−08 | 3.41E+05 | 1.00E−02 | 1.53E−08 | 3.07E+05 |
| SAD10320_P01_F01 | 2.78E−07 | 1.12E+05 | 3.10E−02 | 2.21E−08 | 1.99E+05 | 4.40E−03 | 2.78E−08 | 2.31E+05 |
| SAD10320_P01_G01 | 5.91E−07 | 5.62E+04 | 3.32E−02 | N.B. | | | 8.70E−08 | 1.65E+05 |
| SAD10320_P01_H01 | 2.27E−08 | 3.79E+05 | 8.60E−03 | N.B. | | | 2.20E−07 | 9.44E+04 |
| SAD10320_P01_C02 | 3.37E−08 | 2.85E+05 | 9.60E−03 | N.B. | | | 1.22E−08 | 2.27E+05 |
| SAD10320_P01_E02 | 2.54E−07 | 1.44E+05 | 3.66E−02 | 3.75E−08 | 3.76E+05 | 1.41E−02 | 1.79E−08 | 3.26E+05 |
| SAD10320_P01_F02 | 4.97E−08 | 3.23E+05 | 1.60E−02 | N.B. | | | 2.24E−07 | 8.65E+04 |
| SAD10320_P01_G02 | 2.75E−07 | 1.03E+05 | 2.83E−02 | N.B. | | | 2.66E−07 | 7.40E+04 |
| SAD10320_P01_H02 | 2.97E−07 | 1.15E+05 | 3.41E−02 | 1.43E−08 | 2.51E+05 | 3.58E−03 | 1.08E−08 | 3.09E+05 |
| SAD10320_P01_A03 | 1.02E−07 | 2.28E+05 | 2.33E−02 | 1.20E−07 | 1.71E+05 | 2.04E−02 | 3.75E−07 | 7.24E+04 |
| SAD10320_P01_B03 | 6.91E−08 | 2.58E+05 | 1.78E−02 | 1.70E−07 | 1.19E+05 | 2.02E−02 | 2.39E−07 | 8.55E+04 |
| SAD10320_P01_C03 | 4.04E−08 | 4.25E+05 | 1.71E−02 | N.B. | | | 1.73E−07 | 1.13E+05 |
| SAD10320_P01_D03 | 2.33E−07 | 1.39E+05 | 3.23E−02 | 1.02E−08 | 2.06E+05 | 2.10E−03 | 9.56E−09 | 2.76E+05 |
| SAD10320_P01_E03 | 1.05E−07 | 3.14E+05 | 3.30E−02 | 2.14E−08 | 3.10E+05 | 6.64E−03 | 1.69E−07 | 1.32E+05 |
| SAD10320_P01_G03 | 2.55E−07 | 1.28E+05 | 3.27E−02 | N.B. | | | 1.87E−07 | 1.15E+05 |
| SAD10320_P01_H03 | 1.95E−07 | 1.49E+05 | 2.91E−02 | 5.52E−08 | 2.37E+05 | 1.31E−02 | 1.03E−08 | 2.82E+05 |
| SAD10320_P02_A04 | 1.97E−07 | 1.39E+05 | 2.73E−02 | N.B. | | | 2.05E−07 | 8.89E+04 |
| SAD10320_P02_B04 | 8.92E−08 | 2.65E+05 | 2.36E−02 | N.B. | | | 1.39E−07 | 1.26E+05 |
| SAD10320_P02_H04 | 9.38E−08 | 2.88E+05 | 2.70E−02 | 2.82E−08 | 3.02E+05 | 8.50E−03 | 4.50E−07 | 6.18E+04 |
| SAD10320_P02_A05 | 8.47E−08 | 2.63E+05 | 2.23E−02 | N.B. | | | 1.24E−07 | 1.35E+05 |
| SAD10320_P02_B05 | 2.60E−07 | 1.30E+05 | 3.38E−02 | N.B. | | | 9.00E−09 | 2.99E+05 |
| SAD10320_P02_C05 | 4.33E−08 | 3.48E+05 | 1.51E−02 | N.B. | | | 3.56E−07 | 5.80E+04 |
| SAD10320_P02_E05 | P.F. | | | N.B. | | | 1.45E−08 | 2.19E+05 |
| SAD10320_P02_A06 | P.F. | | | N.B. | | | 1.16E−08 | 2.28E+05 |
| SAD10320_P02_D06 | P.F. | | | N.B. | | | 2.34E−08 | 2.65E+05 |
| SAD10320_P02_E06 | P.F. | | | 1.20E−08 | 4.17E+05 | 4.99E−03 | 3.46E−07 | 8.75E+04 |
| SAD10320_P02_F06 | 3.24E−07 | 9.92E+04 | 3.22E−02 | 4.08E−08 | 1.87E+05 | 7.61E−03 | 1.67E−07 | 1.02E+05 |
| SAD10320_P02_G06 | 3.14E−07 | 1.31E+05 | 4.12E−02 | 4.59E−08 | 3.70E+05 | 1.70E−02 | 1.17E−08 | 3.31E+05 |
| SAD10320_P02_H06 | P.F. | | | N.B. | | | 1.05E−08 | 2.68E+05 |
| SAD10320_P03_B07 | 1.20E−07 | 1.51E+05 | 1.82E−02 | N.B. | | | 9.49E−09 | 2.36E+05 |
| SAD10320_P03_E07 | P.F. | | | N.B. | | | 3.38E−08 | 2.07E+05 |
| SAD10320_P03_H07 | N.B. | | | N.B. | | | N.B. | |
| SAD10320_P03_C08 | P.F. | | | N.B. | | | 3.23E−07 | 4.69E+04 |
| SAD10320_P03_D08 | P.F. | | | N.B. | | | 7.76E−09 | 2.49E+05 |
| SAD10320_P03_F08 | P.F. | | | N.B. | | | 1.50E−08 | 1.79E+05 |
| SAD10320_P03_H08 | P.F. | | | N.B. | | | 1.81E−07 | 8.27E+04 |
| SAD10320_P03_A09 | P.F. | | | N.B. | | | 1.46E−08 | 1.99E+05 |
| SAD10320_P03_C09 | P.F. | | | N.B. | | | 3.16E−08 | 1.61E+05 |
| SAD10320_P03_F09 | P.F. | | | N.B. | | | 3.70E−08 | 1.95E+05 |
| SAD10320_P04_A10 | P.F. | | | N.B. | | | 1.43E−07 | 1.01E+05 |
| SAD10320_P04_C10 | P.F. | | | N.B. | | | 1.68E−07 | 1.11E+05 |
| SAD10320_P04_D10 | 1.37E−07 | 1.17E+05 | 1.60E−02 | N.B. | | | 3.09E−07 | 5.54E+04 |
| SAD10320_P04_E10 | P.F. | | | N.B. | | | 1.24E−08 | 2.07E+05 |
| SAD10320_P04_F10 | P.F. | | | N.B. | | | 8.51E−09 | 2.81E+05 |
| SAD10320_P04_G10 | P.F. | | | N.B. | | | 3.52E−07 | 5.38E+04 |
| SAD10320_P04_A11 | P.F. | | | N.B. | | | 2.94E−08 | 2.52E+05 |
| SAD10320_P04_D11 | P.F. | | | N.B. | | | 1.69E−08 | 2.00E+05 |
| SAD10320_P04_E11 | N.B. | | | N.B. | | | 2.01E−08 | 1.48E+05 |
| SAD10320_P04_F11 | P.F. | | | N.B. | | | 9.55E−09 | 2.01E+05 |
| SAD10320_P04_G11 | P.F. | | | N.B. | | | 1.57E−08 | 1.76E+05 |
| SAD10320_P04_A12 | P.F. | | | N.B. | | | 2.40E−07 | 6.97E+04 |
| SAD10320_P04_D12 | P.F. | | | N.B. | | | 1.39E−08 | 1.86E+05 |
| SAD10320_P04_E12 | P.F. | | | N.B. | | | 7.98E−08 | 1.36E+05 |
| SAD10320_P04_F12 | N.B. | | | N.B. | | | 2.33E−08 | 1.11E+05 |
| SAD10319_P05_A01 | 1.04E−07 | 1.86E+05 | 1.94E−02 | N.B. | | | 1.58E−07 | 1.59E+05 |
| SAD10319_P05_A02 | 9.66E−08 | 2.70E+05 | 2.61E−02 | N.B. | | | 1.92E−07 | 1.28E+05 |
| SAD10319_P05_A03 | 3.39E−08 | 4.78E+05 | 1.62E−02 | N.B. | | | 4.16E−08 | 3.06E+05 |
| SAD10319_P05_A05 | 6.04E−08 | 5.20E+05 | 3.14E−02 | N.B. | | | 3.26E−08 | 3.63E+05 |
| SAD10319_P05_B02 | 7.89E−08 | 3.29E+05 | 2.59E−02 | N.B. | | | 1.59E−08 | 2.59E+05 |
| SAD10319_P05_B03 | 6.45E−08 | 4.16E+05 | 2.68E−02 | 4.82E−08 | 3.48E+05 | 1.67E−02 | 2.24E−07 | 1.54E+05 |
| SAD10319_P05_B04 | 6.41E−08 | 4.16E+05 | 2.67E−02 | N.B. | | | 8.81E−09 | 2.93E+05 |
| SAD10319_P05_B05 | 4.47E−08 | 4.44E+05 | 1.99E−02 | N.B. | | | 1.40E−08 | 4.67E+05 |
| SAD10319_P05_C01 | 7.11E−08 | 3.16E+05 | 2.24E−02 | N.B. | | | 3.80E−08 | 3.41E+05 |
| SAD10319_P05_C03 | 6.84E−08 | 3.10E+05 | 2.12E−02 | N.B. | | | 1.24E−08 | 2.63E+05 |
| SAD10319_P05_C05 | 6.69E−08 | 3.62E+05 | 2.42E−02 | N.B. | | | 2.71E−08 | 3.03E+05 |
| SAD10319_P05_D01 | 1.69E−07 | 1.79E+05 | 3.03E−02 | N.B. | | | 3.59E−08 | 2.59E+05 |
| SAD10319_P05_D02 | 2.94E−07 | 1.42E+05 | 4.18E−02 | N.B. | | | 1.44E−07 | 1.68E+05 |
| SAD10319_P05_D03 | 7.40E−08 | 3.18E+05 | 2.36E−02 | N.B. | | | 1.66E−07 | 1.40E+05 |
| SAD10319_P05_D04 | 4.75E−08 | 4.30E+05 | 2.04E−02 | N.B. | | | 1.12E−07 | 1.96E+05 |
| SAD10319_P05_D05 | 8.23E−08 | 3.31E+05 | 2.73E−02 | N.B. | | | 4.69E−08 | 3.37E+05 |
| SAD10319_P05_E04 | 4.33E−08 | 3.37E+05 | 1.46E−02 | N.B. | | | 1.23E−08 | 3.04E+05 |
| SAD10319_P05_F01 | 3.03E−08 | 4.23E+05 | 1.28E−02 | N.B. | | | 1.25E−07 | 1.72E+05 |
| SAD10319_P05_G01 | 5.93E−08 | 4.23E+05 | 2.51E−02 | N.B. | | | 1.36E−07 | 1.74E+05 |
| SAD10319_P05_G02 | 4.30E−08 | 4.96E+05 | 2.13E−02 | N.B. | | | 4.40E−08 | 3.55E+05 |
| SAD10319_P05_G03 | 3.35E−07 | 1.27E+05 | 4.25E−02 | 1.72E−07 | 2.55E+05 | 4.39E−02 | 4.64E−08 | 3.60E+05 |
| SAD10319_P05_G04 | 4.03E−08 | 3.39E+05 | 1.37E−02 | N.B. | | | 1.24E−08 | 3.14E+05 |
| SAD10319_P05_H06 | 1.62E−07 | 1.72E+05 | 2.78E−02 | N.B. | | | 3.41E−08 | 3.04E+05 |
| SAD10319_P06_A07 | N.B. | | | N.B. | | | N.B. | |
| SAD10319_P06_A10 | 1.04E−07 | 2.10E+05 | 2.18E−02 | N.B. | | | 1.61E−07 | 1.52E+05 |
| SAD10319_P06_A11 | 2.18E−08 | 3.55E+05 | 7.74E−03 | N.B. | | | 1.02E−08 | 2.74E+05 |
| SAD10319_P06_B10 | 5.00E−08 | 3.52E+05 | 1.76E−02 | N.B. | | | 6.34E−09 | 3.15E+05 |
| SAD10319_P06_B11 | 2.31E−07 | 1.33E+05 | 3.07E−02 | N.B. | | | 3.55E−08 | 2.85E+05 |
| SAD10319_P06_C10 | 8.77E−08 | 2.36E+05 | 2.07E−02 | N.B. | | | 1.03E−08 | 3.07E+05 |
| SAD10319_P06_C12 | 1.10E−07 | 2.17E+05 | 2.39E−02 | N.B. | | | 3.20E−08 | 2.36E+05 |
| SAD10319_P06_D12 | 7.06E−08 | 3.90E+05 | 2.76E−02 | N.B. | | | 5.30E−08 | 3.63E+05 |
| SAD10319_P06_E08 | 1.24E−07 | 2.68E+05 | 3.33E−02 | N.B. | | | 2.86E−08 | 3.67E+05 |
| SAD10319_P06_E09 | 3.53E−08 | 4.58E+05 | 1.62E−02 | N.B. | | | 3.41E−08 | 3.57E+05 |
| SAD10319_P06_E10 | 8.63E−09 | 4.14E+05 | 3.58E−03 | N.B. | | | 1.98E−08 | 2.90E+05 |
| SAD10319_P06_F07 | 1.39E−07 | 1.71E+05 | 2.38E−02 | N.B. | | | 2.83E−07 | 8.84E+04 |
| SAD10319_P06_F10 | 7.82E−08 | 3.13E+05 | 2.45E−02 | N.B. | | | 1.91E−08 | 3.47E+05 |
| SAD10319_P06_G09 | 5.08E−08 | 3.01E+05 | 1.53E−02 | N.B. | | | 7.42E−09 | 3.48E+05 |
| SAD10319_P06_G11 | 4.18E−08 | 3.83E+05 | 1.60E−02 | N.B. | | | 2.79E−08 | 3.31E+05 |
| SAD10319_P06_H07 | 1.04E−08 | 3.74E+05 | 3.90E−03 | N.B. | | | 4.11E−08 | 2.19E+05 |
| SAD10319_P06_H08 | 9.89E−08 | 2.93E+05 | 2.89E−02 | N.B. | | | 1.39E−07 | 1.66E+05 |
| SAD10319_P06_H10 | 1.15E−08 | 4.14E+05 | 4.74E−03 | N.B. | | | 2.15E−07 | 1.24E+05 |
| SAD10319_P06_H11 | 9.27E−09 | 3.81E+05 | 3.53E−03 | N.B. | | | 2.33E−07 | 1.22E+05 |
| LAD9953_P01_H01 | 3.99E−08 | 3.50E+05 | 1.39E−02 | 5.83E−08 | 3.59E+05 | 2.10E−02 | 6.14E−08 | 2.86E+05 |
| LAD9954_P01_B02 | 1.63E−07 | 1.68E+05 | 2.73E−02 | 1.13E−07 | 2.63E+05 | 2.98E−02 | 3.89E−07 | 7.78E+04 |
| LAD9955_P01_G02 | 1.05E−08 | 3.32E+05 | 3.48E−03 | 3.71E−08 | 3.31E+05 | 1.23E−02 | 5.29E−07 | 5.34E+04 |
| LAD9956_P01_C03 | N.B. | | | N.B. | | | 1.87E−09 | 4.89E+05 |
| LAD9959_P01_E04 | 4.57E−09 | 4.17E+05 | 1.91E−03 | 4.78E−08 | 3.78E+05 | 1.80E−02 | 4.45E−09 | 3.91E+05 |
| LAD9960_P01_D05 | N.B. | | | N.B. | | | N.B. | |
| LAD9963_P01_E06 | 3.56E−09 | 6.03E+05 | 2.14E−03 | N.B. | | | 2.42E−09 | 3.58E+05 |
| LAD9964_P01_C07 | 1.10E−09 | 3.56E+05 | 3.92E−04 | 4.25E−09 | 3.92E+05 | 1.67E−03 | 1.50E−09 | 4.25E+05 |
| LAD9966_P01_A08 | 1.98E−07 | 1.34E+05 | 2.66E−02 | 5.08E−08 | 2.67E+05 | 1.36E−02 | 9.80E−09 | 3.58E+05 |

| | | | | | PSR | |
|---|---|---|---|---|---|---|
| | Cyno CD3 pH 6.0 | Cyno CD3 pH 7.4 | | | normalized | PSR |
| Clone # | koff | KD | kon | koff | MFI | Score |
| SAD10318_P01_D01 | 1.84E−02 | N.B. | | | 106.85 | 0.04 |
| SAD10318_P01_E01 | 1.49E−02 | N.B. | | | 106.69 | 0.04 |
| SAD10318_P01_A03 | 1.54E−02 | N.B. | | | 109.86 | 0.05 |
| SAD10318_P02_C04 | 9.83E−03 | 9.77E−08 | 1.75E+05 | 1.71E−02 | 132.21 | 0.10 |
| SAD10318_P02_A05 | 8.53E−04 | 3.24E−08 | 2.32E+05 | 7.51E−03 | 252.57 | 0.13 |
| SAD10319_P03_E07 | 1.31E−02 | N.B. | | | 94.38 | 0.01 |
| SAD10320_P01_B01 | 1.11E−02 | N.B. | | | 112.55 | 0.05 |
| SAD10320_P01_E01 | 1.46E−02 | N.B. | | | 109.18 | 0.05 |
| SAD10320_P02_D05 | 7.66E−04 | 2.93E−08 | 2.68E+05 | 7.87E−03 | 123.53 | 0.08 |
| SAD10320_P02_H05 | 1.12E−03 | 9.90E−08 | 1.76E+05 | 1.74E−02 | 131.9 | 0.10 |
| (ADI-48652) | | | | | | |
| LAD9965_P01_H07 | 8.33E−03 | 1.15E−07 | 2.08E+05 | 2.39E−02 | 341.62 | 0.16 |
| SAD10319_P03_C07 | 1.78E−02 | N.B. | | | 3195.94 | 0.532 |
| LAD5224_P03_A01 | 4.90E−04 | 7.08E−09 | 3.46E+05 | 2.45E−03 | 2930.88 | 0.492 |
| SAD10318_P01_F01 | 1.68E−02 | N.B. | | | 966.39 | 0.209 |
| SAD10318_P01_C03 | 2.21E−02 | 6.77E−08 | 2.23E+05 | 1.51E−02 | 8340.98 | 0.920 |
| SAD10318_P01_E03 | 3.89E−03 | N.B. | | | 3888.12 | 0.637 |
| SAD10318_P02_B04 | 2.63E−03 | N.B. | | | 1444.56 | 0.273 |
| SAD10318_P02_D04 | 1.63E−02 | N.B. | | | 1789.14 | 0.320 |
| SAD10318_P02_E04 | 1.82E−02 | 5.74E−08 | 2.82E+05 | 1.62E−02 | 1707.85 | 0.309 |
| SAD10318_P02_F05 | 2.42E−02 | 6.40E−08 | 2.57E+05 | 1.65E−02 | 5954.14 | 0.806 |
| SAD10318_P02_G05 | 2.59E−02 | 6.49E−08 | 3.53E+05 | 2.29E−02 | 2186.89 | 0.379 |
| SAD10318_P02_B06 | 9.05E−03 | N.B. | | | 7212.32 | 0.876 |
| SAD10318_P02_D06 | 1.86E−02 | N.B. | | | 2313.88 | 0.398 |
| SAD10318_P02_E06 | 1.83E−02 | N.B. | | | 8302.93 | 0.919 |
| SAD10318_P02_G06 | 2.16E−02 | 1.01E−07 | 3.18E+05 | 3.22E−02 | 7435.47 | 0.886 |
| SAD10318_P03_B07 | 4.82E−03 | N.B. | | | 4829.08 | 0.724 |
| SAD10318_P03_F07 | 2.15E−03 | N.B. | | | 4533.75 | 0.700 |
| SAD10318_P03_G07 | 1.83E−03 | N.B. | | | 1611.89 | 0.296 |
| SAD10318_P03_A08 | 6.00E−03 | N.B. | | | 711.16 | 0.174 |
| SAD10318_P03_B08 | 3.03E−03 | N.B. | | | 3156.39 | 0.526 |
| SAD10318_P03_G08 | 5.44E−03 | N.B. | | | 5617.87 | 0.783 |
| SAD10318_P03_F09 | 1.39E−03 | N.B. | | | 588.33 | 0.158 |
| SAD10318_P03_G09 | 5.59E−03 | N.B. | | | 998.08 | 0.213 |
| SAD10318_P04_D10 | 2.51E−03 | N.B. | | | 2579.89 | 0.438 |
| SAD10318_P04_E10 | 2.45E−03 | N.B. | | | 1465.46 | 0.276 |
| SAD10318_P04_F10 | 4.36E−03 | N.B. | | | 6953.75 | 0.864 |
| SAD10318_P04_H10 | 2.84E−03 | N.B. | | | 541.41 | 0.151 |
| SAD10318_P04_A11 | | N.B. | | | 8475.24 | 0.924 |
| SAD10318_P04_E11 | 2.56E−03 | N.B. | | | 2696.05 | 0.456 |
| SAD10318_P04_F11 | 1.83E−03 | N.B. | | | 4244.97 | 0.677 |
| SAD10318_P04_G11 | 2.15E−03 | N.B. | | | 2967.69 | 0.497 |
| SAD10318_P04_F12 | | N.B. | | | 6479.03 | 0.838 |
| SAD10318_P04_G12 | 2.50E−03 | N.B. | | | 607.37 | 0.160 |
| SAD10318_P04_H12 | 2.54E−03 | N.B. | | | 489.06 | 0.144 |
| SAD10319_P01_A01 | 3.28E−02 | N.B. | | | 12595.42 | 0.981 |
| SAD10319_P01_C01 | 4.02E−03 | N.B. | | | 10382.82 | 0.963 |
| SAD10319_P01_D01 | 9.46E−03 | N.B. | | | 11570.57 | 0.975 |
| SAD10319_P01_E01 | 2.81E−02 | N.B. | | | 8875.40 | 0.935 |
| SAD10319_P01_F01 | 1.79E−02 | N.B. | | | 9035.92 | 0.939 |
| SAD10319_P01_G01 | 2.49E−03 | N.B. | | | 11014.25 | 0.970 |
| SAD10319_P0l_A02 | 1.65E−02 | N.B. | | | 10609.41 | 0.965 |
| SAD10319_P01_C02 | 2.51E−02 | N.B. | | | 14703.85 | 0.987 |
| SAD10319_P01_D02 | 6.86E−03 | N.B. | | | 1104.87 | 0.227 |
| SAD10319_P01_E02 | 2.48E−02 | 1.45E−07 | 2.45E+05 | 3.55E−02 | 11649.96 | 0.975 |
| SAD10319_P01_F02 | 5.22E−03 | N.B. | | | 9918.89 | 0.956 |
| SAD10319_P01_H02 | 3.14E−02 | N.B. | | | 11677.80 | 0.975 |
| SAD10319_P01_B03 | 2.45E−02 | N.B. | | | 9527.89 | 0.949 |
| SAD10319_P01_C03 | 1.96E−02 | N.B. | | | 12864.36 | 0.982 |
| SAD10319_P01_D03 | 2.21E−03 | N.B. | | | 12525.77 | 0.980 |
| SAD10319_P02_A04 | 2.84E−02 | N.B. | | | 6185.14 | 0.820 |
| SAD10319_P02_B04 | 2.03E−02 | N.B. | | | 12957.80 | 0.982 |
| SAD10319_P02_C04 | 2.15E−02 | N.B. | | | 11399.52 | 0.973 |
| SAD10319_P02_D04 | 3.22E−03 | N.B. | | | 13848.24 | 0.985 |
| SAD10319_P02_E04 | 2.15E−02 | 6.41E−08 | 3.24E+05 | 2.08E−02 | 14297.04 | 0.986 |
| SAD10319_P02_F04 | 3.17E−02 | 1.17E−07 | 2.57E+05 | 3.01E−02 | 12927.52 | 0.982 |
| SAD10319_P02_H04 | 3.13E−03 | N.B. | | | 13710.09 | 0.985 |
| SAD10319_P02_A05 | 2.69E−02 | N.B. | | | 13622.12 | 0.984 |
| SAD10319_P02_B05 | 3.88E−02 | N.B. | | | 15510.92 | 0.988 |
| SAD10319_P02_C05 | 6.97E−03 | N.B. | | | 13558.13 | 0.984 |
| SAD10319_P02_D05 | 2.96E−03 | N.B. | | | 7092.88 | 0.871 |
| SAD10319_P02_F05 | 2.62E−02 | N.B. | | | 12207.03 | 0.979 |
| SAD10319_P02_G05 | 4.75E−03 | N.B. | | | 12832.35 | 0.982 |
| SAD10319_P02_H05 | 1.90E−02 | N.B. | | | 13554.59 | 0.984 |
| SAD10319_P02_A06 | 3.27E−02 | 3.02E−08 | 3.83E+05 | 1.15E−02 | 13214.36 | 0.983 |
| SAD10319_P02_B06 | 5.31E−03 | N.B. | | | 11753.86 | 0.976 |
| SAD10319_P02_C06 | 2.30E−02 | 5.13E−08 | 4.96E+05 | 2.55E−02 | 18024.91 | 0.989 |
| SAD10319_P02_D06 | 2.12E−02 | N.B. | | | 9677.15 | 0.952 |
| SAD10319_P02_E06 | 2.51E−03 | 6.06E−08 | 4.40E+05 | 2.67E−02 | 16828.70 | 0.989 |
| SAD10319_P02_F06 | 1.22E−02 | 1.34E−07 | 2.47E+05 | 3.31E−02 | 13621.05 | 0.984 |
| SAD10319_P02_G06 | 2.42E−02 | N.B. | | | 14664.07 | 0.987 |
| SAD10319_P02_H06 | 1.88E−02 | 7.80E−08 | 2.20E+05 | 1.71E−02 | 12408.54 | 0.980 |
| SAD10319_P03_G07 | 1.87E−02 | N.B. | | | 469.51 | 0.142 |
| SAD10319_P03_H07 | 2.01E−02 | N.B. | | | 14249.51 | 0.986 |
| SAD10319_P03_D08 | 2.00E−02 | N.B. | | | 12086.73 | 0.978 |
| SAD10319_P03_E08 | 1.97E−02 | N.B. | | | 1182.04 | 0.238 |
| SAD10319_P03_G08 | 1.68E−02 | N.B. | | | 12995.09 | 0.982 |
| SAD10319_P03_H08 | 3.48E−03 | N.B. | | | 14218.49 | 0.986 |
| SAD10319_P03_F09 | 1.99E−02 | N.B. | | | 12221.31 | 0.979 |
| SAD10319_P03_G09 | 2.19E−02 | N.B. | | | 13882.32 | 0.985 |
| SAD10319_P04_A10 | 2.62E−02 | N.B. | | | 15945.11 | 0.988 |
| SAD10319_P04_B10 | 1.27E−02 | 5.88E−08 | 3.38E+05 | 1.99E−02 | 14000.13 | 0.985 |
| SAD10319_P04_C10 | 1.24E−03 | N.B. | | | 14340.11 | 0.986 |
| SAD10319_P04_G10 | 1.50E−02 | 6.26E−08 | 2.91E+05 | 1.82E−02 | 10206.29 | 0.960 |
| SAD10319_P04_B11 | 2.73E−03 | N.B. | | | 11397.82 | 0.973 |
| SAD10319_P04_C11 | 3.17E−03 | N.B. | | | 12612.64 | 0.981 |
| SAD10319_P04_D11 | 3.39E−03 | N.B. | | | 12771.61 | 0.981 |
| SAD10319_P04_E11 | 2.39E−02 | N.B. | | | 14750.42 | 0.987 |
| SAD10319_P04_F11 | 2.02E−03 | N.B. | | | 13416.98 | 0.984 |
| SAD10319_P04_G11 | 3.17E−03 | N.B. | | | 7622.78 | 0.894 |
| SAD10319_P04_B12 | | N.B. | | | 15406.32 | 0.988 |
| SAD10319_P04_C12 | 1.96E−02 | N.B. | | | 11966.44 | 0.977 |
| SAD10319_P04_D12 | 1.20E−02 | N.B. | | | 11989.11 | 0.977 |
| SAD10319_P04_E12 | 3.07E−03 | N.B. | | | 12187.23 | 0.979 |
| SAD10319_P04_F12 | 1.62E−02 | N.B. | | | 2421.46 | 0.414 |
| SAD10319_P04_H12 | 4.70E−03 | N.B. | | | 13324.27 | 0.983 |
| SAD10320_P01_F01 | 6.42E−03 | N.B. | | | 5311.73 | 0.761 |
| SAD10320_P01_G01 | 1.44E−02 | N.B. | | | 560.62 | 0.154 |
| SAD10320_P01_H01 | 2.07E−02 | N.B. | | | 3157.88 | 0.526 |
| SAD10320_P01_C02 | 2.77E−03 | N.B. | | | 2863.73 | 0.481 |
| SAD10320_P01_E02 | 5.85E−03 | 6.86E−08 | 3.04E+05 | 2.08E−02 | 7659.32 | 0.896 |
| SAD10320_P01_F02 | 1.94E−02 | N.B. | | | 667.21 | 0.168 |
| SAD10320_P01_G02 | 1.97E−02 | N.B. | | | 601.03 | 0.160 |
| SAD10320_P01_H02 | 3.34E−03 | N.B. | | | 868.35 | 0.196 |
| SAD10320_P01_A03 | 2.71E−02 | 7.84E−08 | 2.22E+05 | 1.74E−02 | 11115.10 | 0.971 |
| SAD10320_P01_B03 | 2.04E−02 | 7.51E−08 | 2.12E+05 | 1.59E−02 | 3130.48 | 0.522 |
| SAD10320_P01_C03 | 1.95E−02 | N.B. | | | 644.83 | 0.165 |
| SAD10320_P01_D03 | 2.63E−03 | N.B. | | | 690.14 | 0.172 |
| SAD10320_P01_E03 | 2.23E−02 | N.B. | | | 5363.88 | 0.764 |
| SAD10320_P01_G03 | 2.15E−02 | N.B. | | | 651.23 | 0.166 |
| SAD10320_P01_H03 | 2.91E−03 | N.B. | | | 479.13 | 0.143 |
| SAD10320_P02_A04 | 1.82E−02 | N.B. | | | 1289.55 | 0.252 |
| SAD10320_P02_B04 | 1.76E−02 | N.B. | | | 711.54 | 0.174 |
| SAD10320_P02_H04 | 2.78E−02 | N.B. | | | 670.12 | 0.169 |
| SAD10320_P02_A05 | 1.68E−02 | N.B. | | | 544.78 | 0.152 |
| SAD10320_P02_B05 | 2.69E−03 | N.B. | | | 461.81 | 0.141 |
| SAD10320_P02_C05 | 2.07E−02 | N.B. | | | 508.90 | 0.147 |
| SAD10320_P02_E05 | 3.18E−03 | N.B. | | | 2084.14 | 0.363 |
| SAD10320_P02_A06 | 2.64E−03 | N.B. | | | 399.07 | 0.132 |
| SAD10320_P02_D06 | 6.21E−03 | N.B. | | | 728.55 | 0.177 |
| SAD10320_P02_E06 | 3.03E−02 | P.F. | | | 813.34 | 0.188 |
| SAD10320_P02_F06 | 1.70E−02 | 1.41E−07 | 1.18E+05 | 1.66E−02 | 747.20 | 0.179 |
| SAD10320_P02_G06 | 3.87E−03 | 6.48E−08 | 3.38E+05 | 2.19E−02 | 8416.42 | 0.922 |
| SAD10320_P02_H06 | 2.81E−03 | N.B. | | | 584.38 | 0.157 |
| SAD10320_P03_B07 | 2.24E−03 | N.B. | | | 381.32 | 0.130 |
| SAD10320_P03_E07 | 6.99E−03 | N.B. | | | 1387.75 | 0.266 |
| SAD10320_P03_H07 | | N.B. | | | 411.64 | 0.134 |
| SAD10320_P03_C08 | 1.52E−02 | N.B. | | | 421.69 | 0.135 |
| SAD10320_P03_D08 | 1.94E−03 | N.B. | | | 453.40 | 0.140 |
| SAD10320_P03_F08 | 2.68E−03 | N.B. | | | 354.99 | 0.126 |
| SAD10320_P03_H08 | 1.50E−02 | N.B. | | | 715.32 | 0.175 |
| SAD10320_P03_A09 | 2.90E−03 | N.B. | | | 513.45 | 0.148 |
| SAD10320_P03_C09 | 5.10E−03 | N.B. | | | 426.08 | 0.136 |
| SAD10320_P03_F09 | 7.19E−03 | N.B. | | | 464.19 | 0.141 |
| SAD10320_P04_A10 | 1.44E−02 | N.B. | | | 442.99 | 0.138 |
| SAD10320_P04_C10 | 1.87E−02 | N.B. | | | 551.61 | 0.153 |
| SAD10320_P04_D10 | 1.71E−02 | N.B. | | | 410.79 | 0.134 |
| SAD10320_P04_E10 | 2.58E−03 | N.B. | | | 387.67 | 0.131 |
| SAD10320_P04_F10 | 2.39E−03 | N.B. | | | 556.30 | 0.153 |
| SAD10320_P04_G10 | 1.89E−02 | N.B. | | | 405.64 | 0.133 |
| SAD10320_P04_A11 | 7.41E−03 | N.B. | | | 6465.01 | 0.837 |
| SAD10320_P04_D11 | 3.38E−03 | N.B. | | | 441.27 | 0.138 |
| SAD10320_P04_E11 | 2.97E−03 | N.B. | | | 420.25 | 0.135 |
| SAD10320_P04_F11 | 1.92E−03 | N.B. | | | 358.95 | 0.127 |
| SAD10320_P04_G11 | 2.76E−03 | N.B. | | | 466.52 | 0.141 |
| SAD10320_P04_A12 | 1.68E−02 | N.B. | | | 438.81 | 0.138 |
| SAD10320_P04_D12 | 2.59E−03 | N.B. | | | 333.12 | 0.123 |
| SAD10320_P04_E12 | 1.08E−02 | N.B. | | | 419.76 | 0.135 |
| SAD10320_P04_F12 | 2.59E−03 | N.B. | | | 398.32 | 0.132 |
| SAD10319_P05_A01 | 2.51E−02 | N.B. | | | 4666.39 | 0.779 |
| SAD10319_P05_A02 | 2.46E−02 | N.B. | | | 1790.75 | 0.327 |
| SAD10319_P05_A03 | 1.27E−02 | N.B. | | | 7039.62 | 0.921 |
| SAD10319_P05_A05 | 1.18E−02 | N.B. | | | 4424.65 | 0.758 |
| SAD10319_P05_B02 | 4.11E−03 | N.B. | | | 2320.59 | 0.436 |
| SAD10319_P05_B03 | 3.46E−02 | N.B. | | | 3168.44 | 0.614 |
| SAD10319_P05_B04 | 2.58E−03 | N.B. | | | 1899.18 | 0.348 |
| SAD10319_P05_B05 | 6.54E−03 | N.B. | | | 1510.15 | 0.288 |
| SAD10319_P05_C01 | 1.30E−02 | N.B. | | | 2867.44 | 0.551 |
| SAD10319_P05_C03 | 3.27E−03 | N.B. | | | 2332.50 | 0.439 |
| SAD10319_P05_C05 | 8.21E−03 | N.B. | | | 10599.58 | 0.981 |
| SAD10319_P05_D01 | 9.29E−03 | N.B. | | | 867.37 | 0.200 |
| SAD10319_P05_D02 | 2.41E−02 | N.B. | | | 7439.59 | 0.935 |
| SAD10319_P05_D03 | 2.33E−02 | N.B. | | | 2514.09 | 0.477 |
| SAD10319_P05_D04 | 2.20E−02 | N.B. | | | 5563.17 | 0.847 |
| SAD10319_P05_D05 | 1.58E−02 | N.B. | | | 10322.46 | 0.979 |
| SAD10319_P05_E04 | 3.73E−03 | N.B. | | | 6664.59 | 0.907 |
| SAD10319_P05_F01 | 2.14E−02 | N.B. | | | 2559.09 | 0.486 |
| SAD10319_P05_G01 | 2.36E−02 | N.B. | | | 9442.16 | 0.972 |
| SAD10319_P05_G02 | 1.56E−02 | N.B. | | | 14061.28 | 0.989 |
| SAD10319_P05_G03 | 1.67E−02 | N.B. | | | 3988.13 | 0.718 |
| SAD10319_P05_G04 | 3.89E−03 | N.B. | | | 6306.93 | 0.890 |
| SAD10319_P05_H06 | 1.04E−02 | N.B. | | | 903.57 | 0.205 |
| SAD10319_P06_A07 | | N.B. | | | 9774.23 | 0.975 |
| SAD10319_P06_A10 | 2.46E−02 | N.B. | | | 5273.91 | 0.827 |
| SAD10319_P06_A11 | 2.79E−03 | N.B. | | | 4508.80 | 0.765 |
| SAD10319_P06_B10 | 2.00E−03 | N.B. | | | 4527.38 | 0.767 |
| SAD10319_P06_B11 | 1.01E−02 | N.B. | | | 2873.61 | 0.552 |
| SAD10319_P06_C10 | 3.18E−03 | N.B. | | | 4789.69 | 0.789 |
| SAD10319_P06_C12 | 7.56E−03 | N.B. | | | 8492.41 | 0.959 |
| SAD10319_P06_D12 | 1.92E−02 | N.B. | | | 682.94 | 0.174 |
| SAD10319_P06_E08 | 1.05E−02 | N.B. | | | 5829.53 | 0.864 |
| SAD10319_P06_E09 | 1.22E−02 | N.B. | | | 2799.30 | 0.537 |
| SAD10319_P06_E10 | 5.75E−03 | N.B. | | | 4760.14 | 0.787 |
| SAD10319_P06_F07 | 2.51E−02 | N.B. | | | 3893.41 | 0.709 |
| SAD10319_P06_F10 | 6.63E−03 | N.B. | | | 5273.02 | 0.827 |
| SAD10319_P06_G09 | 2.58E−03 | N.B. | | | 3588.20 | 0.679 |
| SAD10319_P06_G11 | 9.24E−03 | N.B. | | | 272.35 | 0.118 |
| SAD10319_P06_H07 | 9.01E−03 | N.B. | | | 9754.77 | 0.975 |
| SAD10319_P06_H08 | 2.31E−02 | N.B. | | | 3100.70 | 0.600 |
| SAD10319_P06_H10 | 2.67E−02 | N.B. | | | 8924.26 | 0.966 |
| SAD10319_P06_H11 | 2.85E−02 | N.B. | | | 235.23 | 0.113 |
| LAD9953_P01_H01 | 1.76E−02 | 8.67E−08 | 2.42E+05 | 2.09E−02 | 313.67 | 0.141 |
| LAD9954_P01_B02 | 3.03E−02 | 3.39E−08 | 3.16E+05 | 1.07E−02 | 174.62 | 0.109 |
| LAD9955_P01_G02 | 2.82E−02 | 1.27E−07 | 1.47E+05 | 1.87E−02 | 163.20 | 0.106 |
| LAD9956_P01_C03 | 9.13E−04 | N.B. | | | 5488.72 | 0.904 |
| LAD9959_P01_E04 | 1.74E−03 | 8.96E−08 | 2.10E+05 | 1.89E−02 | 473.64 | 0.178 |
| LAD9960_P01_D05 | | N.B. | | | 1266.36 | 0.356 |
| LAD9963_P01_E06 | 8.66E−04 | N.B. | | | 5377.02 | 0.898 |
| LAD9964_P01_C07 | 6.37E−04 | 4.71E−09 | 3.10E+05 | 1.46E−03 | 890.84 | 0.274 |
| LAD9966_P01_A08 | 3.50E−03 | N.B. | | | 110.57 | 0.044 |

TABLE 3
Cell Binding Data and Monovalent Equilibrium Dissociation Constants (Kds)

| | | Human CD3+ Jurkat | | | CHO-S | | K_{D} (nM) | | | |
|---|---|---|---|---|---|---|---|---|---|---|
| | | Cell Binding | | | Cell Binding | | for Human CD3 | | | |
| | | MFI | MFI | MFI pH 6.0/ | MFI | MFI | | | | |
| Clone # | ADI | pH 7.4 | pH 6.0 | MFI pH 7.4 | pH 7.4 | pH 6.0 | pH 6.0 | | pH 7.4 | |
| LAD5224_P03_A01 | ADI-26906 | 5273 | 6038 | 1.1 | 472 | 228 | 1.14 | nM | 2.74 | nM |
| SAD10318_P01_B01 | ADI-48574 | 150 | 3553 | 23.7 | 82 | 298 | 74.6 | nM | 24.2 | nM |
| SAD10318_P01_C01 | ADI-48575 | 81 | 3114 | 38.4 | 83 | 438 | 74.4 | nM | Non-binder | |
| SAD10318_P01_D01 | ADI-48576 | 42 | 849 | 20.1 | 93 | 276 | 76.0 | nM | Non-binder | |
| SAD10318_P01_E01 | ADI-48577 | 73 | 5372 | 73.1 | 84 | 291 | 116 | nM | Non-binder | |
| SAD10318_P01_F01 | ADI-48578 | 36 | 418 | 11.7 | 69 | 197 | 73.2 | nM | Non-binder | |
| SAD10318_P01_G01 | ADI-48579 | 306 | 2069 | 6.8 | 84 | 229 | 40.4 | nM | Non-binder | |
| SAD10318_P01_H01 | ADI-48580 | 41 | 933 | 22.5 | 81 | 1398 | 52.1 | nM | Non-binder | |
| SAD10318_P01_A02 | ADI-48581 | 38 | 1728 | 45.5 | 78 | 311 | 69.7 | nM | Non-binder | |
| SAD10318_P01_B02 | ADI-48582 | 36 | 326 | 9.0 | 74 | 321 | 48.4 | nM | Non-binder | |
| SAD10318_P01_C02 | ADI-48583 | 41 | 157 | 3.8 | 82 | 248 | 36.3 | nM | Non-binder | |
| SAD10318_P01_D02 | ADI-48584 | 91 | 2179 | 23.9 | 79 | 1585 | 61.6 | nM | Non-binder | |
| SAD10318_P01_F02 | ADI-48585 | 45 | 381 | 8.5 | 79 | 274 | 50.7 | nM | Non-binder | |
| SAD10318_P01_G02 | ADI-48586 | 45 | 1090 | 24.3 | 78 | 154 | 57.6 | nM | Non-binder | |
| SAD10318_P01_A03 | ADI-48587 | 66 | 5117 | 77.1 | 75 | 154 | 36.6 | nM | Non-binder | |
| SAD10318_P01_D03 | ADI-48588 | 59 | 4935 | 83.3 | 74 | 142 | 67.6 | nM | Non-binder | |
| SAD10318_P01_F03 | ADI-48589 | 475 | 5237 | 11.0 | 75 | 175 | 70.2 | nM | 131 | nM |
| SAD10318_P01_G03 | ADI-48590 | 32 | 1948 | 60.3 | 69 | 93 | 46.8 | nM | Non-binder | |
| SAD10318_P01_H03 | ADI-48591 | 140 | 4434 | 31.6 | 73 | 98 | 49.2 | nM | Non-binder | |
| SAD10318_P02_C04 | ADI-48592 | 2987 | 5807 | 1.9 | 83 | 339 | 23.5 | nM | 128 | nM |
| SAD10318_P02_G04 | ADI-48593 | 96 | 3785 | 39.4 | 73 | 398 | 53.7 | nM | Non-binder | |
| SAD10318_P02_H04 | ADI-48594 | 1319 | 4262 | 3.2 | 164 | 153 | 54.4 | nM | 77.0 | nM |
| SAD10318_P02_A05 | ADI-48595 | 4956 | 5984 | 1.2 | 117 | 247 | 1.56 | nM | 37.7 | nM |
| SAD10318_P02_B05 | ADI-48596 | 828 | 500 | 0.6 | 75 | 148 | 27.9 | nM | 102 | nM |
| SAD10318_P02_C05 | ADI-48597 | 61 | 1316 | 21.7 | 80 | 743 | 62.7 | nM | Non-binder | |
| SAD10318_P02_D05 | ADI-48598 | 33 | 279 | 8.6 | 69 | 316 | 36.9 | nM | Non-binder | |
| SAD10318_P02_H05 | ADI-48599 | 583 | 1319 | 2.3 | 79 | 273 | 28.7 | nM | 66.6 | nM |
| SAD10318_P02_G06 | ADI-48600 | 1337 | 2900 | 2.2 | 76 | 108 | 80.5 | nM | 82.1 | nM |
| SAD10318_P03_E07 | ADI-48601 | 52 | 1080 | 20.7 | 85 | 318 | 62.0 | nM | Non-binder | |
| SAD10318_P03_F07 | ADI-48602 | 34 | 202 | 5.9 | 78 | 459 | 33.4 | nM | Non-binder | |
| SAD10318_P03_C08 | ADI-48603 | 51 | 177 | 3.5 | 76 | 360 | 36.7 | nM | Non-binder | |
| SAD10318_P03_D08 | ADI-48604 | 43 | 199 | 4.7 | 77 | 219 | 27.5 | nM | Non-binder | |
| SAD10318_P03_E08 | ADI-48605 | 55 | 282 | 5.2 | 84 | 1566 | 50.7 | nM | Non-binder | |
| SAD10318_P03_F08 | ADI-48606 | 76 | 89 | 1.2 | 87 | 280 | 45.6 | nM | Non-binder | |
| SAD10318_P03_H08 | ADI-48607 | 52 | 86 | 1.7 | 76 | 204 | 30.4 | nM | Non-binder | |
| SAD10318_P03_A09 | ADI-48608 | 45 | 203 | 4.5 | 78 | 227 | 61.9 | nM | Non-binder | |
| SAD10318_P03_C09 | ADI-48609 | 37 | 346 | 9.3 | 76 | 267 | 39.2 | nM | Non-binder | |
| SAD10318_P03_D09 | ADI-48610 | 72 | 361 | 5.0 | 82 | 4056 | 33.1 | nM | Non-binder | |
| SAD10318_P03_E09 | ADI-48611 | 47 | 123 | 2.6 | 83 | 263 | 49.2 | nM | Non-binder | |
| SAD10318_P04_C10 | ADI-48612 | 50 | 42 | 0.8 | 76 | 157 | 34.1 | nM | Non-binder | |
| SAD10318_P04_D10 | ADI-48613 | 66 | 52 | 0.8 | 86 | 305 | 29.7 | nM | Non-binder | |
| SAD10318_P04_B11 | ADI-48614 | 58 | 148 | 2.6 | 86 | 302 | 35.5 | nM | Non-binder | |
| SAD10318_P04_C11 | ADI-48615 | 37 | 40 | 1.1 | 73 | 214 | 139 | nM | Non-binder | |
| SAD10318_P04_D11 | ADI-48616 | 41 | 36 | 0.9 | 73 | 159 | 29.9 | nM | Non-binder | |
| SAD10318_P04_H11 | ADI-48617 | 53 | 166 | 3.1 | 85 | 334 | 62.9 | nM | Non-binder | |
| SAD10318_P04_A12 | ADI-48618 | 41 | 170 | 4.2 | 78 | 2495 | 40.0 | nM | Non-binder | |
| SAD10319_P01_A01 | ADI-48619 | 129 | 1746 | 13.5 | 101 | 615 | 49.1 | nM | Non-binder | |
| SAD10319_P01_B01 | ADI-48620 | 28 | 191 | 6.9 | 140 | 130 | 30.3 | nM | Non-binder | |
| SAD10319_P01_C01 | ADI-48621 | 49 | 731 | 14.8 | 103 | 230 | 30.6 | nM | Non-binder | |
| SAD10319_P01_D01 | ADI-48622 | 567 | 3057 | 5.4 | 627 | 299 | 32.5 | nM | Non-binder | |
| SAD10319_P01_H01 | ADI-48623 | 147 | 1757 | 12.0 | 100 | 1276 | 40.5 | nM | Non-binder | |
| SAD10319_P01_F02 | ADI-48624 | 58 | 895 | 15.4 | 94 | 249 | 39.4 | nM | Non-binder | |
| SAD10319_P01_H02 | ADI-48625 | 128 | 1731 | 13.6 | 160 | 1003 | 45.2 | nM | Non-binder | |
| SAD10319_P01_C03 | ADI-48626 | 369 | 3163 | 8.6 | 286 | 272 | 36.9 | nM | Non-binder | |
| SAD10319_P01_F03 | ADI-48627 | 45 | 335 | 7.4 | 86 | 234 | 49.7 | nM | Non-binder | |
| SAD10319_P01_H03 | ADI-48628 | 132 | 1756 | 13.3 | 98 | 444 | 49.3 | nM | Non-binder | |
| SAD10319_P02_A05 | ADI-48629 | 499 | 1778 | 3.6 | 179 | 558 | 37.6 | nM | 427 | nM |
| SAD10319_P02_E05 | ADI-48630 | 5062 | 5540 | 1.1 | 78 | 129 | 23.9 | nM | 54.6 | nM |
| SAD10319_P02_D06 | ADI-48631 | 232 | 3105 | 13.4 | 115 | 650 | 31.8 | nM | 72.7 | nM |
| SAD10319_P02_G06 | ADI-48632 | 107 | 1620 | 15.1 | 106 | 499 | 31.0 | nM | Non-binder | |
| SAD10319_P02_H06 | ADI-48633 | 1448 | 2791 | 1.9 | 105 | 431 | 88.8 | nM | 79.9 | nM |
| SAD10319_P03_C07 | ADI-48634 | 44 | 651 | 14.7 | 80 | 283 | 42.7 | nM | Non-binder | |
| SAD10319_P03_E07 | ADI-48635 | 54 | 623 | 11.6 | 78 | 248 | 40.9 | nM | Non-binder | |
| SAD10319_P03_A08 | ADI-48636 | 35 | 897 | 25.9 | 95 | 190 | 36.4 | nM | Non-binder | |
| SAD10319_P03_E08 | ADI-48637 | 73 | 244 | 3.3 | 80 | 179 | 36.6 | nM | Non-binder | |
| SAD10319_P03_G08 | ADI-48638 | 84 | 1996 | 23.8 | 173 | 276 | 41.4 | nM | Non-binder | |
| SAD10319_P03_B09 | ADI-48639 | 55 | 897 | 16.5 | 74 | 149 | 112 | nM | Non-binder | |
| SAD10319_P03_E09 | ADI-48640 | 56 | 772 | 13.8 | 83 | 250 | 43.7 | nM | Non-binder | |
| SAD10319_P04_G11 | ADI-48641 | 52 | 59 | 1.1 | 84 | 145 | 800 | nM | Non-binder | |
| SAD10319_P04_E12 | ADI-48642 | 55 | 332 | 6.1 | 86 | 335 | 800 | nM | Non-binder | |
| SAD10320_P01_B01 | ADI-48643 | 265 | 4205 | 15.9 | 90 | 302 | 131 | nM | Non-binder | |
| SAD10320_P01_D01 | ADI-48644 | 124 | 860 | 6.9 | 70 | 159 | 30.5 | nM | Non-binder | |
| SAD10320_P01_E01 | ADI-48645 | 41 | 450 | 11.1 | 75 | 195 | 50.1 | nM | Non-binder | |
| SAD10320_P01_A02 | ADI-48646 | 53 | 1267 | 24.1 | 71 | 143 | 35.8 | nM | Non-binder | |
| SAD10320_P01_F03 | ADI-48647 | 141 | 4343 | 30.9 | 68 | 197 | 64.4 | nM | Non-binder | |
| SAD10320_P02_C04 | ADI-48648 | 5138 | 6163 | 1.2 | 94 | 201 | 0.978 | nM | 2.41 | nM |
| SAD10320_P02_E04 | ADI-48649 | 639 | 2315 | 3.6 | 75 | 200 | 253 | nM | Non-binder | |
| SAD10320_P02_D05 | ADI-48650 | 5429 | 6012 | 1.1 | 85 | 247 | 1.29 | nM | 31.7 | nM |
| SAD10320_P02_F05 | ADI-48651 | 4114 | 5625 | 1.4 | 80 | 243 | 34.5 | nM | 102 | nM |
| SAD10320_P02_H05 | ADI-48652 | 4760 | 5751 | 1.2 | 129 | 403 | 2.13 | nM | 59.2 | nM |
| SAD10320_P02_B06 | ADI-48653 | 1032 | 3107 | 3.0 | 84 | 163 | 41.2 | nM | 182 | nM |
| SAD10320_P02_C06 | ADI-48654 | 4888 | 5593 | 1.1 | 102 | 291 | 18.0 | nM | 68.1 | nM |
| SAD10320_P03_D09 | ADI-48655 | 581 | 1183 | 2.0 | 73 | 217 | 36.8 | nM | Non-binder | |
| SAD10320_P03_F09 | ADI-48656 | 38 | 185 | 4.9 | 72 | 268 | 34.7 | nM | Non-binder | |
| SAD10320_P04_E10 | ADI-48657 | 67 | 786 | 11.7 | 79 | 259 | 77.8 | nM | Non-binder | |
| SAD10320_P04_G10 | ADI-48658 | 316 | 113 | 0.4 | 84 | 253 | 34.7 | nM | Non-binder | |
| SAD10320_P04_H10 | ADI-48659 | 75 | 462 | 6.1 | 76 | 223 | 37.3 | nM | Non-binder | |
| SAD10320_P04_B11 | ADI-48660 | 41 | 81 | 2.0 | 80 | 207 | 42.9 | nM | Non-binder | |
| SAD10320_P04_C11 | ADI-48661 | 48 | 98 | 2.0 | 75 | 225 | 38.3 | nM | Non-binder | |
| SAD10320_P04_H11 | ADI-48662 | 44 | 81 | 1.8 | 78 | 252 | 10.9 | nM | Non-binder | |
| LAD9958_P01_A04 | ADI-48663 | 64 | 159 | 2.5 | 84 | 197 | 28.3 | nM | Non-binder | |
| LAD9961_P01_E05 | ADI-48664 | 198 | 452 | 2.3 | 111 | 238 | 32.8 | nM | 57.5 | nM |
| LAD9962_P01_A06 | ADI-48665 | 4898 | 5582 | 1.1 | 1842 | 2055 | 27.7 | nM | 106 | nM |
| LAD9965_P01_H07 | ADI-48666 | 4804 | 5698 | 1.2 | 131 | 168 | 16.4 | nM | 102 | nM |

| | | | | | | | | | |
|---|---|---|---|---|---|---|---|---|---|
| LAD5195_P02_E11 | ADI-26140 | 56 | 55 | 1.0 | 76 | 378 | 1000 | nM | Non-binder |

TABLE 4
Human and Cynomolgus CD3ε Sequences

| | | SEQ |
|---|---|---|
| Name | Sequence | ID NO: |
| Hu CD3ε | QDGNEEMGGITQTPYKVSISGTTVILTCPQYPGSE | 591 |
| Fc | ILWQHNDKNIGGDEDDKNIGSDEDHLSLKEFSEL | |
| | EQSGYYVCYPRGSKPEDANFYLYLRARVCENCM | |
| | EMDGGSDKTHTCPPCPAPELLGGPSVFLFPPKPK | |
| | DTLMISRTPEVTCVVVDVSHEDPEVKFNWYVDG | |
| | VEVHNAKTKPREEQYASTYRVVSVLTVLHQDW | |
| | LNGKEYKCKVSNKALPAPIEKTISKAKGQPREPQ | |
| | VYTLPPSRDELTKNQVSLTCLVKGFYPSDIAVEW | |
| | ESNGQPENNYKTTPPVLDSDGSFFLYSKLTVDKS | |
| | RWQQGNVFSCSVMHEALHNHYTQKSLSLSPGK | |
| Cy CD3ε | QDGNEEMGSITQTPYQVSISGTTVILTCSQHLGSE | 592 |
| Fc | AQWQHNGKNKEDSGDRLFLPEFSEMEQSGYYV | |
| | CYPRGSNPEDASHHLYLKARVCENCMEMDGGS | |
| | DKTHTCPPCPAPELLGGPSVFLFPPKPKDTLMISR | |
| | TPEVTCVVVDVSHEDPEVKFNWYVDGVEVHNA | |
| | KTKPREEQYASTYRVVSVLTVLHQDWLNGKEY | |
| | KCKVSNKALPAPIEKTISKAKGQPREPQVYTLPPS | |
| | RDELTKNQVSLTCLVKGFYPSDIAVEWESNGQPE | |
| | NNYKTTPPVLDSDGSFFLYSKLTVDKSRWQQGN | |
| | VFSCSVMHEALHNHYTQKSLSLSPGK | |

What is claimed is:

1. An anti-cluster of differentiation three (“CD3”) antibody and/or antigen-binding fragment comprising:
(a) a variable heavy (V_{H}) chain polypeptide comprising a V_{H} chain CDR3 (CDRH3) selected from the group consisting of:
i. AX₁DX₂X₃X₄X₅X₆X₇X₈DX₉, wherein X₁ is R or H, wherein X₂ is A, H, M, or Q, wherein X₃ is Y, H, S, G, A, T, V, or R; wherein X₄ is G, H, P, E, or R; wherein X₅ is H or R, wherein X₆ is Y, N, F, H, D, E, S, L, M, I, G, A, Q, or T; wherein X₇ is F or H; wherein X₈ is Y or H; wherein X₉ is V, H, or M; and, optionally, wherein at least one of X₁, X₂, X₃, X₄, X₅, X₆, X₇, X₈, and X₉ is H (SEQ ID NO: 58);
ii. ARDX₁X₂X₃X₄YFYDX₅, wherein X₁ is H or A, wherein X₂ is T, Y, or H, wherein X₃ is G or H, wherein X₄ is H, R, V, or I, wherein X₅ is V or H, and, optionally, wherein at least one of X₁, X₂, X₃, X₄, and X₅ is H (SEQ ID NO: 43);
iii. AX₁DX₂YX₃HX₄FYDV, wherein X₁ is R or H, wherein X₂ is A or H, wherein X₃ is G, H, or P, wherein X₄ is Y, H, D, V, E, S, N, L, M, I, G, A, Q, or T, and, optionally, wherein at least one of X₁, X₂, X₃, and X₄ is H (SEQ ID NO: 1);
iv. ARDX₁YGX₂X₃X₄YDX₅ wherein X₁ is A or H, wherein X₂ is R or H, wherein X₃ is H or Y, wherein X₄ is F or H, wherein X₅ is H or V, and, optionally, wherein at least one of X₁, X₂, X₃, X₄, and X₅ is H (SEQ ID NO: 2):
v. ARDAHX₁X₂YX₃X₄DX₅, wherein X₁ is G, E, or R, wherein X₂ is R or H, wherein X₃ is F or H, wherein X₄ is Y or H, wherein X₅ is V or H, and, optionally, wherein at least one of X₁, X₂, X₃, X₄, and X₅ is H (SEQ ID NO: 3);
vi. ARDAX₁HRX₂FYDV, wherein X₁ is H, Y, S, G, A, T, V, or R, wherein X₂ is Y or H, and, optionally, wherein at least one of X₁ and X₂ is H (SEQ ID NO: 4);
vii. ARDX₁YHRYFYDX₂, wherein X₁ is H or A, wherein X₂ is H, V, or M, and, optionally, wherein at least one of X₁ and X₂ is H (SEQ ID NO: 5);
viii. AX₁DAYX₂X₃X₄HX₅DV, wherein X₁ is R or H, wherein X₂ is G or H, wherein X₃ is H or R, wherein X₄ is N, F, or Y, wherein X₅ is Y or H, and, optionally, wherein at least one of X₁, X₂, X₃, X₄, and X₅ is H (SEQ ID NO: 6);
ix. ARDX₁X₂GRYFYDV, wherein X₁ is M, Q, or H, wherein X₂ is R or H, and, optionally, wherein at least one of X₁ and X₂ is H (SEQ ID NO: 7);
x. ARDX₁X₂X₃RYFYDX₄, wherein X₁ is H or A, wherein X₂ is T, Y, or H, wherein X₃ is G or H, wherein X₄ is V or H, and, optionally, wherein at least one of X₁, X₂, X₃, and X₄ is H (SEQ ID NO: 8);
xi. ARDAX₁X₂X₃X₄FYDX₅, wherein X₁ is T, H, or Y, wherein X₂ is G or H, wherein X₃ is H or R, wherein X₄ is V or Y, wherein X₅ is V or H, and wherein, optionally, at least one of X₁, X₂, X₃, and X₅ is H (SEQ ID NO: 593); and
xii. AX₁DX₂X₃X₄X₅X₆X₇YDX₈, wherein X₁ is R or H, wherein X₂ is H or A, wherein X₃ is H or Y, wherein X₄ is H, G, or P, wherein X₅ is R or H, wherein X₆ is Y, I, or V, wherein X₇ is F or H, wherein X₈ is V or H, and wherein, optionally, at least one of X₁, X₂, X₃, X₄, X₅, X₇, and X₈ is H (SEQ ID NO: 596).
(b) a variable heavy (V_{H}) chain polypeptide comprising a V_{H} chain CDR2 (CDRH2) selected from the group consisting of:
i. X₁IX₂X₃X₄X₅X₆X₇TX₈YSQKFQG, wherein X₁ is W, S, Y, F, G, or D, wherein X₂ is N, T, D, V, or H, wherein X₃ is A, P, or S, wherein X₄ is G, A, S, N, D, L, V, H, Q, T, I, or Y, wherein X₅ is D or T, wherein X₆ is A or G, wherein X₇ is A, N, T, S, L, D, F, Y, or E, wherein X₈ is V, K, T, D, Y, F, A, H, N, L, I, or E, and, optionally, wherein at least one of X₁, X₂, X₃, X₄, X₅, X₆, X₇, and X₈ is H (SEQ ID NO: 59);
ii.
(SEQ ID NO: 9)
WIDLENANTIYDAKFQG;
iii. X₁INPX₂TGX₃TX₄YSQKFQG, wherein X₁ is W or Y, wherein X₂ is A, S, D, G, N, L, V, H, or Q, wherein X₃ is A, T, or S, and wherein X₄ is K, V, T, D, Y, F, or A (SEQ ID NO: 10);
iv. X₁IX₂AGTGX₃TX₄YSQKFQG, wherein X₁ is W, Y, or F, wherein X₂ is T, N, or D, wherein X₃ is A, T, or L, and wherein X₄ is A, K, V, H, T, or N (SEQ ID NO: 11);
v. X₁IDAGTGX₂TX₃YSQKFQG, wherein X₁ is S or W, wherein X₂ is L, N, D, or F, and wherein X₃ is D, Y, or K (SEQ ID NO: 12);
vi. X₁IX₂AGTGATX₃YSQKFQG, wherein X₁ is G, D, or S, wherein X₂ is I or D, and wherein X₃ is K or D (SEQ ID NO: 13);
vii. WINPX₁TGNTX₂YSQKFQG, wherein X₁ is D, T, L, S, or A, and wherein X₂ is D, V, L, or N (SEQ ID NO: 14);
viii. X₁INAGTGX₂TX₃YSQKFQG, wherein X₁ is Y or W, wherein X₂ is N, D, or A, and wherein X₃ is I or V (SEQ ID NO: 15);
ix. X₁INPX₂TGX₃TKYSQKFQG, wherein X₁ is W or Y, wherein X₂ is D, I or Y, and wherein X₃ is D, Y, or E (SEQ ID NO: 16);
x. SIX₁AGTGX₂TKYSQKFQG, wherein X₁ is N or V, and wherein X₂ is A or I (SEQ ID NO: 17);
xi. SINAGTGX₁TX₂YSQKFQG, wherein X₁ is F or N, and wherein X₂ is Y or D (SEQ ID NO: 18);
xii. X₁IX₂X₃GTGX₄TDYSQKFQG, wherein X₁ is D or W, wherein X₂ is N or H, wherein X₃ is A or S, and wherein X₄ is A or N (SEQ ID NO: 19);
xiii. WIDPX₁TGATX₂YSQKFQG, wherein X₁ is N, H, or Y, and wherein X₂ is V or K (SEQ ID NO: 20);
xiv. WIX₁PX₂TGNTKYSQKFQG, wherein X₁ is D or N, and wherein X₂ is L, I, or V (SEQ ID NO: 21);
xv.
(SEQ ID NO: 22)
SINAGDANTKYSQKFQG;
xv. SINAGDANTKYSQKFQG (SEQ ID NO: 22);
xvi. X₁IDPX₂TGATX₃YSQKFQG, wherein X₁ is D or W, wherein X₂ is D or V, and wherein X₃ is E or D (SEQ ID NO: 23);
xvii. WINAGDAATVYSQKFQG (SEQ ID NO: 24); and
xviii. WIDAGTGX₁TX₂YSQKFQG, wherein X₁ is L, F, N, or A and wherein X₂ is T or K (SEQ ID NO: 595).
(c) a variable heavy (V_{H}) chain polypeptide comprising a V_{H} chain CDR1 (CDRH1) selected from the group consisting of:
i.
(SEQ ID NO: 25)
FNIKDYHMH;
ii.
(SEQ ID NO: 26)
SNIKDYYMH;
iii.
(SEQ ID NO: 27)
SNIKDYHMH;
iv. YTFX₁X₂X₃X₄MH, wherein X₁ is A, K, D, Q, E, N, T, L, Y, S, P, G, H or V, wherein X₂ is T, S, or A, wherein X₃ is Y or I, and wherein X₄ is A, D, N, S, Y, T, I, V, L, E, P, R, or G (SEQ ID NO: 28);
v. YTFX₁X₂X₃X₄MH, wherein X₁ is T, D, A, N, or V, wherein X₂ is D, E, G, or Q, wherein X₃ is Y or D, and wherein X₄ is D, A, E, N, S, Y, or V (SEQ ID NO: 29);
vi. YTFTSX₁X₂MH, wherein X₁ is A, D, or T, and wherein X₂ is D, F, A, M, V, or Y (SEQ ID NO: 30);
vii. YTFX₁X₂YX₃MH, wherein X₁ is N or T, X₂ is Q or N, and X₃ is S, T, or A (SEQ ID NO: 31); and
viii. YTFX₁X₂YVMH, wherein X₁ is I or N, and wherein X₂ is K or R (SEQ ID NO: 32);
ix. FNIKDYYMH (SEQ ID NO: 47); and
x. YTFX₁X₂YX₃MH, wherein X₁ is E, S, or T, wherein X₂ is S or D, and wherein X₃ is A or D (SEQ ID NO: 31).
(d) a variable light (VL) chain polypeptide comprising a VL chain CDR3 (CDRL3) selected from the group consisting of:
i. X₁X₂SX₃X₄X₅RX₆, wherein X₁ is H, K, or G, wherein X₂ is Q or H, wherein X₃ is Y or H, wherein X₄ is S, H, D, T, V, M, or L, wherein X₅ is R or H, wherein X₆ is T or H, and, optionally, wherein at least one of X₁, X₂, X₃, X₄, X₅, and X₆ is H (SEQ ID NO: 33);
ii. KQSYX₁X₂RT, wherein X₁ is H, V, K, W, R, L, G, Y, or Q, wherein X₂ is H, L, E, W, G, M, P, T, Q, or V, and, optionally, wherein at least one of X₁ and X₂ is H (SEQ ID NO: 34);
iii. X₁QSX₂HX₃RT, wherein X₁ is K or H, wherein X₂ is H, Y, M, S, L, E, G, or W, wherein X₃ is R or K, and, optionally, wherein at least one of X₁ and X₂ is H(SEQ ID NO: 35);
iv. KQSX₁X₂X₃RT, wherein X₁ is Y or H, wherein X₂ is T, S, V, or K, wherein X₃ is R or H, and, optionally, wherein at least one of X₁ and X₃ is H (SEQ ID NO: 36);
v. KQSX₁X₂X₃RT, wherein X₁ is H or Y, wherein X₂ is T, S, or Q, wherein X₃ is R or H, and, optionally, wherein at least one of X₁ and X₃ is H (SEQ ID NO: 36); and
vi. X₁QSX₂X₃X₄RT, wherein X₁ is K or H, wherein X₂ is Y or H, wherein X₃ is S, H, L, V, or K, wherein X₄ is H, R, or E, and, optionally, wherein at least one of X₁, X₂, X₃, and X₄ is H (SEQ ID NO: 598);
(e) a variable light (VL) chain polypeptide comprising a VL chain CDR2 (CDRL2) of WASTRES (SEQ ID NO: 37); and/or
(f) a variable light (VL) chain polypeptide comprising a VL chain CDR1 (CDRL1) selected from the group consisting of:
i. KSSQSLLX₁X₂X₃X₄GX₅NX₆LA, wherein X₁ is N or H, wherein X₂ is A, R, or T, wherein X₃ is R or H, wherein X₄ is T, P, or E, wherein X₅ is H or K, wherein X₆ is H or Y, and, optionally, wherein at least one of X₁, X₃, X₅, and X₆ is H (SEQ ID NO: 38);
ii. KSSQSLLX₁AX₂THX₃NX₄LA, wherein X₁ is N or H, wherein X₂ is R or H, wherein X₃ is K or H, wherein X₄ is Y or H, and, optionally, wherein at least one of X₁, X₂, X₃, and X₄ is H (SEQ ID NO: 39);
iii.
(SEQ ID NO: 40)
KSSQSLLNASTAKNYLA;
iv.
(SEQ ID NO: 41)
KSSQSLLNARTRTNYLA;
v. KSSQSLLNX₁X₂X₃GX₄NX₅LA, wherein X₁ is S or A, wherein X₂ is R or H, wherein X₃ is E or T, wherein X₄ is H or K, wherein X₅ is H or Y, and, optionally, wherein at least one of X₂, X₄, and X₅ is H (SEQ ID NO: 42);
vi. KSSQSLLNX₁X₂TGX₃NYLA, wherein X₁ is A or S, wherein X₂ is R or H, wherein X₃ is H or K, and, optionally, wherein at least one of X₂ and X₃ is H (SEQ ID NO: 594); and
vii. KSSQSLLX₁AX₂X₃X₄X₅NX₆LA, wherein X₁ is N or H, wherein X₂ is R or H, wherein X₃ is T or E, wherein X₄ is G or H, wherein X₅ is H or K, wherein X₆ is H or Y, and wherein, optionally, at least one of X₁, X₂, X₄, X₅, and X₆ is H (SEQ ID NO: 597).

2. The anti-CD3 antibody and/or antigen-binding fragment according to claim 1, comprising:
(a) a CDRH3 comprising an amino acid sequence ARDX₁X₂X₃X₄YFYDX₅, wherein X₁ is H or A, wherein X₂ is T, Y, or H, wherein X₃ is G or H, X₄ is H, R, V, or I, wherein X₅ is V or H, and, optionally, wherein at least one of X₁, X₂, X₃, X₄, and X₅ is H (SEQ ID NO: 43);
(b) a CDRL3 comprising an amino acid sequence KQSX₁X₂X₃RT, wherein X₁ is Y or H, X₂ is T, S, V, or K, X₃ is R or H, and, optionally, wherein at least one of X₁ and X₃ is H (SEQ ID NO: 36); and/or
(c) a CDRL1 comprising an amino acid sequence KSSQSLLNX₁X₂X₃GX₄NX₅LA, wherein X₁ is S or A, wherein X₂ is R or H, wherein X₃ is E or T, wherein X₄ is H or K, wherein X₅ is H or Y, and, optionally, wherein at least one of X₂, X₄, and X₅ is H (SEQ ID NO: 42).

3. The anti-CD3 antibody or antigen-binding fragment according to claim 1, comprising:
(a) a CDRH3 comprising an amino acid sequence ARDAX₁X₂X₃X₄FYDX₅, wherein X₁ is T, H, or Y, wherein X₂ is G or H, wherein X₃ is H or R, wherein X₄ is V or Y, wherein X₅ is V or H, and wherein, optionally, at least one of X₁, X₂, X₃, and X₅ is H (SEQ ID NO: 593);
(b) a CDRH2 comprising an amino acid sequence WIDLENANTIYDAKFQG (SEQ ID NO: 9);
(c) a CDRH1 comprising an amino acid sequence FNIKDYYMH (SEQ ID NO: 47);
(d) a CDRL3 comprising an amino acid sequence KQSX₁X₂X₃RT, wherein X₁ is H or Y, wherein X₂ is T, S, or Q, wherein X₃ is R or H, and, optionally, wherein at least one of X₁ and X₃ is H (SEQ ID NO: 36);
(e) a CDRL2 comprising an amino acid sequence WASTRES (SEQ ID NO: 37); and/or
(f) a CDRL1 comprising an amino acid sequence KSSQSLLNX₁X₂TGX₃NYLA, wherein X₁ is A or S, wherein X₂ is R or H, wherein X₃ is H or K, and, optionally, wherein at least one of X₂ and X₃ is H (SEQ ID NO: 594).

4. The anti-CD3 antibody or antigen-binding fragment according to claim 1, comprising:
(a) a CDRH3 comprising an amino acid sequence AX₁DX₂X₃X₄X₅X₆X₇YDX₈, wherein X₁ is R or H, wherein X₂ is H or A, wherein X₃ is H or Y, wherein X₄ is H, G, or P, wherein X₅ is R or H, wherein X₆ is Y, I, or V, wherein X₇ is F or H, wherein X₈ is V or H, and wherein, optionally, at least one of X₁, X₂, X₃, X₄, X₅, X₇, and X₈ is H (SEQ ID NO: 596);
(b) a CDRH2 comprising an amino acid sequence selected from the group consisting of:
i. WIDLENANTIYDAKFQG (SEQ ID NO: 9); and
ii. WIDAGTGX₁TX₂YSQKFQG, wherein X₁ is L, F, N, or A and wherein X₂ is T or K (SEQ ID NO: 595);
(c) a CDRH1 comprising an amino acid sequence selected from the group consisting of:
i. FNIKDYYMH (SEQ ID NO: 47); and
ii. YTFX₁X₂YX₃MH, wherein X₁ is E, S, or T, wherein X₂ is S or D, and wherein X₃ is A or D (SEQ ID NO: 31);
(d) a CDRL3 comprising an amino acid sequence X₁QSX₂X₃X₄RT, wherein X₁ is K or H, wherein X₂ is Y or H, wherein X₃ is S, H, L, V, or K, wherein X₄ is H, R, or E, and, optionally, wherein at least one of X₁, X₂, X₃, and X₄ is H (SEQ ID NO: 598);
(e) a CDRL2 comprising an amino acid sequence WASTRES (SEQ ID NO: 37); and/or
(f) a CDRL1 comprising an amino acid sequence KSSQSLLX₁AX₂X₃X₄X₅NX₆LA, wherein X₁ is N or H, wherein X₂ is R or H, wherein X₃ is T or E, wherein X₄ is G or H, wherein X₅ is H or K, wherein X₆ is H or Y, and wherein, optionally, at least one of X₁, X₂, X₄, X₅, and X₆ is H (SEQ ID NO: 597).

5. The anti-CD3 antibody or antigen-binding fragment according to claim 1, wherein:
(a) said antibody or antigen-binding fragment elicits T cell activation or T cell killing while displaying a decreased propensity to elicit cytokine production to levels capable of inducing cytokine release syndrome;
(b) the antibody or antigen-binding fragment comprises a multispecific antibody;
(c) the antibody or antigen-binding fragment comprises a bispecific antibody;
(d) the antibody or antigen-binding fragment comprises an scFv;
(e) the antibody or antigen-binding fragment comprises at least a second antigen-binding domain that specifically binds to an oncology target; an immune-oncology target; a neurodegenerative disease targets; an autoimmune disorder target; an infectious disease target; a metabolic disease target; a cognitive disorder target; a blood-brain barrier target; or a blood disease target;
(f) the antibody or antigen-binding fragment comprises at least a second antigen-binding domain that specifically binds to an antigen selected from the group consisting of: 17-IA, 4-1BB, 4Dc, 6-keto-PGF1a, 8-iso-PGF2a, 8-oxo-dG, A1 Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RUB, ADAM, ADAM10, ADAM12, ADAM 15, ADAM 17/T ACE, ADAM8, ADAM9, ADAMTS, ADAMTS4, ADAMTS5, Addressins, aFGF, ALCAM, ALK, ALK-1, ALK-7, alpha-1-antitrypsin, alpha-V/beta-1 antagonist, ANG, Ang, APAF-1, APE, APJ, APP, APRIL, AR, ARC, ART, Artemin, anti-Id, ASPARTIC, Atrial natriuretic factor, av/b3 integrin, Axl, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bel, BCMA, BDNF, b-ECGF, bFGF, BID, Bik, BFM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, CIO, CA125, CAD-8, Calcitonin, cAMP, carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/Z/P, CBL, CCI, CCK2, CCL, CCL1, CCL11, CCL12, CCL13, CCL 14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD28, CD29, CD30, CD30L, CD32, CD33 (p67 proteins), CD34, CD38, CD40, CD40L, CD44, CD45, CD46, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(1-3)-IGF-I (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, EN A, endothelin receptor, Enkephalinase, eNOS, Eot, eotaxin1, EpCAM, Ephrin B2/EphB4, EPO, ERCC, E-selectin, ET-1, Factor IIa, Factor VII, Factor VIIIc, Factor IX, fibroblast activation protein (FAP), Fas, FcR1, FEN-1, Ferritin, FGF, FGF-19, FGF-2, FGF3, FGF-8, FGFR, FGFR-3, Fibrin, FL, FLIP, Flt-3, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GFAP, GFRa-1, GFR-alpha 1, GFR-alpha2, GFR-alpha3, GITR, Glucagon, Glut 4, glycoprotein IIb/IIIa (GP IIb/IIIa), GM-CSF, gp130, gp72, GRO, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gp120, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MAA), HIV gp120, HIV IIIB gp 120 V3 loop, HLA, HLA-DR, HM1.24, HMFG PEM, HRG, Hrk, human cardiac myosin, human cytomegalovirus (HCMV), human growth hormone (HGH), HVEM, 1-309, IAP, ICAM, ICAM-1, ICAM-3, ICE, ICOS, IFNg, Ig, IgA receptor, IgE, IGF, IGF binding proteins, IGF-1R, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-18, IL-18R, IL-23, interferon (INF)-alpha, INF-beta, INF-gamma, Inhibin, iNOS, Insulin A-chain, Insulin B-chain, Insulin-like growth factor 1, integrin alpha2, integrin alpha3, integrin alpha4, integrin alpha4/beta 1, integrin, alpha4/beta7, integrin alpha5 (alphaV), integrin alpha5/beta1, integrin alpha5/beta3, integrin alpha6, integrin beta1, integrin beta2, interferon gamma, IP-10, 1-TAC, JE, Kallikrein 2, Kallikrein 5, Kallikrein 6, Kallikrein 11, Kallikrein 12, Kallikrein 14, Kallikrein 15, Kallikrein L1, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bpl, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, a metalloprotease, MGDF receptor, MGMT, MHC (HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Muc1), MUC18, Muellerian-inhibiting substance, Mug, MuSK, NAIP, NAP, NCAD, N-Cadherin, NCA 90, NCAM, NCAM, Neprilysin, Neurotrophin-3, -4, or -6, Neurturin, Neuronal growth factor (NGF), NGFR, NGF-beta, nNOS, NO, NOS, Npn, NRG-3, NT, NTN, OB, OGG1, OPG, OPN, OSM, OX40L, OX40R, p150, p95, PADPr, Parathyroid hormone, PARC, PARP, PBR, PBSF, PCAD, P-Cadherin, PCNA, PDGF, PDGF, PDK-1, PECAM, PEM, PF4, PGE, PGF, PGI2, PGJ2, PIN, PLA2, placental alkaline phosphatase (PLAP), PIGF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, Relaxin A-chain, Relaxin B-chain, renin, respiratory syncytial virus (RSV) F, RSV Fgp, Ret, Rheumatoid factors, RLIP76, RPA2, RSK, S100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta RI (ALK-5), TGF-beta RII, TGF-beta RIIb, TGF-beta RIII, TGF-beta1, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha, TNF-alpha beta, TNF-beta2, TNFc, TNF-RI, TNF-RII, TNFRSF10A (TRAIL R1 Apo-2, DR4), TNFRSFIOB (TRAIL R2 DR5, KILLER, TRICK-2A, TRICK-B), TNFRSF10C (TRAIL R3 DcR1, LIT, TRID), TNFRSF10D (TRAIL R4 DcR2, TRUNDD), TNFRSF11A (RANK ODF R, TRANCE R), TNFRSF11B (OPG OC1F, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF 18 (GITR AITR), TNFRSF19 (TROY TAJ, TRADE), TNFRSF19L (RELT), TNFRSFIA (TNF RI CD120a, p55-60), TNFRSFIB (TNF RII CD120b, p75-80), TNFRSF26 (TNFRH3), TNFRSF3 (LTbR TNF RIII, TNFC R), TNFRSF4 (OX40 ACT35, TXGP1 R), TNFRSF 5 (CD40 p50), TNFRSF6 (Fas Apo-1, APT1, CD95), TNFRSF6B (DcR3 M68, TR6), TNFRSF7 (CD27), TNFRSF8 (CD30), TNFRSF9 (4-1BB CD137, ILA), TNFRSF21 (DR6), TNFRSF22 (DcTRAIL R2 TNFRH2), TNFRST23 (DcTRAIL R1 TNFRH1), TNFRSF25 (DR3 Apo-3, LARD, TR-3, TRAMP, WSL-1), TNFSF10 (TRAIL Apo-2 Ligand, TL2), TNFSF11 (TRANCE/RANK Ligand ODF, OPG Ligand), TNFSF12 (TWEAK Apo-3 Ligand, DR3 Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TLIA/VEGI), TNFSF18 (GITR Ligand AITR Ligand, TL6), TNFSFIA (TNF-a Conectin, DIF, TNFSF2), TNFSF1B (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD30 Ligand CD153), TNFSF9 (4-1BB Ligand CD137 Ligand), TP-1, t-PA, Tpo, TRAIL, TRAIL R, TRAIL-R1, TRAIL-R2, TRANCE, transferring receptor, TRF, Trk, TROP-2, TSG, TSLP, tumor-associated antigen CA 125, tumor-associated antigen expressing Lewis Y related carbohydrate, TWEAK, TXB2, Ung, uPAR, uPAR-1, Urokinase, VCAM, VCAM-1, VECAD, VE-Cadherin, VE-cadherin-2, VEFGR-1 (fit-1), VEGF, VEGFR, VEGFR-3 (fit-4), VEGI, VFM, Viral antigens, VLA, VLA-1, VLA-4, VNR integrin, von Willebrands factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, X1AP, XPD, CTLA4 (cytotoxic T lymphocyte antigen-4), PD1 (programmed cell death protein 1), PD-L1 (programmed cell death ligand 1), LAG-3 (lymphocyte activation gene-3), TIM-3 (T cell immunoglobulin and mucin protein-3), hormone receptors and growth factors;
(g) the antibody or antigen-binding fragment comprises at least a second antigen-binding domain that specifically binds to an antigen selected from the group consisting of: BCMA, CTLA4 (cytotoxic T lymphocyte antigen-4), PD1 (programmed cell death protein 1), PD-L1 (programmed cell death ligand 1), LAG-3 (lymphocyte activation gene-3), TIM-3, CD20, CD2, CD19, Her2, EGFR, EpCAM, FcyRIIIa (CD16), FcyRIIa (CD32a), FcyRIIb (CD32b), FcyRI (CD64), Toll-like receptors (TLRs), TLR4, TLR9, cytokines, IL-2, IL-5, IL-13, IL-6, IL-17, IL-12, IL-23, TNFa, TGFb, cytokine receptors, IL-2R, chemokines, chemokine receptors, growth factors, VEGF, and HGF;
(h) the antibody or antigen-binding fragment is comprised in a chimeric antigen receptor (CAR), which optionally comprises at least one transmembrane domain, and at least one intracellular domain from a T-cell receptor, optionally a CD3ζ subunit, and at least one co-stimulatory domain;
(i) the antibody or antigen-binding fragment comprises an scFv2-Fc2 and/or scFv-IgG;
(j) the antibody or antigen-binding fragment comprises an IgG constant domain; and/or
(k) the antibody or antigen-binding fragment comprises at least a second antigen-binding domain that specifically binds to an antigen, wherein said antibody comprises a multispecific format selected from the group consisting of: Fab-Fc-scFv, “bottle-opener”, Mab-scFv, Mab-Fv, Dual scFv, central Fv, central scFv, one-arm central scFv, Fab-Fab, Fab-Fv, mAb-Fv, mAb-Fab, DART, BiTE, common light chain-IgG, TandAb, Cross-Mab, SEED, BEAT, TrioMab, and DuetMab.

6. The anti-CD3 antibody and/or antigen-binding fragment according to claim 1, which binds to CD3 or CD3-expressing cells with a greater binding affinity at pH 6.0 than at pH 7.4.

7. An isolated or recombinant nucleic acid sequence encoding an antibody or antigen-binding fragment according to claim 1.

8. An expression vector comprising an isolated or recombinant nucleic acid according to claim 7.

9. A host cell transfected, transformed, or transduced with a nucleic acid according to claim 7, or an expression vector comprising said nucleic acid, wherein the host cell is optionally a mammalian cell or a yeast cell.

10. A pharmaceutical composition comprising an antibody or antigen-binding fragment according to claim 1 or a host cell which expresses said antibody or antigen-binding fragment, and a pharmaceutically acceptable carrier and/or excipient.

11. A method of treating a disorder in a mammal in need of such treatment, wherein the disorder comprises a proliferative disorder, an oncological disorder, an immuno-oncological disorder, a neurological disorder, a neurodegenerative disorder, or an autoimmune disorder, comprising administering an effective amount of at least one antibody or antigen-binding fragment according to claim 1 or a host cell which expresses said antibody or antigen-binding fragment, optionally an immune cell, further optionally a T or NK cell.

12. The method according to claim 11, wherein the method further comprises administering to the mammal an additional therapeutic agent, optionally wherein the mammal is a human.
